SEQUENCE LISTING (5 sequences)

SEQ ID NO: 1 - DNA, 25 bp, synthetic construct
  source 1..25: mol_type = other DNA
caccgctggttctatgcacgtctcc

SEQ ID NO: 2 - DNA, 20 bp, synthetic construct
  source 1..20: mol_type = other DNA
aaactcatgagcagtctgca

SEQ ID NO: 3 - DNA, 22 bp, synthetic construct
  source 1..22: mol_type = other DNA
aggagatcttcagtttcggagg

SEQ ID NO: 4 - DNA, 20 bp, synthetic construct
  source 1..20: mol_type = other DNA
gccttccgtgtccccactgc

SEQ ID NO: 5 - DNA, 17 bp, synthetic construct
  source 1..17: mol_type = other DNA
tgagggggccctcgacg

(19) United States
(12) Patent Application Publication
(10) Pub. No.: US 2025/0059218 A1
(43) Pub. Date: Feb. 20, 2025

(54) ENPP1 Inhibitors and Methods of Modulating Immune Response

(71) Applicant: The Board of Trustees of the Leland Stanford Junior University, Stanford, CA (US)

(72) Inventors: Lingyin Li, Stanford, CA (US); Mark Smith, Stanford, CA (US); Jacqueline Ann Carozza, Stanford, CA (US); Volker Boehnert, Stanford, CA (US)

(21) Appl. No.: 18/893,468

(22) Filed: Sep. 23, 2024

Related U.S. Application Data
(63) Continuation of application No. 17/423,389, filed on Jul. 15, 2021, now abandoned.
(60) Provisional application No. 62/800,283, filed on Feb. 1, 2019.
(60) Provisional application No. 62/814,745, filed on Mar. 6, 2019.

(51) Int. Cl. C07F 9/6512; A61K 31/44; A61K 31/4706; A61K 31/517; A61K 31/675; A61K 31/69; A61K 45/06; A61P 35/00; C07D 215/46; C07D 401/04; C07F 5/02; C07F 9/60; C07F 9/6558

(52) U.S. Cl. CPC C07F 9/65128; A61K 31/44; A61K 31/4706; A61K 31/517; A61K 31/675; A61K 31/69; A61K 45/06; A61P 35/00; C07D 215/46; C07D 401/04; C07F 5/02; C07F 9/60; C07F 9/65583

(57) ABSTRACT

Compounds, compositions and methods are provided for the inhibition of ENPP1. Aspects of the subject methods include contacting a sample with an ENPP1 inhibitor compound to inhibit the cGAMP hydrolysis activity of ENPP1. In some cases, the ENPP1 inhibitor compound is cell impermeable. ENPP1 inhibitor compounds can act extracellularly to block the degradation of cGAMP. Also provided are pharmaceutical compositions and methods for treating cancer. Aspects of the methods include administering to a subject a therapeutically effective amount of an ENPP1 inhibitor to treat the subject for cancer. In certain cases, the cancer is a solid tumor cancer. Also provided are methods of administering radiation therapy to a subject in conjunction with administering an ENPP1 inhibitor to the subject. The radiation therapy can be administered in the subject methods at a dosage and/or frequency effective to reduce radiation damage to the subject, but still instigate an immune response.

### CROSS-REFERENCE TO RELATED APPLICATIONS

This application is a continuation of U.S. application Ser. No. 17/423,389 filed Jul. 15, 2021, which application is a U.S. National Stage Application of PCT Application No. PCT/US2020/015968 filed Jan. 30, 2020, which application, pursuant to 35 U.S.C. § 119(e), claims priority to and the benefit of U.S. Provisional Application Ser. No. 62/800,283 filed Feb. 1, 2019 and U.S. Provisional Application Ser. No. 62/814,745 filed Mar. 6, 2019, the disclosure of which applications are incorporated by reference in its entirety.

### GOVERNMENT RIGHTS

This invention was made with Government support under contracts CA190896 and CA228044 awarded by the National Institutes of Health and contract W81XWH-18-1-0041 awarded by the Department of Defense. The Government has certain rights in the invention.

### REFERENCE TO AN ELECTRONIC SEQUENCE LISTING

The contents of the electronic sequence listing (STAN-1591CON_SEQ_LIST.xml; Size: 5,553 bytes; and Date of Creation: Nov. 5, 2024) is herein incorporated by reference in its entirety.

### INTRODUCTION

Cyclic guanosine monophosphate-adenosine monophosphate (cGAMP) activates the Stimulator of Interferon Genes (STING) pathway, which is an important anti-cancer innate immune pathway. The cGAS-cGAMP-STING pathway gets activated in presence of cytoplasmic DNA either due to microbial infection or patho-physiological condition, including cancer and autoimmune disorder. Cyclic GMP-AMP synthase (cGAS) belongs to the nucleotidyltransferase family and is a universal DNA sensor that is activated upon binding to cytosolic dsDNA to produce the signaling molecule (2′-5′, 3′-5′) cyclic GMP-AMP (or 2′,3′-cGAMP or cyclic guanosine monophosphate-adenosine monophosphate, cGAMP). Acting as a second messenger during microbial infection, 2′,3′-cGAMP binds and activates STING, leading to production of type I interferon (IFN) and other co-stimulatory molecules that trigger the immune response. Besides its role in infectious disease, the STING pathway has emerged as a target for cancer immunotherapy and autoimmune diseases. Ectonucleotide pyrophosphatase/phosphodiesterase 1 (ENPP1) is the dominant hydrolase of cGAMP that can degrade cGAMP. ENPP1 is a member of the ecto-nucleotide pyrophosphatase/phosphodiesterase (ENPP) family, and is a type II transmembrane glycoprotein comprising two identical disulfide-bonded subunits. ENPP1 has broad specificity to cleave a variety of substrates, including phosphodiester bonds of nucleotides and nucleotide sugars, and pyrophosphate bonds of nucleotides and nucleotide sugars. ENPP1 may function to hydrolyze nucleoside 5′ triphosphates to their corresponding monophosphates and may also hydrolyze diadenosine polyphosphates.

### SUMMARY

Compounds, compositions and methods are provided for the inhibition of ENPP1. Aspects of the subject methods include contacting a sample with an ENPP1 inhibitor compound to inhibit the cGAMP hydrolysis activity of ENPP1. In some cases, the ENPP1 inhibitor compound is cell impermeable. ENPP1 inhibitor compounds can act extracellularly to block the degradation of cGAMP. Also provided are pharmaceutical compositions and methods for treating cancer. Aspects of the methods include administering to a subject a therapeutically effective amount of an ENPP1 inhibitor to treat the subject for cancer. In certain cases, the cancer is a solid tumor cancer. Also provided are methods of administering radiation therapy to a subject in conjunction with administering an ENPP1 inhibitor to the subject. The radiation therapy can be administered in the subject methods at a dosage and/or frequency effective to reduce radiation damage to the subject, but still instigate an immune response.

These and other advantages and features of the disclosure will become apparent to those persons skilled in the art upon reading the details of the compositions and methods of use, which are more fully described below.

### BRIEF DESCRIPTION OF THE DRAWINGS

The invention is best understood from the following detailed description when read in conjunction with the accompanying figures. The patent or application file contains at least one figure executed in color. It is emphasized that, according to common practice, the various features of the figures are not to-scale. On the contrary, the dimensions of the various features are arbitrarily expanded or reduced for clarity. Included in the drawings are the following figures. It is understood that the figures, described below, are for illustration purposes only. The figures are not intended to limit the scope of the present teachings in any way.

FIG. 1, panels A to J, show experimental results that demonstrate cGAMP is exported from 293T cGAS ENPP1^{-/-} cells as a soluble factor.

FIG. 2, panels A to C, show experimental results that demonstrate ENPP1 can regulate extracellular cGAMP.

FIG. 3, panels A to F, illustrate the structure and activity in various cell assays of an exemplary ENPP1 inhibitor (compound 1).

FIG. 4, panels A to E, show experimental results that indicate cancer cells express cGAS and continuously export cGAMP in culture.

FIG. 5, panels A to I, show experimental results that indicate sequestration of extracellular cGAMP decreases tumor-associated dendritic cells in a tumor cGAS and host STING dependent manner.

FIG. 6, panels A to D, show experimental results that indicate ENPP1^{-/-} tumors recruit innate immune infiltration, are less aggressive, and more susceptible to IR and anti-CTLA-4 (cytotoxic T-lymphocyte-associated antigen 4) therapy.

FIG. 7, panels A to C, show experimental results that indicate ENPP1 inhibition synergizes with IR treatment and anti-CTLA-4 to exert anti-tumor effects.

FIG. 8, panels A to D, illustrate use of an LC-MS/MS method and 293T cGAS ENPP1^{low} and 293T cGAS ENPP1^{-/-} cell lines to assess ENPP1 hydrolysis activity and cGAMP levels.

FIG. 9, panel A to B, shows an experimental schematic and results that illustrate CD14⁺ Primary human peripheral blood mononuclear cells (PBMCs) respond to extracellular cGAMP.

FIG. 10, panels A to B, show experimental results comparing the ENPP1 inhibitory activity of compound 1 and compound QS1, and showing activity of QS1 in a cell assay.

FIG. 11, panels A to F, show experimental results indicating exemplary ENPP1 inhibitor compound 1 (STF-1084) is cell impermeable, specific to ENPP1, and nontoxic.

FIG. 12, panels A to E, show experimental results that indicate cancer cells continuously export cGAMP in culture.

FIG. 13, panels A to D, show experimental results that indicate sequestration of extracellular cGAMP decreases tumor-associated dendritic cells in a tumor cGAS and host STING dependent manner.

FIG. 14, panels A to F, show experimental results that indicate established ENPP1^{-/-} tumors lead to increased tumor-associated dendritic cells, are less aggressive, and more susceptible to IR and anti-CTLA-4 therapy.

FIG. 15, shows a graph of data that demonstrates ENPP1 inhibition (e.g., using compound 1; STF-1084) synergizes with IR treatment to increase tumor-associated dendritic cells.

FIG. 16 shows a schematic illustrating different modes of cGAMP transmission from the synthesizing cell to target cells.

FIG. 17 shows a schematic illustrating cGAMP is a cancer danger signal secreted by cancer cells in vivo.

FIG. 18A to FIG. 18C shows data illustrating that an exemplary ENPP1 inhibitor (compound 1) can increase the amount of extracellular cGAMP present in a cell system.

FIG. 19A to FIG. 19B show an experimental schematic and results that illustrate exemplary ENPP1 inhibitor (compound 1) can increase cGAMP-stimulated interferon transcription.

FIG. 20A to FIG. 20B shows data illustrating that an exemplary ENPP1 inhibitor (compound 1) can increase the number of tumor-associated dendritic cells in a mouse tumor model.

FIG. 21A to FIG. 21C show experimental results that illustrate ENPP1 inhibition synergizes with IR treatment and anti-CTLA-4 to exert anti-tumor effects.

FIG. 22 shows a schematic illustrating that ENPP1 is an innate immune checkpoint that regulates the immunotransmitter cGAMP.

### DEFINITIONS

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

The phrases “for example,” “for instance,” “such as,” or “including” are meant to introduce examples that further clarify more general subject matter. These examples are provided only as an aid for understanding the disclosure, and are not meant to be limiting in any fashion.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

The terms “treatment,” “treating,” and the like, refer to obtaining a desired pharmacologic and/or physiologic effect, such as reduction of tumor burden. The effect may be prophylactic in terms of completely or partially preventing a disease or symptom thereof and/or may be therapeutic in terms of a partial or complete cure for a disease and/or adverse effect attributable to the disease. “Treatment” is meant to cover any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development, and (c) relieving the disease, i.e., causing regression of the disease (e.g., reduction in of tumor burden).

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

The terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

The terms “pharmaceutically acceptable excipient,” “pharmaceutically acceptable diluent,” “pharmaceutically acceptable carrier,” and “pharmaceutically acceptable adjuvant” refer to an excipient, diluent, carrier, or adjuvant that is useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

The term “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

The phrase “having the formula” or “having the structure” is not intended to be limiting and is used in the same way that the term “comprising” is commonly used. The term “independently selected from” is used herein to indicate that the recited elements, e.g., R groups or the like, can be identical or different.

The terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

The term “alkyl” refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “substituted alkyl” is meant to include an alkyl group as defined herein wherein one or more carbon atoms in the alkyl chain have been optionally replaced with a heteroatom such as —O—, —N—, —S—, —S(O)n- (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO2-alkyl, —SO2-aryl, —SO2-heteroaryl, and —NRaRb, wherein R′ and R″ may be the same or different and are chosen from hydrogen, optionally substituted alkyl, cycloalkyl, alkenyl, cycloalkenyl, alkynyl, aryl, heteroaryl and heterocyclic.

The term “alkenyl” refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” refers to an alkyl group bound through a single, terminal ether linkage; that is, an “alkoxy” group may be represented as —O-alkyl where alkyl is as defined above. A “lower alkoxy” group refers to an alkoxy group containing 1 to 6 carbon atoms, and includes, for example, methoxy, ethoxy, n-propoxy, isopropoxy, t-butyloxy, etc. Substituents identified as “C1-C6 alkoxy” or “lower alkoxy” herein may, for example, may contain 1 to 3 carbon atoms, and as a further example, such substituents may contain 1 or 2 carbon atoms (i.e., methoxy and ethoxy). The designations “—OMe” and “MeO—” refer to a methoxy group.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “aryl”, unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. Aryl is intended to include stable cyclic, heterocyclic, polycyclic, and polyheterocyclic unsaturated C₃-C₁₄ moieties, exemplified but not limited to phenyl, biphenyl, naphthyl, pyridyl, furyl, thiophenyl, imidazoyl, pyrimidinyl, and oxazoyl, which may further be substituted with one to five members selected from the group consisting of hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry). If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

The term “alkylene” refers to a di-radical alkyl group. Unless otherwise indicated, such groups include saturated hydrocarbon chains containing from 1 to 24 carbon atoms, which may be substituted or unsubstituted, may contain one or more alicyclic groups, and may be heteroatom-containing. “Lower alkylene” refers to alkylene linkages containing from 1 to 6 carbon atoms. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

Similarly, the terms “alkenylene,” “alkynylene,” “arylene,” “aralklene,” and “alkanylene” refer to di-radical alkenyl, alkynyl, aryl, aralkyl, and alkaryl groups, respectively.

The term “amino” refers to the group —NRR′ wherein R and R′ are independently hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

“Carboxyl,” “carboxy” or “carboxylate” refers to —CO₂H or salts thereof.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocycloalkyl” refers to a cycloalkyl substituent that is heteroatom-containing, the terms “heterocyclic” or “heterocycle” refer to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The terms “heterocycle,” “heterocyclic” and “heterocyclyl” refer to a saturated or unsaturated group having a single ring or multiple condensed rings, including fused bridged and spiro ring systems, and having from 3 to 15 ring atoms, including 1 to 4 hetero atoms. These ring heteroatoms are selected from nitrogen, sulfur and oxygen, wherein, in fused ring systems, one or more of the rings can be cycloalkyl, heterocycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂-moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxy amino, alkoxyamino, nitro, —SO— alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. A hydrocarbyl may be substituted with one or more substituent groups. The term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl). C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

By the term “functional groups” is meant chemical groups such as halo, hydroxyl, sulfhydryl, C1-C24 alkoxy, C2-C24 alkenyloxy, C2-C24 alkynyloxy. C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH₂), mono-substituted C1-C24 alkylcarbamoyl (—(CO)—NH(C1-C24 alkyl)), di-substituted alkylcarbamoyl (—(CO)—N(C1-C24 alkyl)₂), mono-substituted arylcarbamoyl (—(CO)—NH-aryl), thiocarbamoyl (—(CS)—NH₂), carbamido (—NH—(CO)—NH₂), cyano (—C═N), isocyano (—N+≡C—), cyanato (—O—C═N), isocyanato (—O—N+≡C—), isothiocyanato (—S—C≡N), azido (—N≡N+═N—), formyl (—(CO)—H), thioformyl (—(CS)—H), amino (—NH₂), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO₂-alkyl), C5-C20 arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

By “linking” or “linker” as in “linking group,” “linker moiety,” etc., is meant a linking moiety that connects two groups via covalent bonds. The linker may be linear, branched, cyclic or a single atom. Examples of such linking groups include alkyl, alkenylene, alkynylene, arylene, alkarylene, aralkylene, and linking moieties containing functional groups including, without limitation: amido (—NH—CO—), ureylene (—NH—CO—NH—), imide (—CO—NH—CO—), epoxy (—O—), epithio (—S—), epidioxy (—O—O—), carbonyldioxy (—O—CO—O—), alkyldioxy (—O—(CH2)n-O—), epoxyimino (—O—NH—), epimino (—NH—), carbonyl (—CO—), etc. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, poly(ethylene glycol) unit(s) (e.g., —(CH₂—CH₂—O)—); ethers, thioethers, amines, alkyls (e.g., (C₁-C₁₂)alkyl), which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable. Any convenient orientation and/or connections of the linkers to the linked groups may be used.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, =N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO2R⁷⁰, —SO₂O⁻ M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻ M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰—CO₂⁻ M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R^{80′}s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺, an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —NO₂, —SO₂R⁷⁰, —SO₃⁻ M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻ M⁺, —P(O)(OR⁷⁰)₂, —C(O)R⁷⁰, —C(S)R⁷⁰, —C(NR⁷⁰)R⁷⁰, —CO₂⁻ M⁺, —CO₂,R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻ M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻ M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰ or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰) R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —N R⁷⁰C(NR⁷⁰)R⁷⁰ and —NR⁷⁰(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Definitions of other terms and concepts appear throughout the detailed description.

### DETAILED DESCRIPTION

As summarized above, aspects of the present disclosure include compounds, compositions and methods for the inhibition of ENPP1. Aspects of the methods include contacting a sample with an ENPP1 inhibitor compound to inhibit cGAMP hydrolysis activity of ENPP1. These compounds, compositions and methods find use in a variety of applications in which inhibition of ENPP1 is desired.

Also provided are pharmaceutical compositions and methods for treating cancer using the subject ENPP1 inhibitor compounds. Aspects of the methods include administering to a subject a therapeutically effective amount of an ENPP1 inhibitor compound to inhibit the hydrolysis of cGAMP and treat the subject for cancer.

### ENPP1-Inhibitor Compounds

The subject ENPP1 inhibitor compounds can include a core structure based on an aryl or heteroaryl ring system, e.g., a quinazoline or quinoline group, which is linked to a hydrophilic head group. The linker between the aryl or heteroaryl ring system and the hydrophilic head group can include a monocyclic aryl, heteroaryl, carbocycle or heterocycle and one or more acyclic linking moieties. The quinazoline or quinoline core structure can be substituted with the linker at the 4-position. The aryl or heteroaryl ring system is optionally further substituted. This disclosure includes compounds having a quinoline core structure that is substituted with the linker at the 4-position and with a cyano group at the 3-position. In some cases, the linker includes a 1,4-disubstituted 6-membered aryl or heteroaryl cyclic group, such as phenyl, or substituted phenyl. In certain cases, the linker includes a 1,4-disubstituted 6-membered saturated heterocycle or carbocycle, such as a N1,4-disubstituted piperidine ring or N1,N4-disubstituted piperazine ring. Further aspects of the subject ENPP1 inhibitor compounds are described below and by Li et al. in PCT application No. PCT/US2018/050018, filed Sep. 7, 2018, the disclosure of which is herein incorporated by reference in its entirety.

The term “hydrophilic head group” refers to a group linked to the core aryl or heteroaryl ring system that is hydrophilic and well solvated in aqueous environments, e.g., physiological conditions, and has low permeability to cell membranes. In some cases, by low permeability to cell membranes is meant a permeability coefficient of 10⁻⁴ cm/s or less, such as 10⁻⁵ cm/s or less, 10⁻⁶ cm/s or less, 10⁻⁷ cm/s or less, 10⁻⁸ cm/s or less, 10⁻⁹ cm/s or less, or even less, as measured via any convenient methods of passive diffusion for an isolated hydrophilic head group through a membrane (e.g., cell monolayers such as the colorectal Caco-2 or renal MDCK cell lines). See e.g., Yang and Hinner, Methods Mol Biol. 2015; 1266: 29-53. The hydrophilic head group can impart improved water solubility and reduced cell permeability upon the molecule to which it is attached. The hydrophilic head group may be any convenient hydrophilic group that is well solvated in aqueous environments and which has low permeability to membranes. In certain instances, the hydrophilic group is a discrete functional group (e.g., as described herein) or a substituted version thereof. In general, charged groups, or larger uncharged polar groups or have low permeability. In some cases, the hydrophilic head group is charged, e.g., positively or negatively charged. In some embodiments, the hydrophilic head group is itself not cell permeable and imparts cell impermeability upon the subject compound. It is understood that a hydrophilic headgroup, or a prodrug form thereof, can be selected to provide for a desired cell permeability of the subject compound. In certain cases, the hydrophilic head group is a neutral hydrophilic group. In some cases, the hydrophilic head group is included in a prodrug form and as such includes a promoiety that can be removed in vivo. In certain instances, the subject compound is cell permeable.

The hydrophilic head group can be any convenient group capable of binding or chelating zinc ions, or a prodrug form thereof. In certain cases, the hydrophilic head group is a phosphorus containing group. Phosphorus-containing groups of interest which may be utilized in the subject ENPP1 inhibitors include, but are not limited to, phosphonic acid or phosphonate, phosphonate ester, phosphate, phosphate ester, thiophosphate, thiophosphate ester, phosphoramidate and thiophosphoramidate, or a salt thereof, or a prodrug form thereof (e.g., as described herein).

Exemplary ENPP1 inhibitor compounds of interest including quinazoline and isoquinoline ring systems are set forth in formulae (I)—(XVb) and the compound structures of Tables 1-2.

In some cases, the subject ENPP1 inhibitor compound is of formula (I):

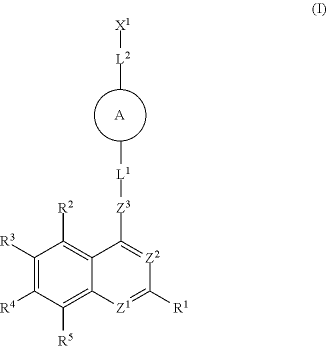

wherein,
X¹ is a hydrophilic head group (e.g., as described herein);
A is a ring system selected from aryl, substituted aryl, heteroaryl, substituted heteroaryl, cycloalkyl, substituted cycloalkyl, heterocycle and substituted heterocycle;
L¹ and L² are independently covalent bond or linker;
Z³ is absent or selected from NR²², O and S;
Z² is CR¹² or N;
Z¹ is CR¹¹ or N;
R¹ is selected from H, alkyl, substituted alkyl, alkenyl, substituted alkenyl, alkylaryl, substituted alkylaryl, alkylheteroaryl, substituted alkylheteroaryl, alkenylaryl (e.g., ethenylaryl), substituted alkenylaryl, alkenylheteroaryl (e.g., ethenylheteroaryl), substituted alkenylheteroaryl, aryl, substituted aryl, heteroaryl, substituted heteroaryl, heterocycle and substituted heterocycle;
R¹¹ and R¹² are independently selected from H, cyano, trifluoromethyl, halogen, alkyl and substituted alkyl;
R²² is selected from H, alkyl and substituted alkyl; and
R² to R⁵ are independently selected from H, OH, alkyl, substituted alkyl, alkenyl, substituted alkenyl, alkoxy, substituted alkoxy, —OCF₃, halogen, cyano, amine, substituted amine, amide, heterocycle and substituted heterocycle; or wherein R² and R³, R³ and R⁴, or R⁴ and R⁵ together with the carbon atoms to which they are attached provide a fused ring (e.g., 5- or 6-membered monocyclic ring) selected from heterocycle, substituted heterocycle, cycloalkyl, substituted cycloalkyl, aryl and substituted aryl;
or a pro-drug, pharmaceutically acceptable salt or solvate thereof.

In certain embodiments of formula (I), Z³ is absent. In certain embodiments of formula (I), Z³ is NR²², wherein R²² is selected from H, C₍₁₋₆₎alkyl and substituted C₍₁₋₆₎alkyl. In certain cases Z³ is NH. In certain cases, Z³ is NR²² and R²² is C₍₁₋₆₎alkyl, e.g., methyl, ethyl, propyl, pentyl or hexyl. In certain cases, Z³ is NR²² and R²² is substituted C₍₁₋₆₎alkyl. In certain cases of formula (I), Z³ is O. In certain cases of formula (I), Z³ is S.

In some instances of formula (I), Z¹ is CR¹¹ and R¹¹ is selected from hydrogen, cyano, trifluoromethyl, halogen, alkyl and substituted alkyl hydrogen. In some cases, the alkyl or substituted alky is C₁₋₅ alkyl. In some instances of formula (I), Z¹ is CR¹¹ and R¹¹ is hydrogen. In some cases, R¹¹ is cyano. In some cases, R¹¹ is trifluoromethyl. In some cases, R¹¹ is halogen, e.g., Br, I, Cl or F. In some cases, R¹¹ is alkyl, e.g., C₁₋₅ alkyl. In some cases, R¹¹ is substituted alkyl, e.g., substituted C₁₋₅ alkyl.

In some instances of formula (I), Z² is CR¹² and R¹² is selected from hydrogen, cyano, trifluoromethyl, halogen, alkyl and substituted alkyl hydrogen. In some cases, the alkyl or substituted alky is C₁₋₅ alkyl. In some instances of formula (I), Z² is CR¹² and R¹² is hydrogen. In some cases, R¹² is cyano. In some cases, R¹² is trifluoromethyl. In some cases, R¹² is halogen, e.g., Br, I, Cl or F. In some cases, R¹² is alkyl, e.g., C₁₋₅ alkyl. In some cases, R¹² is substituted alkyl, e.g., substituted C₁₋₅ alkyl.

In certain embodiments of formula (I), at least one of Z¹ and Z² is N. In certain embodiments of formula (I), Z¹ is CR¹¹ and Z² is N. In certain cases of formula (I), Z¹ is N and Z² is CR¹². In certain instances of formula (I), Z¹ is CR¹¹ and Z² is CR¹². In certain cases of formula (I), Z¹ is N and Z² is N.

In certain embodiments of formula (I), L¹ and L² are each covalent bonds. In certain cases, L¹ and L² are each linkers. In certain cases, L¹ is a covalent bond and L² is a linker. In certain cases, L¹ is a linker and L² is a covalent bond. Any convenient linkers can be utilized to link A to X and/or A to Z³ (e.g., as described herein). In some cases, A is linked to X via a covalent bond. In certain cases, A is linked to X via a linear linker of 1-12 atoms in length, such as 1-10, 1-8 or 1-6 atoms in length, e.g., 1, 2, 3, 4, 5 or 6 atoms in length. The linker L² can be a (C₁₋₆)alkyl linker or a substituted (C₁₋₆)alkyl linker, optionally substituted with a heteroatom or linking functional group, such as an ester (—CO₂—), amido (CONH), carbamate (OCONH), ether (—O—), thioether (—S—) and/or amino group (—NR— where R is H or alkyl). In some cases, A is linked to Z³ via a covalent bond. In certain cases, A is linked to Z³ via a linear linker of 1-12 atoms in length, such as 1-10, 1-8 or 1-6 atoms in length, e.g., 1, 2, 3, 4, 5 or 6 atoms in length. The linker L¹ can be a (C₁₋₆)alkyl linker or a substituted (C₁₋₆)alkyl linker, optionally substituted with a heteroatom or linking functional group, such as keto (CO), ester (—CO₂—), amido (CONH), carbamate (OCONH), ether (—O—), thioether (—S—) and/or amino group (—NR— where R is H or alkyl). When Z³ is NR²², the linker L¹ can include a terminal keto (C═O) group that together with Z³ provides an amido group (NR¹²CO) linkage. When Z³¹ is O or S, the linker L¹ can include a terminal keto (C═O) group that together with Z³¹ provides an ester or thioester group linkage.

In certain embodiments of formula (I), Z¹ is phosphorus-containing group capable of binding zinc ion, or a prodrug form thereof.

In certain instances of formula (I), Z¹ is selected from NR²², O and S. As such, the subject ENPP1 inhibitor compound of formula (I) can be described by formula (11):

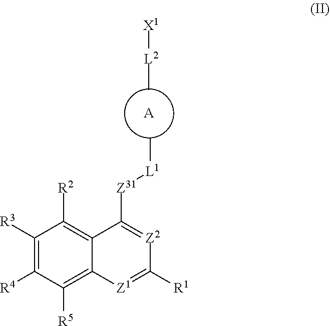

wherein Z³¹ is selected from NR²², O and S.

In certain embodiments of formula (II), Z³¹ is NR²², wherein R²² is selected from H, C₍₁₋₆₎alkyl and substituted C₍₁₋₆₎alkyl. In certain cases Z³¹ is NH. In certain cases, Z³¹ is NR²² and R²² is C₍₁₋₆₎alkyl, e.g., methyl, ethyl, propyl, pentyl or hexyl. In certain cases, Z³¹ is NR²² and R²² is substituted C₍₁₋₆₎alkyl. In certain cases of formula (I), Z³¹ is O. In certain cases of formula (I), Z³¹ is S.

In some instances of formula (II), Z¹ is CR¹¹ and R¹¹ is selected from hydrogen, cyano, trifluoromethyl, halogen, alkyl and substituted alkyl hydrogen. In some cases, the alkyl or substituted alky is C₁₋₅ alkyl. In some instances of formula (II), Z¹ is CR¹¹ and R¹¹ is hydrogen. In some cases, R¹¹ is cyano. In some cases, R¹¹ is trifluoromethyl. In some cases, R¹¹ is halogen, e.g., Br, I, Cl or F. In some cases, R¹¹ is alkyl, e.g., C₁₋₅ alkyl. In some cases, R¹¹ is substituted alkyl, e.g., substituted C₁₋₅ alkyl.

In some instances of formula (II), Z² is CR¹² and R¹² is selected from hydrogen, cyano, trifluoromethyl, halogen, alkyl and substituted alkyl hydrogen. In some cases, the alkyl or substituted alky is C₁₋₅ alkyl. In some instances of formula (II), Z² is CR¹² and R¹² is hydrogen. In some cases, R¹² is cyano. In some cases, R¹² is trifluoromethyl. In some cases, R¹² is halogen, e.g., Br, I, Cl or F. In some cases, R¹² is alkyl, e.g., C₁₋₅ alkyl. In some cases, R¹² is substituted alkyl, e.g., substituted C₁₋₅ alkyl.

In certain embodiments of formula (II), at least one of Z¹ and Z² is N. In certain embodiments of formula (I), Z¹ is CR¹¹ and Z² is N. In certain cases of formula (I), Z¹ is N and Z² is CR¹². In certain instances of formula (I), Z¹ is CR¹¹ and Z² is CR¹². In certain cases of formula (I). Z¹ is N and Z² is N.

In certain embodiments of formula (II), L¹ and L² are each covalent bonds. In certain cases, L¹ and L² are each linkers. In certain cases, L¹ is a covalent bond and L² is a linker. In certain cases, L¹ is a linker and L² is a covalent bond. Any convenient linkers can be utilized to link A to X and/or A to Z³ (e.g., as described herein). In some cases, A is linked to X via a covalent bond. In certain cases, A is linked to X via a linear linker of 1-12 atoms in length, such as 1-10, 1-8 or 1-6 atoms in length, e.g., 1, 2, 3, 4, 5 or 6 atoms in length. The linker L² can be a (C₁₋₆)alkyl linker or a substituted (C₁₋₆)alkyl linker, optionally substituted with a heteroatom or linking functional group, such as keto (CO), ester (—CO₂—), amido (CONH), carbamate (OCONH), ether (—O—), thioether (—S—) and/or amino group (—NR— where R is H or alkyl). In some cases, A is linked to Z³ via a covalent bond. In certain cases, A is linked to Z¹ via a linear linker of 1-12 atoms in length, such as 1-10, 1-8 or 1-6 atoms in length, e.g., 1, 2, 3, 4, 5 or 6 atoms in length. The linker L¹ can be a (C₁₋₆)alkyl linker or a substituted (C₁₋₆)alkyl linker, optionally substituted with a heteroatom or linking functional group, such as keto (C═O), ester (—CO₂—), amido (CONH), carbamate (OCONH), ether (—O—), thioether (—S—) and/or amino group (—NR— where R is H or alkyl). When Z³¹ is NR²², the linker L¹ can include a terminal keto (C═O) group that together with Z³¹ provides an amido group (NR²²CO) linkage. When Z³¹ is O or S, the linker L¹ can include a terminal keto (C═O) group that together with Z³¹ provides an ester or thioester group linkage.

In some cases of formula (II), the subject ENPP1 inhibitor compound is of formula (III):

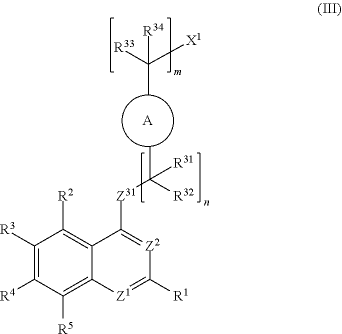

wherein:
each R³¹ to R³⁴ is independently selected from H, halogen, alkyl and substituted alkyl, or R³¹ and R³² or R³³ and R³⁴ are cyclically linked and together with the carbon atom to which they are attached provide a cycloalkyl, substituted cycloalkyl, heterocyclyl or substituted heterocyclyl ring; and
n and in are each independently an integer from 0 to 6 (e.g., 0-3).

In certain embodiments of formula (III), Z³¹ is NR²², wherein R²² is selected from H, C₍₁₋₆₎alkyl and substituted C₍₁₋₆₎alkyl. In certain cases Z³¹ is NH. In certain cases, Z³¹ is NR²² and R²² is C₍₁₋₆₎alkyl, e.g., methyl, ethyl, propyl, pentyl or hexyl. In certain cases, Z³¹ is NR²² and R²² is substituted C₍₁₋₆₎alkyl. In certain cases of formula (III), Z³¹ is O. In certain cases of formula (III), Z³¹ is S.

In formula (IT), when Z³¹ is NR²², the linker L¹ can include a terminal keto (C═O) group that together with Z³¹ provides an amido group (NR²²CO) linkage. As such, in some cases of formula (II), the subject ENPP1 inhibitor compound is of formula (IIIa):

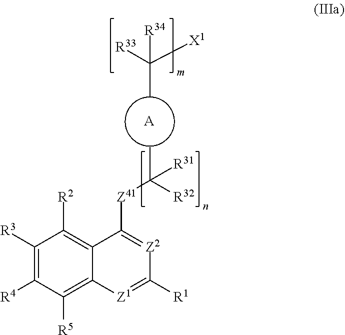

wherein:
Z⁴¹ is —NR²²C(═O)—;
each R³¹ to R³⁴ is independently selected from H, halogen, alkyl and substituted alkyl, or R³¹ and R³² or R³³ and R³⁴ are cyclically linked and together with the carbon atom to which they are attached provide a cycloalkyl, substituted cycloalkyl, heterocyclyl or substituted heterocyclyl ring; and
n and m are each independently an integer from 0 to 6 (e.g., 0-3).

In some instances of formulae (III)—(IIIa), Z¹ is CR¹¹ and R¹¹ is selected from hydrogen, cyano, trifluoromethyl, halogen, alkyl and substituted alkyl hydrogen. In some cases, the alkyl or substituted alky is C₁₋₅ alkyl. In some instances of formulae (III)—(IIIa), Z¹ is CR¹¹ and R¹¹ is hydrogen. In some cases, R¹¹ is cyano. In some cases, R¹¹ is trifluoromethyl. In some cases, R¹¹ is halogen, e.g., Br, I, Cl or F. In some cases, R¹¹ is alkyl, e.g., C₁₋₅ alkyl. In some cases, R¹¹ is substituted alkyl, e.g., substituted C₁₋₅ alkyl.

In some instances of formulae (III)—(IIIa), Z² is CR¹² and R¹² is selected from hydrogen, cyano, trifluoromethyl, halogen, alkyl and substituted alkyl hydrogen. In some cases, the alkyl or substituted alky is C₁₋₅ alkyl. In some instances of formulae (III)—(IIIa), Z² is CR¹² and R¹² is hydrogen. In some cases, R¹² is cyano. In some cases, R¹² is trifluoromethyl. In some cases, R¹² is halogen, e.g., Br, I, Cl or F. In some cases, R¹² is alkyl, e.g., C₁₋₅ alkyl. In some cases, R¹² is substituted alkyl, e.g., substituted C₁₋₅ alkyl.

In certain embodiments of formulae (III)—(IIIa), at least one of Z¹ and Z² is N. In certain embodiments of formulae (III)—(IIIa), Z¹ is CR¹¹ and Z² is N. In certain cases of formulae (III)—(IIIa). Z¹ is N and Z² is CR¹². In certain instances of formulae (III)—(IIIa), Z¹ is CR¹¹ and Z² is CR¹². In certain cases of formulae (III)—(IIIa), Z¹ is N and Z² is N.

In certain embodiments of formulae (III)—(IIIa), R³¹ to R³⁴ are each hydrogen. In certain embodiments, at least one of R³¹ to R³⁴ is a halogen. In certain embodiments, at least one of R³¹ to R³⁴ is alkyl. In certain embodiments, at least one of R³¹ to R³⁴ is substituted alkyl. In certain cases, one of R³¹ to R³⁴ is halogen and the remainder are selected from hydrogen, halogen, alkyl and substituted alkyl. In certain cases, one of R³¹ to R³⁴ is alkyl and the remainder are selected from hydrogen, halogen, alkyl and substituted alkyl. In certain cases, one R³¹ to R³⁴ is substituted alkyl and the remainder are selected from hydrogen, halogen, alkyl and substituted alkyl. In certain cases, one of R³¹ to R³⁴ is halogen and the remainder are hydrogen. In certain cases, one of R³¹ to R³⁴ is alkyl and the remainder are hydrogen. In certain cases, one R³¹ to R³⁴ is substituted alkyl and the remainder are hydrogen.

In certain embodiments of formulae (III)—(IIIa), n is an integer from 0 to 3. In certain cases n is 0. In certain cases, n is 1. In certain cases, n is 2. In certain cases n is 3. In certain embodiments of formulae (III)—(IIIa), m is an integer from 0 to 3. In certain cases, m is 0. In certain cases, m is 1. In certain cases, m is 2. In certain cases, m is 3. In certain cases, n is 0 and m is 1. In certain cases, n is 0 and m is 2. In certain case, n is 0 and m is 3. In certain cases, n is 1 and m is 0. In certain cases, n is 1 and m is 1. In certain cases, n is 1 and m is 2. In certain cases, n is 1 and m is 3. In certain cases, n is 2 and m is 0. In certain cases, n is 2 and m is 1. In certain cases, n is 2 and m is 2. In certain cases, n is 2 and m is 3. In certain cases, n is 3 and m is 0. In certain cases, n is 3 and m is 1. In certain cases, n is 3 and m is 2. In certain cases, n is 3 and m is 3. In certain cases, n+m is an integer from 0 to 3. In certain cases, n+m is 0. In certain cases, n+m is 1. In certain cases, n+m is 2. In certain cases, n+m is 3.

In some embodiments of any of formulae (I) to (IIIa), the ring system A is selected from phenyl, substituted phenyl, pyridyl, substituted pyridyl, pyrimidine, substituted pyrimidine, piperidine, substituted piperidine, piperazine, substituted piperazine, pyridazine, substituted pyridazine, cyclohexyl and substituted cyclohexyl. In certain cases, the ring system A is phenyl or substituted phenyl. In some cases, the ring system A is pyridyl or substituted pyridyl. In some cases, the ring system A is pyrimidine or substituted pyrimidine. In some cases, the ring system A is piperidine or substituted piperidine. In some cases, the ring system A is piperazine or substituted piperazine. In some cases, the ring system A is cyclohexyl or substituted cyclohexyl.

In some embodiments, the ring system A is described by the formula (A1):

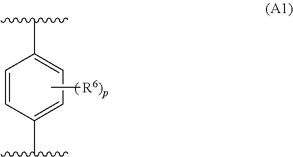

wherein:
each R⁶ is selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide; and
p is an integer from 0 to 4.

In certain cases, A1 is phenylene. In certain cases, A1 is a mono-substituted phenylene. In certain cases, A1 is a di-substituted phenylene. In certain cases, A1 is a tri-substituted phenylene. In certain cases, A1 is a tetra-substituted phenylene. In certain cases, the substitutents of the phenylene are selected from lower alkyl (e.g., methyl, ethyl, propyl, butyl, pentyl and hexyl) and halogen (e.g., F, Cl, I or Br).

In some embodiments, A1 ring is described by the formula (Ala):

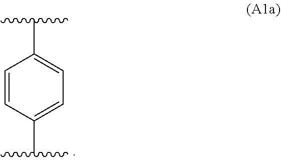 [SMILES: *c1ccc(*)cc1]

In some embodiments the ring system A is described by the formula (A2):

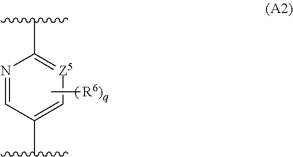

wherein:
Z⁵ is selected from N and CR⁶;
each R⁵ is selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide; and
q is an integer from 0 to 2.

In certain cases, A2 is pyridyl. In certain cases, A2 is a substituted pyridyl. In some cases, the pyridyl is a mono-substituted pyridyl. In other cases, the pyridyl is a di-substituted pyridyl. In other cases, the pyridyl is a tri-substituted pyridyl. In certain cases, Z⁵ is N, such that A2 is a pyrimidyl. In some cases, A2 is a substituted pyrimidyl. In some cases, the pyrimidyl is mono-substituted. In some cases, the pyrimidyl is di-substituted. In certain embodiments of A2, the substituents are selected from lower alkyl (e.g., methyl, ethyl, propyl, butyl, pentyl and hexyl), trifluoromethyl and halogen (e.g., F, Cl, I or Br).

In some embodiments, the ring system A is described by the formula (A3):

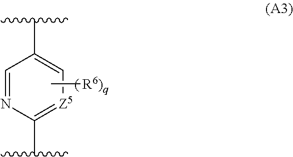

wherein:
Z⁵ is selected from N and CR⁶;
each R⁶ is selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide; and
q is an integer from 0 to 2.

In certain cases, A3 is pyridyl. In certain cases, A3 is a substituted pyridyl. In some cases, the pyridyl is a mono-substituted pyridyl. In other cases, the pyridyl is a di-substituted pyridyl. In other cases, the pyridyl is a tri-substituted pyridyl. In certain cases, Z⁵ is N, such that A3 is a pyrimidyl. In some cases, A3 is a substituted pyrimidyl. In some cases, the pyrimidyl is mono-substituted. In some cases, the pyrimidyl is di-substituted. In certain embodiments of A3, the substituents are selected from lower alkyl (e.g., methyl, ethyl, propyl, butyl, pentyl and hexyl), trifluoromethyl and halogen (e.g., F, Cl, I or Br).

In some embodiments, the ring system A is described by the formula (A4):

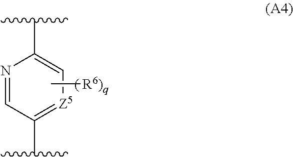

wherein:
Z⁵ is N;
each R⁶ is selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide; and
q is an integer from 0 to 2.

In some cases, A4 is a substituted pyrimidyl. In some cases, the pyrimidyl is mono-substituted. In some cases, the pyrimidyl is di-substituted. In certain embodiments of A4, the substituents are selected from lower alkyl (e.g., methyl, ethyl, propyl, butyl, pentyl and hexyl), trifluoromethyl and halogen (e.g., F, Cl, I or Br).

In some cases of formula (III)—(IIIa), the ENPP1 inhibitor compound is of formula (IV)—(IVa):

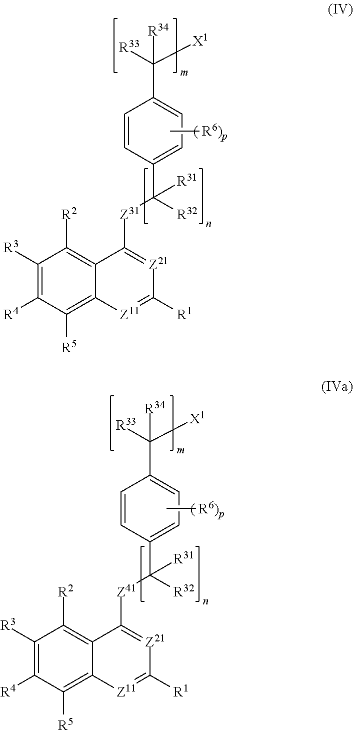

wherein:
Z³¹ is selected from NR²², O and S;
Z⁴¹ is —NR²²C(═O)—;
Z¹¹ and Z²¹ are independently selected from N and C(CN),
each R³¹ to R³⁴ is independently selected from H, halogen, alkyl and substituted alkyl, or R³¹ and R³² or R³³ and R³⁴ are cyclically linked and together with the carbon atom to which they are attached provide a cycloalkyl, substituted cycloalkyl, heterocyclyl or substituted heterocyclyl ring;
each R⁶ is independently selected from H, alkyl, substituted alkyl, hydroxy, alkoxy, substituted alkoxy, trifluoromethyl and halogen;
p is an integer from 0 to 4; and
n and m are each independently an integer from 0 to 6 (e.g., 0-3).

In certain embodiments of formulae (V)—(IVa), Z³¹ is NR²², wherein R²² is selected from H, C₍₁₋₆₎alkyl and substituted C₍₁₋₆₎alkyl. In certain cases Z³¹ is NH. In certain cases, Z³¹ is NR²² and R²² is C₍₁₋₆₎alkyl, e.g., methyl, ethyl, propyl, pentyl or hexyl. In certain cases, Z³¹ is NR²² and R²² is substituted C₍₁₋₆₎alkyl. In certain cases of formulae (IV)—(IVa), Z³¹ is O. In certain cases of formulae (IV)—(IVa), Z³¹ is S.

In certain embodiments of formulae (IV)—(IVa), at least one of Z¹¹ and Z²¹ is N. In certain embodiments of formulae (IV)—(IVa), Z³¹ is C(CN) and Z²¹ is N. In certain cases of formulae (IV)—(IVa), Z¹¹ is N and Z²¹ is C(CN). In certain instances of formulae (IV)—(IVa), Z³¹ is C(CN) and Z²¹ is C(CN). In certain cases of formulae (IV)—(IVa), Z¹¹ is N and Z²¹ is N.

In certain embodiments of formulae (IV)—(IVa), R³¹ to R³⁴ are each hydrogen. In certain embodiments, at least one of R³¹ to R³⁴ is a halogen. In certain embodiments, at least one of R³¹ to R³⁴ is alkyl. In certain embodiments, at least one of R³¹ to R³⁴ is substituted alkyl. In certain cases, one of R³¹ to R³⁴ is halogen and the remainder are selected from hydrogen, halogen, alkyl and substituted alkyl. In certain cases, one of R³¹ to R³⁴ is alkyl and the remainder are selected from hydrogen, halogen, alkyl and substituted alkyl. In certain cases, one of R³ to R³⁴ is substituted alkyl and the remainder are selected from hydrogen, halogen, alkyl and substituted alkyl. In certain cases, one of R³¹ to R³⁴ is halogen and the remainder are hydrogen. In certain cases, one of R³¹ to R³⁴ is alkyl and the remainder are hydrogen. In certain cases, one of R³¹ to R³⁴ is substituted alkyl and the remainder are hydrogen.

In certain embodiments of formulae (IV)—(IVa), n is an integer from 0 to 3. In certain cases n is 0. In certain cases, n is 1. In certain cases, n is 2. In certain cases n is 3. In certain embodiments of formulae (IV)—(IVa), m is an integer from 0 to 3. In certain cases, m is 0. In certain cases, m is 1. In certain cases, in is 2. In certain cases, in is 3. In certain cases, n is 0 and in is 1. In certain cases, n is 0 and m is 2. In certain case, n is 0 and m is 3. In certain cases, n is 1 and m is 0. In certain cases, n is 1 and m is 1. In certain cases, n is 1 and m is 2. In certain cases, n is 1 and m is 3. In certain cases, n is 2 and m is 0. In certain cases, n is 2 and m is 1. In certain cases, n is 2 and in is 2. In certain cases, n is 2 and m is 3. In certain cases, n is 3 and m is 0. In certain cases, n is 3 and m is 1. In certain cases, n is 3 and m is 2. In certain cases, n is 3 and m is 3. In certain cases, n+m is an integer from 0 to 3. In certain cases, n+m is 0. In certain cases, n+m is 1. In certain cases, n+m is 2. In certain cases, n+m is 3.

In some cases of formulae (IVa), n is 0 and in is 0-2, such as m is 1 or 2.

In some cases of formulae (IV)—(IVa), the ENPP1 inhibitor compound is of formulae (V)—(Va):

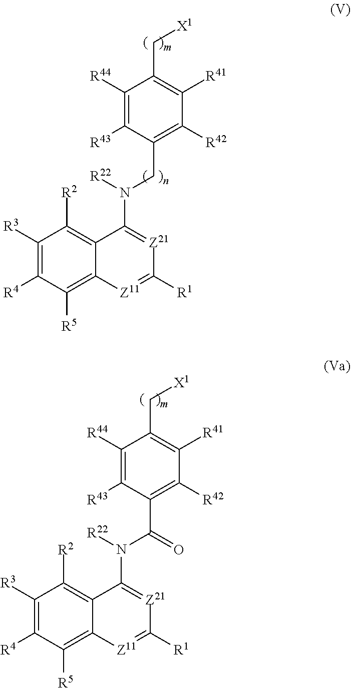

wherein:
R⁴¹ to R⁴⁴ are independently selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide.

In certain embodiments of formulae (V)—(Va), at least one of Z¹¹ and Z²¹ is N. In certain embodiments of formulae (V)—(Va), Z¹¹ is C(CN) and Z²¹ is N. In certain cases of formulae (V)—(Va), Z¹¹ is N and Z²¹ is C(CN). In certain instances of formulae (V)—(Va), Z¹¹ is C(CN) and Z²¹ is C(CN). In certain cases of formulae (V)—(Va), Z¹¹ is N and Z²¹ is N.

In some cases of formulae (V)—(Va), the subject ENPP1 inhibitor compound is of one of formulae (VIa)-(VId):

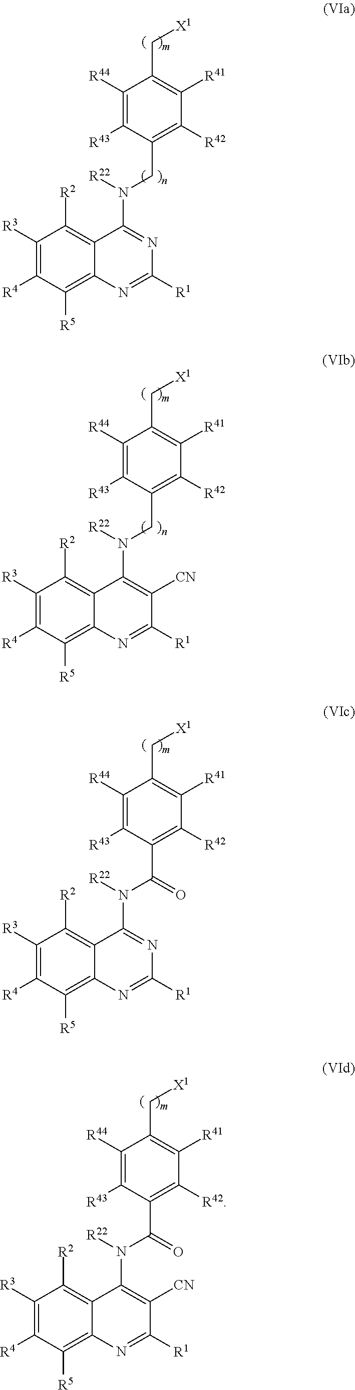

In certain embodiments of formulae (VIa)-(VId). R⁴¹ to R⁴⁴ are each hydrogen. In certain embodiments, at least one of R⁴¹ to R⁴⁴ is alkyl or substituted alkyl. In certain embodiments, at least one of R⁴¹ to R⁴⁴ is hydroxy. In certain embodiments, at least one of R⁴¹ to R⁴⁴ is alkoxy or substituted alkoxy. In certain cases, at least one of R⁴¹ to R⁴⁴ is trifluoromethyl. In certain cases, at least one of R⁴¹ to R⁴⁴ is halogen. In certain cases, at least one of R⁴¹ to R⁴⁴ is acyl or substituted acyl. In certain cases, at least one of R⁴¹ to R⁴⁴ is carboxy. In certain cases, at least one of R⁴¹ to R⁴⁴ is carboxyamide or substituted carboxyamide. In certain cases, at least one of R⁴¹ to R⁴⁴ is sulfonyl or substituted sulfonyl. In certain cases, at least one of R⁴¹ to R⁴⁴ is sulfonamide and substituted sulfonamide. In certain cases, one of R³¹ to R³⁴ is hydrogen and the remainder are selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide. In certain cases, two of R³¹ to R³⁴ are hydrogen and the remainder are selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide. In certain cases, three of R³¹ to R³⁴ are hydrogen and the remainder are selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide.

In certain embodiments of formulae (VIa)-(VId), n is an integer from 0 to 3. In certain cases n is 0. In certain cases, n is 1. In certain cases, n is 2. In certain cases n is 3. In certain embodiments of any of formulae (VIa)-(VId), m is an integer from 0 to 3. In certain cases, m is 0. In certain cases, m is 1. In certain cases, in is 2. In certain cases, in is 3. In certain cases, n is 0 and in is 1. In certain cases, n is 0 and m is 2. In certain case, n is 0 and m is 3. In certain cases, n is 1 and m is 0. In certain cases, n is 1 and in is 1. In certain cases, n is 1 and m is 2. In certain cases, n is 1 and m is 3. In certain cases, n is 2 and m is 0. In certain cases, n is 2 and m is 1. In certain cases, n is 2 and m is 2. In certain cases, n is 2 and m is 3. In certain cases, n is 3 and m is 0. In certain cases, n is 3 and m is 1. In certain cases, n is 3 and m is 2. In certain cases, n is 3 and m is 3. In certain cases, n+m is an integer from 0 to 3. In certain cases, n+m is 0. In certain cases, n+m is 1. In certain cases, n+m is 2. In certain cases, n+m is 3.

In certain embodiments of any of formulae (VIa)-(VId). R²² is hydrogen. In certain cases, R²² is alkyl. In certain cases, R²² is substituted alkyl. In certain cases, the alkyl or substituted alkyl is C₍₁₋₆₎alkyl.

In certain embodiments of any of formulae (I)—(VId), R¹ is selected from hydrogen, alkylaryl, substituted alkylaryl, alkylheteroaryl, substituted alkylheteroaryl, alkenylaryl (e.g., ethenylaryl), substituted alkenylaryl, alkenylheteroaryl (e.g., ethenylheteroaryl), substituted alkenylheteroaryl, aryl, substituted aryl, heteroaryl and substituted heteroaryl.

In certain cases of formulae (I)—(VId), R¹ is hydrogen. In certain cases. R¹ is aryl or substituted aryl. In certain cases. R¹ is heteroaryl or substituted heteroaryl. In certain cases. R¹ is alkylaryl or substituted alkylaryl. In certain cases, R¹ is alkylheteroaryl or substituted alkylheteroaryl. In certain cases, R¹ is alkenylaryl, or substituted alkenylaryl. In certain cases, R¹ is ethenylaryl. In certain cases. R¹ is substituted ethenylaryl. In some cases, R¹ is ethenylheteroaryl. In certain cases. R¹ is alkenylheteroaryl or substituted alkenylheteroaryl. In some cases, R¹ is substituted ethenylheteroaryl.

In some cases of formula (VIa)-(VId), the ENPP1 inhibitor compound is of one of formulae (VIIa)-(VIIb):

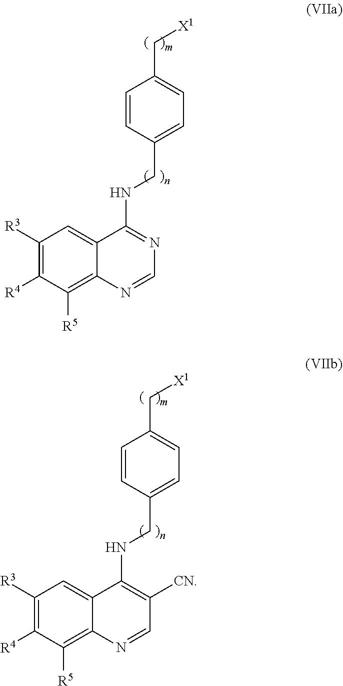

In certain embodiments of any of formulae (I)—(VIIb), R² to R⁵ are independently selected from H, OH, alkyl, substituted alkyl, alkoxy, substituted alkoxy, —OCF₃, halogen, cyano, amine, substituted amine, amide, heterocycle and substituted heterocycle.

In certain embodiments of any of formulae (I)—(VIIb), R² to R⁵ are independently selected from hydrogen, OH, C₍₁₋₆₎alkoxy, —OCF₃, C₍₁₋₆₎alkylamino, di-C₍₁₋₆₎alkylamino, F, Cl, Br and CN.

In certain cases, at least one of R² to R⁵ is hydrogen. In certain cases, at least two of R² to R⁵ are hydrogen. In certain cases, each of R² to R⁵ is hydrogen. In certain cases, at least one of R² to R⁵ is hydroxy. In certain cases, at least one of R² to R⁵ is alkyl or substituted alkyl. In certain cases, at least one of R² to R⁵ is alkoxy or substituted alkoxy. In certain cases, the alkoxy or substituted alkoxy is a C₍₁₋₆₎alkoxy, e.g., methoxy, ethoxy, propoxy, butoxy, pentoxy, hexoxy. In certain cases, at least one of R² to R⁵ is methoxy. In certain cases, at least one of R² to R⁵ is —OCF₃. In certain cases, at least one of R² to R⁵ is halogen. In certain cases, the halogen is fluoride. In certain cases, the halogen is chloride. In certain cases, the halogen is bromide. In certain cases, at least one of R² to R⁵ is cyano. In certain cases, at least one of R² to R⁵ is amine or substituted amine. In certain cases, at least one of R² to R⁵ is C₍₁₋₆₎alkylamino. In certain cases, at least one of R² to R⁵ is di-C₍₁₋₆₎alkylamino. In certain cases, at least one of R² to R⁵ is amide. In certain cases, at least one of R² to R⁵ is heterocycle or substituted heterocycle.

In some instances of formulae (I)—(VIIb), R³ and R⁴ are independently alkoxy; and R² and R⁵ are both hydrogen. In certain cases, the alkoxy is methoxy. In some cases, R³ is alkoxy; and R², R⁴ and R⁵ are hydrogen. In some cases, R⁴ is alkoxy; and R², R³ and R⁵ are each hydrogen. In certain cases, R², R³ and R⁴ are hydrogen and R⁵ is alkoxy. In certain cases, the alkoxy is a C₍₁₋₆₎alkoxy. In certain cases, the alkoxy is methoxy. In certain cases, the alkoxy is ethoxy. In certain cases, the alkoxy is propoxy. In certain cases, the alkoxy is butoxy. In certain cases, the alkoxy is pentoxy. In certain cases, the alkoxy is hexyloxy.

In some cases of formulae (VIc)-(VId), R⁴¹-R⁴⁴ are each independently H, halogen. C₍₁₋₆₎alkyl or C₍₁₋₆₎alkoxy. In some cases of formulae (VIc)-(VId), m is 1 or 2. In some cases of formulae (VIc)-(VId), R2 is H, and R³ to R⁵ are independently selected from hydrogen, C₍₁₋₆₎alkoxy, F, Cl and C₍₁₋₆₎alkyl.

In some cases of formulae (VIIa)-(VIIb), the subject ENPP1 inhibitor compound is of one of formulae (VIIc)-(VIII):

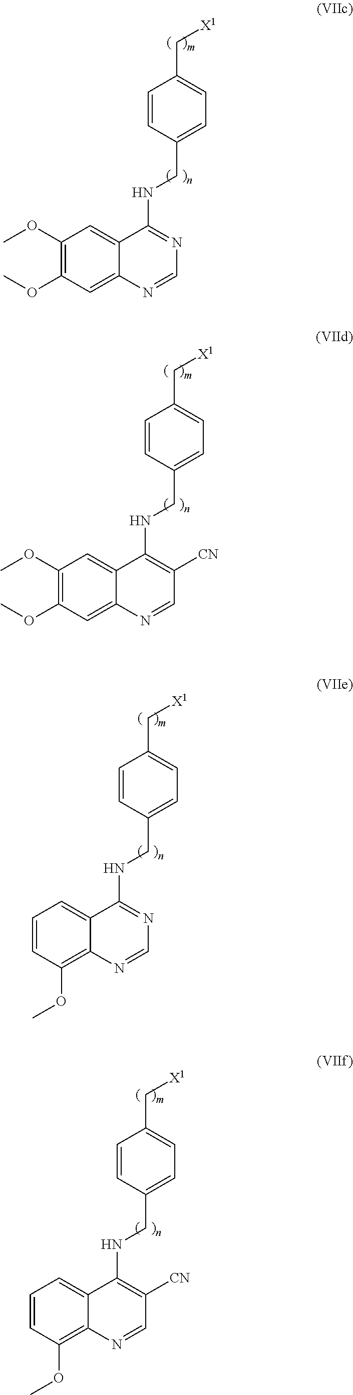

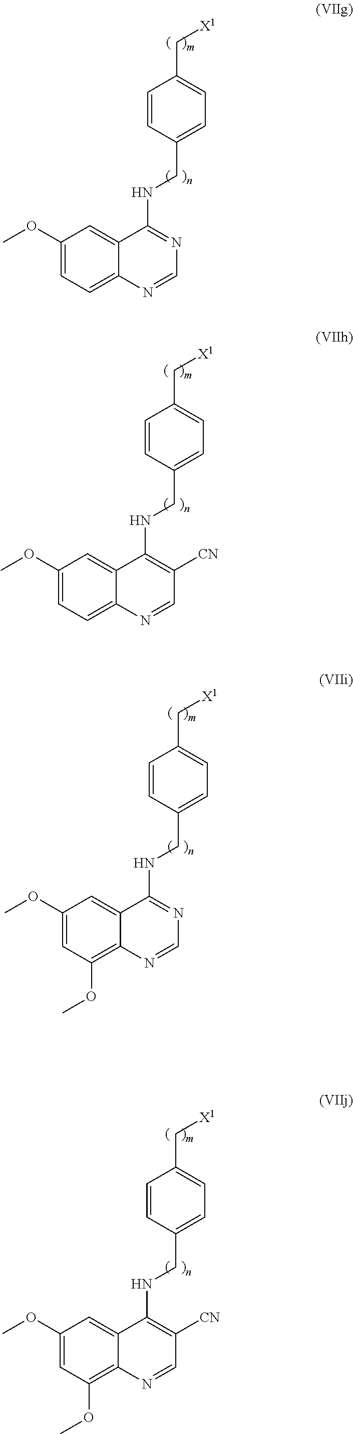

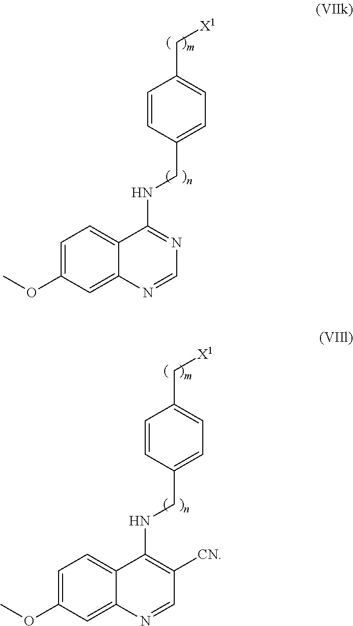

In some cases of formula (VIa), the ENPP1 inhibitor compound is of formula (VIIm):

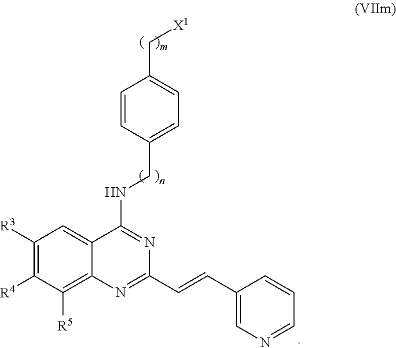

In certain embodiments of formula (VIIm), R² to R⁵ are independently selected from H, OH, alkyl, substituted alkyl, alkoxy, substituted alkoxy, —OCF₃, halogen, cyano, amine, substituted amine, amide, heterocycle and substituted heterocycle. In certain embodiments of formula (VIIm). R² to R⁵ are independently selected from hydrogen. OH, C₍₁₋₆₎alkoxy, —OCF₃, C₍₁₋₆₎alkylamino, di-C₍₁₋₆₎alkylamino, F, Cl, Br and CN. In certain embodiments of formula (VIIm), n+m=1. In certain embodiments of formula (VIIm), n+m=2. In certain embodiments of formula (VIIm), n is 1 and m is 0.

In certain cases of formula (VIIm), at least one of R³ to R⁵ is hydrogen. In certain cases, at least two of R³ to R⁵ are hydrogen. In certain cases, each of R³ to R⁵ is hydrogen. In certain cases, at least one of R³ to R⁵ is hydroxy. In certain cases, at least one of R³ to R⁵ is alkyl or substituted alkyl. In certain cases, at least one of R³ to R⁵ is alkoxy or substituted alkoxy. In certain cases of formula (VIIm), the alkoxy or substituted alkoxy is a C₍₁₋₆₎alkoxy, e.g., methoxy, ethoxy, propoxy, butoxy, pentoxy, hexoxy. In certain cases, at least one of R³ to R⁵ is methoxy. In certain cases of formula (VIIm), at least one of R³ to R⁵ is —OCF₃. In certain cases, at least one of R³ to R⁵ is halogen. In certain cases, the halogen is fluoride. In certain cases, the halogen is chloride. In certain cases, the halogen is bromide. In certain cases, at least one of R³ to R⁵ is cyano. In certain cases, at least one of R³ to R⁵ is amine or substituted amine. In certain cases, at least one of R³ to R⁵ is C₍₁₋₆₎alkylamino. In certain cases, at least one of R³ to R⁵ is di-C₍₁₋₆₎alkylamino. In certain cases of formula (VIIm), at least one of R³ to R⁵ is amide. In certain cases, at least one of R³ to R⁵ is heterocycle or substituted heterocycle.

In some instances of formula (VIIm). R³ and R⁴ are independently alkoxy; and R² and R⁵ are both hydrogen. In certain cases, the alkoxy is methoxy. In some cases, R³ is alkoxy; and R², R⁴ and R⁵ are hydrogen. In some cases, R⁴ is alkoxy; and R², R³ and R⁵ are each hydrogen. In certain cases of formula (VIIm), R², R³ and R⁴ are hydrogen and R⁵ is alkoxy. In certain cases, the alkoxy is a C₍₁₋₆₎alkoxy. In certain cases, the alkoxy is methoxy. In certain cases, the alkoxy is ethoxy. In certain cases, the alkoxy is propoxy. In certain cases, the alkoxy is butoxy. In certain cases, the alkoxy is pentoxy. In certain cases, the alkoxy is hexyloxy.

In certain embodiments of formula (VIIm), n is 0-3 and m is 0-3. In some instances of formula (VIIm), m is 0. In certain cases, m is 1. In certain cases, m is 2. In certain cases, m is 3. In certain cases, n is 0 and m is 1. In certain cases, n is 0 and m is 2. In certain case, n is 0 and m is 3. In certain cases, n is 1 and m is 0. In certain cases, n is 1 and m is 1. In certain cases, n is 1 and m is 2. In certain cases, n is 1 and m is 3. In certain cases, n is 2 and m is 0. In certain cases, n is 2 and m is 1. In certain cases, n is 2 and m is 2. In certain cases, n is 2 and m is 3. In certain cases, n is 3 and m is 0. In certain cases, n is 3 and m is 1. In certain cases, n is 3 and m is 2. In certain cases, n is 3 and m is 3. In certain cases, n+m is an integer from 0 to 3. In certain cases, n+m is 0. In certain cases, n+m is 1. In certain cases, n+m is 2. In certain cases, n+m is 3.

In certain instances of the ENPP1 inhibitor compounds of formula (I), Z³ is absent. In certain embodiments of formula (I), Z³ is absent, Z² is CR¹², R¹² is cyano, and the compound is described by formula (X):

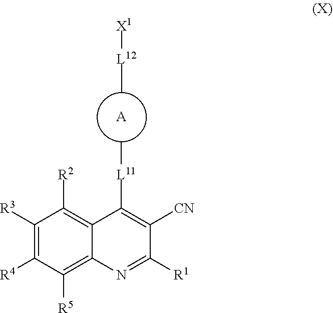

wherein L¹¹ and L¹² are independently covalent bond or linker. In some instances of formula (X), L¹¹ is covalent bond.

In some embodiments of formula (X), the ring system A is selected from phenyl, substituted phenyl, pyridyl, substituted pyridyl, pyrimidine, substituted pyrimidine, piperidine, substituted piperidine, piperazine, substituted piperazine, pyridazine, substituted pyridazine, cyclohexyl and substituted cyclohexyl. In certain cases, the ring system A is phenyl or substituted phenyl. In some cases, the ring system A is pyridyl or substituted pyridyl. In some cases, the ring system A is pyrimidine or substituted pyrimidine. In some cases, the ring system A is piperidine or substituted piperidine. In some cases, the ring system A is piperazine or substituted piperazine. In some cases, the ring system A is cyclohexyl or substituted cyclohexyl.

In some embodiments, the ring system A is described by any one of formulae (A1)-(A4), (e.g., as described herein):

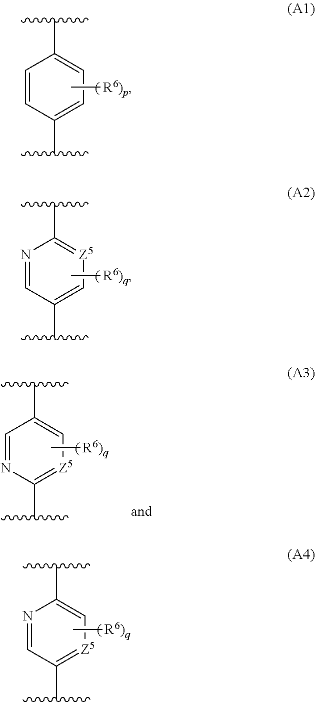

wherein:
Z⁵ is selected from N and CR⁶;
each R⁶ is selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide;
p is an integer from 0 to 4; and
q is an integer from 0 to 2.

In some embodiments the A ring is described by the formula (A5):

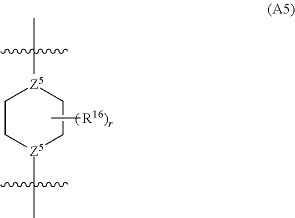

wherein:
each Z⁵ is independently selected from N and CR¹⁶;
each R¹⁶ is independently selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide; and
r is an integer from 0 to 8.

In certain cases, A5 is piperidine or substituted piperidine. In certain cases, A5 is piperazine or substituted piperazine. In certain cases, A5 is a cyclohexyl or a substituted cyclohexyl. In certain embodiments of A5, r is greater than 0, such as 1, 2, 3, 4, 5, 6, 7 or 8. In some cases, A5 includes one R¹⁶ group. In some cases, A5 includes two R¹⁶ groups. In some cases, A5 includes three R¹⁶ groups. In some cases, A5 includes four R¹⁶ groups. In certain embodiments, the substituents are selected from lower alkyl (e.g., methyl, ethyl, propyl, butyl, pentyl and hexyl), trifluoromethyl and halogen (e.g., F, Cl, I or Br).

In certain embodiments, the A ring has any one of the formulae (A5a)-(A5c):

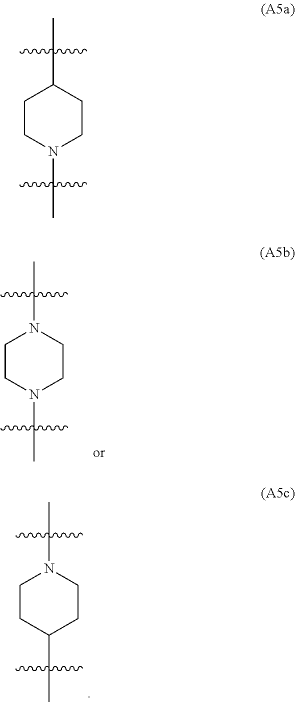 [SMILES: *C1CCN(*)CC1 ; *N1CCN(*)CC1 ; *C1CCN(*)CC1]

In certain embodiments, the A ring is a cyclohexyl having the relative configuration of formula (A5d) or (A5e):

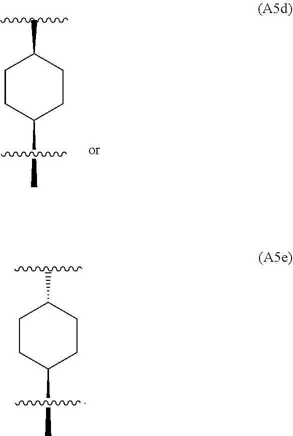

In certain cases of formula (X), the subject ENPP1 inhibitor compound is of the formula (XI):

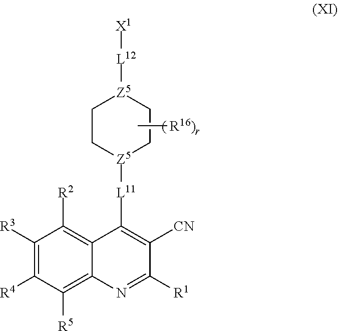

wherein:
each Z⁵ is independently selected from N and CR¹⁶;
each R¹⁶ is independently selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide; and
r is an integer from 0 to 8.

In certain embodiments of formula (XI), at least one Z⁵ is N. In certain embodiments of formula (XI), one Z⁵ is N and the other Z⁵ is CR¹⁶. In certain cases of formula (XI), both Z⁵ groups are CR¹⁶. In certain cases of formula (XI), both Z⁵ groups are N.

In certain embodiments of a compound of any one of formulae (X)—(XI). L¹¹ and L¹² are each covalent bonds. In certain cases, L¹¹ and L¹² are each linkers. In certain cases, L¹¹ is a covalent bond and L¹² is a linker. In certain cases, L¹¹ is a linker and L¹² is a covalent bond. Any convenient linkers can be utilized as L¹¹ and L¹². In some cases, L¹¹ is a covalent bond. In certain cases, L¹¹ is a linear linker of 1-12 atoms in length, such as 1-10, 1-8 or 1-6 atoms in length, e.g., 1, 2, 3, 4, 5 or 6 atoms in length. The linker L¹¹ can be a (C₁₋₆)alkyl linker or a substituted (C₁₋₆)alkyl linker, optionally substituted with a heteroatom or linking functional group, such as an ester (—CO₂—), amido (CONH), carbamate (OCONH), ether (—O—), thioether (—S—) and/or amino group (—NR— where R is H or alkyl). In some cases, L¹² is a covalent bond. In certain cases, L¹² is a linker of 1-12 atoms in length, such as 1-10, 1-8 or 1-6 atoms in length, e.g., 1, 2, 3, 4, 5 or 6 atoms in length. The linker L¹² can be a (C₁₋₆)alkyl linker or a substituted (C₁₋₆)alkyl linker, optionally substituted with a heteroatom or linking functional group, such as an ester (—CO₂—), amido (CONH), carbamate (OCONH), ether (—O—), thioether (—S—) and/or amino group (—NR— where R is H or alkyl).

In some cases of formula (XI), the subject ENPP1 inhibitor compound is of the formula (XII):

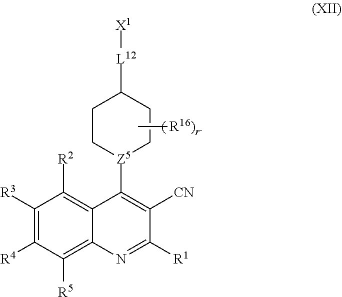

In certain embodiments of the compound of formula (XII), Z⁵ is CR¹⁶, wherein R¹⁶ is selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide. In certain cases of the compound of formula (XII), Z⁵ is N.

In certain embodiments of a compound of formula (XII). L¹² is a covalent bond. In certain cases, L¹² is a linker. Any convenient linkers can be utilized as L¹². In certain cases, L¹² is a linear linker of 1-12 atoms in length, such as 1-10, 1-8 or 1-6 atoms in length, e.g., 1, 2, 3, 4, 5 or 6 atoms in length. The linker L¹² can be a (C₁₋₆)alkyl linker or a substituted (C₁₋₆)alkyl linker, optionally substituted with a heteroatom or linking functional group, such as an ester (—CO₂—), amido (CONH), carbamate (OCONH), ether (—O—), thioether (—S—) and/or amino group (—NR— where R is H or alkyl).

In some cases of formula (XII), the subject ENPP1 inhibitor compound is of the formula (XIII):

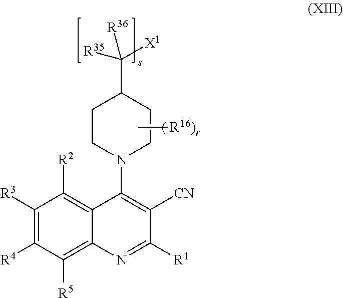

Wherein
R³⁵ and R³⁶ are each independently selected from H, halogen, alkyl and substituted alkyl, or R³⁵ and R³⁶ are cyclically linked and together with the carbon atom to which they are attached provide a cycloalkyl, substituted cycloalkyl, heterocyclyl or substituted heterocyclyl ring, and
s is an integer from 0 to 6 (e.g., 0 to 3).

In certain embodiments of formula (XIII). R³⁵ and R³⁶ are each hydrogen. In certain embodiments, at least one of R³⁵ or R³⁶ is a halogen. In certain embodiments, at least one of R³⁵ or R³⁶ is alkyl. In certain embodiments, at least one of R³⁵ or R³⁶ is substituted alkyl. In certain cases, R³⁵ is halogen and R³⁶ is selected from hydrogen, halogen, alkyl and substituted alkyl. In certain cases. R³⁵ is alkyl and R³⁶ is selected from hydrogen, halogen, alkyl and substituted alkyl. In certain cases, R³⁵ is substituted alkyl and R³⁶ is selected from hydrogen, halogen, alkyl and substituted alkyl. In certain cases, R³⁵ is halogen and R³⁶ is hydrogen. In certain cases. R³⁵ is alkyl and R³⁶ is hydrogen. In certain cases. R³⁵ is substituted alkyl and R³⁶ is hydrogen.

In certain embodiments of formula (XIII), s is an integer from 0 to 3. In certain cases s is 0. In certain cases, s is 1. In certain cases, s is 2. In certain cases s is 3.

In some cases of formula (XIII), the subject ENPP1 inhibitor compound is of the formula (XIV):

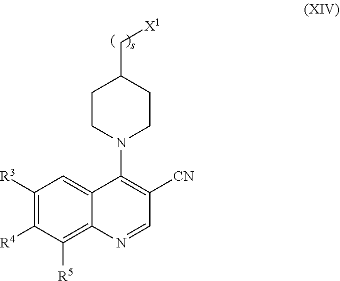

wherein s is an integer from 0 to 6 (e.g., 0 to 3).

In certain embodiments of formula (XIII), s is an integer from 0 to 3. In certain cases s is 0. In certain cases, s is 1. In certain cases, s is 2. In certain cases s is 3.

In certain embodiments of any of formulae (X)—(XIV). R² to R⁵ are independently selected from H, OH, alkyl, substituted alkyl, alkoxy, substituted alkoxy, —OCF₃, halogen, cyano, amine, substituted amine, amide, heterocycle and substituted heterocycle.

In certain embodiments of any of formulae (X)—(XIV), R² to R⁵ are independently selected from hydrogen, OH, C₍₁₋₆₎alkoxy, —OCF₃, C₍₁₋₆₎alkylamino, di-C₍₁₋₆₎alkylamino, F, Cl, Br and CN.

In certain cases of any of formulae (X)—(XIV), at least one of R² to R⁵ is hydrogen. In certain cases, at least two of R² to R⁵ are hydrogen. In certain cases, at least three of R² to R⁵ are hydrogen. In certain cases, each of R² to R⁵ is hydrogen. In certain cases, at least one of R² to R⁵ is hydroxy. In certain cases, at least one of R² to R⁵ is alkyl or substituted alkyl. In certain cases, at least one of R² to R⁵ is alkoxy or substituted alkoxy. In certain cases, the alkoxy or substituted alkoxy is a C₍₁₋₆₎alkoxy, e.g., methoxy, ethoxy, propoxy, butoxy, pentoxy, hexoxy. In certain cases at least one of R² to R⁵ is methoxy. In certain cases, at least one of R² to R⁵ is —OCF₃. In certain cases, at least one of R² to R⁵ is halogen. In certain cases, the halogen is fluoride. In certain cases, the halogen is chloride. In certain cases, the halogen is bromide. In certain cases, at least one of R² to R⁵ is cyano. In certain cases, at least one of R² to R⁵ is amine or substituted amine. In certain cases, at least one of R² to R⁵ is C₍₁₋₆₎alkylamino. In certain cases, at least one of R² to R⁵ is di-C₍₁₋₆₎alkylamino. In certain cases, at least one of R² to R⁵ is amide. In certain cases, at least one of R² to R⁵ is heterocycle or substituted heterocycle.

In some instances of any of formulae (X)—(XIV), R³ and R⁴ are independently alkoxy; and R² and R⁵ are both hydrogen. In some cases, R³ is alkoxy; and R², R⁴ and R⁵ are hydrogen. In some cases, R⁴ is alkoxy; and R², R³ and R⁵ are each hydrogen. In certain cases. R². R³ and R⁴ are hydrogen and R⁵ is alkoxy. In certain cases, the alkoxy is a C₍₁₋₆₎alkoxy. In certain cases, the alkoxy is methoxy. In certain cases, the alkoxy is ethoxy. In certain cases, the alkoxy is propoxy. In certain cases, the alkoxy is butoxy. In certain cases, the alkoxy is pentoxy. In certain cases, the alkoxy is hexyloxy.

In some cases of formula (XIV), the subject ENPP1 inhibitor compound is of one of formulae (XIVa)-(XIVe):

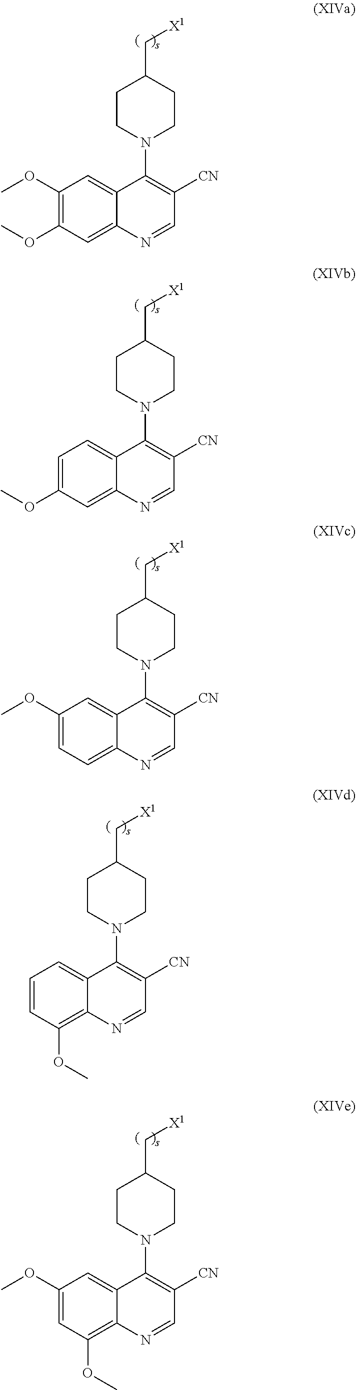

wherein s is an integer from 0 to 6 (e.g., 0 to 3).

In some cases of formula (I), the subject ENPP1 inhibitor compound is of the formula (XVa) or (XVb):

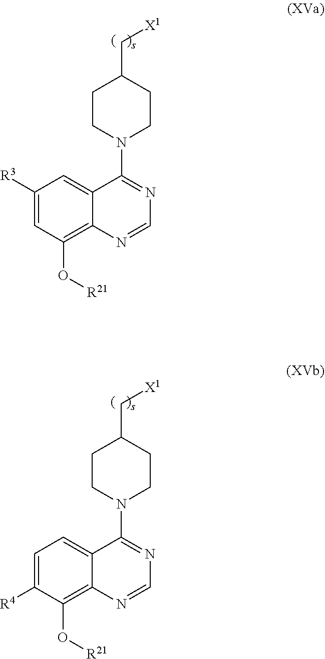

wherein:
s is 0 to 3;
R²¹ is C₍₁₋₆₎alkyl or substituted C₍₁₋₆₎alkyl; and
R³ and R⁴ are selected from Cl and F.

In some cases of formula (XVa)-(XVb), R²¹ is selected from methyl, ethyl, n-propyl and isopropyl. In certain cases, R²¹ is methyl. In some cases of formula (XVa)-(XVb), R³ and R⁴ are Cl. In certain instances. R³ and R⁴ are F. In some cases of formula (XVa)-(XVb), s is 2. In certain instances, s is 1. In some embodiments of formulae (XVa)-(XVb), s is 2; R²¹ is methyl or isopropyl; and R³ and R⁴ are selected from Cl and F.

In some instances of formulae (XVa)-(XVb), the subject ENPP1 inhibitor compound is of one of the following structures, or a prodrug thereof (e.g., as described herein):

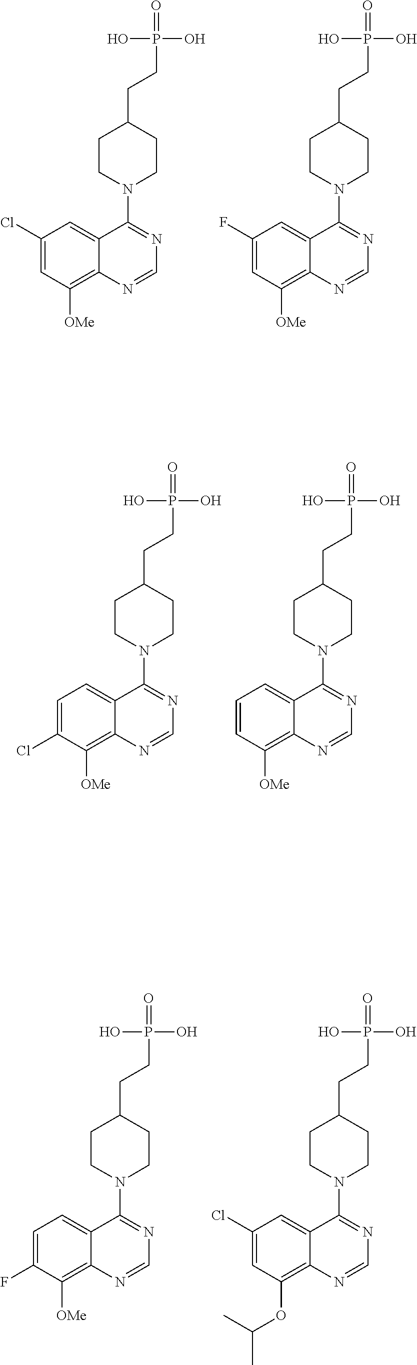 [SMILES: COc1cc(Cl)cc2c(N3CCC(CCP(=O)(O)O)CC3)ncnc12 ; COc1cc(F)cc2c(N3CCC(CCP(=O)(O)O)CC3)ncnc12 ; COc1c(Cl)ccc2c(N3CCC(CCP(=O)(O)O)CC3)ncnc12 ; COc1cccc2c(N3CCC(CCP(=O)(O)O)CC3)ncnc12 ; COc1c(F)ccc2c(N3CCC(CCP(=O)(O)O)CC3)ncnc12 ; CC(C)Oc1cc(Cl)cc2c(N3CCC(CCP(=O)(O)O)CC3)ncnc12]

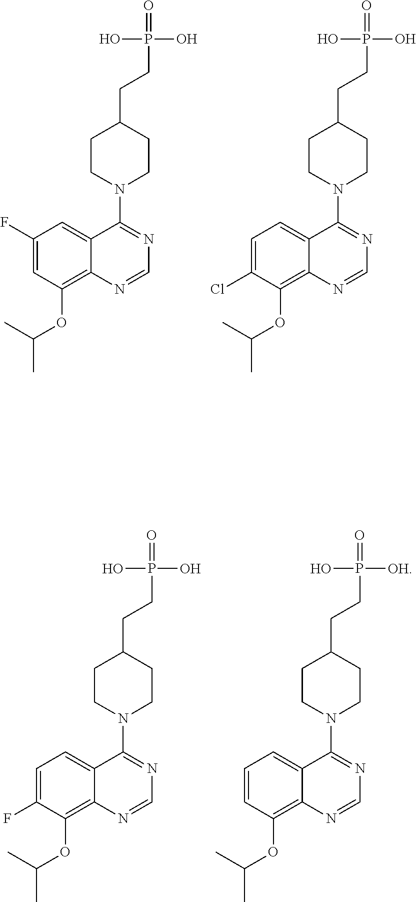 [SMILES: CC(C)Oc1cc(F)cc2c(N3CCC(CCP(=O)(O)O)CC3)ncnc12 ; CC(C)Oc1c(Cl)ccc2c(N3CCC(CCP(=O)(O)O)CC3)ncnc12 ; CC(C)Oc1c(F)ccc2c(N3CCC(CCP(=O)(O)O)CC3)ncnc12 ; CC(C)Oc1cccc2c(N3CCC(CCP(=O)(O)O)CC3)ncnc12]

As described above. X¹ is a hydrophilic head group or a prodrug version thereof. Any embodiments of a hydrophilic head group described herein can be incorporated into any one of the embodiments of formulae (I)—(XVb) described herein. In some embodiments of formulae (I)—(XVb), X¹ is a hydrophilic head group comprising a charged group capable of binding zinc ion, or a prodrug form thereof. In certain cases, the hydrophilic head group capable of binding zinc ion is a phosphorus containing functional group (e.g., as described herein).

In some embodiments of formulae (I)—(XVb), the hydrophilic head group (X¹) is selected from phosphonic acid or phosphonate, phosphonate ester, phosphate, phosphate ester, thiophosphate, thiophosphate ester, phosphoramidate, thiophosphoramidate, sulfonate, sulfonic acid, sulfate, hydroxamic acid, keto acid, amide and carboxylic acid. In some embodiments of any one of formulae (I)—(XVb), the hydrophilic head group is phosphonic acid, phosphonate, or a salt thereof. In some embodiments of any one of formulae (I)—(XVb), the hydrophilic head group is phosphate or a salt thereof. In some embodiments of any one of formulae (I)—(XVb), the hydrophilic head group is phosphonate ester or phosphate ester. In some embodiments of any one of formulae (I)—(XVb), the hydrophilic head group is a thiophosphate. In some embodiments of any one of formulae (I)—(XVb), the hydrophilic head group is a thiophosphate ester. In some embodiments of any one of formulae (I)—(XVb), the hydrophilic head group is a phosphoramidate. In some embodiments of any one of formulae (I)—(XVb), the hydrophilic head group is a thiophosphoramidate.

Particular examples of hydrophilic head groups of interest which can be incorporated into any one of the embodiments of formulae (I)—(XVb) described herein include, but are not limited to, a head group comprising a first moiety selected from phosphates (RPO₄H⁻), phosphonates (RPO₃H⁻), boric acid (RBO₂ H₂), carboxylates (RCO₂⁻), sulfates (RSO₄⁻), sulfonates (RSO₃⁻), amines (RNH₃⁺), glycerols, sugars such as lactose or derived from hyaluronic acid, polar amino acids, polyethylene oxides and oligoethyleneglycols, that is optionally conjugated to a residue of a second moiety selected from choline, ethanolamine, glycerol, nucleic acid, sugar, inositol, amino acid or amino acid ester (e.g., serine) and lipid (e.g., fatty acid or hydrocarbon chain, such as a C₈-C₃₀ saturated or unsaturated hydrocarbon). The head group may contain various other modifications, for instance, in the case of the oligoethyleneglycols and polyethylene oxide (PEG) containing head groups, such PEG chain may be terminated with a methyl group or have a distal functional group for further modification. Examples of hydrophilic head groups also include, but are not limited to, thiophosphate, phosphocholine, phosphoglycerol, phosphoethanolamine, phosphoserine, phosphoinositol, ethylphosphosphorylcholine, polyethyleneglycol, polyglycerol, melamine, glucosamine, trimethylamine, spermine, spermidine, and conjugated carboxylates, sulfates, boric acid, sulfonates, sulfates and carbohydrates.

In some instances of any one of formulae (I)—(XVb), the hydrophilic head group X¹ is of formula (XVI):

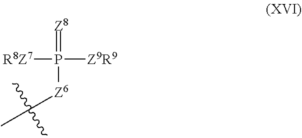

wherein:
Z⁶ is absent or selected from O and CH₂;
Z⁷ and Z⁹ are each independently selected from O and NR¹⁰ wherein R¹⁰ is H, alkyl or substituted alkyl;
Z⁸ is selected from O and S; and
R⁸ and R⁹ are each independently selected from H, alkyl, substituted alkyl, alkenyl, substituted alkenyl, aryl, substituted aryl, heteroaryl, substituted heteroaryl, acyl, substituted acyl, non-aromatic heterocycle, substituted non-aromatic heterocycle, cycloalkyl, substituted cycloalkyl and promoiety.

In some embodiments of formula (XVI), Z⁶ is absent. In other cases, Z⁶ is CH₂. In other cases, Z⁶ is oxygen. In some embodiments of formula (XVI), Z⁷ is oxygen and Z⁹ is NR¹⁰. In some cases, Z⁷ is NR¹⁰ and Z⁹ is oxygen. In some cases, both Z⁷ and Z⁹ are oxygen. In other cases, both Z⁷ and Z⁹ are NR¹⁰. In some cases, Z⁸ is oxygen. In other cases, Z⁸ is sulfur.

In some embodiments of formula (XVI), Z⁷, Z⁸ and Z⁹ are all oxygen atoms and Z⁶ is absent or CH₂. In other cases, Z⁸ is a sulfur atom. Z⁷ and Z⁹ are both oxygen atoms and Z⁶ is absent or CH₂. In other cases, Z⁸ is a sulfur atom, Z⁶, Z⁷ and Z⁹ are all oxygen atoms. In some cases, Z⁸ is an oxygen atom, Z⁷ is NR¹⁰, Z⁹ is an oxygen atom and Z⁶ is absent or CH₂. In other cases, Z⁸ is an oxygen atom, Z⁷ is NR¹⁰, Z⁶ and Z⁹ are both oxygen atoms. In other cases, Z⁸ is an oxygen atom, Z⁷ and Z⁹ are each independently NR¹⁰ and Z⁶ is an oxygen atom. In yet other cases, Z³¹ is an oxygen atom. Z⁷ and Z⁹ are each independently NR¹⁰ and Z⁶ is absent or CH₂. In some cases, Z⁷ and Z⁹ are each the same. In other cases, Z⁷ and Z⁹ are different. It is understood that the group of formula (XVI) may include one or more tautomeric forms of the structure depicted and that all such forms, and salts thereof, are meant to be included.

In some embodiments of formula (XVI), at least one of Z⁷ and Z⁹ is NR¹⁰. In some cases, R¹⁰ is hydrogen. In some cases, R¹⁰ is alkyl. In some other cases. R¹⁰ is substituted alkyl. In some cases, both Z⁷ and Z⁹ are NR¹⁰. In some cases, both Z⁷ and Z⁹ are NR¹⁰ and each R¹⁰, R⁸ and R⁹ are independently hydrogen. In some cases, both Z⁷ and Z⁹ are NR²², each R¹⁰ is an alkyl group, and R⁸ and R⁹ are each hydrogen. In some cases, both Z⁷ and Z⁹ are NR¹⁰, each R¹⁰ is a substituted alkyl group (e.g., an alkyl group substituted with an ester or a carboxyl group), and R⁸ and R⁹ are each hydrogen.

In some embodiments of formula (XVI). R⁸ and R⁹ are both hydrogen atoms. In some cases, at least one of R⁸ and R⁹ is a substituent other than hydrogen. In other cases, both R⁸ and R⁹ are substituents other than hydrogen. In some cases, at least one of R⁸ and R⁹ is an alkyl or substituted alkyl. In some cases, at least one of R⁸ and R⁹ is alkenyl or substituted alkenyl. In some other cases, at least one of R⁸ and R⁹ is aryl or substituted aryl. In some cases, at least one of R⁸ and R⁹ is acyl or substituted acyl. In some cases, at least one of R⁸ and R⁹ is heteroaryl or substituted heteroaryl. In some cases, at least one of R⁸ and R⁹ is cycloalkyl or substituted cycloalkyl. In some cases, R⁸ and R⁹ are both alkyl groups (e.g., lower alkyl). In some cases, R⁸ and R⁹ are both substituted alkyl groups (e.g., a C₍₁₋₆₎alkyl, substituted with alkoxy, substituted alkoxy, ester or carboxyl group). In some cases, at least one of R⁸ and R⁹ includes a promoiety. In certain cases, both R⁸ and R⁹ are phenyl groups. In some cases, R⁸ and R⁹ are the same. In other cases, R⁸ and R⁹ are different.

In some instances of any one of formulae (I)—(XVb), the hydrophilic head group X¹ is selected from any one of formulae (XVIa) to (XVIf):

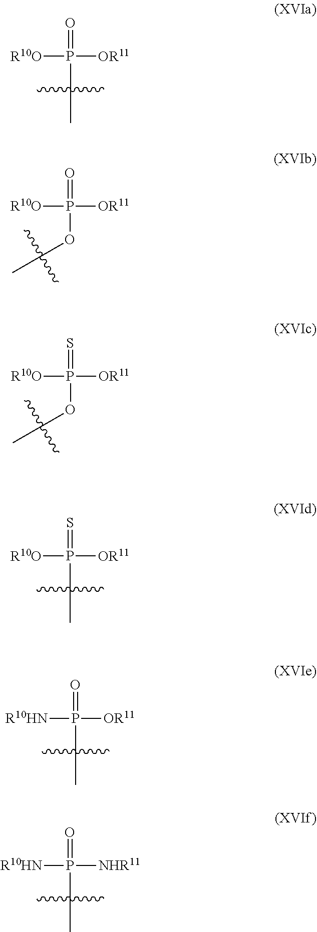

wherein:
R¹⁰ and R¹¹ are each independently selected from H, alkyl, substituted alkyl, alkoxy, substituted alkoxy, aryl, substituted aryl, heteroaryl, substituted heteroaryl, acyl, substituted acyl, carboxyl, substituted carboxyl and promoiety (e.g., as described herein).

In some embodiments of formulae (XVIa) to (XVIf). R¹⁰ and R¹¹ are both hydrogen atoms. In some cases, at least one of R¹⁰ and R¹¹ is a substituent other than hydrogen. In other cases, both R¹⁰ and R¹¹ are substituents other than hydrogen. In some cases, R¹⁰ and R¹¹ are the same. In other cases. R¹⁰ and R¹¹ are different. In some cases, at least one of R¹⁰ and R¹¹ is an alkyl or substituted alkyl. In some cases, at least one of R¹⁰ and R¹¹ is aryl or substituted aryl. In some cases, both of R¹⁰ and R¹¹ are alkyl or substituted alkyl. In some cases, both of R¹⁰ and R¹¹ are aryl or substituted aryl. In some cases, both of R¹⁰ and R¹¹ are acyl or substituted acyl. In some cases, R¹⁰ and R¹¹ are both lower alkyl groups. In some cases, R¹⁰ and R¹¹ are both substituted alkyl groups (e.g., a C₍₁₋₆₎alkyl, substituted with alkoxy, substituted alkoxy, ester or carboxyl group). In some cases, at least one of R¹⁰ and R¹¹ includes a promoiety. In certain cases, both R¹⁰ and R¹¹ are phenyl groups.

In certain instances of formulae (XVIa) to (XVId), at least one of R¹⁰ and R¹¹ includes a cleavable group or a self-immolative promoiety. A self-immolative group can be a disulfide linked promoiety or a self immolative ester containing promoiety. In some cases, R¹⁰ and/or R¹¹ includes a disulfide linked promoiety of formula: —CH₂CH₂—SS—R² where R² is alkyl or substituted alkyl. In certain instances. R¹² is a C₈-C₃₀ saturated or unsaturated hydrocarbon chain. In some cases, R¹⁰ and/or R¹¹ includes a promoiety of formula: —CH₂OCOR¹³ where R¹³ is H, alkyl or substituted alkyl. In some cases, R¹⁰ and/or R¹¹ includes a promoiety of formula: —CH₂C(R¹⁴)₂CO₂R¹⁴ where each R¹⁴ is independently H, alkyl or substituted alkyl.

In some instances of any one of formulae (I)—(XVb), the hydrophilic head group X¹, or prodrug form thereof, is selected from:

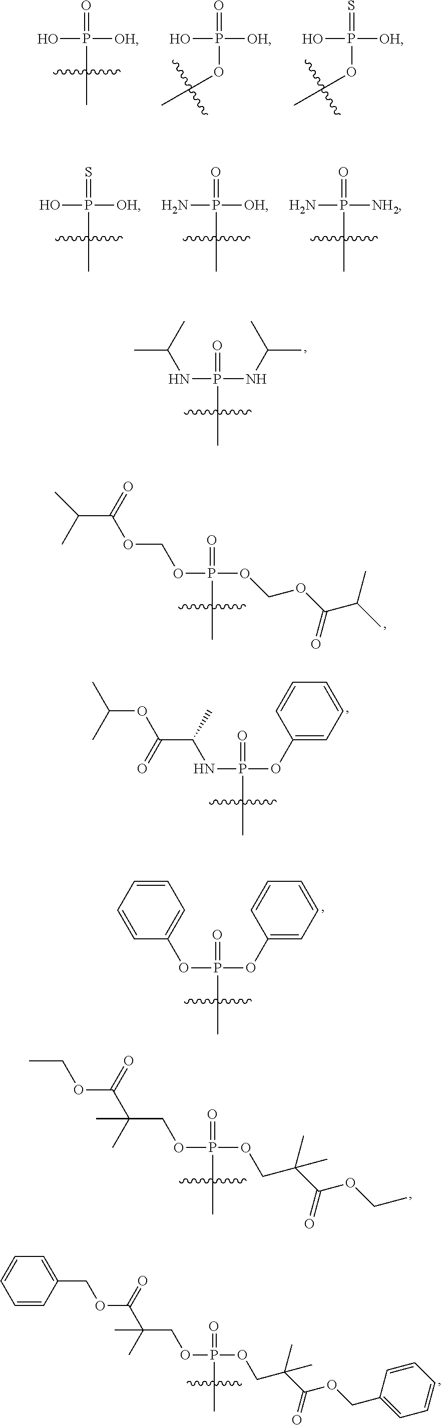

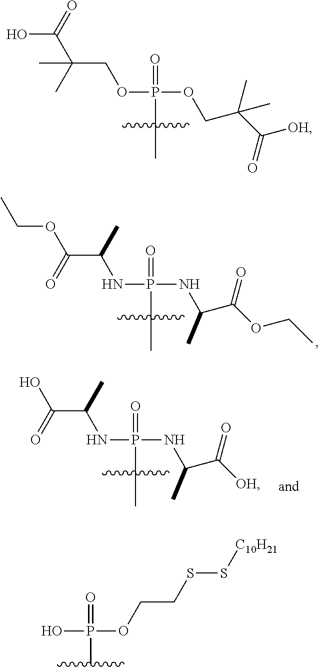

or a pharmaceutically acceptable salt thereof.

In some instances of any one of formulae (I)—(XVb), the hydrophilic head group X¹ is of the formula (XVI):

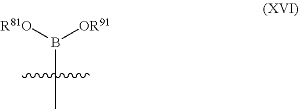

wherein:
R⁸¹ and R⁹¹ are each independently selected from H, alkyl, substituted alkyl, alkenyl, substituted alkenyl, alkoxy, substituted alkoxy, aryl, substituted aryl, an acyl group, an ester, an amide, heterocycle, substituted heterocycle cycloalkyl and substituted cycloalkyl or R⁸¹ and R⁹¹ together with the atoms to which they are attached form a group selected from heterocycle and substituted heterocycle.

In some embodiments of formula (XVI), R⁸¹ and R⁹¹ are both hydrogen atoms. In other cases, both R⁸¹ and R⁹¹ are substituents other than hydrogen.

In some instances of any one of formulae (I)—(XVb), the hydrophilic head group X¹ is of the formula (XVII):

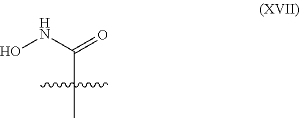 [SMILES: *C(=O)NO]

In some instances of any one of formulae (I)—(XVb), the hydrophilic head group X¹ is of the formula (XVIII):

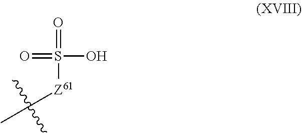

wherein:
Z⁶¹ is absent or selected from O and CH₂.

In some embodiments of formula (XVIII), the hydrophilic head group is selected from one of the following groups:

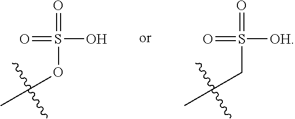 [SMILES: *OS(=O)(=O)O ; "or" ; *CS(=O)(=O)O]

In some instances of any one of formulae (I)—(XVb), the hydrophilic head group X¹ is of the formula (XIX):

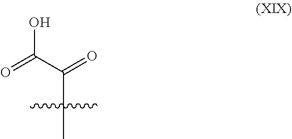 [SMILES: *C(=O)C(=O)O]

In some instances of any one of formulae (I)—(XVb), the hydrophilic head group X¹ is of the formula (XX):

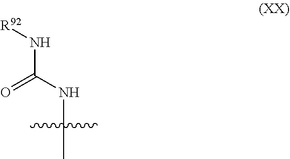

wherein:
R⁹² is selected from H, alkyl, substituted alkyl, alkenyl, substituted alkenyl, alkoxy, substituted alkoxy, aryl, substituted aryl, an acyl group, an ester, an amide, heterocycle, substituted heterocycle cycloalkyl and substituted cycloalkyl.

In some embodiments of formula (XX). R⁹² is hydrogen. In other cases. R⁹² is a substituent other than hydrogen. In certain embodiments, R⁹² is alkyl or substituted alkyl. In certain embodiments of formula (XX), the hydrophilic head group is of the structure:

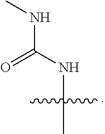 [SMILES: *NC(=O)NC]

In some instances of any one of formulae (I)—(XVb), the hydrophilic head group X¹ is of the formula (XXI):

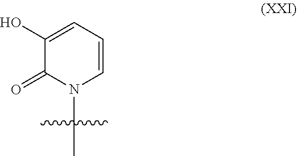 [SMILES: *n1cccc(O)c1=O]

It will be understood that any of the hydroxyl and amine groups in group X¹ of any of formulae (I)—(XVb) may be optionally further substituted with any convenient group, e.g., an alkyl group, a substituted alkyl group, a phenyl group, a substituted phenyl group, an ester group and the like. It will be understood that any convenient alternative hydrophilic group can be utilized as group X¹ in a compound of any of formulae (I)-XVb).

In certain embodiments, the ENPP1 inhibitor compound is described by one of the structures of Table 1, or a prodrug thereof (e.g., as described herein), or a pharmaceutically acceptable salt thereof.

TABLE 1
ENPP1 inhibitor compounds

| No. | Structure |
|---|---|
| 2 | 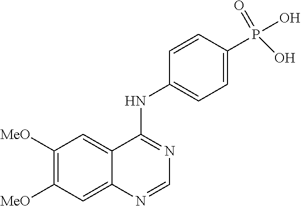 [SMILES: COc1cc2ncnc(Nc3ccc(P(=O)(O)O)cc3)c2cc1OC] |
| 3 | 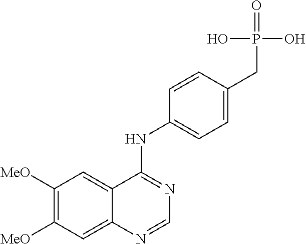 [SMILES: COc1cc2ncnc(Nc3ccc(CP(=O)(O)O)cc3)c2cc1OC] |
| 4 | 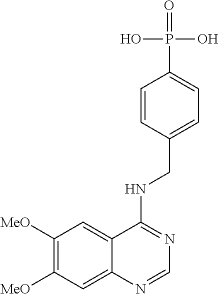 [SMILES: COc1cc2ncnc(NCc3ccc(P(=O)(O)O)cc3)c2cc1OC] |
| 5 | 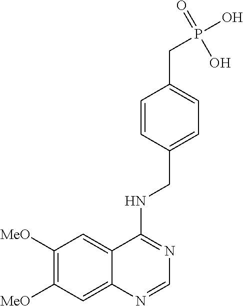 [SMILES: COc1cc2ncnc(NCc3ccc(CP(=O)(O)O)cc3)c2cc1OC] |
| 6 | 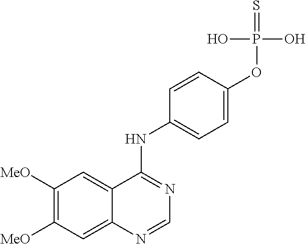 [SMILES: COc1cc2ncnc(Nc3ccc(OP(O)(O)=S)cc3)c2cc1OC] |
| 7 | 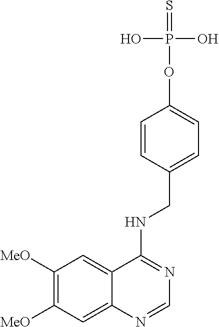 [SMILES: COc1cc2ncnc(NCc3ccc(OP(O)(O)=S)cc3)c2cc1OC] |
| 8 | 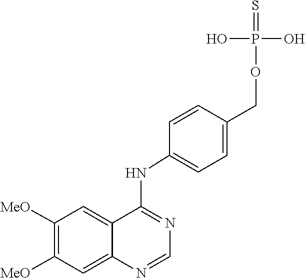 [SMILES: COc1cc2ncnc(Nc3ccc(COP(O)(O)=S)cc3)c2cc1OC] |
| 9 | 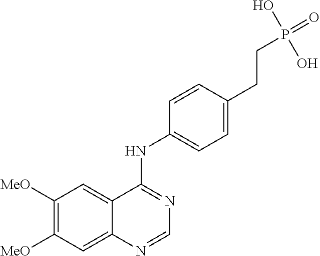 [SMILES: COc1cc2ncnc(Nc3ccc(CCP(=O)(O)O)cc3)c2cc1OC] |
| 10 | 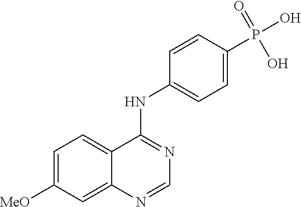 [SMILES: COc1ccc2c(Nc3ccc(P(=O)(O)O)cc3)ncnc2c1] |
| 11 | 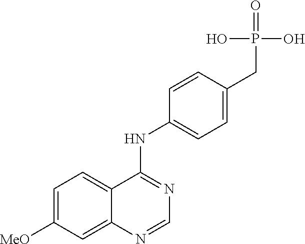 [SMILES: COc1ccc2c(Nc3ccc(CP(=O)(O)O)cc3)ncnc2c1] |
| 12 | 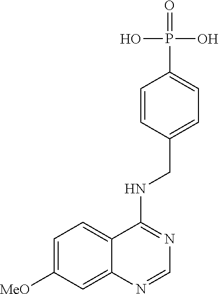 [SMILES: COc1ccc2c(NCc3ccc(P(=O)(O)O)cc3)ncnc2c1] |
| 13 | 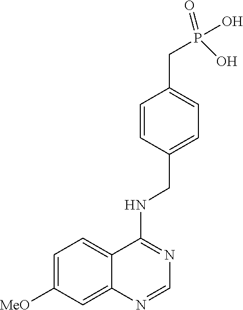 [SMILES: COc1ccc2c(NCc3ccc(CP(=O)(O)O)cc3)ncnc2c1] |
| 14 | 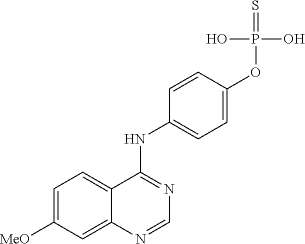 [SMILES: COc1ccc2c(Nc3ccc(OP(O)(O)=S)cc3)ncnc2c1] |
| 15 | 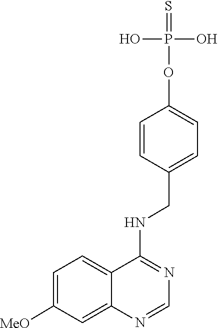 [SMILES: COc1ccc2c(NCc3ccc(OP(O)(O)=S)cc3)ncnc2c1] |
| 16 | 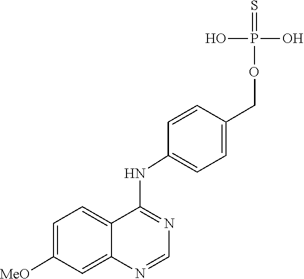 [SMILES: COc1ccc2c(Nc3ccc(COP(O)(O)=S)cc3)ncnc2c1] |
| 17 | 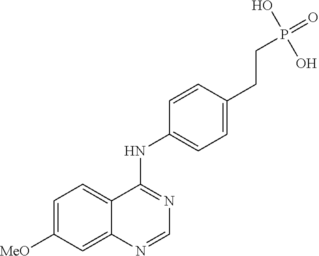 [SMILES: COc1ccc2c(Nc3ccc(CCP(=O)(O)O)cc3)ncnc2c1] |
| 18 | 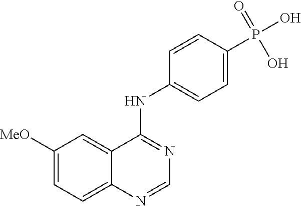 [SMILES: COc1ccc2ncnc(Nc3ccc(P(=O)(O)O)cc3)c2c1] |
| 19 | 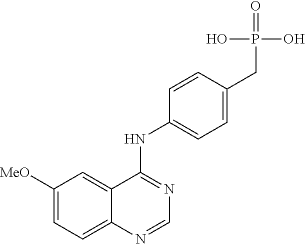 [SMILES: COc1ccc2ncnc(Nc3ccc(CP(=O)(O)O)cc3)c2c1] |
| 20 | 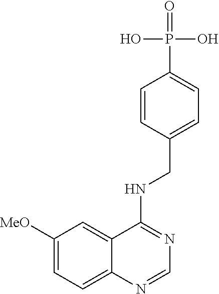 [SMILES: COc1ccc2ncnc(NCc3ccc(P(=O)(O)O)cc3)c2c1] |
| 21 | 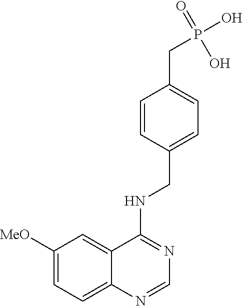 [SMILES: COc1ccc2ncnc(NCc3ccc(CP(=O)(O)O)cc3)c2c1] |
| 22 | 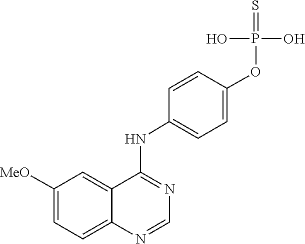 [SMILES: COc1ccc2ncnc(Nc3ccc(OP(O)(O)=S)cc3)c2c1] |
| 23 | 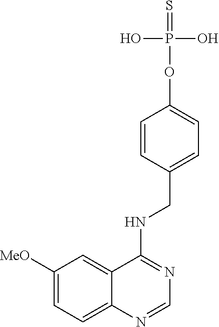 [SMILES: COc1ccc2ncnc(NCc3ccc(OP(O)(O)=S)cc3)c2c1] |
| 24 | 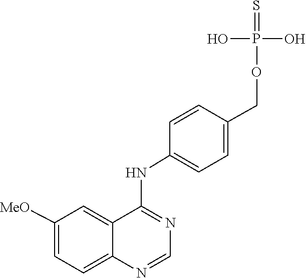 [SMILES: COc1ccc2ncnc(Nc3ccc(COP(O)(O)=S)cc3)c2c1] |
| 25 | 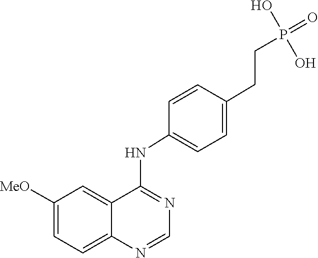 [SMILES: COc1ccc2ncnc(Nc3ccc(CCP(=O)(O)O)cc3)c2c1] |
| 26 | 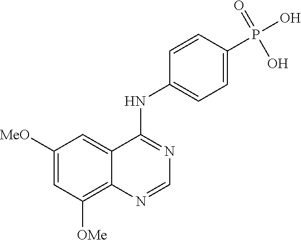 [SMILES: COc1cc(OC)c2ncnc(Nc3ccc(P(=O)(O)O)cc3)c2c1] |
| 27 | 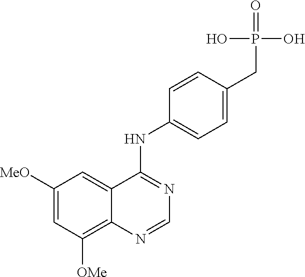 [SMILES: COc1cc(OC)c2ncnc(Nc3ccc(CP(=O)(O)O)cc3)c2c1] |
| 28 | 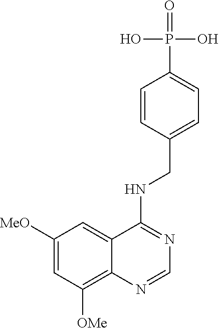 [SMILES: COc1cc(OC)c2ncnc(NCc3ccc(P(=O)(O)O)cc3)c2c1] |
| 29 | 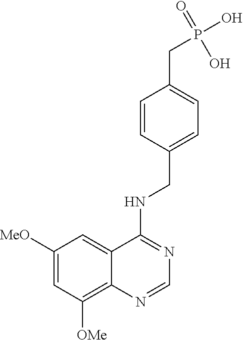 [SMILES: COc1cc(OC)c2ncnc(NCc3ccc(CP(=O)(O)O)cc3)c2c1] |
| 30 | 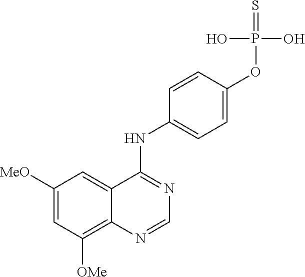 [SMILES: COc1cc(OC)c2ncnc(Nc3ccc(OP(O)(O)=S)cc3)c2c1] |
| 31 | 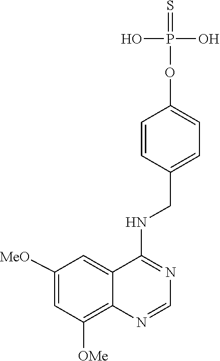 [SMILES: COc1cc(OC)c2ncnc(NCc3ccc(OP(O)(O)=S)cc3)c2c1] |
| 32 | 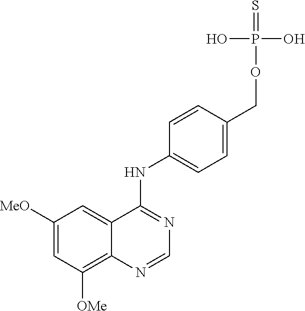 [SMILES: COc1cc(OC)c2ncnc(Nc3ccc(COP(O)(O)=S)cc3)c2c1] |
| 33 | 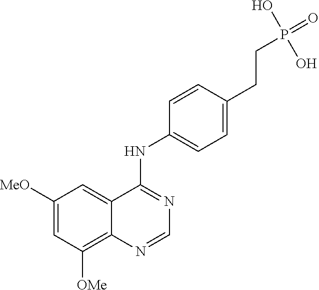 [SMILES: COc1cc(OC)c2ncnc(Nc3ccc(CCP(=O)(O)O)cc3)c2c1] |
| 34 | 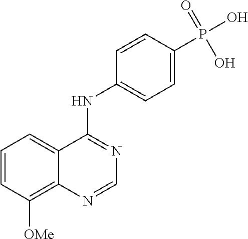 [SMILES: COc1cccc2c(Nc3ccc(P(=O)(O)O)cc3)ncnc12] |
| 35 | 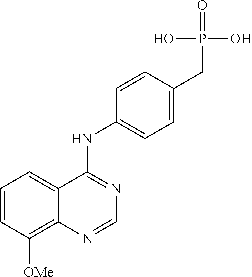 [SMILES: COc1cccc2c(Nc3ccc(CP(=O)(O)O)cc3)ncnc12] |
| 36 | 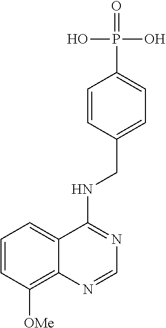 [SMILES: COc1cccc2c(NCc3ccc(P(=O)(O)O)cc3)ncnc12] |
| 37 | 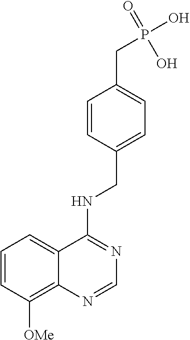 [SMILES: COc1cccc2c(NCc3ccc(CP(=O)(O)O)cc3)ncnc12] |
| 38 | 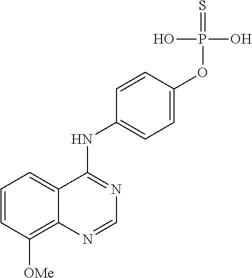 [SMILES: COc1cccc2c(Nc3ccc(OP(O)(O)=S)cc3)ncnc12] |
| 39 | 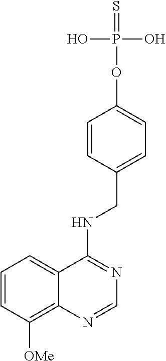 [SMILES: COc1cccc2c(NCc3ccc(OP(O)(O)=S)cc3)ncnc12] |
| 40 | 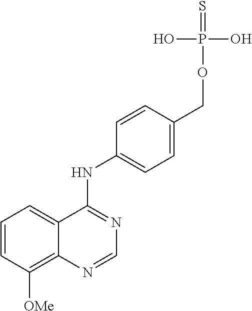 [SMILES: COc1cccc2c(Nc3ccc(COP(O)(O)=S)cc3)ncnc12] |
| 41 | 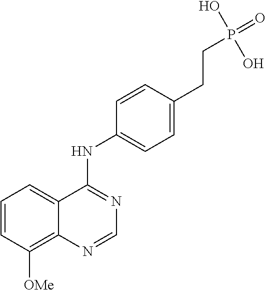 [SMILES: COc1cccc2c(Nc3ccc(CCP(=O)(O)O)cc3)ncnc12] |
| 52 | 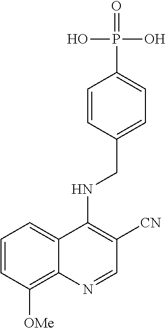 [SMILES: COc1cccc2c(NCc3ccc(P(=O)(O)O)cc3)c(C#N)cnc12] |
| 53 | 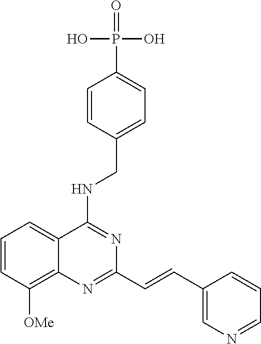 [SMILES: COc1cccc2c(NCc3ccc(P(=O)(O)O)cc3)nc(/C=C/c3cccnc3)nc12] |
| 101 | 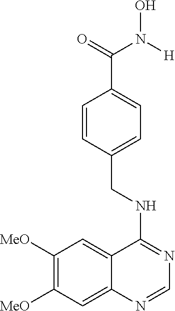 [SMILES: COc1cc2ncnc(NCc3ccc(C(=O)NO)cc3)c2cc1OC] |
| 102 | 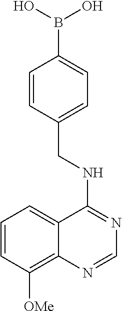 [SMILES: COc1cccc2c(NCc3ccc(B(O)O)cc3)ncnc12] |
| 103 | 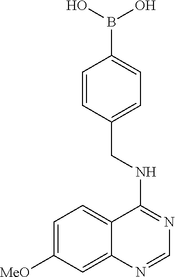 [SMILES: COc1ccc2c(NCc3ccc(B(O)O)cc3)ncnc2c1] |
| 104 | 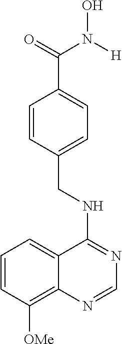 [SMILES: COc1cccc2c(NCc3ccc(C(=O)NO)cc3)ncnc12] |

In certain embodiments, the ENPP1 inhibitor compound is described by one of the structures of Table 2, or a prodrug thereof (e.g., as described herein), or a pharmaceutically acceptable salt thereof.

TABLE 2
ENPP1 inhibitor compounds

| No. | Structure |
|---|---|
| 42 | 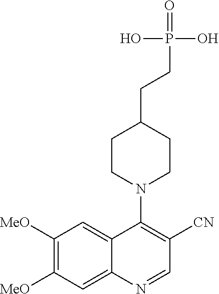 [SMILES: COc1cc2ncc(C#N)c(N3CCC(CCP(=O)(O)O)CC3)c2cc1OC] |
| 43 | 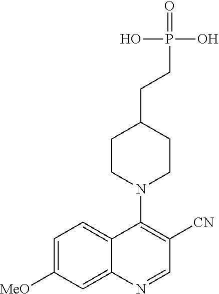 [SMILES: COc1ccc2c(N3CCC(CCP(=O)(O)O)CC3)c(C#N)cnc2c1] |
| 44 | 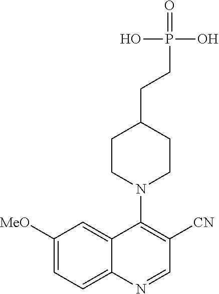 [SMILES: COc1ccc2ncc(C#N)c(N3CCC(CCP(=O)(O)O)CC3)c2c1] |
| 45 | 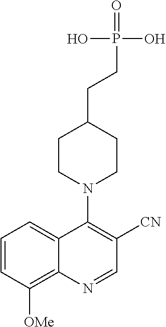 [SMILES: COc1cccc2c(N3CCC(CCP(=O)(O)O)CC3)c(C#N)cnc12] |
| 46 | 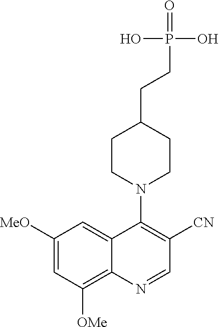 [SMILES: COc1cc(OC)c2ncc(C#N)c(N3CCC(CCP(=O)(O)O)CC3)c2c1] |
| 47 | 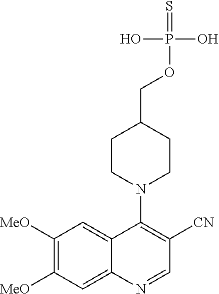 [SMILES: COc1cc2ncc(C#N)c(N3CCC(COP(O)(O)=S)CC3)c2cc1OC] |
| 48 | 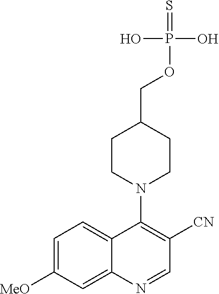 [SMILES: COc1ccc2c(N3CCC(COP(O)(O)=S)CC3)c(C#N)cnc2c1] |
| 49 | 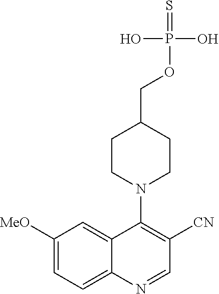 [SMILES: COc1ccc2ncc(C#N)c(N3CCC(COP(O)(O)=S)CC3)c2c1] |
| 50 | 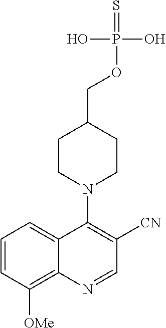 [SMILES: COc1cccc2c(N3CCC(COP(O)(O)=S)CC3)c(C#N)cnc12] |
| 51 | 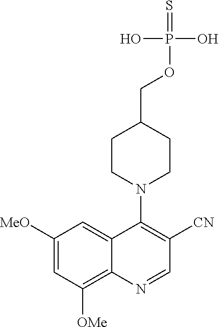 [SMILES: COc1cc(OC)c2ncc(C#N)c(N3CCC(COP(O)(O)=S)CC3)c2c1] |
| 201 | 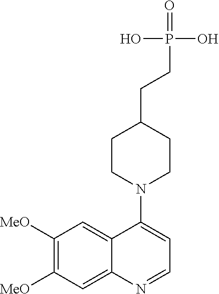 [SMILES: COc1cc2nccc(N3CCC(CCP(=O)(O)O)CC3)c2cc1OC] |
| 202 | 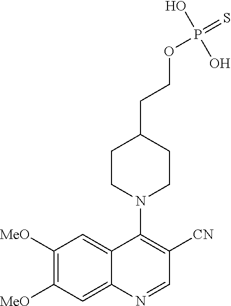 [SMILES: COc1cc2ncc(C#N)c(N3CCC(CCOP(O)(O)=S)CC3)c2cc1OC] |
| 203 | 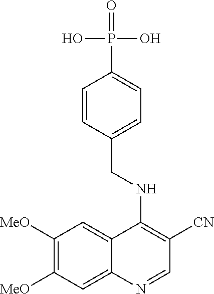 [SMILES: COc1cc2ncc(C#N)c(NCc3ccc(P(=O)(O)O)cc3)c2cc1OC] |
| 204 | 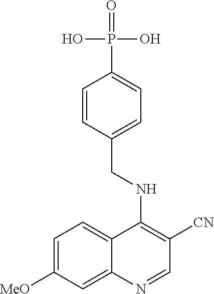 [SMILES: COc1ccc2c(NCc3ccc(P(=O)(O)O)cc3)c(C#N)cnc2c1] |
| 205 | 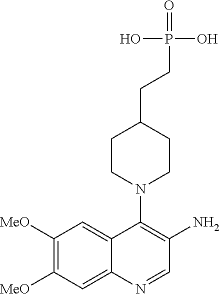 [SMILES: COc1cc2ncc(N)c(N3CCC(CCP(=O)(O)O)CC3)c2cc1OC] |
| 206 | 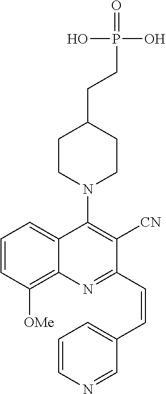 [SMILES: COc1cccc2c(N3CCC(CCP(=O)(O)O)CC3)c(C#N)c(/C=C\c3cccnc3)nc12] |
| 207 | 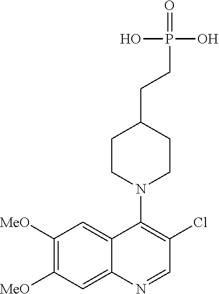 [SMILES: COc1cc2ncc(Cl)c(N3CCC(CCP(=O)(O)O)CC3)c2cc1OC] |
| 208 | 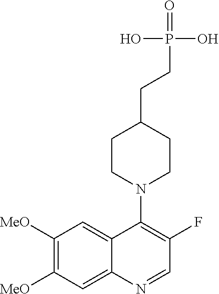 [SMILES: COc1cc2ncc(F)c(N3CCC(CCP(=O)(O)O)CC3)c2cc1OC] |
| 209 | 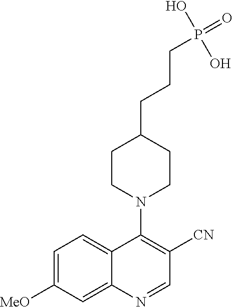 [SMILES: COc1ccc2c(N3CCC(CCCP(=O)(O)O)CC3)c(C#N)cnc2c1] |
| 210 | 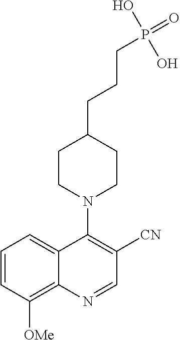 [SMILES: COc1cccc2c(N3CCC(CCCP(=O)(O)O)CC3)c(C#N)cnc12] |
| 211 | 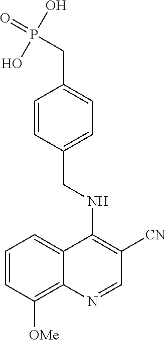 [SMILES: COc1cccc2c(NCc3ccc(CP(=O)(O)O)cc3)c(C#N)cnc12] |
| 212 | 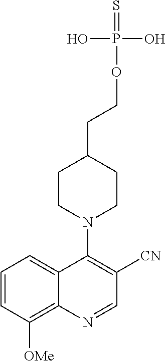 [SMILES: COc1cccc2c(N3CCC(CCOP(O)(O)=S)CC3)c(C#N)cnc12] |
| 213 | 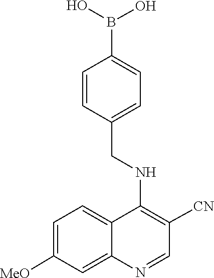 [SMILES: COc1ccc2c(NCc3ccc(B(O)O)cc3)c(C#N)cnc2c1] |
| 214 | 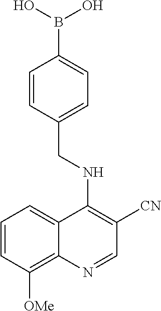 [SMILES: COc1cccc2c(NCc3ccc(B(O)O)cc3)c(C#N)cnc12] |
| 215 | 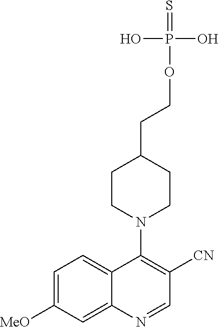 [SMILES: COc1ccc2c(N3CCC(CCOP(O)(O)=S)CC3)c(C#N)cnc2c1] |
| 216 | 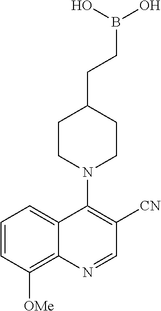 [SMILES: COc1cccc2c(N3CCC(CCB(O)O)CC3)c(C#N)cnc12] |
| 217 | 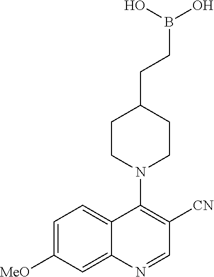 [SMILES: COc1ccc2c(N3CCC(CCB(O)O)CC3)c(C#N)cnc2c1] |
| 218 | 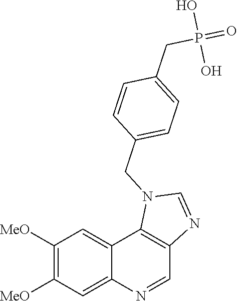 [SMILES: COc1cc2ncc3ncn(Cc4ccc(CP(=O)(O)O)cc4)c3c2cc1OC] |
| 219 | 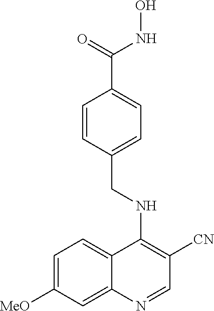 [SMILES: COc1ccc2c(NCc3ccc(C(=O)NO)cc3)c(C#N)cnc2c1] |
| 220 | 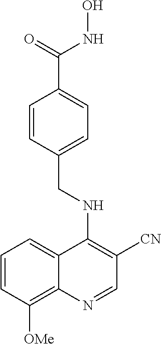 [SMILES: COc1cccc2c(NCc3ccc(C(=O)NO)cc3)c(C#N)cnc12] |
| 221 | 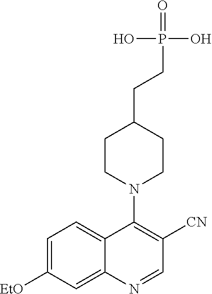 [SMILES: CCOc1ccc2c(N3CCC(CCP(=O)(O)O)CC3)c(C#N)cnc2c1] |
| 222 | 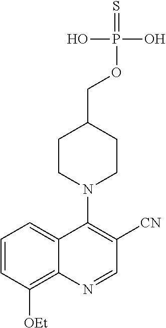 [SMILES: CCOc1cccc2c(N3CCC(COP(O)(O)=S)CC3)c(C#N)cnc12] |
| 223 | 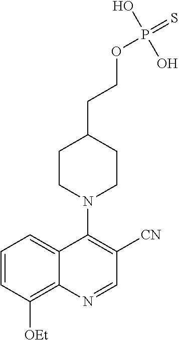 [SMILES: CCOc1cccc2c(N3CCC(CCOP(O)(O)=S)CC3)c(C#N)cnc12] |
| 224 | 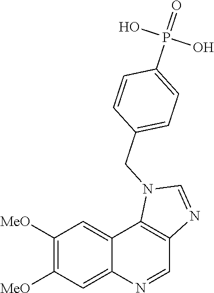 [SMILES: COc1cc2ncc3ncn(Cc4ccc(P(=O)(O)O)cc4)c3c2cc1OC] |
| 225 | 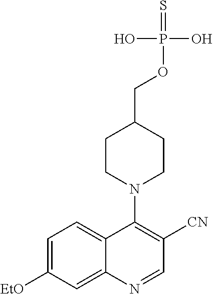 [SMILES: CCOc1ccc2c(N3CCC(COP(O)(O)=S)CC3)c(C#N)cnc2c1] |
| 226 | 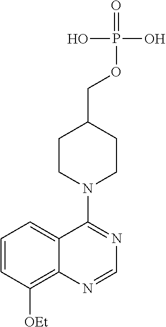 [SMILES: CCOc1cccc2c(N3CCC(COP(=O)(O)O)CC3)ncnc12] |
| | 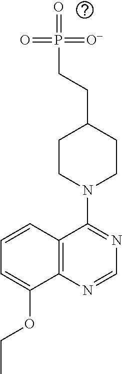 |

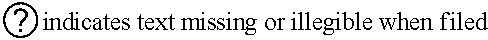

In certain embodiments, the ENPP1 inhibitor compound is described by one of the structures of Table 3, or a prodrug thereof (e.g., as described herein), or a pharmaceutically acceptable salt thereof.

TABLE 3
ENPP1 inhibitor compounds

| No. | Structure |
|---|---|
| 54 | 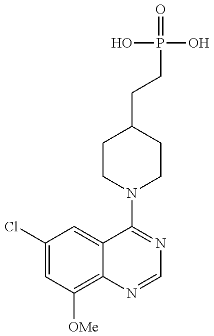 [SMILES: COc1cc(Cl)cc2c(N3CCC(CCP(=O)(O)O)CC3)ncnc12] |
| 55 | 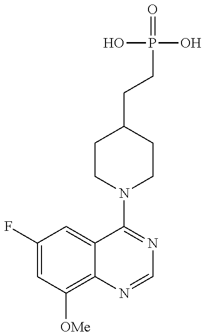 [SMILES: COc1cc(F)cc2c(N3CCC(CCP(=O)(O)O)CC3)ncnc12] |
| 56 | 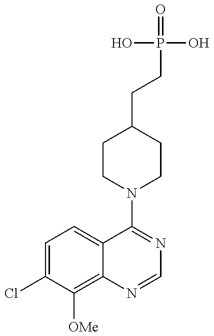 [SMILES: COc1c(Cl)ccc2c(N3CCC(CCP(=O)(O)O)CC3)ncnc12] |
| 57 | 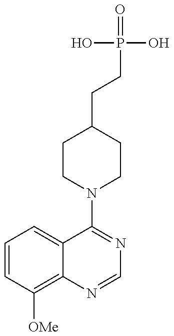 [SMILES: COc1cccc2c(N3CCC(CCP(=O)(O)O)CC3)ncnc12] |
| 58 | 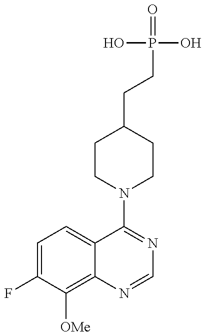 [SMILES: COc1c(F)ccc2c(N3CCC(CCP(=O)(O)O)CC3)ncnc12] |
| 59 | 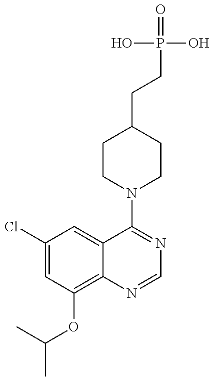 [SMILES: CC(C)Oc1cc(Cl)cc2c(N3CCC(CCP(=O)(O)O)CC3)ncnc12] |
| 60 | 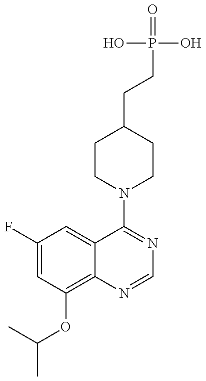 [SMILES: CC(C)Oc1cc(F)cc2c(N3CCC(CCP(=O)(O)O)CC3)ncnc12] |
| 61 | 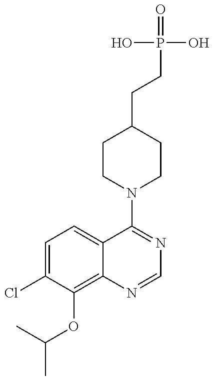 [SMILES: CC(C)Oc1c(Cl)ccc2c(N3CCC(CCP(=O)(O)O)CC3)ncnc12] |
| 62 | 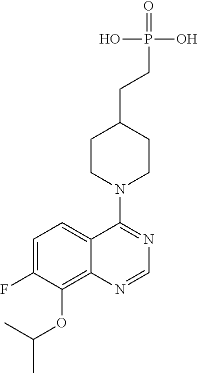 [SMILES: CC(C)Oc1c(F)ccc2c(N3CCC(CCP(=O)(O)O)CC3)ncnc12] |
| 63 | 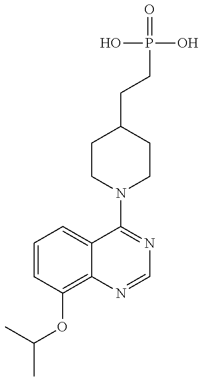 [SMILES: CC(C)Oc1cccc2c(N3CCC(CCP(=O)(O)O)CC3)ncnc12] |

In certain embodiments, the compound is described by the structure of one of the compounds of Tables 1-3 (herein, reference to Tables 1-3 includes Table 3a). It is understood that any of the compounds shown in Tables 1-3 may be present in a salt form. In some cases, the salt form of the compound is a pharmaceutically acceptable salt. It is understood that any of the compounds shown in Tables 1-3 may be present in a prodrug form.

In some embodiments, the compound is described by the structure of one of the compounds of Table 3a.

TABLE 3a

| | |
|---|---|
| 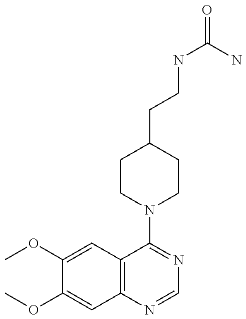 [SMILES: COc1cc2ncnc(N3CCC(CCNC(N)=O)CC3)c2cc1OC] | 4 |
| 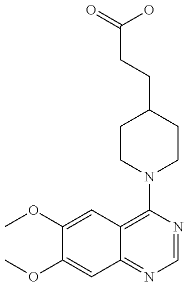 [SMILES: COc1cc2ncnc(N3CCC(CCC(=O)O)CC3)c2cc1OC] | 6 |
| 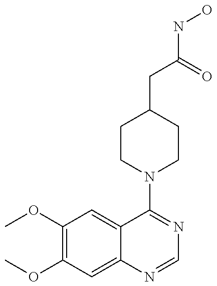 [SMILES: COc1cc2ncnc(N3CCC(CC(=O)NO)CC3)c2cc1OC] | AA |
| 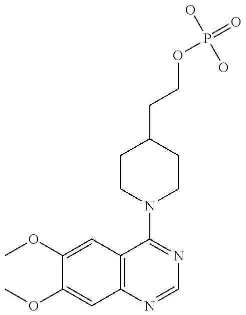 [SMILES: COc1cc2ncnc(N3CCC(CCOP(=O)(O)O)CC3)c2cc1OC] | 10 |
| 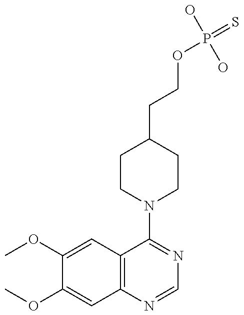 [SMILES: COc1cc2ncnc(N3CCC(CCOP(O)(O)=S)CC3)c2cc1OC] | 12 |
| 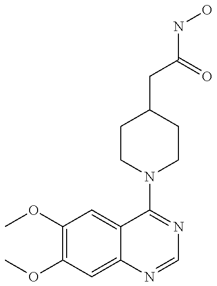 [SMILES: COc1cc2ncnc(N3CCC(CC(=O)NO)CC3)c2cc1OC] | 8 |
| 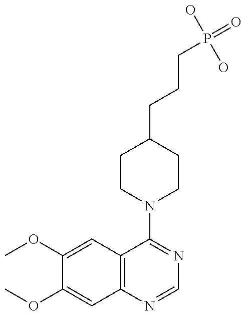 [SMILES: COc1cc2ncnc(N3CCC(CCCP(=O)(O)O)CC3)c2cc1OC] | 14 |
| 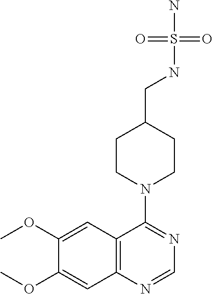 [SMILES: COc1cc2ncnc(N3CCC(CNS(N)(=O)=O)CC3)c2cc1OC] | BB |
| 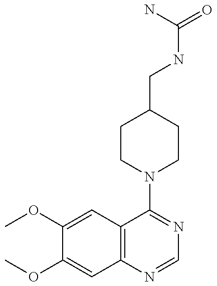 [SMILES: COc1cc2ncnc(N3CCC(CNC(N)=O)CC3)c2cc1OC] | 5 |
| 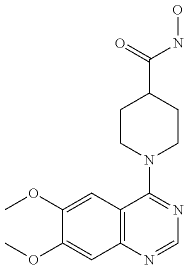 [SMILES: COc1cc2ncnc(N3CCC(C(=O)NO)CC3)c2cc1OC] | 9 |
| 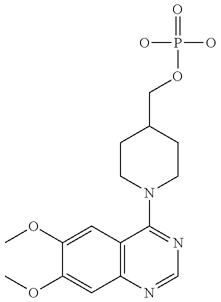 [SMILES: COc1cc2ncnc(N3CCC(COP(=O)(O)O)CC3)c2cc1OC] | 11 |
| 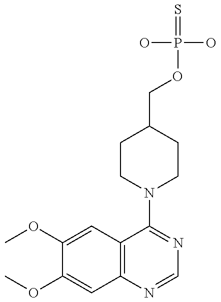 [SMILES: COc1cc2ncnc(N3CCC(COP(O)(O)=S)CC3)c2cc1OC] | 13 |
| 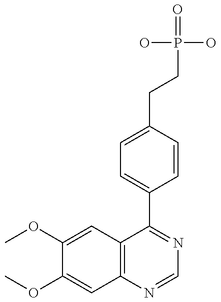 [SMILES: COc1cc2ncnc(-c3ccc(CCP(=O)(O)O)cc3)c2cc1OC] | 20 |
| 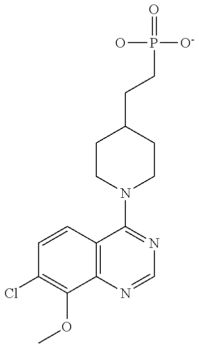 [SMILES: COc1c(Cl)ccc2c(N3CCC(CCP(=O)([O-])O)CC3)ncnc12] | 43 |
| 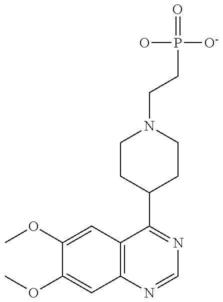 [SMILES: COc1cc2ncnc(C3CCN(CCP(=O)([O-])O)CC3)c2cc1OC] | 18 |
| 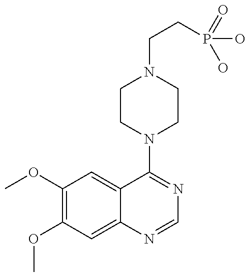 [SMILES: COc1cc2ncnc(N3CCN(CCP(=O)(O)O)CC3)c2cc1OC] | 19 |
| 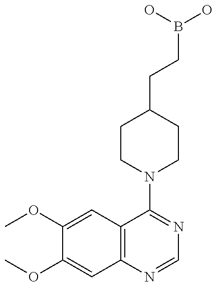 [SMILES: COc1cc2ncnc(N3CCC(CCB(O)O)CC3)c2cc1OC] | 7 |
| 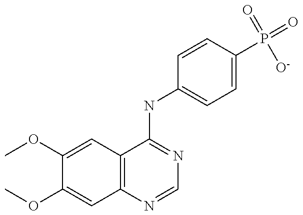 | 21 |
| 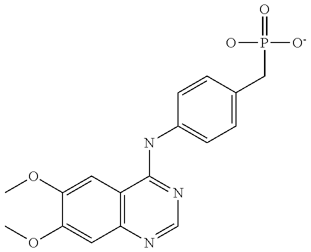 [SMILES: COc1cc2ncnc(Nc3ccc(CP(=O)([O-])O)cc3)c2cc1OC] | 22 |
| 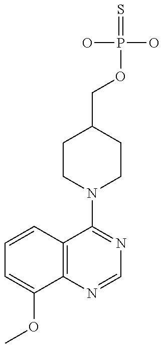 [SMILES: COc1cccc2c(N3CCC(COP(O)(O)=S)CC3)ncnc12] | 53 |
| 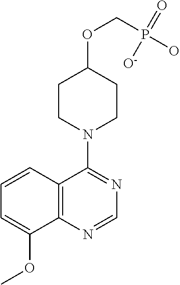 [SMILES: COc1cccc2c(N3CCC(OCP(=O)([O-])O)CC3)ncnc12] | 54 |
| 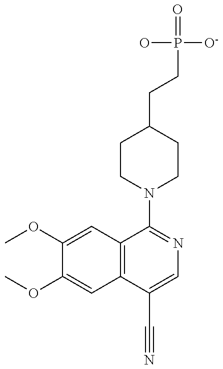 [SMILES: COc1cc2c(C#N)cnc(N3CCC(CCP(=O)([O-])O)CC3)c2cc1OC] | 52 |
| 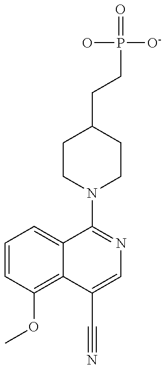 [SMILES: COc1cccc2c(N3CCC(CCP(=O)([O-])O)CC3)ncc(C#N)c12] | CC |
| 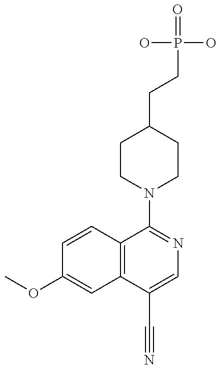 [SMILES: COc1ccc2c(N3CCC(CCP(=O)(O)O)CC3)ncc(C#N)c2c1] | DD |
| 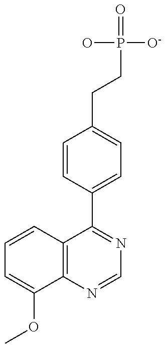 [SMILES: COc1cccc2c(-c3ccc(CCP(=O)([O-])O)cc3)ncnc12] | 55 |
| 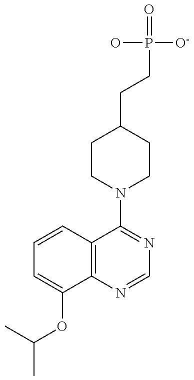 [SMILES: CC(C)Oc1cccc2c(N3CCC(CCP(=O)([O-])O)CC3)ncnc12] | 34 |
| 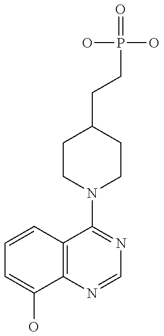 [SMILES: O=P(O)(O)CCC1CCN(c2ncnc3c(O)cccc23)CC1] | 36 |
| 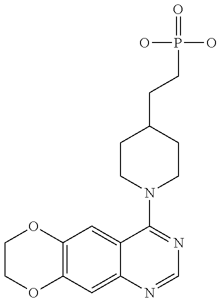 [SMILES: O=P(O)(O)CCC1CCN(c2ncnc3cc4c(cc23)OCCO4)CC1] | 39 |
| 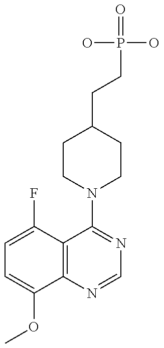 [SMILES: COc1ccc(F)c2c(N3CCC(CCP(=O)(O)O)CC3)ncnc12] | 40 |
| 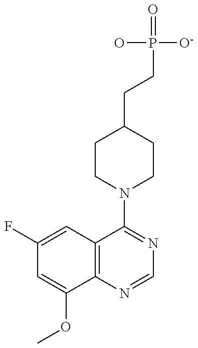 [SMILES: COc1cc(F)cc2c(N3CCC(CCP(=O)([O-])O)CC3)ncnc12] | 41 |
| 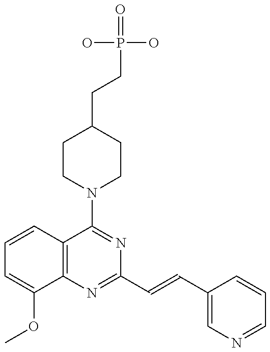 [SMILES: COc1cccc2c(N3CCC(CCP(=O)(O)O)CC3)nc(/C=C/c3cccnc3)nc12] | 45 |
| 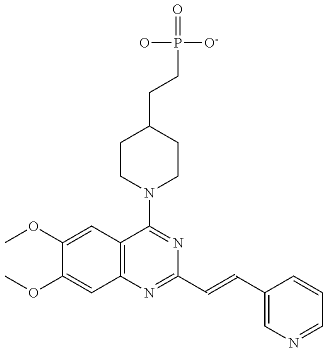 [SMILES: COc1cc2nc(/C=C/c3cccnc3)nc(N3CCC(CCP(=O)([O-])O)CC3)c2cc1OC] | 44 |
| 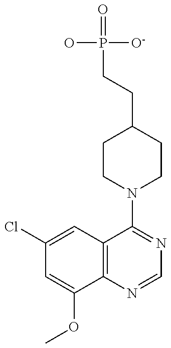 [SMILES: COc1cc(Cl)cc2c(N3CCC(CCP(=O)([O-])O)CC3)ncnc12] | 42 |

Aspects of the present disclosure include ENPP1 inhibitor compounds (e.g., as described herein), salts thereof (e.g., pharmaceutically acceptable salts), and/or solvate, hydrate and/or prodrug forms thereof. In addition, it is understood that, in any compound described herein having one or more chiral centers, if an absolute stereochemistry is not expressly indicated, then each center may independently be of R-configuration or S-configuration or a mixture thereof. It will be appreciated that all permutations of salts, solvates, hydrates, prodrugs and stereoisomers are meant to be encompassed by the present disclosure.

In some embodiments, the subject ENPP1 inhibitor compounds, or a prodrug form thereof, are provided in the form of pharmaceutically acceptable salts. Compounds containing an amine or nitrogen containing heteroaryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

In some embodiments, the subject compounds are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in viva. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)). In some cases, the promoiety is attached to a hydrophilic head group of the subject compounds. In some cases, the promoiety is attached to a hydroxy or carboxylic acid group of the subject compounds. In certain cases, the promoiety is an acyl or substituted acyl group. In certain cases, the promoiety is an alkyl or substituted alkyl group, e.g., that forms an ester functional group when attached to a hydrophilic head group of the subject compounds, e.g., a phosphonate ester, a phosphate ester, etc.

In some embodiments, the subject compound is a phosphonate ester or phosphate ester prodrug that can be transformed to a compound including a phosphonic acid or phosphonate, or a phosphate head group.

In some embodiments, the subject compounds, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, the subject compounds are provided by oral dosing and absorbed into the bloodstream. In some embodiments, the oral bioavailability of the subject compounds is 30% or more. Modifications may be made to the subject compounds or their formulations using any convenient methods to increase absorption across the gut lumen or their bioavailability.

In some embodiments, the subject compounds are metabolically stable (e.g., remain substantially intact in vivo during the half-life of the compound). In certain embodiments, the compounds have a half-life (e.g., an in vivo half-life) of 5 minutes or more, such as 10 minutes or more, 12 minutes or more, 15 minutes or more, 20 minutes or more, 30 minutes or more, 60 minutes or more, 2 hours or more, 6 hours or more, 12 hours or more, 24 hours or more, or even more.

### Methods of Inhibiting ENPP1

As summarized above, aspects of the present disclosure include ENPP1 inhibitors, and methods of inhibition using the same. ENPP1 is a member of the ecto-nucleotide pyrophosphatase/phosphodiesterase (ENPP) family. As such, aspects of the subject methods include inhibition of the hydrolase activity of ENPP1 against cGAMP. The inventors discovered that cGAMP can have significant extracellular biological functions, which can be enhanced by blocking extracellular degradation of cGAMP, e.g., hydrolysis by its degradation enzyme ENPP1. In certain instances, the ENPP1 target of inhibition is extracellular, and the subject ENPP1 inhibiting compounds are cell-impermeable, and thus are not capable of diffusion into cells. As such, the subject methods can provide for selective extracellular inhibition of ENPP1's hydrolase activity and increased extracellular levels of cGAMP. As such, in some cases, the ENPP1 inhibiting compounds are compounds that inhibit the activity of ENPP1 extracellularly. Experiments conducted by the inventors indicate that inhibiting the activity of ENPP1 increases extracellular cGAMP and may consequently boost the STING pathway.

By inhibiting an ENPP1 it is meant that the activity of the enzyme is decreased by 10% or more, such as 20% or more, 30% or more, 40% or more, 50% or more, 60% or more, 70% or more, 80% or more, 90% or more, 95% or more (e.g., relative to a control in any convenient in vitro inhibition assay). In some cases, inhibiting an ENPP1 means decreasing the activity of the enzyme by a factor of 2 or more, such as 3 or more, 5 or more, 10 or more, 100 or more, or 1000 or more, relative to its normal activity (e.g., relative to a control as measured by any convenient assay).

In some cases, the method is a method of inhibiting ENPP1 in a sample. The term “sample” as used herein relates to a material or mixture of materials, typically, although not necessarily, in fluid form, containing one or more components of interest.

In some embodiments, there is provided a method of inhibiting ENPP1, the method comprising contacting a sample with a cell impermeable ENPP1 inhibitor to inhibit cGAMP hydrolysis activity of ENPP1. In some cases, the sample is a cellular sample. In some cases, the sample comprises cGAMP. In certain cases, the cGAMP levels are elevated in the cellular sample (e.g., relative to a control sample not contacted with the inhibitor). The subject methods can provide for increased levels of cGAMP. By “increased level of cGAMP” is meant a level of cGAMP in a cellular sample contacted with a subject compound, where the cGAMP level in the sample is increased by 10% or more, such as 20% or more, 30% or more, 40% or more, 50% or more, 60% or more, 70% or more, 80% or more, 90% or more, 100% or more, or even more, relative to a control sample that is not contacted with the agent.

In certain embodiments the ENPP1 inhibitor is an inhibitor as defined herein. In some embodiments, the ENPP1 inhibitor is an inhibitor according to any one of formulae (I)—(XVb) (e.g., as described herein). In some cases, the ENPP1 inhibitor is any one of compounds of Tables 1-3 (e.g., as described herein). In some cases, the ENPP1 inhibitor is cell impermeable.

In some embodiments the ENPP1 inhibitor is configured to be cell permeable. In some embodiments, there is provided a method of inhibiting ENPP1, the method comprising contacting a sample with a cell permeable ENPP1 inhibitor to inhibit ENPP1.

In some embodiments, the subject compounds have an ENPP1 inhibition profile that reflects activity against additional enzymes. In some embodiments, the subject compounds specifically inhibit ENPP1 without undesired inhibition of one or more other enzymes.

In some embodiments, the compounds of the disclosure interfere with the interaction of cGAMP and ENPP1. For example, the subject compounds may act to increase the extracellular cGAMP by inhibiting the hydrolase activity of ENPP1 against cGAMP. Without being bound to any particular theory, it is thought that increasing extracellular cGAMP activates the STING pathway.

In some embodiments, the subject compounds inhibit ENPP1, as determined by an inhibition assay, e.g., by an assay that determines the level of activity of the enzyme either in a cell-free system or in a cell after treatment with a subject compound, relative to a control, by measuring the IC₅₀ or EC₅₀ value, respectively. In certain embodiments, the subject compounds have an IC⁵⁰ value (or EC₅₀ value) of 10 μM or less, such as 3 μM or less, 1 μM or less, 500 nM or less, 300 nM or less, 200 nM or less, 100 nM or less, 50 nM or less, 30 nM or less, 10 nM or less, 5 nM or less, 3 nM or less, 1 nM or less, or even lower.

As summarized above, aspects of the disclosure include methods of inhibiting ENPP1. A subject compound (e.g., as described herein) may inhibit at activity of ENPP1 in the range of 10% to 100%, e.g., by 10% or more, 20% or more, 30% or more, 40% or more, 50% or more, 60% or more, 70% or more, 80% or more, or 90% or more. In certain assays, a subject compound may inhibit its target with an IC₅₀ of 1×10⁻⁶ M or less (e.g., 1×10⁻⁶ M or less, 1×10⁻⁷ M or less, 1×10⁻⁸ M or less, 1×10⁻⁹ M or less, 1×10⁻¹⁰ M or less, or 1×10⁻¹¹ M or less).

The protocols that may be employed in determining ENPP1 activity are numerous, and include but are not limited to cell-free assays, e.g., binding assays; assays using purified enzymes, cellular assays in which a cellular phenotype is measured, e.g., gene expression assays; and in vivo assays that involve a particular animal (which, in certain embodiments may be an animal model for a condition related to the target pathogen).

In some embodiments, the subject method is an in vitro method that includes contacting a sample with a subject compound that specifically inhibits ENPP1. In certain embodiments, the sample is suspected of containing ENPP1 and the subject method further comprises evaluating whether the compound inhibits ENPP1.

In certain embodiments, the subject compound is a modified compound that includes a label, e.g., a fluorescent label, and the subject method further includes detecting the label, if present, in the sample, e.g., using optical detection.

In certain embodiments, the compound is modified with a support or with affinity groups that bind to a support (e.g. biotin), such that any sample that does not bind to the compound may be removed (e.g., by washing). The specifically bound ENPP1, if present, may then be detected using any convenient means, such as, using the binding of a labeled target specific probe, or using a fluorescent protein reactive reagent.

In another embodiment of the subject method, the sample is known to contain ENPP1.

In some embodiments, the method is a method of reducing cancer cell proliferation, where the method includes contacting the cell with an effective amount of a subject ENPP1 inhibitor compound (e.g., as described herein) to reduce cancer cell proliferation. In certain cases, the subject ENPP1 inhibitor compounds can act intracellularly. The method can be performed in combination with a chemotherapeutic agent (e.g., as described herein). The cancer cells can be in vitro or in vivo. In certain instances, the method includes contacting the cell with an ENPP1 inhibitor compound (e.g., as described herein) and contacting the cell with a chemotherapeutic agent. Any convenient cancer cells can be targeted.

### Methods of Treatment

Aspects of the present disclosure include methods for inhibiting the hydrolase activity of ENPP1 against cGAMP provides for increased levels of cGAMP and/or downstream modulation (e.g., activation) of the STING pathway. The inventors have discovered that cGAMP can be present in the extracellular space and that ENPP1 can control extracellular levels of cGAMP. The inventors have also discovered that cGAMP can have significant extracellular biological functions in vivo. The results described and demonstrated herein indicate that ENPP1 inhibition according to the subject methods can modulate STING activity in vivo, and thus find use in the treatment of a variety of diseases, e.g., as a target for cancer immunotherapy. As such, the subject methods can provide for selective extracellular inhibition of ENPP1 activity (e.g., hydrolase activity of cGAMP) to increase extracellular levels of cGAMP and activate the stimulator of interferon genes (STING) pathway. In some instances, the subject method is a method for increasing a STING mediated response in a subject. In some instances, the subject method is a method for modulating an immune response in a subject.

A “STING mediated response” refers to any response that is mediated by STING, including, but not limited to, immune responses, e.g., to bacterial pathogens, viral pathogens, and eukaryotic pathogens. See, e.g., Ishikawa et al. Immunity 29: 538-550 (2008); Ishikawa et al. Nature 461: 788-792 (2009); and Sharma et al. Immunity 35: 194-207 (2011). STING also functions in certain autoimmune diseases initiated by inappropriate recognition of self DNA (see, e.g., Gall et al. Immunity 36: 120-131 (2012), as well as for the induction of adaptive immunity in response to DNA vaccines (see, e.g., Ishikawa et al. Nature 461: 788-792 (2009). By increasing a STING mediated response in a subject is meant an increase in a STING mediated response in a subject as compared to a control subject (e.g., a subject who is not administered a subject compound). In some cases, the subject is human and the subject compounds and methods provide for activation of human STING. In some cases, the STING mediated response includes modulation of an immune response. In some instances, the subject method is a method of modulating an immune response in a subject.

In some cases, the STING mediated response includes increasing the production of an interferon (e.g., a type I interferon (IFN), type III interferon (IFN)) in a subject. Interferons (IFNs) are proteins having a variety of biological activities, e.g., antiviral, immunomodulating and antiproliferative, IFNs are relatively small, species-specific, single chain polypeptides, produced by mammalian cells in response to exposure to a variety of inducers such as viruses, poly peptides, mitogens and the like. Interferons protect animal tissues and cells against viral attack and are an important host defense mechanism. Interferons may be classified as Type-I, Type-II and Type-III interferons. Mammalian Type-I interferons of interest include IFN-α (alpha), IFN-β (beta), IFN-κ (kappa), IFN-δ (delta), IFN—ε (epsilon), IFN-τ (tau), IFN-ω (omega), and IFN-ζ (zeta, also known as limitin).

Interferons find use in the treatment of a variety of cancers since these molecules have anti-cancer activity that acts at multiple levels. Interferon proteins can directly inhibit the proliferation of human tumor cells. In some cases, the anti-proliferative activity is also synergistic with a variety of approved chemotherapeutic agents such as cisplatin, 5FU and paclitaxel. The immunomodulatory activity of interferon proteins can also lead to the induction of an anti-tumor immune response. This response includes activation of NK cells, stimulation of macrophage activity and induction of MHC class I surface expression, leading to the induction of anti-tumor cytotoxic T lymphocyte activity. In addition, interferons play a role in cross-presentation of antigens in the immune system. Moreover, some studies further indicate that IFN-β protein may have anti-angiogenic activity. Angiogenesis, new blood vessel formation, is critical for the growth of solid tumors. IFN-β may inhibit angiogenesis by inhibiting the expression of pro-angiogenic factors such as bFGF and VEGF. Interferon proteins may also inhibit tumor invasiveness by modulating the expression of enzymes, such as collagenase and elastase, which are important in tissue remodeling.

Aspects of the methods include administering to a subject with cancer a therapeutically effective amount of an ENPP1 inhibitor to treat the subject for cancer. In some instances, the subject is one who is diagnosed with or suspected of having cancer. Any convenient ENPP1 inhibitors can be used in the subject methods of treating cancer. In certain cases, the ENPP1 inhibitor compound is a compound as described herein. In certain cases, the ENPP1 inhibitor is a cell impermeable compound. In certain cases, the ENPP1 inhibitor is a cell permeable compound. In certain cases, the cancer is a solid tumor cancer. In certain embodiments, the cancer is selected from adrenal, liver, kidney, bladder, breast, colon, gastric, ovarian, cervical, uterine, esophageal, colorectal, prostate, pancreatic, lung (both small cell and non-small cell), thyroid, carcinomas, sarcomas, glioblastomas, melanoma and various head and neck tumors. In some cases, the cancer is breast cancer. In some embodiments, the cancer is lymphoma.

Aspects of the methods include administering to a subject a therapeutically effective amount of a cell impermeable ENPP1 inhibitor to inhibit the hydrolysis of cGAMP and treat the subject for cancer. In certain cases the cancer is a solid tumor cancer. In certain embodiments, the cancer is selected from adrenal, liver, kidney, bladder, breast, colon, gastric, ovarian, cervical, uterine, esophageal, colorectal, prostate, pancreatic, lung (both small cell and non-small cell), thyroid, carcinomas, sarcomas, glioblastomas, melanoma and various head and neck tumors. In certain embodiments, the cancer is breast cancer. In some instances, the cancer is lymphoma.

In some embodiments of the methods disclosed herein, the cell impermeable ENPP1 inhibitor is an inhibitor of any one of formulae (I)—(XVb) (e.g., as described herein). In some cases, the ENPP1 inhibitor is a compound of Tables 1-3 or a prodrug form thereof (e.g., as described herein).

In some embodiments of the methods disclosed herein, the ENPP1 inhibitor is cell permeable.

As such, aspects of the method include contacting a sample with a subject compound (e.g., as described above) under conditions by which the compound inhibits ENPP1. Any convenient protocol for contacting the compound with the sample may be employed. The particular protocol that is employed may vary, e.g., depending on whether the sample is in vitro or in vivo. For in vitro protocols, contact of the sample with the compound may be achieved using any convenient protocol. In some instances, the sample includes cells that are maintained in a suitable culture medium, and the complex is introduced into the culture medium. For in vivo protocols, any convenient administration protocol may be employed. Depending upon the potency of the compound, the cells of interest, the manner of administration, the number of cells present, various protocols may be employed.

In some embodiments, the subject method is a method of treating a subject for cancer. In some embodiments, the subject method includes administering to the subject an effective amount of a subject compound (e.g., as described herein) or a pharmaceutically acceptable salt thereof. The subject compound may be administered as part of a pharmaceutical composition (e.g., as described herein). In certain instances of the method, the compound that is administered is a compound of one of formulae formulae (I)—(XVb) (e.g., as described herein). In certain instances of the method, the compound that is administered is described by one of the compounds of Tables 1-3.

In some embodiments, an “effective amount” is an amount of a subject compound that, when administered to an individual in one or more doses, in monotherapy or in combination therapy, is effective to inhibit ENPP1 by about 20% (20% inhibition), at least about 30% (30% inhibition), at least about 40% (40% inhibition), at least about 50% (50% inhibition), at least about 60% (60% inhibition), at least about 70% (70% inhibition), at least about 80% (80% inhibition), or at least about 90% (90% inhibition), compared to the ENPP1 activity in the individual in the absence of treatment with the compound, or alternatively, compared to the ENPP1 activity in the individual before or after treatment with the compound.

In some embodiments, a “therapeutically effective amount” is an amount of a subject compound that, when administered to an individual in one or more doses, in monotherapy or in combination therapy, is effective to decrease tumor burden in the subject by about 20%, at least about 30% at least about 40%, at least about 50%, at least about 60%, at least about 70%, at least about 80%, or at least about 90%, compared to tumor burden in the individual in the absence of treatment with the compound, or alternatively, compared to the tumor burden in the subject before or after treatment with the compound. As used herein the term “tumor burden” refers to the total mass of tumor tissue carried by a subject with cancer.

In some embodiments, a “therapeutically effective amount” is an amount of a subject compound that, when administered to an individual in one or more doses, in monotherapy or in combination therapy, is effective to reduce the dose of radiotherapy required to observe tumor shrinkage in the subject by about 20%, at least about 30%, at least about 40%, at least about 50%, at least about 60%, at least about 70%, at least about 80%, or at least about 90%, compared to the dose of radiotherapy required to observe tumor shrinkage in the individual in the absence of treatment with the compound.

In some embodiments, a “therapeutically effective amount” of a compound is an amount that, when administered in one or more doses to an individual having cancer, is effective to achieve a 1.5-log, a 2-log, a 2.5-log, a 3-log, a 3.5-log, a 4-log, a 4.5-log, or a 5-log reduction in tumor size.

In some embodiments, an effective amount of a compound is an amount that ranges from about 50 ng/ml to about 50 μg/ml (e.g., from about 50 ng/ml to about 40 μg/ml, from about 30 ng/ml to about 20 μg/ml, from about 50 ng/ml to about 10 μg/ml, from about 50 ng/ml to about 1 μg/ml, from about 50 ng/ml to about 800 ng/ml, from about 50 ng/ml to about 700 ng/ml, from about 50 ng/ml to about 600 ng/ml, from about 50 ng/ml to about 500 ng/ml, from about 50 ng/ml to about 400 ng/ml, from about 60 ng/ml to about 400 ng/ml, from about 70 ng/ml to about 300 ng/ml, from about 60 ng/ml to about 100 ng/ml, from about 65 ng/ml to about 85 ng/ml, from about 70 ng/ml to about 90 ng/ml, from about 200 ng/ml to about 900 ng/ml, from about 200 ng/ml to about 800 ng/ml, from about 200 ng/ml to about 700 ng/ml, from about 200 ng/ml to about 600 ng/ml, from about 200 ng/ml to about 500 ng/ml, from about 200 ng/ml to about 400 ng/ml, or from about 200 ng/ml to about 300 ng/ml).

In some embodiments, an effective amount of a compound is an amount that ranges from about 10 pg to about 10) mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 μg, from about 1 μg to about 10 μg, from about 10 μg to about 50 μg, from about 50 μg to about 150 μg, from about 150 μg to about 250 μg, from about 250 μg to about 500 μg, from about 500 μg to about 750 μg, from about 750 μg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of a compound is administered. In other embodiments, multiple doses are administered. Where multiple doses are administered over a period of time, the compound can be administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

Administration of a therapeutically effective amount of a subject compound to an individual with cancer can result in one or more of: 1) a reduction in tumor burden: 2) a reduction in the dose of radiotherapy required to effect tumor shrinkage; 3) a reduction in the spread of a cancer from one cell to another cell in an individual: 4) a reduction of morbidity or mortality in clinical outcomes; 5) shortening the total length of treatment when combined with other anti-cancer agents; and 6) an improvement in an indicator of disease response (e.g., a reduction in one or more symptoms of cancer). Any of a variety of methods can be used to determine whether a treatment method is effective. For example, a biological sample obtained from an individual who has been treated with a subject method can be assayed.

Any of the compounds described herein can be utilized in the subject methods of treatment. In certain instances, the compound is of one of formulae (I)—(XVb) (e.g., as described herein). In certain cases, the compound is one of the compounds of Tables 1-3 or a prodrug form thereof. In some cases, the compound that is utilized in the subject methods is not cell permeable. In some cases, the compound that is utilized in the subject methods has poor cell permeability.

In some embodiments, the compound specifically inhibits ENPP1. In some embodiments, the compound modulates the activity of cGAMP. In some embodiments, the compound interferes with the interaction of ENPP1 and cGAMP. In some embodiments, the compound results in activation of the STING pathway.

In some embodiments, the subject is mammalian. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys). The subject may be in need of treatment for cancer. In some instances, the subject methods include diagnosing cancer, including any one of the cancers described herein. In some embodiments, the compound is administered as a pharmaceutical preparation.

In certain embodiments, the ENPP1 inhibitor compound is a modified compound that includes a label, and the method further includes detecting the label in the subject. The selection of the label depends on the means of detection. Any convenient labeling and detection systems may be used in the subject methods, see e.g., Baker, “The whole picture,” Nature, 463, 2010, p 977-980. In certain embodiments, the compound includes a fluorescent label suitable for optical detection. In certain embodiments, the compound includes a radiolabel for detection using positron emission tomography (PET) or single photon emission computed tomography (SPECT). In some cases, the compound includes a paramagnetic label suitable for tomographic detection. The subject compound may be labeled, as described above, although in some methods, the compound is unlabeled and a secondary labeling agent is used for imaging.

### Combination Therapies

The subject compounds can be administered to a subject alone or in combination with an additional, i.e., second, active agent. Combination therapeutic methods where the subject ENPP1 inhibitor compounds may be used in combination with a second active agent or an additional therapy, e.g., radiation therapy. The terms “agent,” “compound,” and “drug” are used interchangeably herein. For example, ENPP1 inhibitor compounds can be administered alone or in conjunction with one or more other drugs, such as drugs employed in the treatment of diseases of interest, including but not limited to, immunomodulatory diseases and conditions and cancer. In some embodiments, the subject method further includes coadministering concomitantly or in sequence a second agent, e.g., a small molecule, a chemotherapeutic, an antibody, an antibody fragment, an antibody-drug conjugate, an aptamer, a protein, or a checkpoint inhibitor. In some embodiments, the method further includes performing radiation therapy on the subject.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be adminstered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug or additional therapy with a pharmaceutical composition of the present disclosure means administration of the compound and second agent or additional therapy at such time that both the known drug and the composition of the present invention will have a therapeutic effect. Such concomitant administration may involve concurrent (i.e. at the same time), prior, or subsequent administration of the drug with respect to the administration of a subject compound. Routes of administration of the two agents may vary, where representative routes of administration are described in greater detail below. A person of ordinary skill in the art would have no difficulty determining the appropriate timing, sequence and dosages of administration for particular drugs or therapies and compounds of the present disclosure.

In some embodiments, the compounds (e.g., a subject compound and the at least one additional compound or therapy) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about 10 minutes or less of each other, such as 5 minutes or less, or 1 minute or less of each other.

Also provided are pharmaceutical preparations of the subject compounds and the second active agent. In pharmaceutical dosage forms, the compounds may be administered in the form of their pharmaceutically acceptable salts, or they may also be used alone or in appropriate association, as well as in combination, with other pharmaceutically active compounds.

In conjunction with any of the subject methods, the ENPP1 inhibitor compounds (e.g., as described herein) (or pharmaceutical compositions comprising such compounds) can be administered in combination with another drug designed to reduce or prevent inflammation, treat or prevent chronic inflammation or fibrosis, or treat cancer. In each case, the ENPP1 inhibitor compound can be administered prior to, at the same time as, or after the administration of the other drug. In certain cases, the cancer is selected from adrenal, liver, kidney, bladder, breast, colon, gastric, ovarian, cervical, uterine, esophageal, colorectal, prostate, pancreatic, lung (both small cell and non-small cell), thyroid, carcinomas, sarcomas, glioma, glioblastomas, melanoma and various head and neck tumors.

For the treatment of cancer, the ENPP1 inhibitor compounds can be administered in combination with a chemotherapeutic agent selected from the group consisting of alkylating agents, nitrosoureas, antimetabolites, antitumor antibiotics, plant (vinca) alkaloids, steroid hormones, taxanes, nucleoside analogs, steroids, anthracyclines, thyroid hormone replacement drugs, thymidylate-targeted drugs. Chimeric Antigen Receptor/T cell therapies. Chimeric Antigen Receptor/NK cell therapies, apoptosis regulator inhibitors (e.g., B cell CLL/lymphoma 2 (BCL-2) BCL-2-like 1 (BCL-XL) inhibitors), CARP-1/CCAR1 (Cell division cycle and apoptosis regulator 1) inhibitors, colony-stimulating factor-1 receptor (CSF1R) inhibitors, CD47 inhibitors, cancer vaccine (e.g., a Th17-inducing dendritic cell vaccine, or a genetically modified tyrosinase such as Oncept®) and other cell therapies.

Specific chemotherapeutic agents of interest include, but are not limited to, Gemcitabine, Docetaxel, Bleomycin, Erlotinib, Gefitinib, Lapatinib, Imatinib, Dasatinib, Nilotinib, Bosutinib, Crizotinib, Ceritinib, Trametinib, Bevacizumab, Sunitinib, Sorafenib, Trastuzumab, Ado-trastuzumab emtansine, Rituximab, Ipilimumab, Rapamycin, Temsirolimus, Everolimus, Methotrexate, Doxorubicin, Abraxane, Folfirinox, Cisplatin, Carboplatin, 5-fluorouracil, Teysumo, Paclitaxel, Prednisone, Levothyroxine, Pemetrexed, navitoclax, and ABT-199. Peptidic compounds can also be used. Cancer chemotherapeutic agents of interest include, but are not limited to, dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD).

In some embodiments, the ENPP1 inhibitor compounds can be administered in combination with a chemotherapeutic agent to treat cancer. In certain cases, the chemotherapeutic agent is Gemcitabine. In some cases, the chemotherapeutic agent is Docetaxel. In some cases, the chemotherapeutic agent is Abraxane.

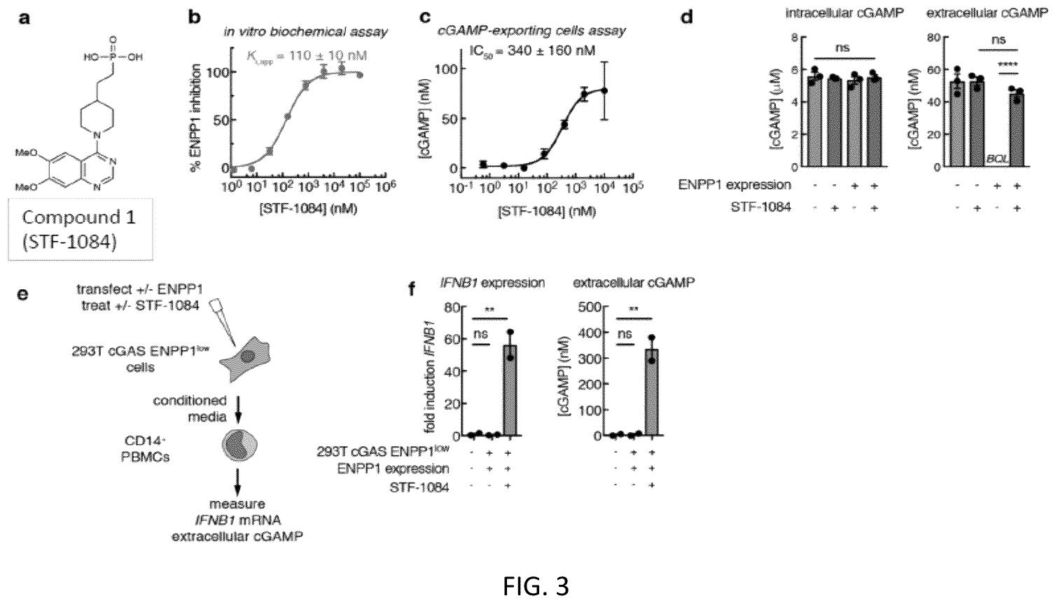

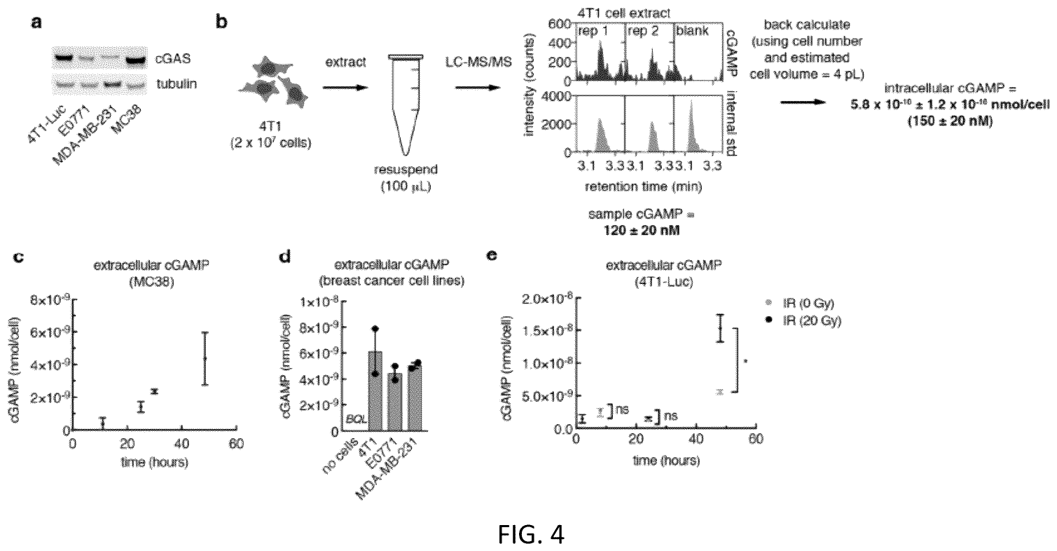

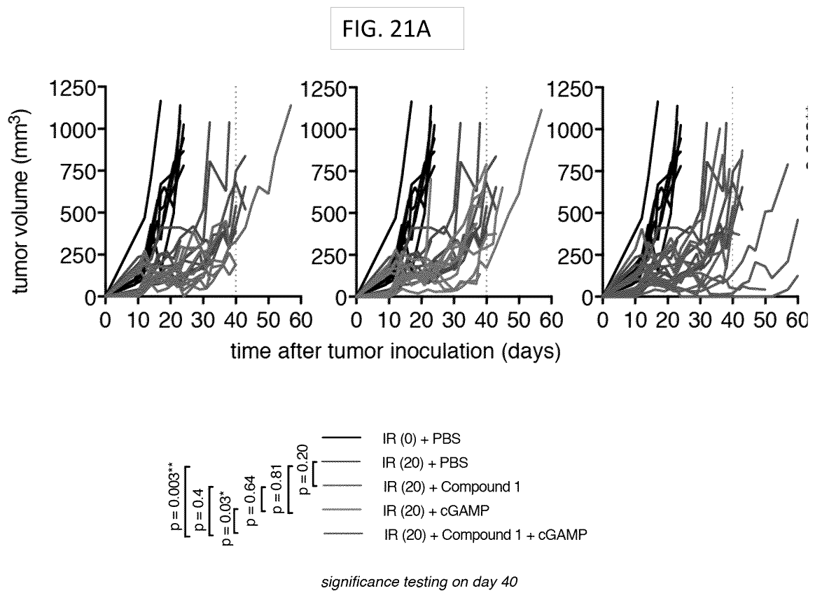

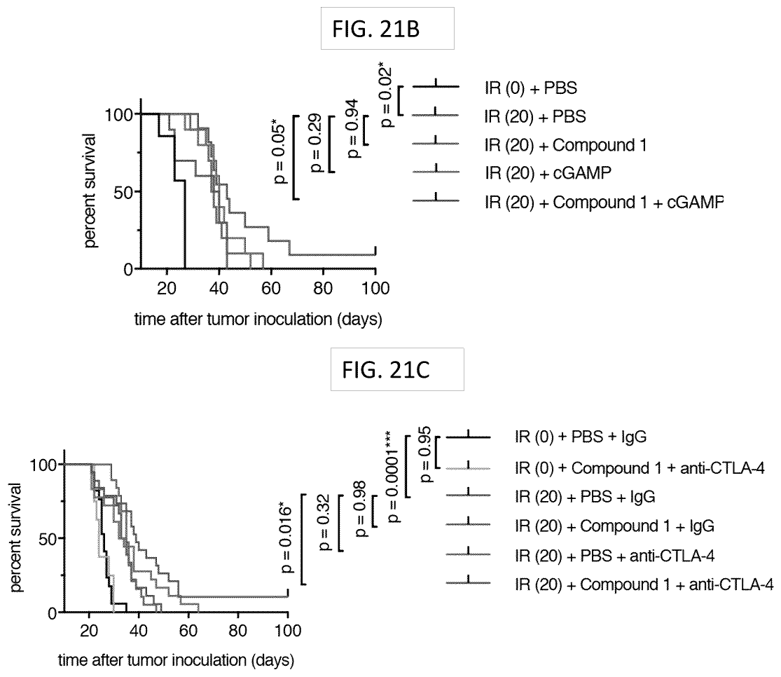

For the treatment of cancer (e.g., solid tumor cancer), the ENPP1 inhibitor compound can be administered in combination an immunotherapeutic agent. An immunotherapeutic agent is any convenient agent that finds use in the treatment of disease by inducing, enhancing, or suppressing an immune response. In some cases, the immunotherapeutic agent is an immune checkpoint inhibitor. For example, FIG. 21A-4C illustrates that an exemplary ENPP1 inhibitor can act synergistically with an immune checkpoint inhibitor in a mouse model. Any convenient checkpoint inhibitors can be utilized, including but not limited to, cytotoxic T-lymphocyte-associated antigen 4 (CTLA-4) inhibitors, programmed death 1 (PD-1) inhibitors and PD-L1 inhibitors. In certain instances, the checkpoint inhibitor is selected from a cytotoxic T-lymphocyte-associated antigen 4 (CTLA-4) inhibitor, a programmed death 1 (PD-1) inhibitor and a PD-L1 inhibitor. Exemplary checkpoint inhibitors of interest include, but are not limited to, ipilimumab, pembrolizumab and nivolumab. In certain embodiments, for treatment of cancer and/or inflammatory disease, the immunomodulatory polypeptide(s) can be administered in combination with a colony-stimulating factor-1 receptor (CSF1R) inhibitor. CSF1R inhibitors of interest include, but are not limited to, emactuzumab.

Any convenient cancer vaccine therapies and agents can be used in combination with the subject ENPP1 inhibitor compounds, compositions and methods. For treatment of cancer, e.g., ovarian cancer, the ENPP1 inhibitor compounds can be administered in combination with a vaccination therapy, e.g., a dendritic cell (DC) vaccination agent that promotes Th1/Th17 immunity. Th17 cell infiltration correlates with markedly prolonged overall survival among ovarian cancer patients. In some cases, the ENPP1 inhibitor compound finds use as adjuvant treatment in combination with Th17-inducing vaccination.

Also of interest are agents that are CARP-1/CCAR1 (Cell division cycle and apoptosis regulator 1) inhibitors, including but not limited to those described by Rishi et al., Journal of Biomedical Nanotechnology, Volume 11, Number 9, September 2015, pp. 1608-1627(20), and CD47 inhibitors, including, but not limited to, anti-CD47 antibody agents such as Hu5F9-G4.

In certain instances, the combination provides an enhanced effect relative to either component alone; in some cases, the combination provides a supra-additive or synergistic effect relative to the combined or additive effects of the components. A variety of combinations of the subject compounds and the chemotherapeutic agent may be employed, used either sequentially or simultaneously. For multiple dosages, the two agents may directly alternate, or two or more doses of one agent may be alternated with a single dose of the other agent, for example. Simultaneous administration of both agents may also be alternated or otherwise interspersed with dosages of the individual agents. In some cases, the time between dosages may be for a period from about 1-6 hours, to about 6-12 hours, to about 12-24 hours, to about 1-2 days, to about 1-2 week or longer following the initiation of treatment.

Combination with cGAMP-Inducing Chemotherapeutics

Aspects of the present disclosure include methods of treating cancer, where the ENPP1 inhibitor compounds (or pharmaceutical compositions comprising such compounds) can be administered in combination with a chemotherapeutic that is capable of inducing production of cGAMP in vivo. When a subject is exposed to an effective amount of a particular chemotherapeutic, the production of 2′3′-cGAMP can be induced in the subject. The induced levels of cGAMP can be maintained and/or enhanced when the subject ENPP1 inhibitor compounds are co-administered to prevent the degradation of the cGAMP, e.g., enhanced by comparison to levels achieved with either agent alone. Any convenient chemotherapeutic agents which can lead to DNA damage and can induce cGAMP production by the dying cells due to overwhelmed repair or degradation mechanisms can be used in the subject combination therapeutic methods, such as alkylating agents, nucleic acid analogues, and intercalating agents. In some cases, the cGAMP-inducing chemotherapeutic is an anti-mitotic agent. An anti-mitotic agent is an agent that acts by damaging DNA or binding to microtubules. In some cases, the cGAMP-inducing chemotherapeutic is an antineoplastic agent.

Cancers of interest which may be treated using the subject combination therapies include, but are not limited to, adrenal, liver, kidney, bladder, breast, colon, gastric, ovarian, cervical, uterine, esophageal, colorectal, prostate, pancreatic, lung (both small cell and non-small cell), thyroid, carcinomas, sarcomas, glioma, glioblastomas, melanoma and various head and neck tumors. In some cases, the cancer is breast cancer. In certain instances, the cancer is glioma or glioblastoma.

Chemotherapeutic of interest include, but are not limited to. Uracil analogues, Fluorouracil prodrug, Thymidylate Synthase inhibitors, Deoxycytidine analogue, DNA synthesis inhibitor (e.g. leading to S-phase apoptosis), Folate analogue, Dehydrofolate Reductase inhibitor, Anthracycline, intercalating agent. (e.g., leading to double strand breaks). Topoisomerase IIa inhibitor, Taxane, microtubule disassembly inhibitor (e.g. leading to G2/M phase arrest/apoptosis), microtubule assembly inhibitor, microtubule function stabilizers (e.g. leading to G2/M-phase apoptosis), tubulin polymerization promoters, tubulin binding agent (e.g. leading to apoptosis by M-phase arrest) Epothilone B analogue, Vinka alkaloid, Nitrogen mustard, Nitrosourea, DNA alkylater (e.g., leading to interstrand crosslinks, apoptosis via p53), VEGF inhibitor, anti-angiogenic antibody, HER2 inhibitor, Quinazoline HER2 inhibitor, EGFR inhibitor, tyrosine kinase inhibitor, Sirolimus analogue, mTORC1 inhibitor (e.g., in breast cancer combination with Exemestane=Aromastase inhibitor inhibiting Estrogen production), Triazene, Dacarbazine prodrug, Methylhydrazine.

Exemplary breast cancer chemotherapeutic of interest include, but are not limited to, Capecitabine, Carmofur, Fluorouracil, Tegafur, Gemcitabine, Methotrexate, Doxorubicin, Epirubicin, Docetaxel, Ixabepilone, Vindesine, Vinorelbine, Cyclophosphamide, Bevacicumab, Pertuzumab, Trastuzumab, Lapatinib and Everolimus, Exemplary Glioma/Glioblastoma related antineoplastic drugs: include, but are not limited to, Carmustine, Lomustine, Temozolomide, Procarbazine, Vincristine and Bevacicumab. Exemplary DNA damaging chemotherapeutic agents of interest include, but are not limited to. Melphalan, Cisplatin, and Etoposide, Fluorouracil, Gemcitabine.

### Combination Radiation Therapy

Alternatively, for the methods of treating cancer, the ENPP1 inhibitor compounds (or pharmaceutical compositions comprising such compounds) can be administered in combination with radiation therapy. In certain embodiments, the methods include administering radiation therapy to the subject. Again, the ENPP1 inhibitor compound can be administered prior to, or after the administration of the radiation therapy. As such, the subject methods can further include administering radiation therapy to the subject. The combination of radiation therapy and administration of the subject compounds can provide a synergistic therapeutic effect. When a subject is exposed to radiation of a suitable dosage and/or frequency during radiation therapy (RT), the production of 2′3′-cGAMP can be induced in the subject. These induced levels of cGAMP can be maintained and/or enhanced when the subject ENPP1 inhibitor compounds are co-administered to prevent the degradation of the cGAMP, e.g., enhanced by comparison to levels achieved with RT alone. For example. FIG. 21A illustrates that an exemplary ENPP1 inhibitor can act synergistically with Radiation therapy (RT) to decrease tumor burden in a mouse model. As such, aspects of the subject methods include administration of a reduced dosage and/or frequency/regimen of radiation treatment as compared to a therapeutically effective dosage and/or frequency/regimen of radiation treatment alone. In some cases, the radiation therapy is administered in combination with the subject compounds at a dosage and/or frequency effective to reduce risk of radiation damage to the subject, e.g., radiation damage that would be expected to occur under a therapeutically effective dosage and/or frequency/regimen of radiation treatment alone.

In some cases, the method includes administering an ENPP1 inhibitor to the subject before radiation therapy. In some cases, the method includes administering an ENPP1 inhibitor to the subject following exposure of the subject to radiation therapy. In certain cases, the method includes sequential administration of radiation therapy, followed by an ENPP1 inhibitor, followed by a checkpoint inhibitor to a subject in need thereof.

### Utility

The compounds and methods of the invention, e.g., as described herein, find use in a variety of applications. Applications of interest include, but are not limited to: research applications and therapeutic applications. Methods of the invention find use in a variety of different applications including any convenient application where inhibition of ENPP1 is desired.

The subject compounds and methods find use in a variety of research applications. The subject compounds and methods may be used in the optimization of the bioavailability and metabolic stability of compounds.

The subject compounds and methods find use in a variety of therapeutic applications. Therapeutic applications of interest include those applications in cancer treatment. As such, the subject compounds find use in the treatment of a variety of different conditions in which the inhibition and/or treatment of cancer in the host is desired. For example, the subject compounds and methods may find use in treating a solid tumor cancer (e.g., as described herein).

### Pharmaceutical Compositions

The herein-discussed compounds can be formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy.” 20th edition. Lippincott. Williams, & Wilkins. Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

In some embodiments, the subject compound and a second active agent (e.g., as described herein), e.g. a small molecule, a chemotherapeutic, an antibody, an antibody fragment, an antibody-drug conjugate, an aptamer, or a protein, etc. are administered to individuals in a formulation (e.g., in the same or in separate formulations) with a pharmaceutically acceptable excipient(s). In some embodiments, the second active agent is a checkpoint inhibitor, e.g., a cytotoxic T-lymphocyte-associated antigen 4 (CTLA-4) inhibitor, a programmed death 1 (PD-1) inhibitor, or a PD-L1 inhibitor.

In another aspect of the present invention, a pharmaceutical composition is provided, comprising, or consisting essentially of, a compound of the present invention, or a pharmaceutically acceptable salt, isomer, tautomer or prodrug thereof, and further comprising one or more additional active agents of interest. Any convenient active agents can be utilized in the subject methods in conjunction with the subject compounds. In some instances, the additional agent is a checkpoint inhibitor. The subject compound and checkpoint inhibitor, as well as additional therapeutic agents as described herein for combination therapies, can be administered orally, subcutaneously, intramuscularly, intranasally, parenterally, or other route. The subject compound and second active agent (if present) may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ. In certain cases, the therapeutic agents can be administered intranasally. In some cases, the therapeutic agents can be administered intratumorally.

In some embodiments, the subject compound and a chemotherapeutic agent are administered to individuals in a formulation (e.g., in the same or in separate formulations) with a pharmaceutically acceptable excipient(s). The chemotherapeutic agents include, but are not limited to alkylating agents, nitrosoureas, antimetabolites, antitumor antibiotics, plant (vinca) alkaloids, and steroid hormones. Peptidic compounds can also be used. Suitable cancer chemotherapeutic agents include dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. Suitable cancer chemotherapeutic agents also include maytansinoids and active analogs and derivatives thereof (see, e.g., EP 1391213; and Liu et al (19%) Proc. Natl. Acad. Sci. USA 93:8618-8623): duocarmycins and active analogs and derivatives thereof (e.g., including the synthetic analogues. KW-2189 and CB 1-TM1); and benzodiazepines and active analogs and derivatives thereof (e.g., pyrrolobenzodiazepine (PBD).

The subject compound and second chemotherapeutic agent, as well as additional therapeutic agents as described herein for combination therapies, can be administered orally, subcutaneously, intramuscularly, parenterally, or other route. The subject compound and second chemotherapeutic agent may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

The subject compounds may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

### EXAMPLES

The following examples are put forth so as to provide those of ordinary skill in the art with a complete disclosure and description of how to make and use embodiments of the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present disclosure. All such modifications are intended to be within the scope of the claims appended hereto.

### Example 1: Synthesis of Compounds

Compounds may be synthesized using any convenient method. Methods which can be adapted for use in preparing compounds of this disclosure includes the exemplary synthetic methods described in Examples 1a-1c, and those methods described by Li et al. in PCT application No. PCT/US2018/050018, filed Sep. 7, 2018, the disclosure of which is herein incorporated by reference in its entirety. Many general references providing commonly known chemical synthetic schemes and conditions useful for synthesizing the disclosed compounds are also available (see, e.g., Smith and March, March's Advanced Organic Chemistry: Reactions. Mechanisms, and Structure, Fifth Edition, Wiley-Interscience, 2001; or Vogel. A Textbook of Practical Organic Chemistry. Including Qualitative Organic Analysis, Fourth Edition, New York: Longman, 1978). Reactions may be monitored by thin layer chromatography (TLC). LC/MS and reaction products characterized by LC/MS and ¹H NMR. Intermediates and final products may be purified by silica gel chromatography or by HPLC.

### Example 1a: Exemplary Synthetic Scheme Compound 1

The synthesis of Compound 1, which can be adapted for use in preparing compounds of this disclosure, is set out below:

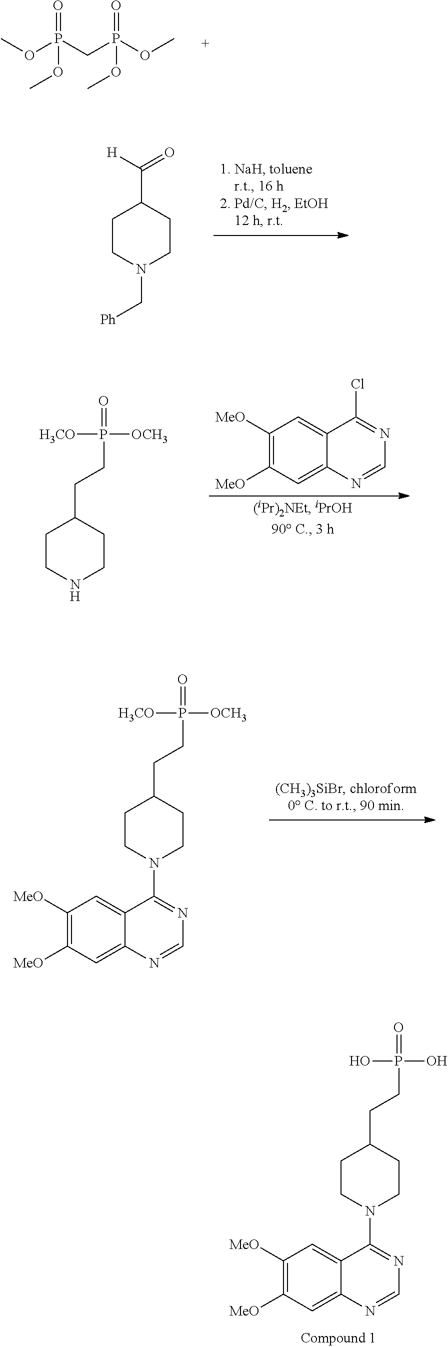

### Preparation of dimethyl (2-(piperidin-4-yl)ethyl)phosphonate

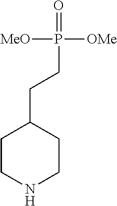 [SMILES: COP(=O)(CCC1CCNCC1)OC]

Sodium hydride (2.16 g, 54.11 mmol) was carefully added to a stirred solution of bis(dimethoxyphosphoryl)methane (11.42 g, 49.19 mmol) in toluene (100 mL) at room temperature. The reaction mixture was then placed under an atmosphere of nitrogen and a solution of 1-benzylpiperidine-4-carbaldehyde (10 g, 49.19 mmol) in toluene (50 mL) was slowly added keeping the temperature below 40° C. The resulting mixture was left to stir at room temperature for 16 h and then quenched by the addition of aqueous saturated ammonium chloride solution. The organic phase was separated, washed with brine, dried (MgSO₄) and evaporated to dryness. Chromatography (120 g SiO₂; 5 to 100% gradient of EtOAc in hexanes) provided dimethyl (E)-(2-(1-benzylpiperidin-4-yl)vinyl)phosphonate (6.2 g, 16%) as a colorless oil.

To a mixture of dimethyl (E)-(2-(1-benzylpiperidin-4-yl)vinyl)phosphonate (3.7 g, 12.0 mmol) in ethanol (40 mL) was added Pd/C (1.1 g, 10.3 mmol). The mixture was placed under an atmosphere of hydrogen and stirred at room temperature for 12 h, filtered and evaporated to dryness under reduced pressure to give dimethyl (2-(piperidin-4 yl)ethyl)phosphonate (2.7 g, 100%) as colorless oil.

### Preparation of dimethyl (2-(1-(6,7-dimethoxyquinazolin-4-yl)piperidin-4-yl)ethyl)phosphonate

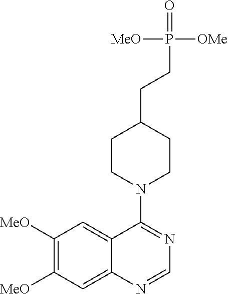 [SMILES: COc1cc2ncnc(N3CCC(CCP(=O)(OC)OC)CC3)c2cc1OC]

Diisopropylethylamine (0.6 g, 8.9 mmol) was added to a mixture of dimethyl (2-(piperidin-4-yl)ethyl)phosphonate (1.1 g, 4.9 mmol) and 4-chloro-6,7-dimethoxyquinazoline (1.0 g, 4.5 mmol) in isopropyl alcohol (20 mL). After stirring at 90° C. for 3 h, the reaction mixture was cooled and evaporated to dryness. Purification of silica gel (5% MeOH in dichloromethane) provided dimethyl (2-(1-(6,7-dimethoxyquinazolin-4-yl)piperidin-4-yl)ethyl)phosphonate (755 mg, 37/0) as oil. LC-MS: m/z=410.25 [M+H]⁺

¹H NMR (500 MHz, CDCl₃) δ 8.65 (s, 1H), 7.23 (s, 1H), 7.09 (s, 1H), 4.19 (dq, J=14.0, 2.9, 2.4 Hz, 2H), 4.02 (s, 3H), 3.99 (s, 3H), 3.77 (s, 3H), 3.75 (s, 3H), 3.05 (td, J=12.8, 2.3 Hz, 2H), 1.93-1.77 (m, 4H), 1.67 (ddd, J=14.1, 9.5, 5.9 Hz, 3H), 1.46 (qd, J=12.2, 3.7 Hz, 2H).

### Preparation of dimethyl (2-(1-(6,7-dimethoxyquinazolin-4-yl)piperidin-4yl)ethyl)phosphonic Acid (Compound 1)

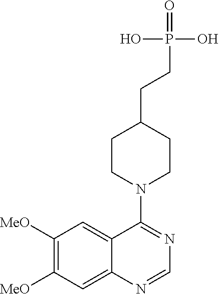 [SMILES: COc1cc2ncnc(N3CCC(CCP(=O)(O)O)CC3)c2cc1OC]

Bromotrimethylsilane (3.67 g, 24 mmol) was added to a cooled solution of dimethyl (2-(1-(6,7-dimethoxyquinazolin-4-yl)piperidin-4-yl)ethyl)phosphonate (3.25 g, 7.94 mmol) in chloroform (60 mL) that was cooled by an ice bath. The reaction mixture was allowed to warm to room temperature and after 90 minutes was quenched by the addition of methanol (20 mL). The mixture was evaporated to dryness under reduced pressure and then solvated in methanol (100 mL). The reaction mixture was concentrated to half volume, filtered to remove precipitate, and then evaporated to dryness. The residue was crystalized with dichloromethane, filtered and dried under vacuum to give dimethyl (2-(1-(6,7-dimethoxyquinazolin-4-yl)piperidin-4-yl)ethyl)phosphonic acid (2.1 g, 69%).

LC-MS: m/z=381.8 [M+H]⁺

¹H NMR (500 MHz, DMSO-d₆) δ 8.77 (s, 1H), 7.34 (s, 1H), 7.23 (s, 1H), 4.71 (d, J=13.1 Hz, 2H), 3.99 (s, 3H), 3.97 (s, 3H), 3.48 (t, J=12.7 Hz, 2H), 3.18 (s, 1H), 1.97-1.90 (m, 2H), 1.62-1.43 (m, 4H), 1.40-1.27 (m, 2H).

### Example 1b: Synthesis of Compound 5 (Table 1)

The synthetic scheme set out below is used to prepare Compound 5:

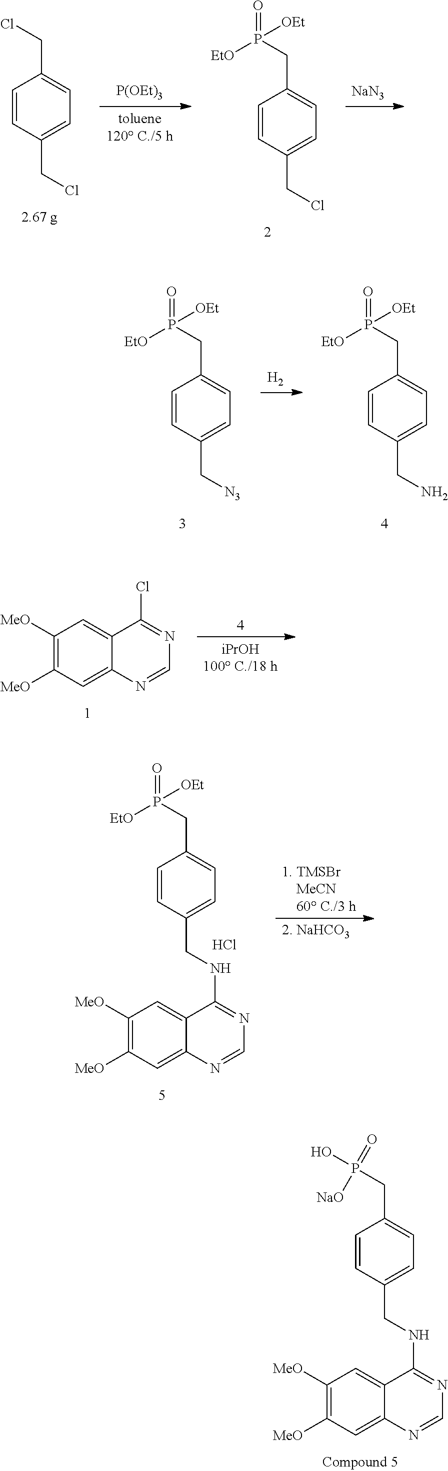

### Example 1c: Synthesis of Compound 6 (Table 1)

The synthetic scheme set out below is used to prepare Compound 6:

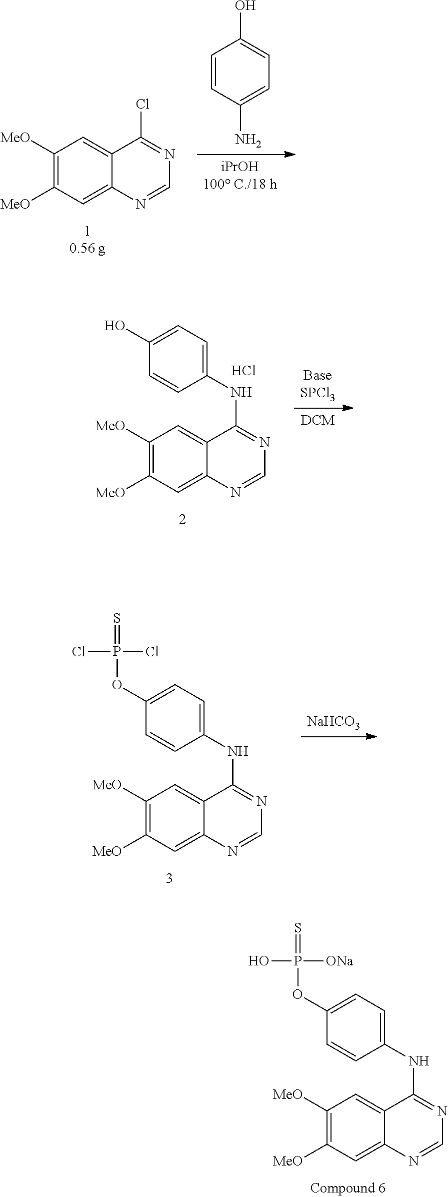

Chemical Synthesis: Reactions were performed under ambient atmosphere unless otherwise noted. Qualitative TLC analysis was performed on 250 mm thick, 60 Å, glass backed, F254 silica (Silicycle, Quebec City, Canada). Visualization was accomplished with UV light and exposure to p-anisaldehyde or KMnO₄ stain solutions followed by heating. All solvents used were ACS grade Sure-Seal, and all other reagents were used as received unless otherwise noted. The synthesis of 4-chloroquinazolines and 4-chloro3-quinonline nitriles that are not commercially available are described in the supplemental information along with the amine building blocks. Flash chromatography was performed on a Teledyne Isco purification system using silica gel flash cartridges (SiliCycle®, SiliaSep™ 40-63 μm, 60 Å). HPLC was performed on an Agilent 1260 Infinity preparative scale purification system using an Agilent PrepHT Zorbax Eclipse XDB-C18 reverse-phase column (21.2×250 mm). Structure determination was performed using ¹H spectra that were recorded on a Bruker AV-500 spectrometer, and low-resolution mass spectra (ESI-MS) that were collected on a Shimadzu 20-20 ESI LCMS instrument. Structure determination was performed using ¹H spectra that were recorded on either a Bruker AV-500 or AV-400 spectrometer, and low-resolution mass spectra (ESI-MS) that were collected on a Shimadzu 20-20 ESI LCMS instrument. Final compound purity was >95%, as determined by HPLC-MS. All final compound ¹H spectra were consistent with the expected structures.

Synthesis of the ureas 4 and 5.

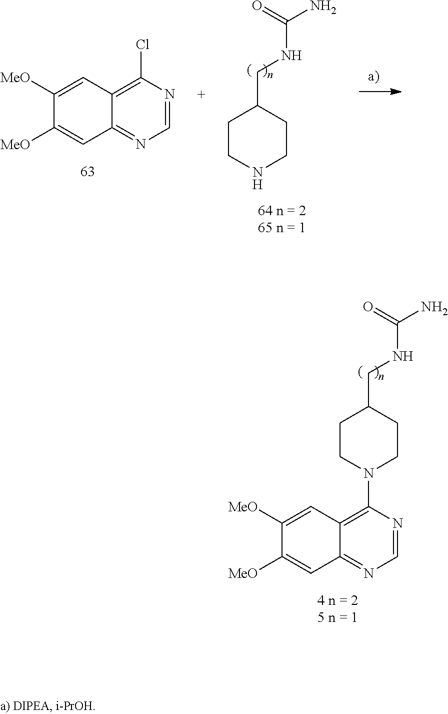

### Preparation of 1-(2-(1-(6,7-dimethoxyquinazolin-4-yl)piperidin-4-yl)ethyl)urea 4 (in Table 3a)

To a solution of 1-(2-(piperidin-4-yl)ethyl)urea 64 (173 mg, 1.01 mmol) in isopropanol (5 mL) was added 4-chloro-6,7-dimethoxyquinazoline 63 (181 mg, 0.81 mmol) and N,N-diisopropylethylamine (391 mg, 3.03 mmol) under nitrogen atmosphere. The mixture was stirred at room temperature for 2 h and then evaporated to dryness under reduced pressure. Purification (prep-HPLC) gave the title compound 4 (172 mg, 47%) as light yellow crystals.

LCMS: [M+H]⁺ m/z 360. ¹H NMR (400 MHz, DMSO-d₆) δ 8.49 (s, 1H), 7.17 (s, 1H), 7.07 (s, 1H), 5.92-5.90 (m, 1H), 5.36 (br s, 2H), 4.13-4.09 (m, 2H), 3.90 (s, 3H), 3.88 (s, 3H), 3.04-2.94 (m, 4H), 1.81-1.78 (m, 2H), 1.62-1.56 (m, 1H) and 1.38-1.33 (m, 4H).

### Preparation of 1-((1-(6,7-dimethoxyquinazolin-4-yl)piperidin-4-yl)methyl)urea 5 (in Table 3a)

To a solution of 1-(piperidin-4-ylmethyl)urea 65 (155 mg, 0.97 mmol) in isopropanol (10 mL) was added 4-chloro-6,7-dimethoxyquinazoline 63 (174 mg, 0.78 mmol) and N,N-diisopropylethylamine (394 mg, 2.9 mmol) under a nitrogen atmosphere. The mixture was stirred at 10° C. for 3 h and then evaporated to dryness under reduced pressure. Chromatography (SiO₂: 0 to 6% MeOH in dichloromethane) to give the desired product 5 (150 mg, 44%) as a white solid.

LCMS: [M+H]⁺ m/z 346.0 ¹H NMR (400 MHz, Methanol-d₄) δ 8.44 (s, 1H), 7.14 (s, 1H), 7.12 (s, 1H), 4.28-4.24 (m, 2H), 3.96 (s, 3H), 3.94 (s, 3H), 3.13-3.07 (m, 4H), 1.94-1.87 (m, 3H) and 1.50-1.41 (m, 2H).

### Preparation of 3-(1-(6,7-dimethoxyquinazolin-4-yl)piperidin-4-yl)propanoic acid 6 (in Table 3a)

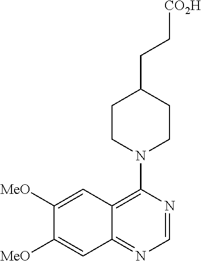 [SMILES: COc1cc2ncnc(N3CCC(CCC(=O)O)CC3)c2cc1OC]

4-Chloro-6,7-dimethoxy-quinazoline 63 (3.14 g, 13.98 mmol) and 3-(4-piperidyl)propanoic acid (2.0 g, 12.72 mmol) were suspended in isopropanol (100 mL) and stirred at 90° C. for 3 h. Once cooled, the mixture was evaporated to dryness under reduced pressure. The residue was then triturated with CH₂Cl₂ (20 mL) to give the title compound 6 (1.87 g, 42%) as a white solid.

¹H NMR (400 MHz, Methanol-d₄) δ

### Preparation of (2-(1-(6,7-dimethoxyquinolin-4-yl)piperidin-4-yl)ethyl)boronic Acid 7 in Table 3a

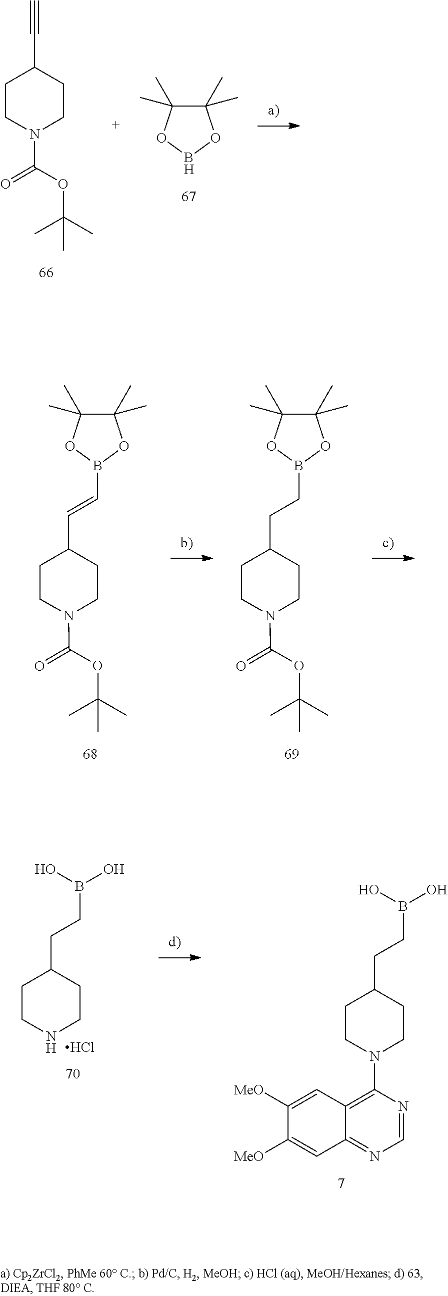

A solution of tert-butyl 4-ethynylpiperidine-1-carboxylate 66 (2.92 g, 13.95 mmol), bis(cyclopentadienyl)zirconium chloride hydride (150 mg, 0.518 mmol) and 4,4,5,5-tetramethyl-1,3,2-dioxaborolane 67 (1.49 g, 11.63 mmol) in solvent was stirred at 60° C. for 16 h and then diluted with ether and evaporated to dryness under reduced pressure. Chromatography (SiO₂; 2-5% ethyl acetate in petroleum ether) provided 68 (4.2 g, 89%). To a mixture of tert-butyl (E)-4-(2-(4,4,5,5-tetramethyl-1,3,2-dioxaborolan-2-yl)vinyl)piperidine-1-carboxylate 68 (4.2 g, 12.46 mmol) and palladium on carbon (840 mg, 20% w/w) in MeOH (500 mL) was placed under an atmosphere of hydrogen and stirred at room temperature for 16 h. The mixture was then filtered through a pad of Celite® and then evaporated to dryness under reduced pressure to afford 69 (4.2 g, 92%). 1M Aqueous HCl solution (4 mL) solution was added to a cooled (0° C.) mixture of 73 (460 mg, 1.36 mmol) in MeOH/hexane (5 mL/5 mL). The mixture was allowed to warm to room temperature and stirred for 3 h and then evaporated to dryness under reduced pressure to afford (2-(piperidin-4-yl)ethyl)boronic acid 70 (180 mg, 68%) as the hydrochloride salt. To a solution of 70 (140 mg, 1.04 mmol) in THF (5 mL) was added 4-chloro-6,7-dimethoxyquinazoline 63 (180 mg, 0.935 mmol) followed by N,N-diisopropylethylamine (360 mg, 1.87 mmol). The mixture was stirred at 80° C. for 16 h and then evaporated to dryness under reduced pressure. Purification (prep-HPLC) gave the title compound as a light yellow solid (105 mg; 37%).

LCMS: [M+H]⁺ m/z 346.3. ¹H NMR (400 MHz, DMSO-d₆) δ 8.67 (s, 1H), 7.26 (s, 1H), 7.25 (s, 1H), 4.62-4.59 (m, 2H), 3.92 (s, 3H), 3.90 (s, 3H), 3.42-3.36 (m, 4H), 2.46 (s, 1H), 1.88-1.86 (m, 2h), 1.29-1.14 (m, 3H) and 0.60-0.56 (m, 2H).

Preparation of the hydroxamic acids 8 and 9.

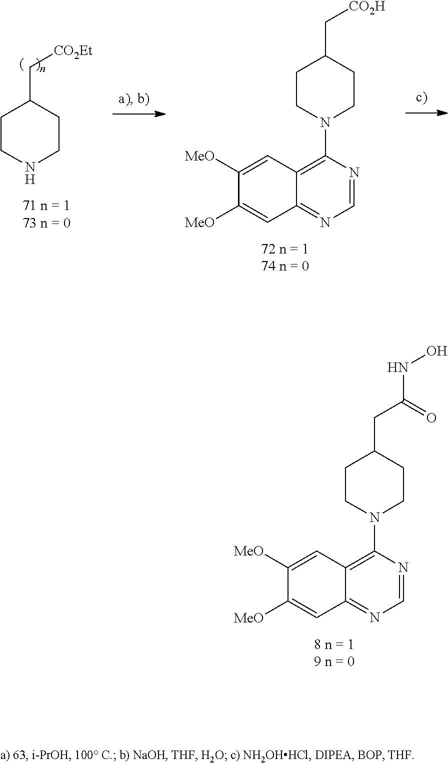

Preparation of 2-(1-(6,7-dimethoxyquinazolin-4-yl)piperidin-4-yl)-N-hydroxyacetamide 8 (in Table 3a)

A mixture of 4-chloro-6,7-dimethoxyquinazoline 63 (600 mg, 2.68 mmol) and ethyl 2-(piperidin-4-yl)acetate 71 (504 mg, 2.95 mmol) in i-PrOH (6 mL) was stirred at 100° C. for 16 h in a sealed tube. Then the reaction mixture was concentrated under reduced pressure and the residue was purified by silica gel chromatography to give ethyl 2-(1-(6,7-dimethoxyquinazolin-4-yl)piperidin-4-yl)acetate (750 mg, 77%). 2M NaOH solution in H₂O (1 mL) was added to a mixture of ethyl 2-(1-(6,7-dimethoxyquinazolin-4-yl)piperidin-4-yl)acetate (250 mg, 0.696 mmol) in THF (10 mL). The mixture was stirred at room temperature for 16 h and then quenched by the addition of 1M HCl solution. The organic phase was extracted with ethyl acetate, washed with brine, dried (Na₂SO₄) and evaporated to dryness under reduced pressure to give the acid 72 (200 mg, 86%) as a white solid.

To a mixture of the acid 72 (300 mg, 0.906 mmol) in THF (10 mL) was added NH₂OH·HCl (76 mg, 1.09 mmol), DIEA (468 mg, 3.63 mmol) and (benzotriazole-1-yloxy)tris(dimethylamino)phosphonium hexafluorophosphate (BOP) (481 mg, 1.09 mmol). The mixture was stirred at room temperature for 16 h and then diluted with water, extracted with ethyl acetate, washed with brine solution, dried (Na₂SO₄) and evaporated to dryness under reduced pressure. Chromatography (SiO₂, solvents) to give the title product 8 (180 mg, 77%) as a white solid. LCMS: [M+H]⁺ m/z 347.10. ¹H NMR (400 MHz, D₂O) δ 8.42 (s, 1H), 7.13 (s, 1H), 7.00 (s, 1H), 4.68-4.62 (m, 2H), 3.95 (s, 3H), 3.91 (s, 3H), 3.51-3.45 (m, 2H), 2.21-2.15 (m, 3H), 1.93-1.0 (m, 2H) and 1.45-1.36 (m, 2H).

### Preparation of 1-(6,7-dimethoxyquinazolin-4-yl)-N-hydroxypiperidine-4-carboxamide 9 (in Table 3a)

Was synthesized according to the procedure for 8 but using ethyl piperidine-4-carboxylate 73.

LCMS: [M+H]⁺ m/z 333.25. ¹H NMR (400 MHz, D₂O) δ 8.39 (s, 1H), 7.04 (s, 1H), 6.94 (s, 1H), 4.62-4.58 (m, 2H), 3.91 (s, 3H), 3.86 (s, 3H), 3.47-3.41 (m, 2H), 2.65-2.60 (m, 1H), 1.97-1.94 (m, 2H) and 1.82-1.77 (m, 2H).

General Procedure for compounds 10, 11, 12, 13 and 16 (in Table 3a).

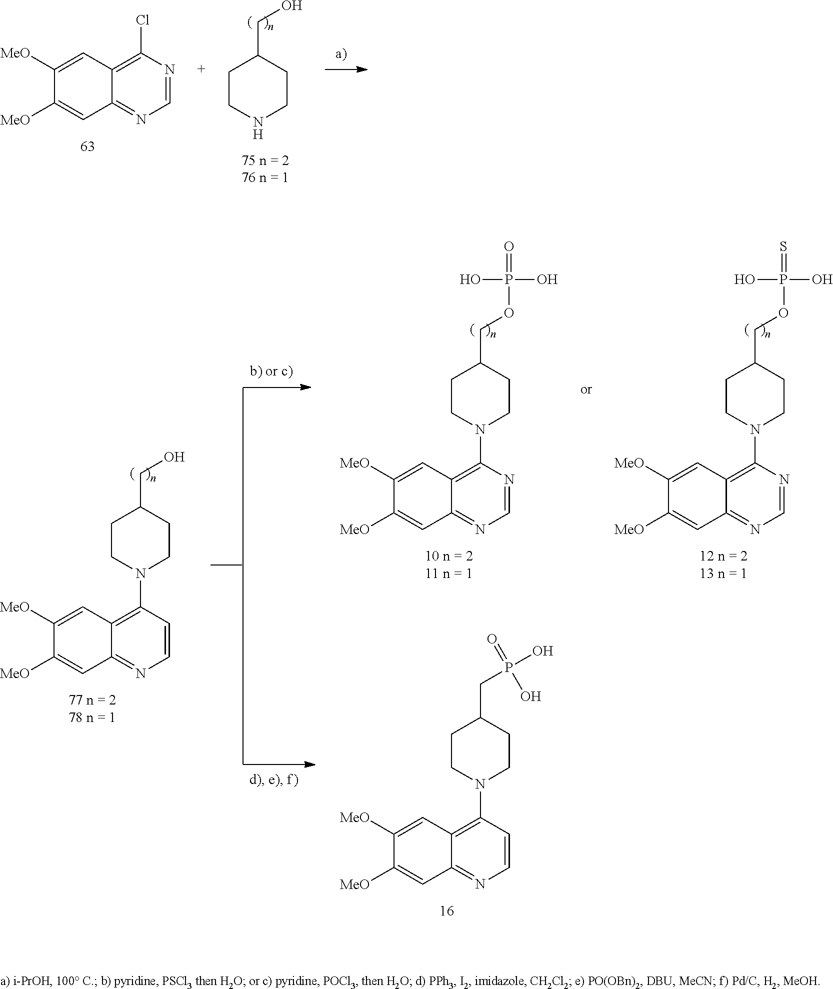

### Preparation of 2-(1-(6,7-dimethoxyquinolin-4-yl)piperidin-4-yl)ethan-1-ol 77

A mixture of 4-chloro-6,7-dimethoxyquinazoline 63 (1.0 g, 4.46 mmol) and piperidin-4-ylethanol 79 (633 mg, 4.91 mmol) in isopropanol (10 mL) was stirred at 100° C. for 16 b in a sealed tube. Upon cooling, the reaction mixture was concentrated under reduced pressure and the residue was purified by silica gel chromatography (SiO₂; EtOAc in petroleum ether) to give 2-(1-(6,7-dimethoxyquinolin-4-yl)piperidin-4-yl)ethan-1-ol 75 (1.3 g, 91%).

### Preparation of (1-(6,7-dimethoxyquinolin-4-yl)piperidin-4-yl)methanol 78

A mixture of 4-chloro-6,7-dimethoxyquinazoline 63 (900 mg, 4.02 mmol) and piperdin-4-ylmethanol 76 (508 mg, 4.42 mmol) in i-PrOH (10 mL) was stirred at 100° C. for 16 h in a sealed tube. Upon cooling, the reaction mixture was evaporated to dryness under reduced pressure. Purification by chromatography (SiO₂: 10 to 80% ethyl acetate in petroleum ether) to give (1-(6,7-dimethoxyquinolin-4-yl)piperidin-4-yl)methanol 78 (1 g, 82%).

### Preparation of 2-(1-(6,7-dimethoxyquinazolin-4-yl)piperidin-4-yl)ethyl Dihydrogen Phosphate 10 (in Table 3a)

2-(1-(6,7-Dimethoxyquinazolin-4-yl)piperidin-4-yl)ethan-1-ol 77 (340 mg, 1.07 mmol) was dissolved in 10 mL dry pyridine, then it was cooled to −15° C. and stirred for 10 min. POCl₃ (821 mg, 5.4 mmol) was added dropwise under a N₂ atmosphere, The reaction temperature was raised to 0° C. slowly, then stirred for another 30 min again. The mixture was poured into sodium hydrogen carbonate solution (800 mg in 250 mL water) at 0° C. The desired compound was extracted with dichloromethane. The organic phase was concentrated and purified with Prep-HPLC to give 2-(1-(6,7-dimethoxyquinazolin-4-yl)piperidin-4-yl)ethyl dihydrogen phosphate 10 (52 mg, 12%) as a white solid.

LCMS: [M+H]⁺ m/z 398. ¹H NMR (400 MHz, DMSO-d₆) δ 8.54 (s, 1H), 7.17 (s, 1H), 7.15 (s, 1H), 4.28-4.16 (m, 2H), 3.93 (s, 8H), 3.13-3.04 (m, 2H), 1.90-1.80 (m, 2H), 1.75 (s, 1H), 1.59 (d, J=6.4 Hz, 2H) and 1.44-1.32 (m, 2H).

### Preparation of (1-(6,7-dimethoxyquinolin-4-yl)piperidin-4-yl)methyl Dihydrogen Phosphate 11 (in Table 3a)

(1-(6,7-Dimethoxyquinolin-4-yl)piperidin-4-yl)methanol 78 (100 mg, 0.33 mmol) was dissolved in dry pyridine (3 mL), then it was cooled to −15° C. and stirred for 10 min. POCl₃ (253 mg, 1.65 mmol) was added dropwise under nitrogen atmosphere. The reaction temperature was raised to 0° C. slowly, then stirred for another 30 min. The mixture was poured into aqueous NaHCO₃ solution (160 mg in 50 mL of water) at 0° C. The desired compound was extracted with dichloromethane and then evaporated to dryness under reduced pressure. Purification by Prep-HPLC afforded (1-(6,7-dimethoxyquinolin-4-yl)piperidin-4-yl)methyl dihydrogen phosphate 11 (70 mg, 55%) as white powder after lyophilization. LCMS: [M+H]⁺ m/z 384.20. ¹H NMR (400 MHz, DMSO-d₆) δ 8.74 (d, J=1.7 Hz, 1H), 7.31 (s, 1H), 7.20 (s, 1H), 4.66 (d, J=13.0 Hz, 1H), 3.97 (m, J=12.6, 1.6 Hz, 8H), 3.76 (t, J=6.6 Hz, 3H), 2.19-2.00 (m, 1H), 1.92 (d, J=13.5 Hz, 2H), 1.45 (dd, J=14.2, 10.7 Hz, 1H).

### Preparation of O-(2-(1-(6,7-dimethoxyquinazolin-4-yl)piperidin-4-yl)ethyl) O,O-Dihydrogen Phosphorothioate 12 (in Table 3a)

To a solution of 2-(1-(6,7-dimethoxyquinolin-4-yl)piperidin-4-yl)ethan-1-ol 77 (150 mg, 0.473 mmol) in dry pyridine (5 mL) was added P(S)C13 (477 mg, 2.84 mmol) at −15° C. After being stirred at 0° C. for 0.5 h, the mixture was poured over a solution of NaHCO₃ (238 mg, 2.84 mmol) in H₂O (50 mL). The mixture was stirred at 0° C. for 2 h. The progress of the reaction mixture was monitored by LCMS. Then the mixture was concentrated under reduced pressure and the residue was purified by prep-HPLC to afford compound 12 (16 mg, 8%) as a light yellow solid. LCMS: [M+H]⁺ m/z 414.05. ¹H NMR (400 MHz, DMSO-d₆) δ 8.62 (s, 1H), 7.19 (d, J=7.7 Hz, 2H), 4.45 (d, J=12.3 Hz, 2H), 3.91 (d, J=11.3 Hz, 10H), 1.86 (d, J=12.2 Hz, 3H), 1.56 (d, J=6.4 Hz, 2H), 1.34 (d, J=10.7 Hz, 2H).

### Preparation of O-((1-(6,7-dimethoxyquinazolin-4-yl)piperidin-4-yl)methyl)O,O-dihydrogen Phosphorothioate 13 (in Table 3a)

To a solution of (1-(6,7-dimethoxyquinolin-4-yl)piperidin-4-yl)methanol 78 (100 mg, 0.330 mmol) in dry pyridine (5 mL) was added P(S)Cl₃ (280 mg, 1.98 mmol) at −15° C. After being stirred at 0° C. for 0.5 h, the mixture was poured over a solution of NaHCO₃ (116 mg, 1.98 mmol) in H₂O (50 mL). The mixture was stirred at 0° C. for 2 h. The mixture was evaporated to dryness under reduced pressure and the residue was purified by prep-HPLC to afford compound 13 (10 mg, 7.6%) as a yellow solid. LCMS: [M+H]⁺ m/z 400.15. ¹H NMR (400 MHz, DMSO-d₆) δ 8.54 (s, 1H), 7.18 (s, 1H), 7.11 (s, 1H), 4.25 (d, J=13.4 Hz, 2H), 3.89 (d, J=9.1 Hz, 6H), 3.76 (s, 2H), 3.10 (d, J=11.8 Hz, 3H), 1.94 (s, 1H), 1.81 (d, J=12.7 Hz, 2H), 1.39 (d, J=11.4 Hz, 1H).

### Preparation of ((1-(6,7-dimethoxyquinazolin-4-yl)piperidin-4-yl)methyl)phosphonic Acid 16

PPh₃ (3.39 g, 15 mmol) and imidazole (1.02 g, 15 mmol) in anhydrous CH₂Cl₂ (40 mL) was stirred in 0° C. for 10 min and then I₂ (3.8 g, 15 mmol) was added. The crude reaction mixture was placed under a nitrogen atmosphere and stirred for a further 10 min and then (1-(6,7-dimethoxyquinazolin-4-yl)piperidin-4-yl)methanol 78 (3.03 g, 10 mmol) was added. The reaction mixture was left to stir at room temperature overnight. The reaction was quenched by the addition of aqueous Na₂S₂O₃. The crude mixture was extracted with CH₂Cl₂, washed with water, brine, dried (Na₂SO₄) and evaporated to dryness under reduced pressure. Recrystallisation from methanol gave 4-(4-(iodomethyl)piperidin-1-yl)-6,7-dimethoxyquinazoline (2.28 g, 56%) as light yellow solid. LCMS: [M+H]⁺ m/z 414.3. ¹H NMR (400 MHz, CDCl₃) δ 8.63 (d, J=1.3 Hz, 1H), 7.28 (s, 1H), 7.07 (s, 1H), 4.23 (s, 2H), 4.00 (s, 6H), 3.19 (d, J=6.5 Hz, 2H), 3.08 (s, 2H), 2.11-2.00 (m, 2H), 1.82 (s, 1H), 1.49 (s, 2H), 1.29-1.20 (m, 1H), 1,8-Diazabicyclo(5.4.0)undec-7-ene (DBU) (9.2 g, 60.5 mmol) was added to a cooled (0° C.) solution of bis(benzyloxy)(oxo)-?4-phosphane (9.5 g, 36.3 mmol) in anhydrous MeCN (40 mL). After 10 min, 4-(4-(iodomethyl)piperidin-1-yl)-6,7-dimethoxyquinazoline (5.0 g, 12.1 mmol) was added. The resulting mixture was stirred for overnight and then evaporated to dryness under reduced pressure. The residue was dissolved in ethyl acetate, washed with water, brine, dried (MgSO₄) and evaporated to dryness under reduced pressure. Purification with FCC [CH₂Cl₂:MeOH (50:1)] provided dibenzyl ((1-(6,7-dimethoxyquinazolin-4-yl)piperidin-4-yl)methyl)phosphonate (1.1 g, 18%) as a colorless viscous oil. LCMS: [M+H]⁺ m/z 548.20. ¹H NMR (400 MHz, CDCl₃) δ 8.57 (s, 1H), 7.89 (s, 1H), 7.39-7.33 (m, 10H), 6.99 (s, 1H), 5.08 (m, 3H), 4.96 (m, 2H), 4.64 (d, J=13.5 Hz, 2H), 4.09 (s, 3H), 3.93 (s, 3H), 3.27 (d, J=12.9 Hz, 2H), 2.05 (d, J=13.9 Hz, 5H), 1.76 (m, 4H), 1.42 (d, J=12.5 Hz, 2H).

A mixture containing dibenzyl ((1-(6,7-dimethoxyquinazolin-4-yl)piperidin-4-yl)methyl)phosphonate (660 mg, 1.2 mmol) and Pd/C (132 mg, 20% w/w) in MeOH (20 mL) was placed under an atmosphere of H₂ and stirred at room temperature. After 4 h, the crude mixture was filtered through a pad of Celite® and the filtrate was evaporated to dryness under reduced pressure. Purification by prep-HPLC afforded ((1-(6,7-dimethoxyquinazolin-4-yl)piperidin-4-yl)methyl)phosphonic acid 16 (125 mg, 28%) as light yellow solid. LCMS: [M+H]⁺ m/z 368.10. ¹H NMR (400 MHz, DMSO-d₆) δ 8.72 (s, 1H), 7.29 (s, 2H), 4.60 (d, J=12.8 Hz, 2H), 3.95 (d, J=11.2 Hz, 6H), 3.46 (s, 2H), 2.09 (s, 3H), 1.61 (s, 2H), 1.42 (s, 2H).

### Preparation of (2-(1-(6,7-dimethoxyquinazolin-4-yl)piperidin-4-yl)propyl)phosphonic Acid 14 (in Table 3a)

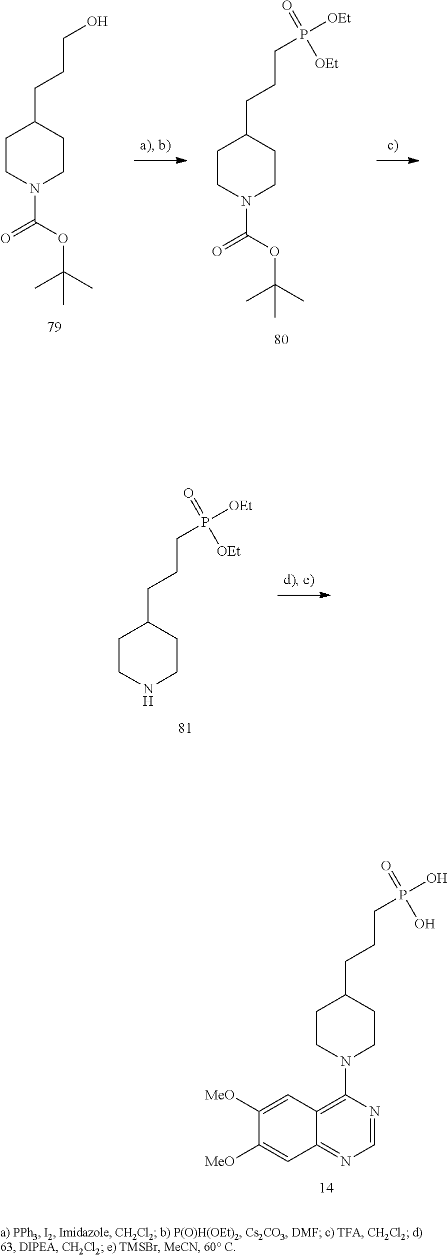 [SMILES: CC(C)(C)OC(=O)N1CCC(CCCO)CC1 ; CCOP(=O)(CCCC1CCN(C(=O)OC(C)(C)C)CC1)OCC ; CCOP(=O)(CCCC1CCNCC1)OCC ; COc1cc2ncnc(N3CCC(CCCP(=O)(O)O)CC3)c2cc1OC]

Iodine (1.35 g, 5.34 mmol) was added to a solution of PPh₃ (1.4 g, 5.34 mmol) and imidazole (0.36 g, 5.34 mmol in CH₂Cl₂ (20 mL). The mixture was stirred at rt for 0.5 h and then a solution of 79 (1.0 g, 4.11 mmol) in CH₂Cl₂ (5 mL) was added dropwise. The reaction mixture was stirred at rt for 4 h and then quenched with saturated Na₂SO3 solution, and extracted with CH₂Cl₂. The organic phase was washed with water, brine, dried (Na₂SO4) and evaporated to dryness under reduced pressure. Chromatography (SiO₂; 5% EtOAc in petroleum ether) to afford tert-butyl 4-(3-iodopropyl)piperidine-1-carboxylate (1.0 g, 68% yield) as light yellow oil.

To a mixture of tert-butyl 4-(3-iodopropyl)piperidine-1-carboxylate (1.0 g, 2.83 mmol in DMF (50 mL) was added diethyl phosphonate (0.58 g, 4.24 mmol) and Cs₂CO₃ (1.84 g, 5.66 mmol). The reaction mixture was stirred under an atmosphere of nitrogen at rt overnight and then quenched by the addition or water. The organic phase was washed with water, brine, dried (Na₂SO₄) and evaporated to dryness under reduced pressure. Chromatography (SiO₂; 20% EtOAc in petroleum ether) gave 80 (0.78 g, 76%) as light yellow oil. LCMS: [M+H]⁺ m/z 364.30.

To a solution of 80 (0.78 g, 2.14 mmol) in CH₂C12 (8 mL) was added TFA (1.5 mL, 21.4 mmol). The mixture was stirred at rt for 4 h and then evaporated to dryness under reduced pressure. To give crude 81 as an oil which was used in the next step without further purification. LCMS: [M+H]⁺ m/z 264.25.

DIPEA (1.37 g, 10.63 mmol) was added to a solution of diethylphosphonate (597 mg, 2.65 mmol) and crude 81 in CH₂Cl₂ (10 mL). The mixture was stirred at rt overnight and then quenched with sat'd aqueous NH₄Cl solution and extracted with CH₂Cl₂. The organic phase was washed with water, brine, dried (Na₂SO₄) and evaporated to dryness under reduced pressure. Chromatography (SiO₂, 5% MeOH in CH₂Cl₂) gave the diethylphosphonate (0.5 g, 39%) intermediate as a yellow oil. This was solvated in MeCN (10 mL) was TMSBr (1.46 mL, 11.07 mmol) was added. The resulting mixture was stirred at 60° C. for 6 h and then cooled to room temperature and evaporated to dryness under reduced pressure. Chromatography (Prep-HPLC) gave 14 (220 mg, 50%) as white solid. LCMS: [M+H]⁺ m/z 396.20. ¹H NMR (400 MHz, Methanol-d₄) δ 8.51 (s, 1H), 7.33 (s, 1H), 7.14 (s, 1H), 4.02 (s, 3H), 3.97 (s, 3H), 3.49 (t, J=12 Hz, 2H), 2.00-1.97 (m, 3H), 1.75-1.66 (m, 5H) and 1.45-1.37 (n, 5H).

### Preparation of (2-(1-(6,7-dimethoxyquinazolin-4-yl)piperidin-4-yl)ethyl)phosphonothioic O,O-acid 17

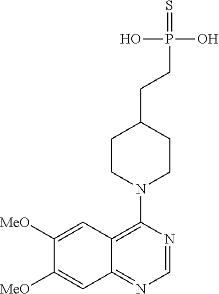 [SMILES: COc1cc2ncnc(N3CCC(CCP(O)(O)=S)CC3)c2cc1OC]

To a stirred solution of O,O-diethyl (2-(piperidin-4-yl)ethyl)phosphonothioate (400 mg, 1.51 mmol) and DIPEA (927 mg, 7.19 mmol) in DMSO (10 mL) was added 4-chloro-6,7-dimethoxy-quinazoline 66 (403 mg, 1.80 mmol). The reaction mixture was placed under a nitrogen atmosphere and then stirred at 80° C. for 16 h. The reaction mixture was cooled to room temperature, diluted with water and extracted with ethyl acetate. The organic phase was dried (Na₂SO₄) and evaporated to dryness under reduced pressure. Purification (SiO₂, 0-100% EtOAc in Hexanes) provided O,O-diethyl(2-(1-(6,7-dimethoxyquinazolin-4-yl)piperidin-4-yl)ethyl)phosphonothioate (380 mg, 46%). A stirred solution of O,O-diethyl(2-(1-(6,7-dimethoxyquinazolin-4-yl)piperidin-4-yl)ethyl)phosphonothioate (45 mg, 0.099 mmol) in TMSI (7 mL) was stirred at 60° C. for 16 h and then cooled to room temperature. The mixture was diluted with water and extracted with ethyl acetate. The organic phase was dried (Na₂SO4) and then evaporated to dryness under reduced pressure. Purification by prep-HPLC afforded (2-(1-(6,7-dimethoxyquinazolin-4-yl)piperidin-4-yl)ethyl)phosphonothioic O,O-acid 17 (13 mg, 32%) as a white solid. LCMS: [M+H]⁺ m/z 396.25. ¹H NMR (400 MHz, DMSO-d₆) δ 8.51 (s, 1H), 7.33 (s, 1H), 7.16 (s, 1H), 4.02 (s, 3H), 3.97 (s, 3H), 3.58 (d, J=10.4 Hz, 3H), 3.48 (t, J=12.0 Hz, 2H), 2.00 (d, J=11.7 Hz, 2H), 1.81 (s, 1H), 1.64 (d, J=17.9 Hz, 2H), 1.61-1.51 (m, 2H), 1.45-1.32 (m, 2H).

### (2-(4-(6,7-Dimethoxyquinazolin-4-yl)piperidin-1-yl)ethyl)phosphonic Acid 18 (in Table 3a)

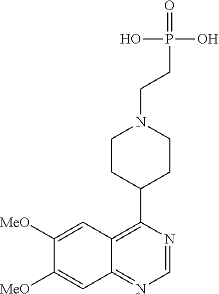 [SMILES: COc1cc2ncnc(C3CCN(CCP(=O)(O)O)CC3)c2cc1OC]

LCMS: [M+H]⁺ m/z 382.25 ¹H NMR (400 MHz, D₂O) δ 8.65 (s, 1H), 6.93 (s, 1H), 6.76 (s, 1H), 3.86 (s, 3H), 3.84 (s, 3H), 3.30-3.22 (m, 1H), 3.18-3.15 (m, 2H), 2.73-2.66 (m, 2H), 2.37-2.32 (m, 2H) and 1.79-1.63 (m, 6H).

### (2-(4-(6,7-Dimethoxyquinazolin-4-yl)piperazin-1-yl)ethyl)phosphonic Acid Hydrogen Bromide Salt 19 (in Table 3a)

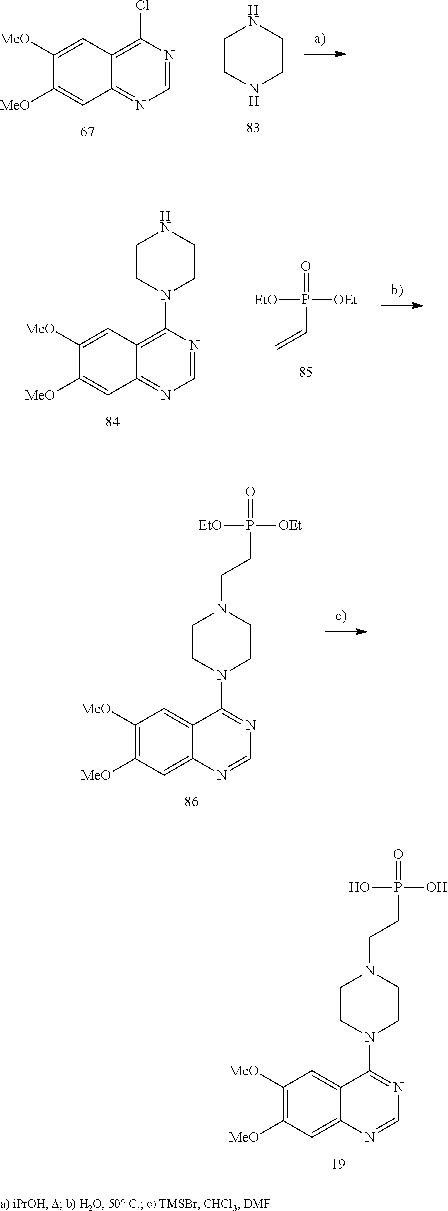

A mixture containing pyrazine 82 and compound 63 in propan-2-ol were heated at reflux for 30 min and then cooled to room temperature. The reaction mixture was quenched with water and extracted into chloroform. The organic phase was separated, washed with water, brine, dried (Na₂SO₄) and evaporated to dryness under reduced pressure. Crystallization from diethyl ether gave 83 as a white solid (1.65 g, 84%). The piperazine 83 (0.31 g, 1.1 mmol) was dissolved in water (20 mL) and vinyl phosphonate 84 (0.19 g, 1.2 mmol) was added. The resulting mixture was heated at 50° C. for 1 h and then cooled to room temperature. Extraction with chloroform, dried (Na₂SO₄) and evaporated to dryness under reduced pressure. Chromatography (SiO₂ 12 g; 15% MeOH in CH₂Cl₂), followed by crystallization from diethyl ether, gave the ethyl ester 85 (0.23 g; 49%) as a white solid. LCMS: [M+H]⁺ m/z 410.10 ¹H NMR (500 MHz, Chloroform-d) δ 8.67 (s, 1H), 7.25 (s, 1H), 7.09 (s, 1H), 4.01 (d, J=18.8 Hz, 6H), 3.77 (d, J=10.9 Hz, 6H), 3.68 (t, J=4.9 Hz, 4H), 3.49 (s, 2H), 2.81-2.66 (m, 6H), 2.11-2.00 (m, 2H). Trimethylsilyl bromide (198 mg, 1.3 mmol) was added to a solution of the 4-[4-(2-diethoxyphosphorylethyl)piperazin-1-yl]-6,7-dimethoxy-quinazoline 85 (600 mg, 3.8 mmol) in chloroform (20 mL) and DMF (5 mL). The resulting solution was left to stir at room temperature for 3 h and then quenched by addition of methanol. The mixture was evaporated to dryness under reduced pressure and crystallized from methanol-diethyl ether to give the desired product 19 (0.23 g, 89%) as the HBr salt. LCMS. [M+H]⁺ m/z 382.8. ¹H NMR (500 MHz, DMSO-d₆) δ 8.97 (s, 1H), 7.96 (s, 1H), 7.42 (s, 1H), 7.35 (s, 1H), 4.02 (s, 3H), 4.00 (s, 3H), 3.34 (t, J=8.6 Hz, 2H), 3.17 (s, 2H), 2.90 (s, 2H), 2.74 (s, 2H), 2.20-2.08 (m, 2H).

### Preparation of (4-(6,7-dimethoxyquinazolin-4-yl)phenethyl)phosphonic Acid 20 (in Table 3a)

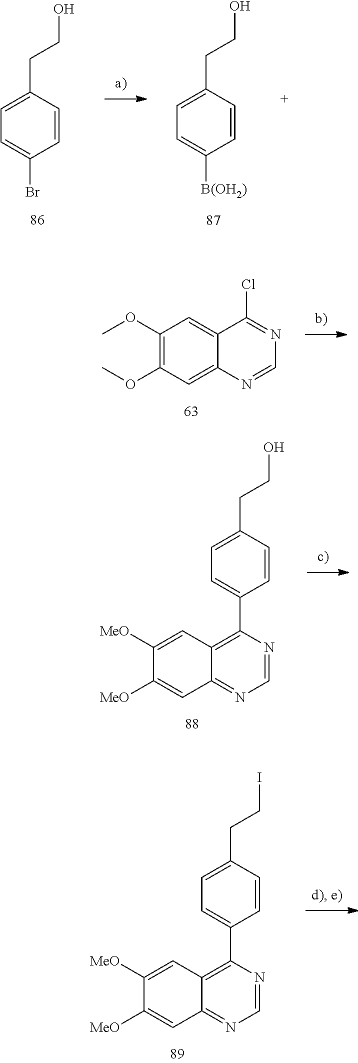

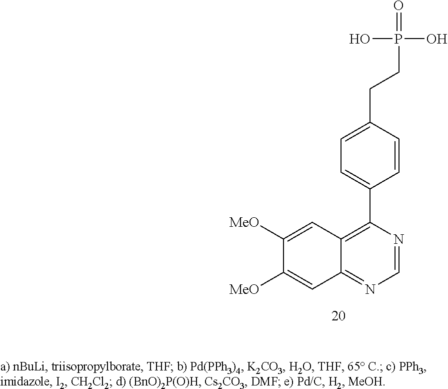 [SMILES: COc1cc2ncnc(-c3ccc(CCP(=O)(O)O)cc3)c2cc1OC]

2.5M n-Butyllithium (24 mL) was added to a solution of 2-(4-bromophenyl)ethan-1-ol 86 (5.0 g, 24.8 mmol) in anhydrous THF (100 mL) at −78° C. under a nitrogen atmosphere. After being stirred for 1 h, triisopropyl borate (8.6 mL) was added to the mixture. The reaction mixture was stirred at room temperature for 1 h and then quenched by the addition of 2M HCl solution (100 mL) and stirred for 1 h. The mixture was extracted with dichloromethane (3×100 mL), dried (Na₂SO₄) and evaporated to dryness under reduced pressure. Chromatography (SiO₂; dichloromethane: methanol, 1:0 to 20:0) gave the boronic acid 87 (1.34 g, 33%) as light yellow solid. This material was then dissolved in a solution of THF (30 mL) and water (10 mL), 4-Chloro-6,7-dimethoxyquinazoline 63 (2.24 g, 10.0 mmol) and potassium carbonate (2.76 g, 20.0 mmol) were added to the solution followed by tetrakis(triphenylphosphine)palladium (0.5 g, 0.43 mmol). The resulting mixture was stirred at 65° C. for 16 h and then diluted with ethyl acetate, washed with brine, dried (Na₂SO₄) and evaporated to dryness under reduced pressure. Chromatography (SiO₂: (methanol in dichloromethane 0 to 10%) gave 88 (1.43 g, 60%) as light yellow solid.

To a solution of triphenylphosphine (2.36 g, 9.0 mmol) in dichloromethane (24 mL) was added imidazole (700 mg, 10.28 mmol) at 0° C. After being stirred for 10 min, I₂ (2.3 g, 9.0 mmol) was added. After being stirred for a further 10 min, compound 89 (1.5 g, 4.8 mmol) in dichloromethane (12 mL). The mixture was allowed to warm to room temperature and stirred for 5 h. Then the mixture was diluted with dichloromethane (36 mL), washed with brine, dried (Na₂SO₄) and evaporated to dryness under reduced pressure. Chromatography (SiO₂:petroleum ether:ethyl acetate 10:1) gave 89 (4.0 g), as colorless oil.

Cesium carbonate (1.426 g, 4.4 mmol) was added to a mixture of crude 89 (930 mg, 2.2 mmol) and dibenzyl phosphonate (884 mg, 3.37 mmol) in DMF (20 mL). The mixture was placed under an atmosphere of nitrogen and stirred at room temperature for 3 h. Once complete, the reaction mixture was filtered and evaporated to dryness under reduced pressure. Chromatography (C18 column: water:acetonitrile, 1:0 to 80:1) followed by lyophilization gave the dibenzyl intermediate (750 mg, 79%) as off-white solid. Dibenzyl (4-(6,7-dimethoxyquinazolin-4-yl)phenethyl)phosphonate (230 mg, 0.41 mmol) was dissolved in MeOH (20 mL). Pd/C (46 mg, 20% w/w) was added and the mixture stirred under a hydrogen atmosphere at room temperature for 24 h and then filtered through Celite®. Chromatography (prep-HPLC under acidic conditions) gave compound 20 (55.4 mg, 36%) as yellow solid. LCMS: [M+H]⁺ m/z 375.0. ¹H NMR (400 MHz, DMSO-d₆)): δ 9.09 (s, 1H), 7.75-7.73 (d, J=8.0 Hz, 2H), 7.45-7.43 (m, 2H), 7.41 (s, 1H), 7.32 (s, 1H), 4.08 (s, 1H), 3.98 (s, 3H), 3.91 (s, 1H), 3.81 (s, 3H), 2.89-2.87 (m, 3H) and 1.89 (m, 2H).

### Synthesis of (4-((6,7-dimethoxyquinolin-4-yl)amino)phenyl)phosphonic Acid Sodium Salt 21 (in Table 3a)

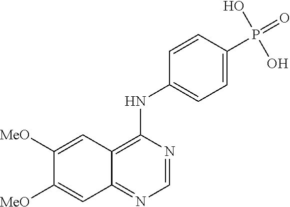 [SMILES: COc1cc2ncnc(Nc3ccc(P(=O)(O)O)cc3)c2cc1OC]

A mixture of 4-chloro-6,7-dimethoxy-quinazoline 67 (0.67 g, 3.0 mmol) and diethyl (4-aminophenyl)phosphonate (0.69 g, 3.0 mmol) in iPrOH (10 mL) was heated at reflux overnight. The solid precipitate was filtered, washed with EtOAc and dried to give diethyl (4-((6,7-dimethoxyquinazolin-4-yl)amino)phenyl)phosphonate (0.92 g, 73% yield) as a white solid. The product was dissolved in MeCN (20 mL) and to this was added trimethylsilylbromide (2.8 mL, 22 mmol). The resulting mixture was stirred at 60° C. for 6 h, cooled and then evaporated to dryness under reduced pressure. The crude residue was quenched by the addition of saturated aqueous NaHCO₃ (adjusted to pH 8). The resulting mixture was purified by prep-HPLC (under neutral conditions) and then lyophilized to give the desired product 21 (300 mg, 35% yield) as an off-white solid. LCMS: [M+H]‘ m’ 362.10. ¹H NMR (400 MHz, D₂O) δ 7.73 (s, 1H), 7.53 (t, J=9.8 Hz, 2H), 7.22 (d, J=7.4 Hz, 2H), 6.39 (s, 1H), 6.16 (s, 1H) and 3.39 (s, 6H).

### Synthesis of (4-((6,7-dimethoxyquinolin-4-yl)amino)benzyl)phosphonic Acid Sodium Salt 22 (in Table 3a)

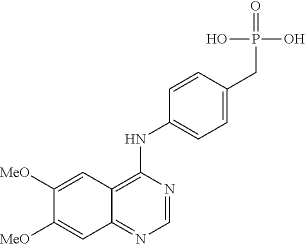 [SMILES: COc1cc2ncnc(Nc3ccc(CP(=O)(O)O)cc3)c2cc1OC]

A mixture of 4-chloro-6,7-dimethoxy-quinazoline 67 (0.34 g, 1.5 mmol) and diethyl (4-aminobenzyl)phosphonate (0.36 g, 3.0 mmol) in iPrOH (10 mL) was heated at reflux overnight. The precipitate was filtered, washed with EtOAc and evaporated to dryness under reduced pressure and then dissolved in acetonitrile (20 mL). To this was added trimethylsilylbromide (0.58 mL, 4.6 mmol). The mixture was stirred at 60° C. for 6 h. After concentration, the residue was treated with sat'd aqueous NaHCO₃ until the solution reached pH 8. The mixture was purified by prep-HPLC (neutral) to give 22 (104 mg, 57%) as an off-white solid. LCMS: [M+H]⁺ m/z 376.10. ¹H NMR (400 MHz, D₂O) δ 7.77 (s, 1H), 7.15 (s, 4H), 6.49 (s, 1H), 6.21 (s, 1H), 3.47 (s, 6H) and 2.70 (d, J=19.5 Hz, 2H).

### (4-(((6,7-Dimethoxyquinolin-4-yl)amino)methyl)phenyl)phosphonic Acid Sodium Salt 23 (Also Referred to as 4 in Table 1)

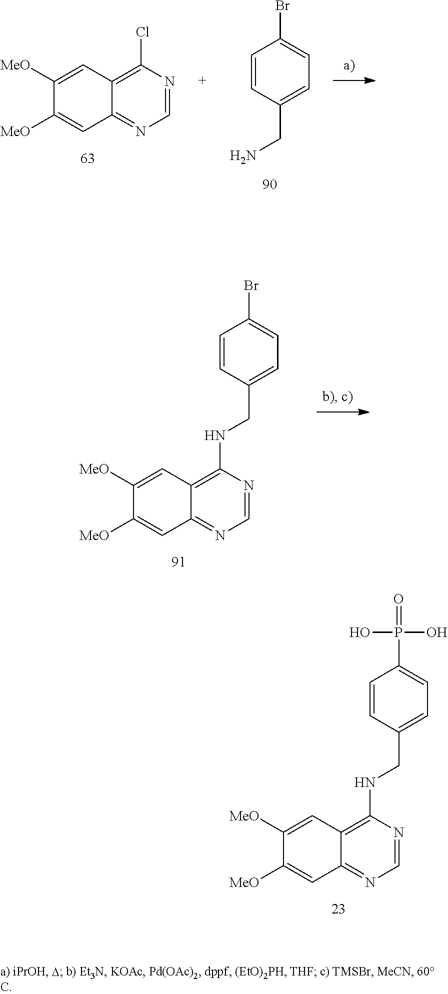 [SMILES: COc1cc2ncnc(Cl)c2cc1OC ; NCc1ccc(Br)cc1 ; COc1cc2ncnc(NCc3ccc(Br)cc3)c2cc1OC ; COc1cc2ncnc(NCc3ccc(P(=O)(O)O)cc3)c2cc1OC]

A mixture of 4-chloro-6,7-dimethoxy-quinazoline 63 (0.93 g, 4.14 mmol) and (4-bromophenyl)methanamine 90 (0.77 g, 4.14 mmol) in iPrOH (10 mL) was heated at reflux overnight. The solid precipitated was filtered, washed with ethyl acetate and evaporated to dryness under reduced pressure to give N-(4-bromobenzyl)-6,7-dimethoxyquinazolin-4-amine hydrochloride 91 (1.5 g, 88%) as a white solid. Triethylamine (0.37 mL, 2.68 mmol) was added to a mixture of KOAc (11 mg, 0.112 mmol), Pd(OAc)₂ (5.5 mg, 0.025 mmol), dppf (27 mg, 0.049 mmol) in THF (10 mL) was purged with nitrogen. Triethylamine (0.37 mL, 2.68 mmol) was added. After stirring at 70° C. for 15 min, a solution of N-(4-bromobenzyl)-6,7-dimethoxyquinazolin-4-amine hydrochloride (0.5 g, 1.22 mmol) and diethyl phosphonate (0.16 g, 1.22 mmol) in THF (10 mL) was added. The reaction was stirred at reflux for 6 h and then partitioned between EtOAc (30 mL) and water (20 mL). The organic phase was separated, washed with water, brine, dried (Na₂SO4) and evaporated to dryness under reduced pressure. Purification by column chromatography (SiO₂; 50% petroleum ether in ethyl acetate) afforded diethyl (4-(((6,7-dimethoxyquinazolin-4-yl)amino)methyl)phenyl) phosphonate (0.2 g, 38%) as yellow solid.

To a solution of diethyl (4-(((6,7-dimethoxyquinazolin-4-yl)amino)methyl)phenyl) phosphonate (0.5 g, 1.16 mmol) in MeCN (20 mL) was added TMSBr (1.45 mL, 11.5 mmol). The mixture was stirred at 60° C. for 6 h, cooled to room temperature and then evaporated under reduced pressure. The residue was quenched with saturated aqueous NaHCO₃ (pH 9) and the resulting mixture purified prep-HPLC (neutral) to afford the title product 23 as an off-white solid (102 mg, 22%). LCMS: [M−H]⁺ m/z: 374.00. ¹H NMR (400 MHz, D₂O) δ 8.00 (s, 1H), 7.61 (s, 2H), 7.29 (d, J=7.6 Hz, 2H), 6.70 (s, 1H), 6.55 (s, 1H), 4.62 (s, 2H), 3.75 (d, J=18.2 Hz, 6H).

### General Procedure Synthesis of Dimethyl (2-(piperidin-4-yl)ethyl)phosphonate 92 and Diethyl (2-(piperidin-4-yl)ethyl)phosphonate 93

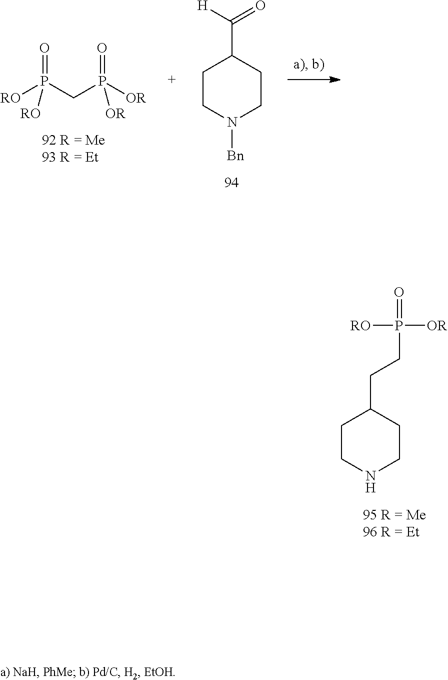

Sodium hydride (1.1 mol. equiv.) is carefully added to a stirred solution of bis(dimethoxyphosphoryl)methane 92 or bis(diethoxyphosphoryl)methane 93 (1 mol. equiv.) in toluene at room temperature. The reaction mixture is then placed under an atmosphere of nitrogen and a solution of 1-benzylpiperidine-4-carbaldehyde 94 (1 mol. equiv.) in toluene was slowly added, keeping the temperature below 40° C. The resulting mixture is left to stir at room temperature for 16 h and then quenched by the addition of aqueous saturated NH₄Cl solution. The organic phase was separated, washed with brine, dried (MgSO₄) and evaporated to dryness. Chromatography (120 g SiO₂; 5 to 100% gradient of EtOAc in Hexanes) provides dimethyl or diethyl (E)-(2-(1-benzylpiperidin-4-yl)vinyl)phosphonates as a colorless oil. To a mixture of dimethyl or diethyl (E)-(2-(1-benzylpiperidin-4-yl)vinyl)phosphonate (1 mol. equiv.) in ethanol is added catalytic Pd/C. The mixture is placed under an atmosphere of hydrogen and stirred at room temperature for 12 h, filtered and evaporated to dryness under reduced pressure to give either the dimethyl or diethyl (2-(piperidin-4-yl)ethyl)phosphonates 95 and 96 as colorless oils.

### General Procedure for the Synthesis of Dibenzyl (2-(piperidin-4-yl)ethyl)phosphonate 100

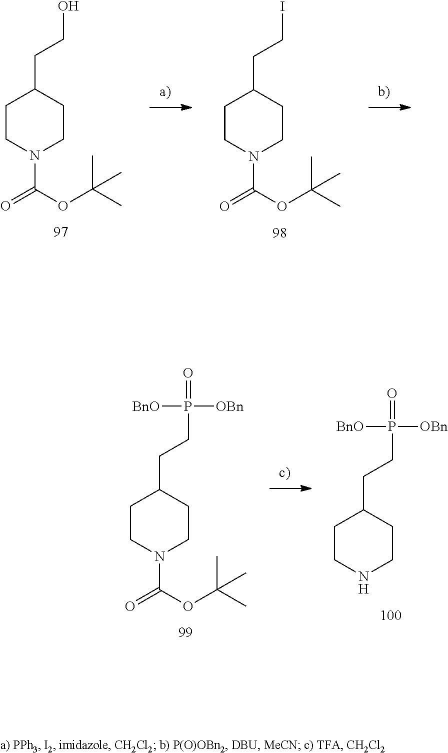 [SMILES: CC(C)(C)OC(=O)N1CCC(CCO)CC1 ; CC(C)(C)OC(=O)N1CCC(CCI)CC1 ; CC(C)(C)OC(=O)N1CCC(CCP(=O)(OCc2ccccc2)OCc2ccccc2)CC1 ; O=P(CCC1CCNCC1)(OCc1ccccc1)OCc1ccccc1]

Iodine (1.5 mol. equiv.) was added to a solution of PPh₃ (1.5 mol. equiv.) and imidazole (1.5 mol. equiv.) in CH₂Cl₂. After stirring for 10 min, a solution of 97 (1.0 mol. equiv.) in CH₂Cl₂ is added dropwise. The mixture is stirred at room temperature for 2 h, filtered through a pad of Celite® and treated with 5% sodium thiosulfate solution. The mixture is extracted with ethyl acetate, washed with brine, dried (Na₂SO₄) and evaporated to dryness under reduced pressure. Chromatography provides 98 as an oil.

DBU (5.0 mol. equiv.) was added to a solution of compound 98 (3.0 mol. equiv.) in MeCN at 40° C. After being stirred for 10 min, a solution of dibenzylphosphonate (1.0 mol equiv.) in MeCN was added dropwise. After stirring for 2 hours, the reaction mixture is evaporated to dryness under reduced pressure and purified by chromatography to yield 99.

A solution of compound 99 (1.0 mol. equiv.) in TFA/DCM is stirred at room temperature for 1 h and then evaporated to dryness under reduced pressure to afford 100 as an oil.

### General Methods to Synthesize (2-(1-(quinazolin-4-yl)piperidin-4-yl)ethyl)phosphonic acids, (2-(1-(quinolin-4-yl)piperidin-4-yl)ethyl)phosphonic Acids and (2-(1-(isoquinolin-1-yl)piperidin-4-yl)ethyl)phosphonic Acids

### Method A:

Diisopropylethylamine (2 mol. equiv.) was added to a mixture of either dimethyl (2-(piperidin-4-yl)ethyl)phosphonate 95 or diethyl (2-(piperidin-4-yl)ethyl)phosphonate % (1.1 mol. equiv.) and a 4-chloroquinazoline, 4-chloroquinoline or 1-chloroisoquinoline (1 mol. equiv.) in isopropyl alcohol (0.1 M reaction concentration). After stirring at 90° C. for 3 h, the reaction mixture was cooled and evaporated to dryness. Purification of silica gel (5% MeOH in dichloromethane) provides the dimethyl or diethyl phosphonates. To a cooled (0° C.) solution of the phosphonate (1 mol. equiv.) in chloroform or dichloromethane (0.5 M reaction concentration) was added trimethylsilyl bromide (3 mol. equiv.). The reaction mixture was allowed to warm to room temperature and, after 90 min, was quenched by the addition of methanol. The mixture was evaporated to dryness under reduced pressure and then solvated in methanol. The reaction mixture was concentrated to half volume, filtered to remove precipitate, and then evaporated to dryness. The residue was crystallized with dichloromethane, filtered and dried under reduced pressure to give the desired phosphonic acid as a bromide salt.

### Method B:

Diisopropylethylamine (3 mol. equiv.) was added to a mixture of either dimethyl (2-(piperidin-4-yl)ethyl)phosphonate 95 or diethyl (2-(piperidin-4-yl)ethyl)phosphonate 96 (1.1 mol. equiv.) and a 4-chloroquinazoline, 4-chloroquinoline or 1-chloroisoquinoline (1 mol. equiv.) in dichloromethane (0.1 M reaction concentration). After stirring at room temperature overnight, the reaction mixture was quenched by the addition of sat'd aqueous NH₄Cl solution. The organic phase was separated and washed with water and brine, dried (Na₂SO₄) and evaporated to dryness under reduced pressure. Purification of silica gel (5% MeOH in dichloromethane) provides the dimethyl or diethyl phosphonates. To a cooled (0° C.) solution of the dimethyl or diethyl phosphonates (7 mol. equiv.) in acetonitrile (0.1 M reaction concentration) was added trimethylsilyl bromide (3 mol. equiv.). The reaction mixture was stirred at 60° C. for 6 h, cooled and evaporated to dryness under reduced pressure and the crude residue quenched by the addition of sat'd aqueous NaHCO₃ solution (until pH 8˜9 was observed). The crude residue purified by prep-HPLC (neutral) to give the phosphonic acid as a sodium salt.

### Method C:

Diisopropylethylamine (3 mol. equiv.) was added to a mixture of either dibenzyl(2-(piperidin-4-yl)ethyl)phosphonate 100 (1.1 mol. equiv.) and a 4-chloroquinazoline, 4-chloroquinoline or 1-chloroisoquinoline (1 mol. equiv.) in dichloromethane (0.1 M reaction concentration). After stirring at room temperature overnight, the reaction mixture was quenched by the addition of sat'd aqueous NH₄Cl solution. The organic phase was separated and washed with water and brine, dried (Na₂SO₄) and evaporated to dryness under reduced pressure. Purification of silica gel (5% MeOH in dichloromethane) provides the dibenzyl phosphonates. A mixture of the dibenzyl phosphonate (1 mol. equiv.) and Pd/C in MeOH is placed under an atmosphere of hydrogen and stirred at room temperature for 2 h. The mixture is then filtered through Celite and evaporated to dryness under reduced pressure to give the phosphonic acids.

### Preparation of (2-(146,7-dimethoxyquinazolin-4-yl)piperidin-4-yl)ethyl)phosphonic Acid (or compound 1)

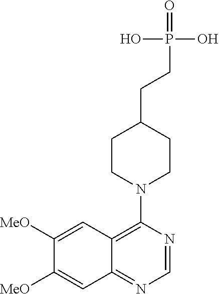 [SMILES: COc1cc2ncnc(N3CCC(CCP(=O)(O)O)CC3)c2cc1OC]

Prepared according to Method A to give 15 (2.1 g, 69%) as an off-white solid. LCMS: [M+H]⁺ m/z 381.8. ¹H NMR (500 MHz, DMSO-d₆) δ 8.77 (s, 1H), 7.34 (s, 1H), 7.23 (s, 1H), 4.71 (d, J=13.1 Hz, 2H), 3.99 (s, 3H), 3.97 (s, 3H), 3.48 (t, J=12.7 Hz, 2H), 3.18 (s, 1H), 1.97-1.90 (m, 2H), 1.62-1.43 (m, 4H), 1.40-1.27 (m, 2H).

### Preparation of (4-(((6,7-dimethoxyquinazolin-4-yl)amino)methyl)benzyl)phosphonic Acid 24 (Also Referred to as 5 in the Tables Herein)

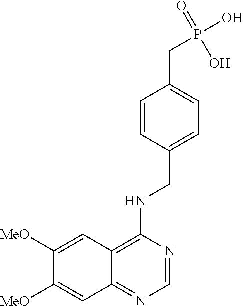 [SMILES: COc1cc2ncnc(NCc3ccc(CP(=O)(O)O)cc3)c2cc1OC]

Prepared according to Method B. The product was isolated by prep-HPLC (9% yield) as an off-white solid. LCMS: [M+H]f m/z 390.15. ¹H NMR (400 MHz, D₂O) δ 8.12 (s, 1H), 7.22 (s, 4H), 7.11 (s, 1H), 6.91 (s, 1H), 4.79 (s, 2H), 4.76 (s, 2H), 3.98 (s, 3H), 3.91 (s, 3H), 2.79 (s, 1H), 2.74 (s, 1H)

### Preparation of (2-(1-(6-methoxyquinazolin-4-yl)piperidin-4-yl)ethyl)phosphonic Acid

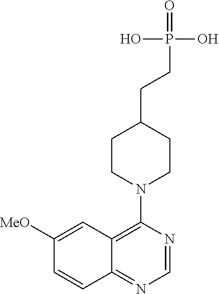 [SMILES: COc1ccc2ncnc(N3CCC(CCP(=O)(O)O)CC3)c2c1]

Prepared according to Method A to give 25 (50% yield) as an off-white solid. LCMS: [M+H]⁺ m/z 352.10. ¹H NMR (400 MHz, Methanol-d₄) δ 8.57 (s, 1H), 7.74-7.73 (m, 1H), 7.68-7.66 (m, 1H), 7.46 (d, 1H), 4.96 (br s, 2H), 3.98, (s, 3H), 3.57 (br s, 2H), 2.65 (s, 2H), 2.07-2.04 (m, 2H), 1.81 (m, 1H), 1.79-1.75 (m, 2H), 1.66-1.63 (m, 2H) and 1.46-1.44 (m, 2H).

### Preparation of (2-(1-(6-hydroxyquinazolin-4-yl)piperidin-4-yl)ethyl)phosphonic Acid 26

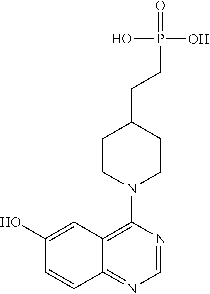 [SMILES: O=P(O)(O)CCC1CCN(c2ncnc3ccc(O)cc23)CC1]

Prepared according to Method C to give 26 (7% yield) as an off-white solid. LCMS: [M+H]⁺ m/z 338.15. ¹H NMR (400 MHz, DMSO-d₆) δ 8.48 (s, 1H), 7.65 (d, J=8.8 Hz, 1H), 7.32 (d, J=8.8 Hz, 1H), 7.18 (s, 1H), 4.21-4.17 (m, 2H), 2.99-2.95 (m, 2H), 1.82-1.79 (m, 2H), 1.53-1.49 (m, 5H) and 1.30-1.19 (m, 2H).

### Preparation of (2-(1-(7-methoxyquinazolin-4-yl)piperidin-4-yl)ethyl)phosphonic Acid 27

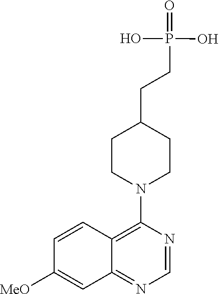 [SMILES: COc1ccc2c(N3CCC(CCP(=O)(O)O)CC3)ncnc2c1]

Prepared according to Method A to give 27 (95% yield) as an off-white solid. LCMS: [M+H]⁺ m/z 352.0 ¹H NMR (500 MHz, Methanol-d₄) δ 8.55 (s, 1H), 8.10 (d, J=10 Hz, 1H), 7.30 (dd, J=10 Hz and 5 Hz, 1H), 7.10 (d, J=5 Hz, 1H), 4.01 (s, 3H), 3.57-3.48 (m, 2H), 2.65 (s, 1H), 2.05-2.02 (m, 2H), 1.94-1.90 (m, 1H), 1.80-1.74 (m, 2H), 1.65-1.60 (m, 2H) and 1.46-1.41 (m, 2H).

### Preparation of (2-(1-(7-ethoxyquinazolin-4-yl)piperidin-4-yl)ethyl)phosphonic Acid 28

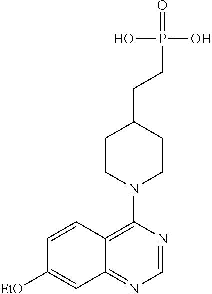 [SMILES: CCOc1ccc2c(N3CCC(CCP(=O)(O)O)CC3)ncnc2c1]

Prepared according to Method B to give 28 as an off-white solid.

### Preparation of (2-(1-(7-Hydroxyquinazolin-4-yl)piperidin-4-yl)ethyl)phosphonic Acid

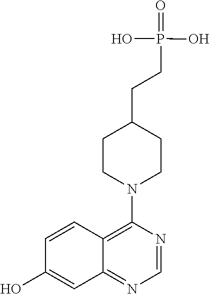 [SMILES: O=P(O)(O)CCC1CCN(c2ncnc3cc(O)ccc23)CC1]

Prepared according to Method B to give 29 (4% yield) as an pale yellow solid. LCMS: [M+H]⁺ m/z 338.25. ¹H NMR (400 MHz, DMSO-d₆) δ 11.49 (s, 1H), 8.64 (s, 1H), 7.96 (d, J=9.2 Hz, 1H), 7.10 (dd, J=9.2 and 2.2 Hz, 1H), 7.00 (d, J=2.2 Hz, 1H), 4.62 (br s, 2H), 3.38 (br s, 2H), 1.87 (d, J=12.7 Hz, 2H), 1.72 (br s, 1H), 1.58-1.38 (m, 4H) and 1.28-1.22 (m, 2H).

### Preparation of (2-(1-(7-aminoquinazolin-4-yl)piperidin-4-yl)ethyl)phosphonic Acid 30

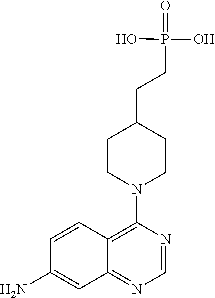 [SMILES: Nc1ccc2c(N3CCC(CCP(=O)(O)O)CC3)ncnc2c1]

Prepared according to Method C to give 30 (32% yield) as a light yellow solid. LCMS: [M+H]⁺ m/z 337.10. ¹H NMR (400 MHz, DMSO-d₆) δ 8.35 (s, 1H), 7.62 (d, J=8.8 Hz, 1H), 6.81 (d, J=8.8 Hz, 1H), 6.61 (br s, 1H), 6.30 (br s, 2H), 4.26-4.20 (m, 2H), 3.10-2.90 (m, 2H), 1.79-1.76 (m, 2H), 1.60-1.30 (m, 5H) and 1.25-1.20 (m, 2H).

### Preparation of (2-(1-(7-isopropoxyquinazolin-4-yl)piperidin-4-yl)ethyl)phosphonic Acid 31

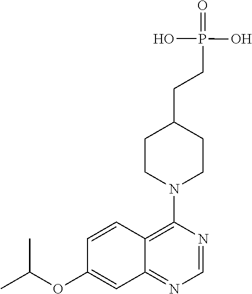 [SMILES: CC(C)Oc1ccc2c(N3CCC(CCP(=O)(O)O)CC3)ncnc2c1]

Prepared according to Method B to give 31 as an light-yellow solid. LCMS: [M+H]⁺ m/z 337.10. ¹H NMR (400 MHz, DMSO-d₆) δ 8.35 (s, 1H), 7.62 (d, J=8.8 Hz, 1H), 6.81 (d, J=9.2 Hz, 1H), 6.66 (s, 1H), 6.30 (br s, 2H), 4.25-4.21 (m, 2H), 3.08-2.96 (m, 2H), 1.81-1.75 (m, 2H), 1.65-1.31 (m, 5H) and 1.27-1.18 (m, 2H).

### Preparation of (2-(1-(8-methoxyquinazolin-4-yl)piperidin-4-yl)ethyl)phosphonic Acid

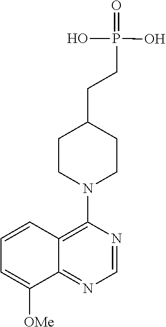 [SMILES: COc1cccc2c(N3CCC(CCP(=O)(O)O)CC3)ncnc12]

Prepared according to Method B to give 32 (32% yield) as an off-white solid. LCMS: [M+H]⁺ m/z 352.15. ¹H NMR (400 MHz, DMSO-d₆) δ 7.92 (s, 1H), 6.92-6.88 (m, 1H), 6.80 (d, J=7.6 Hz, 1H), 6.71 (d, J=8.4 Hz, 1H), 3.76-3.70 (m, 2H), 3.71 (s, 3H), 2.72 (t, J=12 Hz, 2H), 1.64 (d, J=12 Hz, 2H), 1.51-1.28 (m, 5H) and 1.02-0.94 (m, 2H).

### Preparation of (2-(1-(8-ethoxyquinazolin-4-yl)piperidin-4-yl)ethyl)phosphonic Acid 33 (Also Referred to as 226 in the Tables Herein)

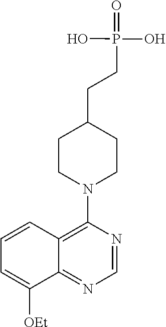 [SMILES: CCOc1cccc2c(N3CCC(CCP(=O)(O)O)CC3)ncnc12]

Prepared according to Method B to give 33 as a white solid. LCMS: [M+H]⁺ m/z 366.20. ¹H NMR (400 MHz, DMSO-d₆) δ ¹H NMR (400 MHz, D₂O) δ 8.17 (s, 1H), 7.21-7.09 (m, 2H), 4.13-3.98 (m, 4H), 2.97 (t, J=12.4 Hz, 2H), 1.82 (d, J=13.0 Hz, 2H), 1.56-1.35 (m, 8H), 1.26 (q, J=11.4 Hz, 2H).

### Preparation of (2-(1-(8-isopropoxyquinazolin-4-yl)piperidin-4-yl)ethyl)phosphonic Acid 34 (in Table 3a)

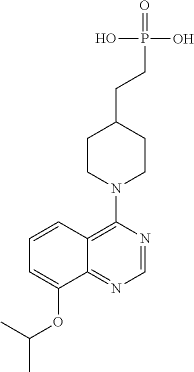 [SMILES: CC(C)Oc1cccc2c(N3CCC(CCP(=O)(O)O)CC3)ncnc12]

Prepared according to Method B to give 34 (43% yield) as an off-white solid. LCMS: [M+H]⁺ m/z ¹H NMR (400 MHz, DMSO-d₆) δ ¹H NMR (400 MHz, D₂O) δ 8.10 (s, 1H), 7.11 (d, J=7.5 Hz, 2H), 7.03 (d, J=7.1 Hz, 1H), 3.94 (d, J=13.0 Hz, 2H), 2.86 (t, J=12.6 Hz, 2H), 1.67 (d, J=13.1 Hz, 2H), 1.40-1.07 (m, 13H).

### Preparation of (2-(1-(8-hydroxyquinazolin-4-yl)piperidin-4-yl)ethyl)phosphonic Acid 36 (in Table 3a)

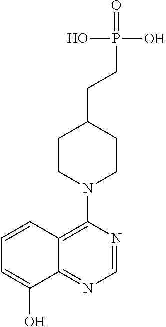 [SMILES: O=P(O)(O)CCC1CCN(c2ncnc3c(O)cccc23)CC1]

Prepared according to Method B to give 35 as an off-white solid.

LCMS: [M+H]⁺ m/z ¹H NMR (400 MHz, DMSO-d₆) δ ¹H NMR (400 MHz, D₂O)

### Preparation of (2-(1-(5,8-Dimethoxyquinazolin-4-yl)piperidin-4-yl)ethyl)phosphonic Acid 36

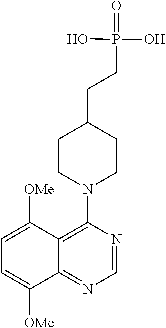 [SMILES: COc1ccc(OC)c2c(N3CCC(CCP(=O)(O)O)CC3)ncnc12]

Prepared according to Method B to give 36 as an off-white solid. LCMS: [M+H]⁺ m/z 382.15. ¹H NMR (400 MHz, DMSO-d₆) δ8.47 (s, 1H), 7.54 (d, J=8.9 Hz, 1H), 7.15 (d, J=8.8 Hz, 1H), 3.95 (d, J=12.0 Hz, 8H), 1.82 (s, 2H), 1.67 (s, 1H), 1.59-1.30 (m, 6H), 1.21 (s, 2H).

### Preparation of (2-(1-(6,8-dimethoxyquinazolin-4-yl)piperidin-4-yl)ethyl)phosphonic Acid 37

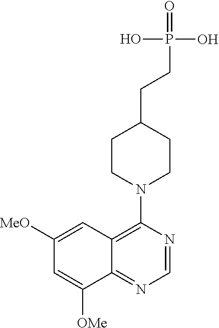 [SMILES: COc1cc(OC)c2ncnc(N3CCC(CCP(=O)(O)O)CC3)c2c1]

Prepared according to Method C to give 37 (15% yield) as an off-white solid. LCMS: [M+H]⁺ m/z 382.15¹H NMR (400 MHz, DMSO-d₆) δ8.54 (s, 1H), 7.11 (s, 1H), 6.86-6.82 (m, 1H), 4.50 (d, J=12.4 Hz, 1H), 4.27 (m, 1H), 3.85 (m, 6H), 3.74 (m, 2H), 3.27 (m, 2H), 1.88-1.85 (m, 2H), 1.66 (m, 1H), 1.54-1.48 (m, 4H) and 1.29 (m, 2H).

### Preparation of (2-(1-(7,8-dimethoxyquinazolin-4-yl)piperidin-4-yl)ethyl)phosphonic Acid 38

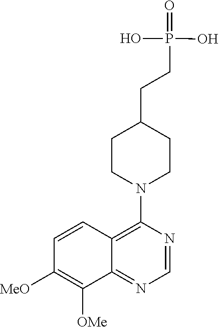 [SMILES: COc1ccc2c(N3CCC(CCP(=O)(O)O)CC3)ncnc2c1OC]

Prepared according to Method C to give 38 (16% yield) as an off-white solid. LCMS: [M+H]⁺ m/z 382.15 ¹H NMR (400 MHz, DMSO-d₆) δ8.60 (s, 1H), 7.90 (d, J=9.6 Hz, 1H), 7.49 (d, J=9.2 Hz, 1H), 4.69 (m, 2H), 4.02 (s, 3H), 3.89 (s, 3H), 3.46 (m, 2H), 1.90 (d, J=12.8 Hz, 2H), 1.75 (m, 1H), 1.53-1.49 (m, 4H) and 1.31-1.28 (m, 2H).

### Preparation of (2-(1-(7,8-dihydro-[1,4]dioxino[2,3-g]quinazolin-4-yl)piperidin-4-yl)ethyl)phosphonic Acid 39 (in Table 3a)

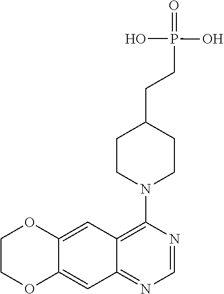 [SMILES: O=P(O)(O)CCC1CCN(c2ncnc3cc4c(cc23)OCCO4)CC1]

Prepared according to Method C to give 39 (7% yield) as an off-white solid. LCMS: [M+H]⁺ m/z 380.15. ¹H NMR (400 MHz, DMSO-d₆) δ8.64 (s, 1H), 7.48 (s, 1H), 7.19 (s, 1H), 4.56 (d, J=11.8 Hz, 2H), 4.45 (d, J=3.0 Hz, 2H), 4.38 (d, J=3.3 Hz, 2H), 3.39 (s, 1H), 3.33 (s, 1H), 1.87 (d, J=12.2 Hz, 2H), 1.71 (s, 1H), 1.58-1.38 (m, 4H), 1.26 (d, J=10.2 Hz, 2H).

### Preparation of (2-(1-(5-fluoro-8-methoxyquinazolin-4-yl)piperidin-4-yl)ethyl)phosphonic Acid 40 (in Table 3a)

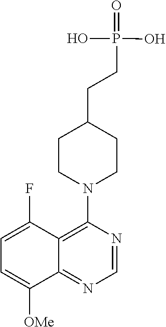 [SMILES: COc1ccc(F)c2c(N3CCC(CCP(=O)(O)O)CC3)ncnc12]

Prepared according to Method C. to give 40 (7% yield) as a light-yellow solid. LCMS: [M+H]⁺ m/z 370.10. ¹H NMR (400 MHz, DMSO-d₆) δ8.55 (s, 1H), 7.44-7.38 (m, 2H), 4.28-4.23 (m, 2H), 3.94 (s, 3H), 3.28-3.18 (m, 2H), 1.82-1.78 (m, 2H), 1.70-1.66 (m, 1H), 1.49-1.23 (m, 4H) and 1.26-1.09 (m, 2H).

### Preparation of (2-(1-(6-fluoro-8-methoxyquinazolin-4-yl)piperidin-4-yl)ethyl)phosphonic Acid 41 (in Table 3a)

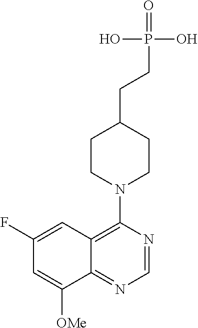 [SMILES: COc1cc(F)cc2c(N3CCC(CCP(=O)(O)O)CC3)ncnc12]

Prepared according to method B to give 41 (44% yield) as a white solid. LCMS: [M+H]⁺ m/z 366.15. ¹H NMR (400 MHz, DMSO-d₆) δ8.02 (d, J=9.2 Hz, 1H), 7.21 (d, J=9.2 Hz, 1H), 7.04 (s, 1H), 3.93 (s, 3H), 2.49 (s, 3H), 1.91-1.88 (m, 2H), 1.74 (m, 1H), 1.53-1.49 (m, 5H), and 1.29-1.27 (m, 3H).

### Preparation of (2-(1-(6-chloro-8-methoxyquinazolin-4-yl)piperidin-4-yl)ethyl)phosphonic Acid 42 (in Table 3a)

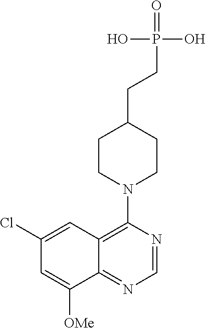 [SMILES: COc1cc(Cl)cc2c(N3CCC(CCP(=O)(O)O)CC3)ncnc12]

Prepared according to Method B to give 42 (42% yield) as a light-yellow solid. LCMS: [M+H]⁺ m/z 386.10. ¹H NMR (400 MHz, D₂O) δ8.03 (s, 1H), 6.91-6.88 (m, 2H), 3.92-3.89 (m, 2H), 3.77 (s, 3H), 2.93-2.87 (m, 2H), 1.70-1.67 (m, 2H) and 1.41-1.12 (m, 7H).

### Preparation of (2-(1-(7-chloro-8-methoxyquinazolin-4-yl)piperidin-4-yl)ethyl)phosphonic Acid 43 (in Table 3a)

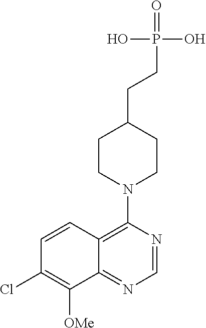 [SMILES: COc1c(Cl)ccc2c(N3CCC(CCP(=O)(O)O)CC3)ncnc12]

Prepared according to Method B to give 43 (50% yield) as a white solid. LCMS: [M+H]1 m/z 386.05. ¹H NMR (400 MHz, D₂O) δ8.07 (s, 1H), 7.10-7.06 (m, 2H), 3.95-3.91 (m, 2H), 3.71 (s, 3H), 2.96-2.90 (m, 2H), 1.71-1.68 (m, 2H) and 1.42-1.01 (m, 7H).

### Preparation of 2-[1-[6,7-dimethoxy-2-[(E)-2-(3-pyridyl)vinyl]quinazolin-4-yl]-4-piperidyl]ethyl-hydroxy-phosphinate 44

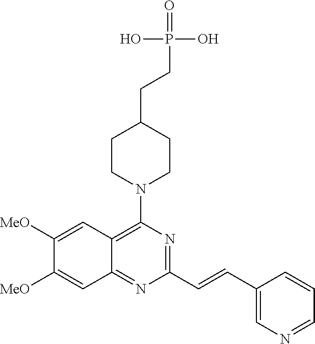 [SMILES: COc1cc2nc(/C=C/c3cccnc3)nc(N3CCC(CCP(=O)(O)O)CC3)c2cc1OC]

Prepared according to Method B to give 44 (49% yield) as a light-yellow solid. LCMS: [M+H]⁺ m/z 485.25. ¹H NMR (400 MHz, D₂O) δ8.09 (s, 1H), 7.94 (s, 1H), 7.36 (d, J=8 Hz, 1H), 7.06 (s, 1H), 6.74 (d, J=16.8 Hz 1H), 6.58 (d, J=3.2 Hz, 1H), 6.40 (d, J=3.2 Hz, 1H), 6.24 (d, J=16.8 Hz, 1H), 3.94-3.91 (m, 2H), 3.84 (s, 3H), 3.67 (s, 3H), 2.96-2.90 (m, 2H), 1.96-1.93 (m, 2H) and 1.56-1.32 (m, 7H).

### Preparation of (E)-(2-(1-(8-Methoxy-2-(2-(pyridin-3-yl)vinyl)quinazolin-4-yl)piperidin-4-yl)ethyl)phosphonic Acid 45

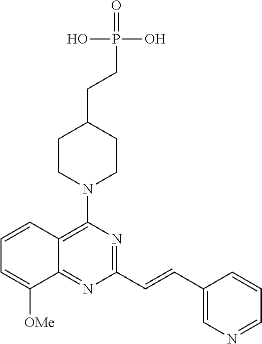 [SMILES: COc1cccc2c(N3CCC(CCP(=O)(O)O)CC3)nc(/C=C/c3cccnc3)nc12]

Prepared according to Method B to give 45 (79% yield) as a yellow solid. LCMS: [M+H]⁺ m/z 455.20. ¹H NMR (400 MHz, Methanol-d₄) δ9.07 (br s, 1H), 8.70 (br s, 1H), 8.62-8.60 (m, 1H), 8.31 (d, 1H), 7.89-7.88 (m, 1H), 7.70-7.54 (m, 4H), 5.20-5.00 (m, 2H) 4.13 (s, 3H), 3.58-3.50 (m, 2H), 2.07-2.02 (m, 2H), 1.88-1.82 (m, 1H), 1.78-1.64 (m, 4H) and 1.51-1.46 (m, 2H).

### Preparation of (2-(1-(6,7-dimethoxyquinolin-4-yl)piperidin-4-yl)ethyl)phosphonic Acid 46

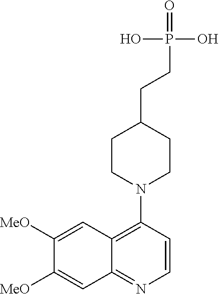 [SMILES: COc1cc2nccc(N3CCC(CCP(=O)(O)O)CC3)c2cc1OC]

Prepared according to Method C to give 46 (22% yield) as a white solid. LCMS. [M+H]⁺ m/z 381.30. ¹H NMR (400 MHz, Methanol-d₄) δ8.35 (d, J=6.8 Hz, 1H), 7.29-7.27 (m, 2H), 7.11 (d, J=6.8 Hz, 1H), 4.27-4.23 (m, 2H), 4.03 (s, 3H), 4.02 (s, 3H), 3.40-3.32 (m, 2H), 2.06-2.03 (m, 4H) 1.82-1.79 (m, 3H) and 1.62-1.48 (m, 2H).

### Preparation of (2-(1-(3-cyano-6,7-dimethoxyquinolin-4-yl)piperidin-4-yl)ethyl)phosphonic Acid 47 Also Referred to as 42 in Table 2)

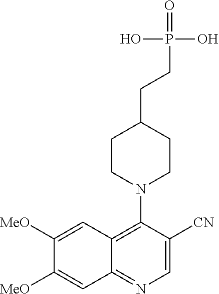 [SMILES: COc1cc2ncc(C#N)c(N3CCC(CCP(=O)(O)O)CC3)c2cc1OC]

Prepared according to Method B to give 47 (47% yield) as an off-white solid. LCMS: [M+H]⁺ m/z 406.20. ¹H NMR (400 MHz, 1D₂O) δ8.00 (s, 1H), 6.62 (s, 1H), 6.40 (s, 1H), 3.75 (s, 3H), 3.66 (s, 3H), 3.18 (d, J=12.3 Hz, 2H), 2.94 (t, J=12.2 Hz, 2H), 1.72 (d, J=12.7 Hz, 2H), 1.43-1.30 (m, 6H), 1.17-1.04 (m, 2H).

### Preparation of (2-(1-(3-cyano-6-methoxyquinolin-4-yl)piperidin-4-yl)ethyl)phosphonic Acid 48 Also Referred to as 44 in Table 2)

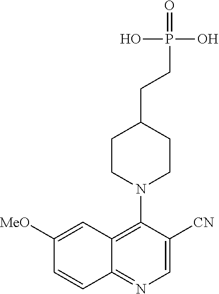 [SMILES: COc1ccc2ncc(C#N)c(N3CCC(CCP(=O)(O)O)CC3)c2c1]

Prepared according to Method B to give 48 (16% yield) as an off-white solid. LCMS. [M+H]⁺ m/z 376.20. ¹H NMR (400 MHz, D₂O) δ8.15 (s, 1H), 7.51 (s, 1H), 7.18 (s, 1H), 6.88 (s, 1H), 3.71 (s, 3H), 3.60-3.51 (m, 2H), 3.15-3.08 (m, 2H), 1.81-1.74 (m, 2H) and 1.41-1.15 (m, 7H).

### Preparation of (2-(1-(3-cyano-7-methoxyquinolin-4-yl)piperidin-4-yl)ethyl)phosphonic Acid 49 (Also Referred to as 43 in Table 2)

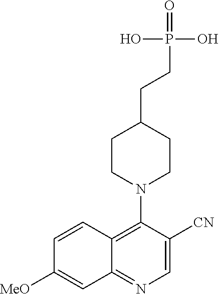 [SMILES: COc1ccc2c(N3CCC(CCP(=O)(O)O)CC3)c(C#N)cnc2c1]

Prepared according to Method B to give 49 (23% yield) as an off-white solid. LCMS: [M+H]⁺ m/z 376.20. ¹H NMR (400 MHz, D₂O) δ7.94 (s, 1H), 7.39 (d, J=9.4 Hz, 1H), 6.84-6.64 (m, 2H), 3.90 (s, 3H), 3.59 (d, J=12.4 Hz, 2H), 3.22 (t, J=12 Hz, 2H), 1.89 (d, J=12.8 Hz, 2H), 1.62-1.45 (m, 5H) and 1.33-1.25 (m, 2H).

### Preparation of (2-(1-(3-cyano-8-methoxyquinolin-4-yl)piperidin-4-yl)ethyl)phosphonic Acid 50 (Also Referred to as 45 in Table 1)

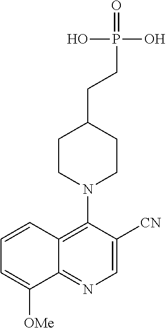 [SMILES: COc1cccc2c(N3CCC(CCP(=O)(O)O)CC3)c(C#N)cnc12]

Prepared according to Method B to give 50 as an off-white solid. LCMS: [M+H]⁺ m/z 376.20. ¹H NMR (400 MHz, D₂O) δ8.03 (s, 1H), 7.27-7.23 (m, 1H), 7.11-7.09 (m, 1H), 7.04-7.02 (m, 1H), 3.90 (s, 3H), 3.43 (br d, J=12.4 Hz, 2H), 3.06 (br t, J=12 Hz, 2H), 1.80 (br d, J=12.8 Hz, 2H), 1.50-1.47 (m, 5H) and 1.31-1.24 (m, 2H).

### Preparation of (2-(1-(6,7-dimethoxyisoquinolin-1-yl)piperidin-4-yl)ethyl)phosphonic Acid 51

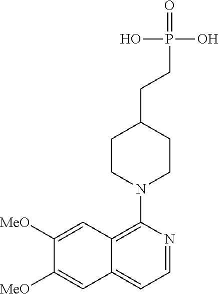 [SMILES: COc1cc2ccnc(N3CCC(CCP(=O)(O)O)CC3)c2cc1OC]

Prepared according to Method B to give 51 (30% yield) as an off-white solid. LCMS: [M+H]⁺ m/z 381.10. ¹H NMR (4(0) MHz, D₂O) δ ¹H NMR (400 MHz, DMSO-d₆): δ 57.79 (d, J=6.4 Hz, 1H), 7.51-7.44 (m, 2H), 7.31 (s, 1H), 3.96 (d, J=4.8 Hz, 6H), 3.23 (m, 4H), 1.88 (m, 2H), 1.55 (m, 2H), 1.48 (m, 1H) and 1.45 (m, 4H).

### Preparation of (24(1-(4-cyano-6,7-dimethoxyisoquinolin-1-yl)piperidin-4-yl)ethyl)phosphonic Acid 52 (in Table 3a)

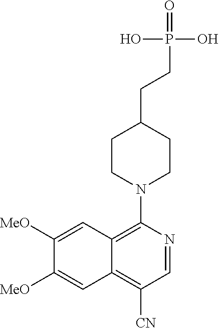 [SMILES: COc1cc2c(C#N)cnc(N3CCC(CCP(=O)(O)O)CC3)c2cc1OC]

Prepared according to Method B to give 52 (50% yield) as an off-white solid. LCMS: [M+H]⁺ m z H NMR (400 MHz, D₂O) δ

### Preparation of O-((1-(8-methoxyquinazolin-4-yl)piperidin-4-yl)methyl)O,O-dihydrogen Phosphorothioate 53 (in Table 3a)

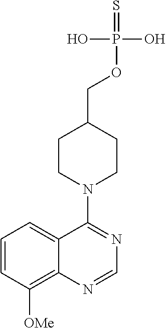 [SMILES: COc1cccc2c(N3CCC(COP(O)(O)=S)CC3)ncnc12]

To a solution of (1-(8-methoxyquinazolin-4-yl)piperidin-4-yl)methanol (prepared in using the same method as compound 80) (500 mg, 1.83 mmol) in pyridine (5 mL) was added phosphorothioyl trichloride (1.6 g, 9.45 mmol) dropwise at −15° C. After being stirred at 0° C. for 1 h, the reaction mixture was added to a solution of sodium bicarbonate (923 mg, 10.98 mmol) in water (20 mL) at 0° C. The resulting mixture was stirred at 0° C. for 2 h, and then evaporated to dryness under reduced pressure. Purification (prep-HPLC) gave 53 (83 mg, in 12%) as a white solid. LCMS: [M+H]⁺ m/z 354.10 ¹H NMR (400 MHz, DMSO-d₆) δ8.59 (s, 1H), 7.59-7.50 (m, 2H), 7.45 (dd, J=6.5, 2.4 Hz, 1H), 4.53 (d, J=12.7 Hz, 2H), 3.97 (s, 3H), 3.80-3.74 (m, 4H), 2.03 (s, 1H), 1.86 (d, J=13.5 Hz, 2H), 1.41 (q, J=11.8 Hz, 2H).

### Preparation of (((1-(8-methoxyquinazolin-4-yl)piperidin-4-yl)oxy)methyl)phosphonic Acid 54 (in Table 3a)

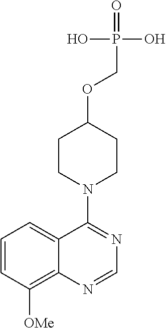 [SMILES: COc1cccc2c(N3CCC(OCP(=O)(O)O)CC3)ncnc12]

Prepared using the same method as compounds 10 and 11. The mixture was purified (prep-HPLC 0.1% TFA) to give 54 as an off-white solid.

LCMS: [M+H]⁺ m/z 353.3. ¹H NMR (400 MHz, D₂O) δ 8.40 (s, 1H), 7.54 (s, 2H), 7.40 (d, J=7.0 Hz, 1H), 4.37 (s, 3H), 4.01-3.88 (m, 9H), 3.71 (d, J=9.3 Hz, 4H), 3.63 (s, 1H), 2.11 (s, 2H), 1.81 (s, 2H).

### Preparation of (4-(8-methoxyquinazolin-4-yl)phenethyl)phosphonic Acid 55 (in Table 3a)

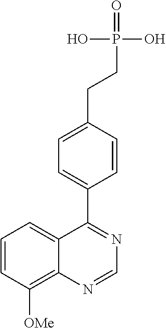 [SMILES: COc1cccc2c(-c3ccc(CCP(=O)(O)O)cc3)ncnc12]

Prepared using the same method as compound 20 as a white solid.

LCMS: [M+H]⁺ m/z 345.10. ¹H NMR (400 MHz, D₂O) δ

### Preparation of (4-(((8-methoxyquinazolin-yl)amino)methyl)phenyl)phosphonic Acid

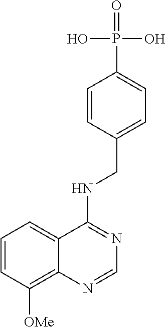 [SMILES: COc1cccc2c(NCc3ccc(P(=O)(O)O)cc3)ncnc12]

Prepared according to the same method as compound 23. LCMS: [M+H]⁺ m/z 346.10. ¹H NMR (400 MHz, D₂O) δ8.20 (s, 1H), 7.62-7.57 (m, 2H), 7.43-7.41 (m, 2H), 7.31-7.28 (m, 2H), 7.23-7.21 (m, 1H) and 2.93 (s, 3H).

### Preparation of (4-(((3-cyano-8-methoxyquinolin-4-yl)amino)methyl)phenyl)phosphonic Acid 57 (Also Referred to as 52 in Table 1)

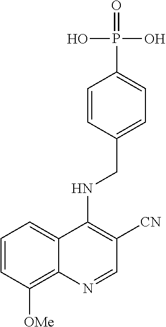 [SMILES: COc1cccc2c(NCc3ccc(P(=O)(O)O)cc3)c(C#N)cnc12]

Prepared according to the same method as compound 23 to give 57 as an off-white solid. LCMS: [M+H]⁺ m/z 370.10. ¹H NMR (400 MHz, D₂O) δ8.02 (s, 1H), 7.48-7.43 (m, 2H, 7.36-7.30 (m, 2H), 7.15-7.09 (m, 3H), 4.77 (s, 2H) and 3.81 (s, 3H).

### Preparation of (4-(((3-cyano-8-methoxyquinolin-4-yl)amino)methyl)benzyl)phosphonic Acid 58 (Also Referred to as 211 in Table 2)

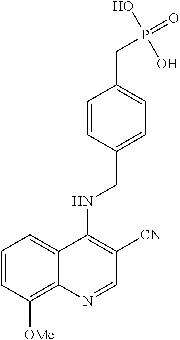 [SMILES: COc1cccc2c(NCc3ccc(CP(=O)(O)O)cc3)c(C#N)cnc12]

Prepared according to the same method as compound 23 to give 58 as a white solid. LCMS: [M+H]⁺ m: 384.15. ¹H NMR (400 MHz, Methanol-d₄) δ8.32 (s, 1H), 7.77 (d, J=8.4 Hz, 1H), 7.50 (t, J=8.4 Hz, 1H), 7.35-7.33 (m, 2H), 7.26 (d, J=7.6 Hz, 1H), 7.20 (d, J=8.4 Hz, 2H), 5.02 (s, 2H), 3.99 (s, 3H) and 2.85 (d, J=20 Hz, 2H).

### Preparation of (3-(1-(3-cyano-8-methoxyquinolin-4-yl)piperidin-4-yl)propyl)phosphonic Acid 59 (Also Referred to as 210 in Table 2)

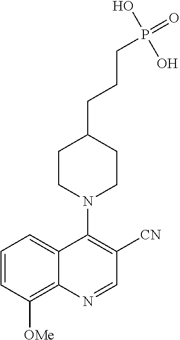 [SMILES: COc1cccc2c(N3CCC(CCCP(=O)(O)O)CC3)c(C#N)cnc12]

Prepared according to the same method as compound 14 to give 59 as a white solid. LCMS: [M+H]⁺ m/z 390.20. ¹H NMR (400 MHz, D₂O) δ8.30 (s, 1H), 7.39 (br s, 2H), 7.19 (br s, 1H), 3.98 (s, 3H), 3.73-3.70 (m, 2H), 3.30 (t, J=12 Hz, 2H), 1.92-1.88 (m, 2H), 1.70-1.45 (m, 3H) and 1.40-1.27 (m, 6H).

### Preparation of (4-(((3-cyano-8-methoxyquinolin-4-yl)amino)methyl)phenyl)boronic Acid 60 (Also Referred to as 214 in Table 2)

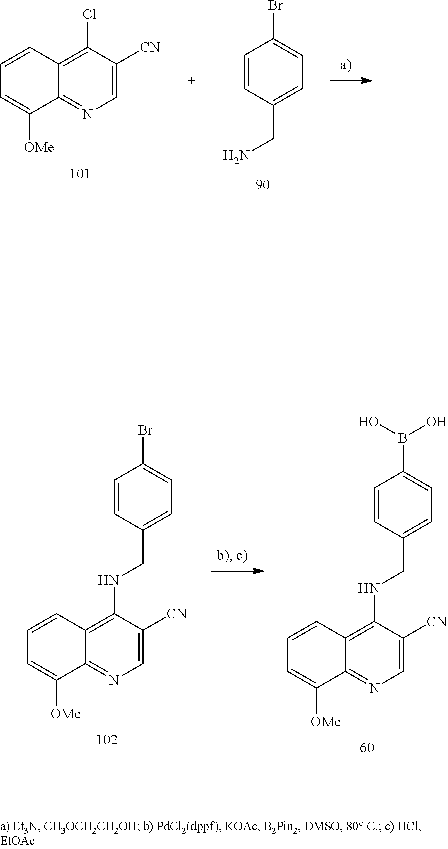 [SMILES: COc1cccc2c(Cl)c(C#N)cnc12 ; NCc1ccc(Br)cc1 ; COc1cccc2c(NCc3ccc(Br)cc3)c(C#N)cnc12 ; COc1cccc2c(NCc3ccc(B(O)O)cc3)c(C#N)cnc12]

To a solution of compound 101 (0.97 g, 5.0 mmol) in 2-methoxyethanol (10 mL) was added (4-bromophenyl)methanamine 90 (1.74 g, 10.0 mmol) and Et₃N (1.51 g, 15 mmol). The mixture was heated at 100° C. overnight, cooled to rt and then evaporated to dryness under reduced pressure. Chromatography (35% EtoAc in petroleum ether) gave 102 (1.5 g, 88%) as white solid. To a solution of compound 102 (69 mg, 0.2 mmol in DMSO (3 mL) was added bis(pinacolato)diboron (61.0 mg, 0.24 mmol), potassium acetate (58.8 mg, 3.0 mmol), Pd(dppf)Cl₂ (7.4 mg, 0.05 mmol). The reaction was degassed by purging with nitrogen and then heated at 80° C. for 48 h. The mixture was cooled to rt, and diluted with ethyl acetate and then filtered through a pad of Celite®. The filtrate was evaporated to dryness under reduced pressure. The residue was dissolved in EtOAc (10 mL) and the HCl (4M, 0.2 mL, 4.0 mmol) solution in EtOAc was added. The mixture was stirred at rt overnight and then evaporated to dryness under reduced pressure. Chromatography [prep-HPLC (TFA)) gave 60 (40.5 mg, 65% over two steps) as white solid. LCMS: [M+H]⁺ m/z 334.15. ¹H NMR (400 MHz, Methanol-d₄) δ 8.69 (s, 1H), 7.90 (d, J=8.4 Hz, 1H), 7.73 (t, J=8.3 Hz, 2H), 7.61 (d, J=8.1 Hz, 2H), 7.41 (dd, J=17.3, 7.8 Hz, 2H), 5.05 (s, 2H), 4.13 (s, 3H).

### Preparation of (2-(1-(3-cyano-8-methoxyquinolin-4-yl)piperidin-4-yl)ethyl)boronic Acid 61 (Also Referred to as 216 in Table 2)

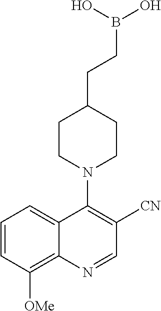 [SMILES: COc1cccc2c(N3CCC(CCB(O)O)CC3)c(C#N)cnc12]

Prepared following the same procedure as compound 7. Compound 61 was isolated as a yellow solid. LCMS: [M+H]⁺ m/z 340.20. ¹H NMR (400 MHz, Methanol-d₄) δ8.81 (s, 1H), 7.83 (d, J=12 Hz, 1H), 7.72 (t, J=8 Hz, 1H), 7.60 (d, J=8 Hz, 1H), 4.51 (d, J=12 Hz, 2H), 3.81 (t, J=12 Hz, 2H), 3.31 (s, 3H), 2.66 (s, 1H), 2.05 (br d, J=12 Hz, 2H), 1.72-1.30 (m, 4H) and 0.91-0.85 (m, 2H).

### Preparation of 4-(((3-cyano-8-methoxyquinolin-4-yl)amino)methyl)-N-hydroxybenzamide 62 (Also Referred to as 220 in Table 2)

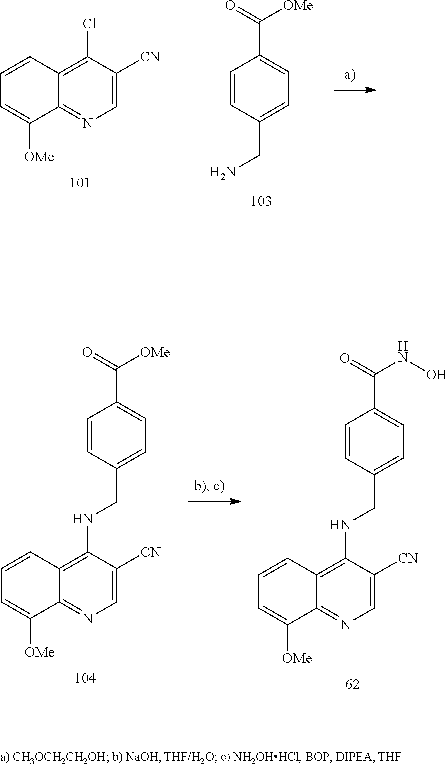 [SMILES: COc1cccc2c(Cl)c(C#N)cnc12 ; COC(=O)c1ccc(CN)cc1 ; COC(=O)c1ccc(CNc2c(C#N)cnc3c(OC)cccc23)cc1 ; COc1cccc2c(NCc3ccc(C(=O)NO)cc3)c(C#N)cnc12]

A solution of 101 (2.0 g, 8.9 mmol) and 103 (1.5 g 8.9 mmol) in 2-methoxyethanol (40 mL) was heated to reflux overnight and then cooled to rt. The reaction mixture was evaporated to dryness under reduced pressure and then triturated with EtOAc filtered and dried to give the crude compound 104 (1.7 g) as a light yellow solid.

To a solution of compound 104 (0.5 g, 1.55 mmol) in THF (20 mL) was added NaOH (0.17 g, 4.65 mmol, dissolved in 2 mL of water). The mixture was heated to 45° C. overnight. The cooled solution was concentrated under reduced pressure and the residue treated with aqueous HCl (2N) until pH 5.5 was realized. The resulting precipitate was filtered and dried to give the crude acid intermediate (0.3 g, 62% yield) as a light yellow solid. The crude acid was dissolved in DMF (10 mL) and then cooled to 0° C. and placed under nitrogen. BOP (0.48 g, 1.06 mmol) and DIPEA (0.50 g, 3.88 mmol) were added followed by HONH₂₋HCl (0.09 g, 1.26 mmol). The mixture was stirred at rt overnight, quenched with water (50 mL) and extracted with EtOAc The organic phase was washed with water, and brine and dried (Na₂SO3) and evaporated to dryness under reduced pressure. Chromatography (5% MeOH in CH₂Cl₂) and then Prep-HPLC (H, 0.1% TFA) gave 62 (34 mg, 10%) as off-white solid. LCMS: [M+H]^{+ 1}H NMR (400 MHz, DMSO-d6) δ 11.19 (s, 1H), 9.06 (s, 1H), 8.55 (s, 1H), 8.45 (d, J=8.7 Hz, 1H), 7.72 (d, J=7.4 Hz, 2H), 7.39 (dd, J=4.6, 2.9 Hz, 2H), 7.29 (dd, J=12.3, 5.0 Hz, 2H), 5.11 (s, 2H), 3.93 (s, 3H).

### Example 2: Assessing Compound Activity

Selected compounds of Tables 1-3 and other derivatives are prepared and assessed in an ENPP1 activity assay using thymidine monophosphate paranitrophenol (TMP-pNP) as a substrate. Enzyme reactions are prepared with TMP-pNP (2 μM), 5-fold dilutions of ENPP1 inhibitor, and purified recombinant mouse ENPP1 (0.5 nM) in 100 mM Tris, 150 mM NaCl, 2 mM CaCl₂, 200 μM ZnCl₂, pH 7.5 at room temperature. Reaction progress is monitored by measuring absorbance at 400 nm of paranitrophenolate produced by the reaction for 20 minutes. Slopes of product formation can be extracted, plotted, and fit to obtain IC₉ values with Graphpad Prism 7.03.

Compounds are also assessed in an ENPP1 enzyme activity assay using cGAMP as a substrate. Methods that can be used to assess the subject compounds include those described by Li et al. in PCT application No. PCT/US2018/050018, filed Sep. 7, 2018. An exemplary method is set forth below.

### Materials:

Mouse ENPP1: Expressed and purified according to Kato et al. PNAS (2012) 109(42):16876-8, cGAMP: Synthesized and purified according to Li et al. Nat. Chem. Biol. (2014) 10:1043-8. Polyphosphate:AMP phosphotransferase (PAP): The PAP gene (GenBank: AB092983.1) was synthesized (Integrated DNA Technologies) and cloned into the pTB146 vector with a His-SUMO C-terminal tag. BL21(DE3) cells transformed with the plasmid were grown and induced at OD600=1 with 0.75 mM IPTG at 16° C. overnight. Cells were resuspended in buffer containing 50 mM Tris pH 7.5, 400 mM NaCl, 10 mM imidazole, 2 mM DTT, protease inhibitor (Roche) and lysed with two freeze-thaw cycles and sonication. All subsequent steps were performed at 4° C. Lysate was cleared by centrifugation at 40,000 ref for 1 hour and the supernatant was incubated with HisPur cobalt resin (Thermo Fisher Scientific) for 2 hours. Resin was washed with twice with 30 mL of buffer containing 50 mM Tris pH 7.5, 150 mM NaCl and the protein was eluted with 50 mM Tris pH 7.5, 150 mM NaCl, 600 mM imidazole. Anion exchange chromatography (HiTrap Q HP) was performed. Myokinase (MilliporeSigma). CellTiterGlo (Promega)

### Exemplary Procedure for ENPP1 Enzyme Activity Assay:

3 nM mouse ENPP1 was incubated with 5 uM cGAMP and 5-fold serial dilutions of compounds in buffer containing 50 mM Tris pH 7.6, 250 nM NaCl, 500 uM CaCl₂, and 1 uM ZnCl₂ (total reaction volume=10 μL) at room temperature for 3 hours, after which the reactions were heat inactivated at 95° C. for 10 minutes. The AMP degradation product was converted to ATP, which was detected using luciferase. To achieve this, an enzyme mixture of polyphosphate:AMP phosphotransferase (PAP) and myokinase was prepared according to Goueli et al. in EP2771480. Briefly, PAP was diluted to 2 mg/mL in buffer containing 50 mM Tris pH 7.5, 0.1% NP-40. Myokinase was diluted to 2 KU/mL in buffer containing 3.2 mM ammonium sulfate pH 6.0, 1 mM EDTA, and 4 mM polyphosphate. The heat-inactivated ENPP1 reaction was incubated with PAP (0.01 μg/μL) and myokinase (0.0075 U/μL) in buffer containing 40 mM Tris pH 7.5, 0.05 mg/mL Prionex, 5 mM MgCl₂, 20 μM polyphosphate, and 0.15 g/L phenol red (for ease of pipetting) for 3 hours (total reaction volume=20 μL). CellTiterGlo (20 uL) was added to the reaction according to manufacturer's protocol and luminescence was measured. Data were normalized to 100% enzyme activity (no compound) and 0% enzyme activity (no enzyme) before being fit to the function 100/(1+([compound]/IC50)).

IC50 values fall in the range indicated by letters A-C, where A represents an IC50 value less than 50 nM, B represents an IC50 value between 50 nM and 100 nM, and C represents an IC50 value greater than 100 nM.

TABLE 4
ENPP1 enzyme activity. IC50 value: A (<50 nM); B (50 nm-100 nM);
C (>100 nM).

| Compound | | |
|---|---|---|
| No. | Structure | IC50 |
| 2 | 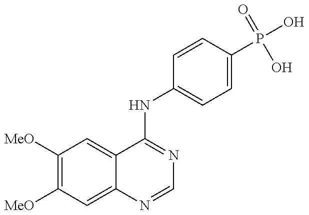 [SMILES: COc1cc2ncnc(Nc3ccc(P(=O)(O)O)cc3)c2cc1OC] | C |
| 3 | 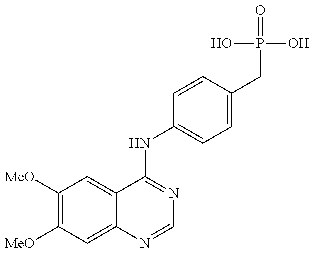 [SMILES: COc1cc2ncnc(Nc3ccc(CP(=O)(O)O)cc3)c2cc1OC] | B |
| 4 | 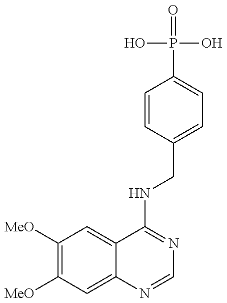 [SMILES: COc1cc2ncnc(NCc3ccc(P(=O)(O)O)cc3)c2cc1OC] | A |
| 7 | 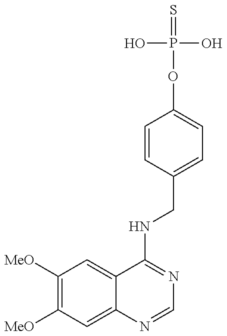 [SMILES: COc1cc2ncnc(NCc3ccc(OP(O)(O)=S)cc3)c2cc1OC] | B |
| 36 | 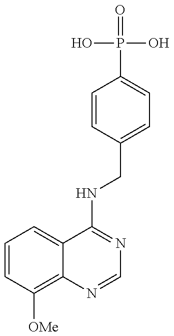 [SMILES: COc1cccc2c(NCc3ccc(P(=O)(O)O)cc3)ncnc12] | A |
| 5 | 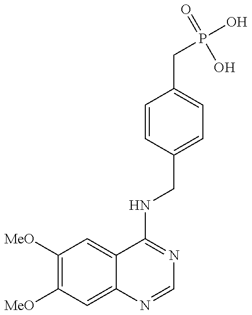 [SMILES: COc1cc2ncnc(NCc3ccc(CP(=O)(O)O)cc3)c2cc1OC] | A |
| 53 | 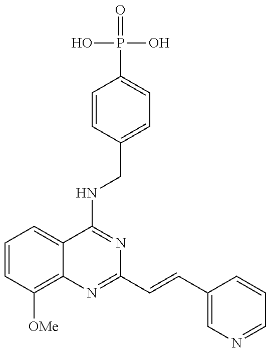 [SMILES: COc1cccc2c(NCc3ccc(P(=O)(O)O)cc3)nc(/C=C/c3cccnc3)nc12] | A |
| 101 | 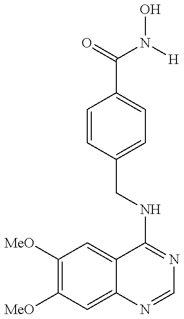 [SMILES: COc1cc2ncnc(NCc3ccc(C(=O)NO)cc3)c2cc1OC] | C |
| 102 | 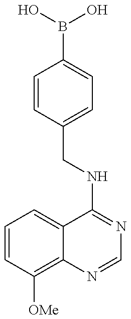 [SMILES: COc1cccc2c(NCc3ccc(B(O)O)cc3)ncnc12] | A |
| 103 | 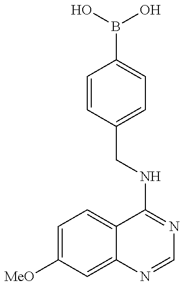 [SMILES: COc1ccc2c(NCc3ccc(B(O)O)cc3)ncnc2c1] | C |
| 104 | 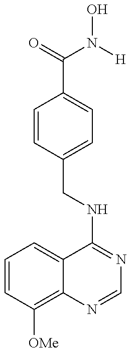 [SMILES: COc1cccc2c(NCc3ccc(C(=O)NO)cc3)ncnc12] | C |

TABLE 2
Compounds

| | | |
|---|---|---|
| 201 | 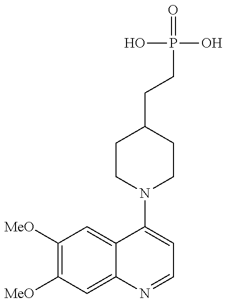 [SMILES: COc1cc2nccc(N3CCC(CCP(=O)(O)O)CC3)c2cc1OC] | C |
| 45 | 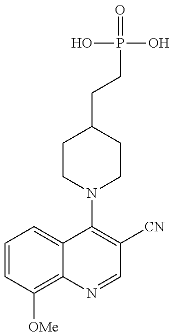 [SMILES: COc1cccc2c(N3CCC(CCP(=O)(O)O)CC3)c(C#N)cnc12] | A |
| 43 | 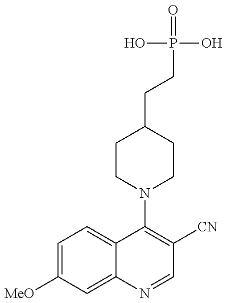 [SMILES: COc1ccc2c(N3CCC(CCP(=O)(O)O)CC3)c(C#N)cnc2c1] | A |
| 42 | 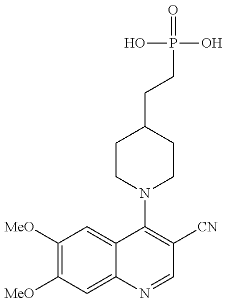 [SMILES: COc1cc2ncc(C#N)c(N3CCC(CCP(=O)(O)O)CC3)c2cc1OC] | A |
| 44 | 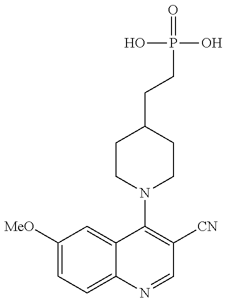 [SMILES: COc1ccc2ncc(C#N)c(N3CCC(CCP(=O)(O)O)CC3)c2c1] | B |
| 46 | 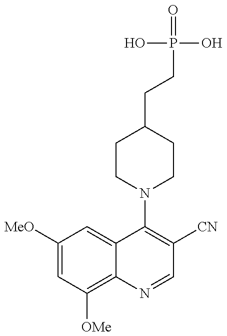 [SMILES: COc1cc(OC)c2ncc(C#N)c(N3CCC(CCP(=O)(O)O)CC3)c2c1] | C |
| 202 | 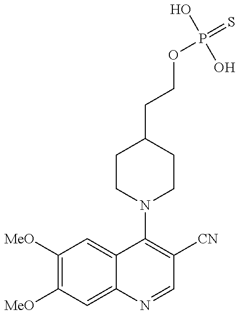 [SMILES: COc1cc2ncc(C#N)c(N3CCC(CCOP(O)(O)=S)CC3)c2cc1OC] | B |
| 52 | 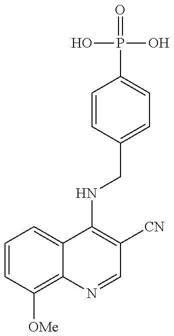 [SMILES: COc1cccc2c(NCc3ccc(P(=O)(O)O)cc3)c(C#N)cnc12] | A |
| 203 | 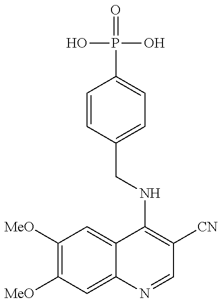 [SMILES: COc1cc2ncc(C#N)c(NCc3ccc(P(=O)(O)O)cc3)c2cc1OC] | A |
| 204 | 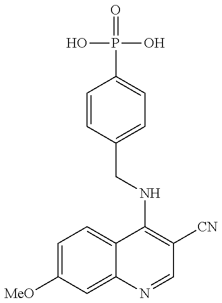 [SMILES: COc1ccc2c(NCc3ccc(P(=O)(O)O)cc3)c(C#N)cnc2c1] | A |
| 205 | 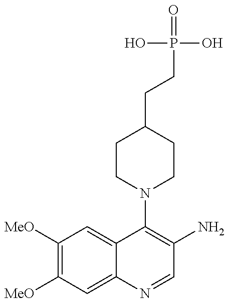 [SMILES: COc1cc2ncc(N)c(N3CCC(CCP(=O)(O)O)CC3)c2cc1OC] | A |
| 206 | 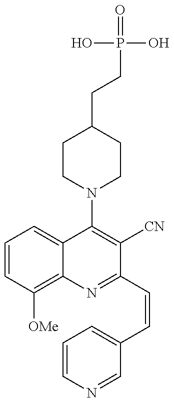 [SMILES: COc1cccc2c(N3CCC(CCP(=O)(O)O)CC3)c(C#N)c(/C=C\c3cccnc3)nc12] | C |
| 207 | 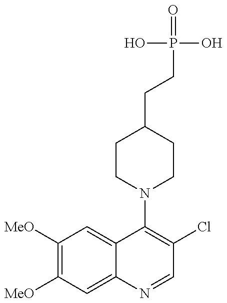 [SMILES: COc1cc2ncc(Cl)c(N3CCC(CCP(=O)(O)O)CC3)c2cc1OC] | C |
| 208 | 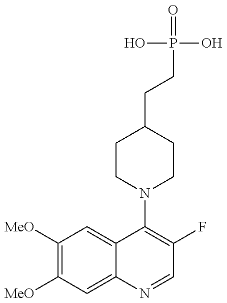 [SMILES: COc1cc2ncc(F)c(N3CCC(CCP(=O)(O)O)CC3)c2cc1OC] | C |
| 209 | 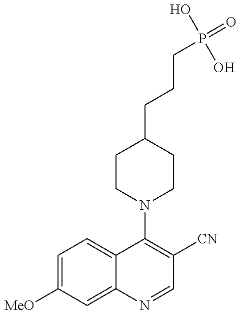 [SMILES: COc1ccc2c(N3CCC(CCCP(=O)(O)O)CC3)c(C#N)cnc2c1] | A |
| 210 | 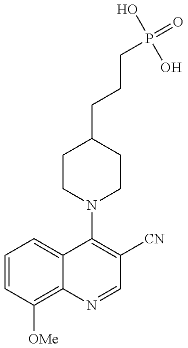 [SMILES: COc1cccc2c(N3CCC(CCCP(=O)(O)O)CC3)c(C#N)cnc12] | A |
| 211 | 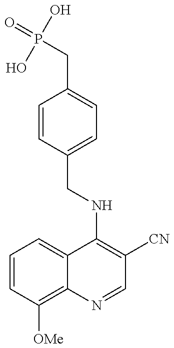 [SMILES: COc1cccc2c(NCc3ccc(CP(=O)(O)O)cc3)c(C#N)cnc12] | A |
| 48 | 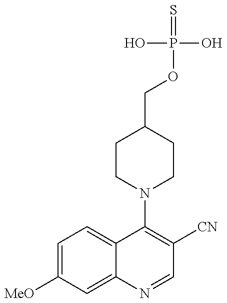 [SMILES: COc1ccc2c(N3CCC(COP(O)(O)=S)CC3)c(C#N)cnc2c1] | C |
| 50 | 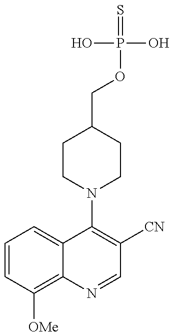 [SMILES: COc1cccc2c(N3CCC(COP(O)(O)=S)CC3)c(C#N)cnc12] | C |
| 212 | 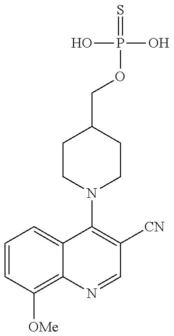 [SMILES: COc1cccc2c(N3CCC(COP(O)(O)=S)CC3)c(C#N)cnc12] | C |
| 213 | 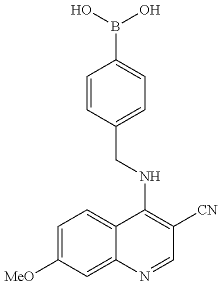 [SMILES: COc1ccc2c(NCc3ccc(B(O)O)cc3)c(C#N)cnc2c1] | C |
| 214 | 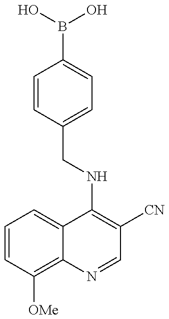 [SMILES: COc1cccc2c(NCc3ccc(B(O)O)cc3)c(C#N)cnc12] | C |
| 215 | 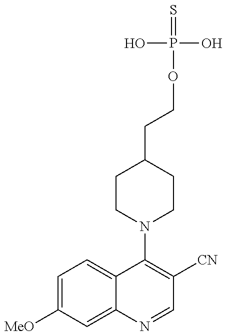 [SMILES: COc1ccc2c(N3CCC(CCOP(O)(O)=S)CC3)c(C#N)cnc2c1] | C |
| 216 | 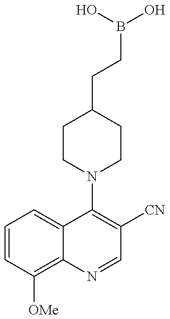 [SMILES: COc1cccc2c(N3CCC(CCB(O)O)CC3)c(C#N)cnc12] | C |
| 217 | 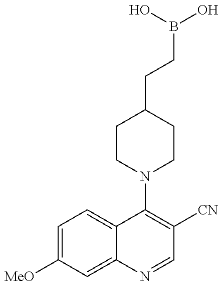 [SMILES: COc1ccc2c(N3CCC(CCB(O)O)CC3)c(C#N)cnc2c1] | C |
| 218 | 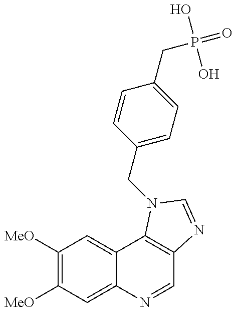 [SMILES: COc1cc2ncc3ncn(Cc4ccc(CP(=O)(O)O)cc4)c3c2cc1OC] | C |
| 219 | 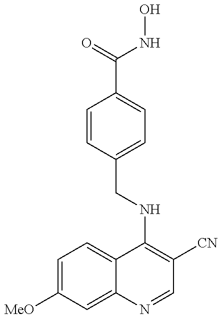 [SMILES: COc1ccc2c(NCc3ccc(C(=O)NO)cc3)c(C#N)cnc2c1] | C |
| 220 | 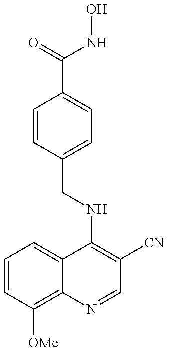 [SMILES: COc1cccc2c(NCc3ccc(C(=O)NO)cc3)c(C#N)cnc12] | C |

### Example 3: Demonstration of Extracellular ENPP1 and Inhibition of Extracellular ENPP1

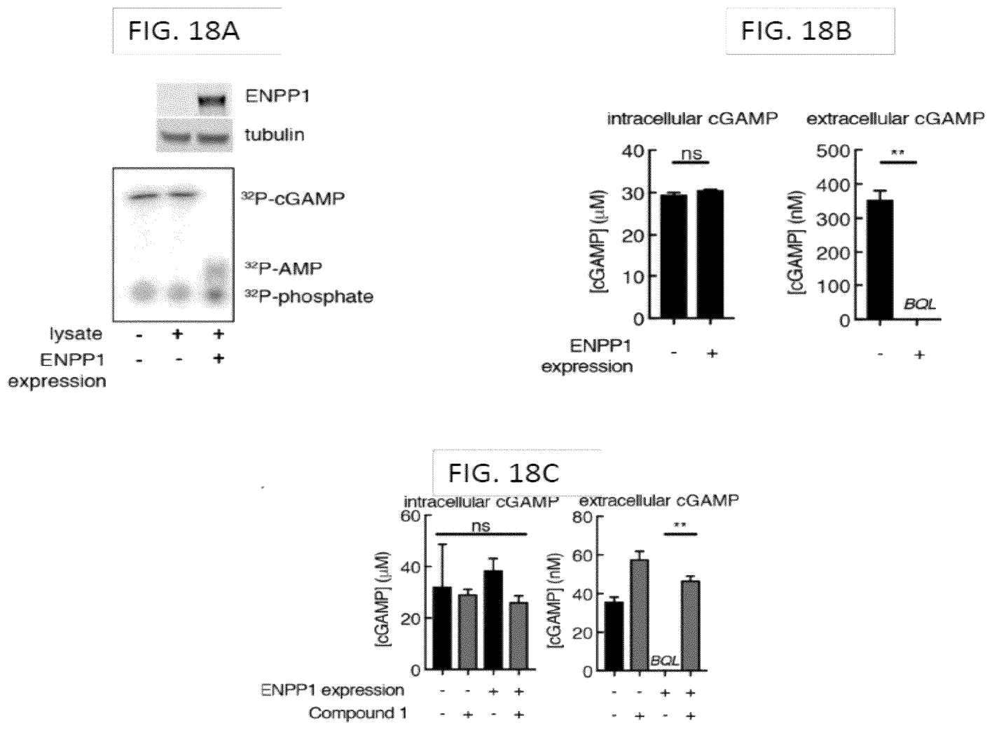

With reference to FIGS. 18A to 18C, it was observed that ENPP1 controls extracellular levels of cGAMP, and that cGAMP levels can be restored by treating cells with an ENPP1 inhibitor (e.g. compound 1).

293T cGAS ENPP1^{-/-} cells were transfected with human ENPP1 expression plasmid and confirmed cGAMP hydrolase activity in whole cell lysates (FIG. 18A). 293T cells were purchased from ATCC and viral transfected to stably express mouse cGAS. 293T mcGAS ENPP1^{-/-} were created by viral transfection of CRISPR sgRNA targeting human ENPP1 (5′ CACCGCTGGTTCTATGCACGTCTCC-3′) (SEQ ID NO:1). 293T mcGAS ENPP1^{-/-} cells were plated in tissue culture treated plates coated with PurCol (Advanced BioMatrix) in DMEM (Corning Cellgro) supplemented with 10% FBS (Atlanta Biologics) (v/v) and 100 U/mL penicillin-streptomycin (ThermoFisher). 12-24 hours following plating, cells were transfected with Fugene 6 (Promega) according to manufacturer's instructions plus indicated concentrations of pcDNA3 plasmid DNA (empty or containing human ENPP1). 24 hours following transfection, cells were lysed for analysis of ENPP1 expression by western blotting (using antibodies rabbit anti-ENPP1 (L520, 1:1000) and mouse anti-tubulin (DM1A, 1:2.000), Cell Signaling Technologies). Whole cell lysates were generated by lysing 1×10⁶ cells in 10 mM Tris, 150 mM NaCl, 1.5 mM MgCl₂. 1% NP-40, pH 9.0. ³²P-cGAMP (5 μM) was incubated with whole cell lysates and degradation monitored as described above in Example 2 (FIG. 18A).

In intact cells. ENPP1 expression depletes extracellular cGAMP, but does not affect the intracellular cGAMP concentration (FIG. 18B). 24 hours following transfection of 293T mcGAS ENPP1^{-/-} with pcDNA3 (empty or containing human ENPP1), the media was removed and replaced with serum-free DMEM supplemented with 1% insulin-transferrin-selenium-sodium pyruvate (ThermoFisher) and 100 U/mL penicillin-streptomycin. 12-24 hours following media change, the media was removed and the cells were washed off the plate with cold PBS. Both the media and cells were centrifuged at 1000 rcf for 10 minutes at 4° C. and prepared for cGAMP concentration measurement by liquid chromatography-tandem mass spectrometry (LC-MS/MS). The cells were lysed in 30 to 100 μL of 50:50 acetonitrile:water supplemented with 500 nM cyclic GMP-¹³C₁₀,¹⁵N₅-AMP as internal standard and centrifuged at 15,000 rcf for 20 minutes at 4° C. to remove the insoluble fraction. Media was removed, supplemented 500 nM cyclic GMP-¹³C₁₀,¹⁵N₅-AMP as internal standard and 20% formic acid. Samples were analyzed for cGAMP, ATP, and GTP content on a Shimadzu HPLC (San Francisco, CA) with an autosampler set at 4° C. and connected to an AB Sciex 4000 QTRAP (Foster City, CA). A volume of 10 μL was injected onto a Biobasic AX LC column, 5 μm, 50×3 mm (Thermo Scientific). The mobile phase consisted of 10 mM ammonium carbonate (A) and 0.1% formic acid in acetonitrile (B). Initial condition was 90% B, maintained for 0.5 min. The mobile phase was ramped to 30% A from 0.5 min to 2.0 min, maintained at 30% A from 2.0 min to 3.5 min, ramped to 90% B from 3.5 min to 3.6 min, and maintained at 90% B from 3.6 min to 5 min. The flow rate was set to 0.6 mL/min. The mass spectrometer was operated in electrode spray positive ion mode with the source temperature set at 500° C. Declustering and collision-induced dissociation were achieved using nitrogen gas. Declustering potential and collision energy were optimized by direct infusion of standards. For each molecule, the MRM transition(s) (m/z), DP (V), and CE (V) are as follows: ATP (508>136, 341, 55), GTP (524>152, 236, 43), cGAMP (675>136, 121, 97; 675>312, 121, 59; 675>152, 121, 73), internal standard cyclic GMP-¹³C₁₀,¹⁵N₅-AMP (690>146, 111, 101; 690>152, 111, 45; 690>327, 111, 47), extraction standard cyclic ¹³C₁₀,¹⁵N₅-GMP-¹³C₁₀,¹⁵N₅-AMP (705>156, 66, 93; 705>162, 66, 73).

Inhibiting ENPP1 blocks degradation of extracellular cGAMP (FIG. 18C). The same experiment was conducted as above, this time also including an ENPP1 inhibitor (compound 1) at 50 μM when the media was changed. With the inhibitor, extracellular cGAMP concentrations in the media were returned to previous levels.

FIG. 18A shows 293T cGAS ENPP1^{-/-} cells that were transfected with empty vector and vector containing human ENPP1 and analyzed after 24 h for ENPP1 protein expression using western blot (top), ENPP1 ³²P-cGAMP hydrolysis activity using thin layer chromatography (TLC) (bottom). FIG. 18B shows intracellular and extracellular cGAMP concentrations using LC-MS/MS. BQL=below quantification limit. Mean±SEM (n=2). **P=0.005 (Student's t test). FIG. 18C shows intracellular and extracellular cGAMP concentrations for 293T cGAS ENPP1^{-/-} cells transfected with empty vector or vector containing human ENPP1 in the presence or absence of 50 μM compound 1. BQL=below quantification limit. Mean±SEM (n=2). **P=0.0013 (Student's t test).

### Example 4: ENPP1 Inhibition Increases cGAMP Activation of Primary CD14+ Monocytes

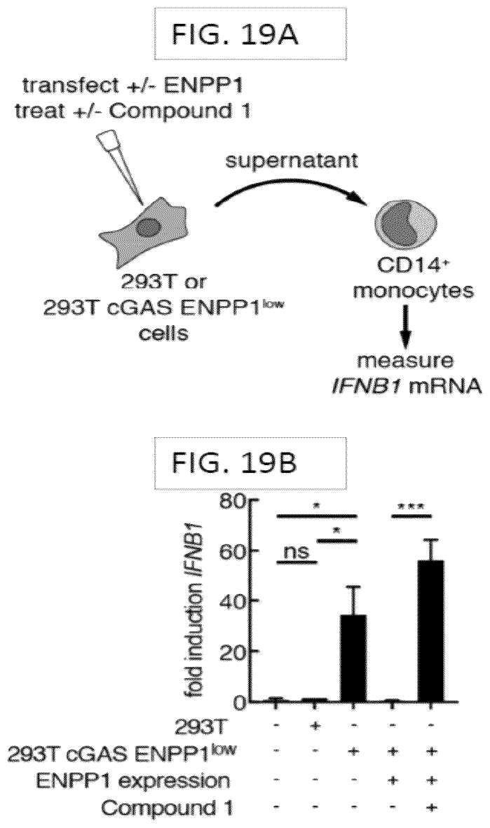

Using an ENPP1 inhibitor (compound 1), it was tested whether cGAMP exported by the 293T cGAS ENPP1^{low} cell line could be detected by antigen presenting cells (APCs) such as human CD14⁺ monocytes (FIG. 19A). 293T cGAS ENPP1^{low} cells were transfected with pcDNA (empty or containing human ENPP1). Primary human peripheral blood mononucleocyte cells (PBMCs) were isolated by subjecting enriched buffy coat from whole blood to a Percoll density gradient. CD14⁺ monocytes were isolated using CD14⁺ MicroBeads (Miltenyi). CD14⁺ monocyctes were cultured in RMPI supplemented with 2% human serum and 100 U/mL penicillin-streptomycin. 8 hours following transfection of 293T cGAS ENPP1^{low} cells, the media was changed to RMPI supplemented with 2% human serum and 100 U/mL penicillin-streptomycin, with or without the exemplary ENPP1 inhibitor compound 1. 24 hours following media change, supernatant from 293T cGAS ENPP1^{low} cells were transferred to CD14⁺ monocytes (FIG. 19A). 24-26 hours following supernatant transfer, total RNA was extracted using Trizol (Thermo Fisher Scientific) and reverse transcribed with Maxima H Minus Reverse Transcriptase (Thermo Fisher Scientific). Real-time RT-PCR was performed in duplicate with AccuPower 2× Greenstar qPCR Master Mix (Bioneer) on a 7900HT Fast Real-Time PCR System (Applied Biosystems). Data were normalized to CD14 expression for each sample. Fold induction was calculated using ΔΔCt. Primers for human IFNB1: fwd (5′-AAACTCATGAGCAGTCTGCA-3′) (SEQ ID NO:2), rev (5′-AGGAGATCTTCAGTTTCGGAGG-3′) (SEQ ID NO:3); human CD14: fwd (5′-GCCTTCCGTGTCCCCACTGC-3′) (SEQ ID NO:4), rev (5′-TGAGGGGGCCCTCGACG-3′) (SEQ ID NO:5).

Supernatant from the cGAS-expressing 293T cGAS ENPP1A cells, but not cGAS-null 293T cells, induced CD14+ INB1 expression, suggesting that extracellular cGAMP exported by cancer cells could be detected by CD14⁺ cells as a signaling factor (FIG. 19B). Transient overexpression of ENPP1 on the 293T cGAS ENPP1^{low} cells caused extracellular cGAMP degradation and reduction of CD14^{low} IFNB1 expression, but addition of compound 1 rescued extracellular cGAMP levels and induced CD14⁺ IFNB1 expression (FIG. 19B).

With reference to FIG. 19A shows a schematic of the supernatant transfer experiment. FIG. 19B shows cGAS-null 293T cells or 293T cGAS ENPP1^{low} cells that were transfected with DNA and incubated in the presence or absence of compound 1. Supernatant from these cells was transferred to primary CD14⁺ human PBMCs, IFNB1 mRNA levels were normalized to CD14 and the fold induction was calculated relative to untreated CD14⁺ cells. Mean±SEM (n=2). *P<0.05, ***P<0.001 (one-way ANOVA).

### Example 5: ENPP1 Inhibition Synergizes with Ionizing Radiation (IR) Treatment to Increase Tumor-Associated Dendritic Cells

It was tested whether cancer cell lines export cGAMP and if ionizing radiation (IR) affects the levels of extracellular cGAMP produced. Ionizing radiation (IR) has been shown to increase cytosolic DNA and activate cGAS-dependent IFN-β production in tumor cells (Bakhoum et al. Nat. Commun. (2015) 6:1-10; and Vanpouille Nat. Commun. (2017) 8:15618). 24 hours after plating, 4T1 cells were treated with 20 Gy IR using a cesium source and the media was changed, supplemented with 50 uM of an ENPP1 inhibitor (compound 1) to inhibit ENPP1 present in cell culture. Media was collected at indicated times, centrifuged at 1000×g to remove residual cells, acidified with 0.5% acetic acid, and supplemented with cyclic-¹³C₁₀,¹⁵₅-GMP-¹³C₁₀,¹⁵N₅-AMP as an extraction standard extraction standard (the appropriate amount for a final concentration of 2 μM in 100 μL). Media was applied to HyperSep Aminopropyl SPE columns (ThermoFisher Scientific) to enrich for cGAMP as described previously (Gao et al., Proc. Natl. Acad. Sci. U.S.A. (2015) 112:E5699-705). Eluents were evaporated to dryness and reconstituted in 50:50 acetonitrile:water supplemented with 500 nM internal standard. The media was submitted for mass spectrometry quantification of cGAMP.

Continuous cGAMP export was detected in the 4T1 cells over 48 hours. At 48 hours, cells treated with IR had significantly higher extracellular cGAMP levels than untreated.

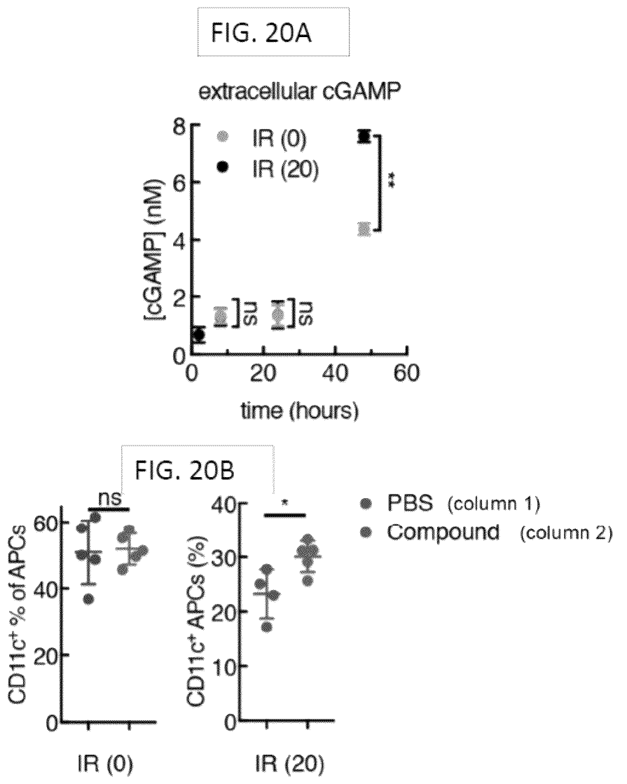

Next, the effect of IR combined with exemplary ENPP1 inhibitor compound 1 on the number of tumor-associated dendritic cells in a mouse 4T1 tumor model was investigated (FIG. 20B). Seven- to nine-week-old female Balb/c mice (Jackson Laboratories) were inoculated with 1×10⁶ 4T1-luciferase tumor cells suspended in 50 μL of PBS into the mammary fat pad. Two days after injection, tumors were irradiated with 20 Gy using a 225 kVp cabinet X-ray irradiator filtered with 0.5 mm Cu (IC 250, Kimtron Inc., CT). Anaesthetized animals were shielded with a 3.2 mm lead shield with a 15×20 mm aperture where the tumor was placed. Mice were intratumorally injected with 100 μL of 1 mM compound 1 in PBS or with PBS alone. On the next day, the tumor was extracted and incubated in RPMI+10% FBS with 20 μg/mL DNase I type IV (Sigma-Aldrich) and 1 mg/mL Collagenase from Clostridium histolyticum (Sigma-Aldrich) at 37° C. for 30 min. Tumors were passed through a 100 μm cell strainer (Sigma-Aldrich) and red blood cells were lysed using red blood cell lysis buffer (155 mM NH₄Cl, 12 mM NaHCO₃, 0.1 mM EDTA) for 5 min at room temperature. Cells were stained with Live/Dead fixable near-IR dead cell staining kit (Thermo Fisher Scientific), Fc-blocked for 10 min using TruStain fcX and subsequently antibody-stained with CD11c, CD45, and I-A/I-E (all Biolegend). Cells were analyzed using an SH800S cell sorter (Sony) or an LSR II (BD Biosciences). Data was analyzed using FlowJo V10 software (Treestar) and Prism 7.04 software (Graphpad) for statistical analysis and statistical significance was assessed using the unpaired t test with Welch's correction.

Intratumoral injection of compound 1 did not change tumor-associated leukocyte compositions compared to the PBS control (FIG. 20B), suggesting that ENPP1 does not play a substantial role in clearing basal level extracellular cGAMP in this tumor model. However, when tumors were pretreated with IR, it was observed that compound 1 increased the tumor associated CD11c⁺ population (FIG. 20B).

The results are illustrated in FIG. 20A and FIG. 20B. FIG. 20A shows extracellular cGAMP produced by 4T1 cells over 48 hours. At time 0, cells were left untreated or treated with 20 Gy IR and refreshed with media supplemented with 50 μM compound 1. Mean±SEM (n=2). **P=0.004 (Student's t test). FIG. 20B shows 4T1 cells (1×10⁶) that were orthotopically injected into BALB/cJ mice on day 0. Tumors were left untreated or treated with 20 Gy IR and intratumorally injected with PBS (n=5 for IR (0 Gy); n=4 for IR (20 Gy)) or compound 1 (n=5) on day 2. Tumors were harvested and analyzed by FACS on day 3. *P=0.047 (Welch's t test).

### Example 6: ENPP1 Inhibition Synergizes with IR Treatment and Anti-CTLA-4 to Exert Anti-Tumor Effects

It was investigated whether immune detection and clearance of tumors could be increased by further increasing extracellular cGAMP in vivo using ionizing radiation (IR) and an exemplary ENPP1 inhibitor, e.g., compound 1.

Seven- to nine-week-old female Balb/c mice (Jackson Laboratories) were inoculated with 5×10⁴ 4T1-luciferase cells suspended in 50 μL of PBS into the mammary fat pad. When tumor volume (determine length²×width/2) reached 80 mm³ to 120 mm³, tumors were irradiated with 20 Gy using a 225 kVp cabinet X-ray irradiator filtered with 0.5 mm Cu (IC 250, Kimtron Inc., CT). Anaesthetized animals were shielded with a 3.2 mm lead shield with a 15×20 mm aperture where the tumor was placed. On day 2, 4 and 7 after IR, 100 μL of 100 μM compound 1 and/or 10 μg cGAMP in PBS or PBS alone were injected intratumorally. Alternatively, 1 mM compound 1 in PBS or PBS alone were injected intratumorally and 200 μg of anti-CTLA-4 antibody or Syrian hamster IgG antibody (both BioXCell) were injected intraperitoneally on day 2, 5, and 7 after IR. Mice from different treatment groups were co-housed in each cage to eliminate cage effects. The experimenter was blinded throughout the entire study. Tumor volumes were recorded every other day. Tumor volumes were analyzed in a generalized estimation equation in order to account for the within mouse correlation. Pair-wise comparisons of the treatment groups at each time point were done using post hoc tests with a Tukey adjustment for multiple comparisons. Animal death was plotted in a Kaplan Meier curve using Graphpad Prism 7.03 and statistical significance was assessed using the Logrank Mantel-Cox test. All animal procedures were approved by the administrative panel on laboratory animal care.

Administration of compound 1 enhanced tumor shrinkage effects of IR treatment, although not significantly (FIG. 21A). Although intratumoral injection of cGAMP had no effect over IR treatment, injection of compound 1 in addition to cGAMP synergistically shrunk tumors, prolonged survival, and achieved a 10% cure rate (FIG. 21A and FIG. 21B).

The synergistic effect with the adaptive immune checkpoint blocker anti-CTLA4 was also tested. Without IR, treatment with anti-CTLA4 and compound 1 had no effect on prolonging survival (FIG. 21C). However, combining IR pretreatment with compound 1 and anti-CTLA-4 exerted significant synergistic effects and achieved a 10% cure rate. Together, these results demonstrate that enhancing extracellular cGAMP by combining IR treatment with ENPP1 inhibition increases tumor immunogenicity and exerts anti-tumor effects.

The results are illustrated in FIG. 21A, which shows tumor shrinkage effects of compound 1 in combination with IR. Established tumors (100±20 mm³) were treated once with 20 Gy IR followed by three intratumoral injections of PBS or treatment on day 2, 4, and 7 after IR (n=9 per treatment group). Mice from different treatment groups were co-housed and the experimenter was blinded. Tumor volumes were analyzed in a generalized estimation equation to account for within mouse correlation. Pair-wise comparisons of the treatment groups at each time point were performed using post hoc tests with a Tukey adjustment for multiple comparisons. FIG. 21B shows Kaplan Meier curves for FIG. 21A, P values determined by the log-rank Mantel-Cox test. FIG. 21C shows, in addition to the same procedure as in FIG. 21B, anti-CTLA 4 or IgG isotype control antibodies that were injected intraperitoneally on days 2, 5, and 7 after IR (n=8 for IR (0)+compound 1+CTLA-4 treatment group; n=17-19 for all other treatment groups). Statistical analysis performed as for FIG. 21B.

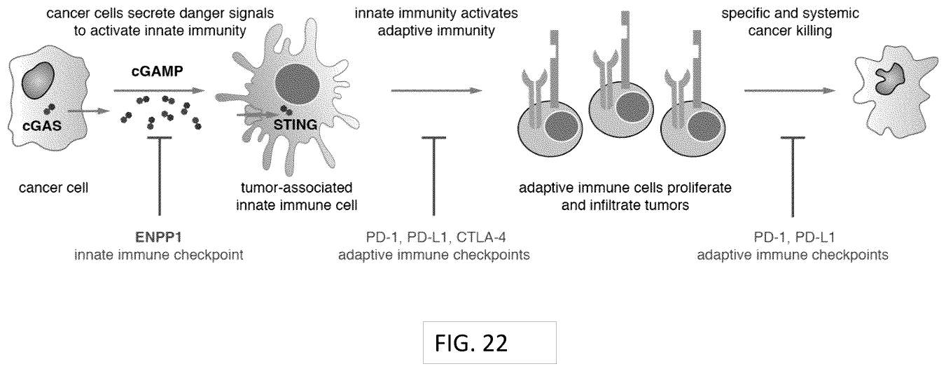

In summary, these results indicate that the cGAMP exists extracellulary and subject ENPP1 inhibitors can act extracellularly; therefore, indicating that the extracellular inhibition of ENPP1 is sufficient for therapeutic effect. ENPP1 qualifies as an innate immune checkpoint. These experiments indicate that inhibiting ENPP1 extracellularly allows cGAMP to potentiate anti-cancer immunity and combine synergistically with immune checkpoint blocking drugs already available as therapies (FIG. 22).

### Example 7: 2′3′-cGAMP is an Immunotransmitter Produced by Cancer Cells and Regulated by ENPP1

### Introduction

2′3′-cyclic GMP-AMP (cGAMP) is characterized as an intracellular second messenger that is synthesized in response to cytosolic dsDNA and activates the innate immune STING pathway. Its extracellular hydrolase ENPP1 hinted at the existence of extracellular cGAMP. Using mass spectrometry, it was detected that cGAMP is continuously exported as a soluble factor by an engineered cell line but then efficiently cleared by ENPP1. By developing a potent, specific, and cell impermeable ENPP1 inhibitor, cGAMP export was detected in cancer cell lines commonly used for mouse tumor models. In tumors, depletion of extracellular cGAMP using neutralizing proteins decreased tumor-associated dendritic cells. Boosting extracellular cGAMP by genetic knockout and pharmacological inhibition of ENPP1 increased tumor-associated dendritic cells, shrunk tumors, and synergized with ionizing radiation and anti-CTLA-4 to cure tumors. In conclusion, cGAMP is an anti-cancer immunotransmitter released by tumors and detected by host innate immunity.

The second messenger 2′3′-cyclic GMP-AMP (cGAMP) plays pivotal roles in anti-viral and anti-cancer innate immunity. It is synthesized by the enzyme cyclic-GMP-AMP synthase (cGAS) in response to double-stranded DNA (dsDNA) in the cytosol, which is a danger signal for intracellular pathogens and damaged or cancerous cells, cGAMP binds and activates its endoplasmic reticulum (ER) surface receptor Stimulator of Interferon Genes (STING) to activate production of Type 1 interferons (IFNs). These potent cytokines trigger downstream innate and adaptive immune responses to clear the threat.

In addition to activating STING within its cell of origin, cGAMP can spread to bystander cells through gap junctions in epithelial cells. This cell-cell communication mechanism alerts adjacent cells of the damaged cell and also, unfortunately, accounts for the spreading of drug-induced liver toxicity and brain metastases. In addition, cytosolic cGAMP can be packaged into budding viral particles and transmitted during the next round of infection. In both transmission modes, cGAMP is never exposed to the extracellular space.

The enzyme responsible for the only detectable cGAMP hydrolase activity is ectonucleotide pyrophosphatase phosphodiesterase 1 (ENPP1) (see e.g., Li, L. et al. Hydrolysis of 2′3′-cGAMP by ENPP1 and design of nonhydrolyzable analogs. Nat. Chem. Biol. 10, 1043-8 (2014)). This is surprising because ENPP1 is annotated as an extracellular enzyme both as a membrane-bound form anchored by a single-pass transmembrane domain and as a cleaved soluble protein in the serum, cGAMP, which has two negative charges and presumably cannot passively cross the cell membrane, can enter cells to activate STING (see e.g., Gao, P. et al. Structure-function analysis of STING activation by c[G(2′,5′) pA(3′,5′)p] and targeting by antiviral DMXAA. Cell 154, 748-762 (2013), and Corrales, L. et al. Direct Activation of STING in the Tumor Microenvironment Leads to Potent and Systemic Tumor Regression and Immunity. Cell Rep. 11, 1018-1030 (2015)), suggesting that there are transport channels for cGAMP. Because it can enter cells, cGAMP analogs are currently being tested in clinical trials to treat metastatic solid tumors via intratumoral injections. Knowing that extracellular cGAMP can be imported and has anti-cancer effects, and that the dominant cGAMP hydrolase is extracellular, it was hypothesized that cGAMP is exported to the extracellular space to signal other cells and is regulated by extracellular degradation.

Herein is demonstrated cGAMP export by cancers and the role of extracellular cGAMP in anti-cancer immune detection. Using genetic knockout and pharmacological inhibition, the role of ENPP1 in controlling extracellular cGAMP concentration, immune infiltration, and tumor progression was also investigated. Together, cGAMP was characterized as an immunotransmitter regulated by ENPP1.

### Materials and Methods

### Reagents, Antibodies, and Cell Lines

[α-³²P]ATP (800 Ci/mmol, 10 mCi/mL, 250 μCi) and [³⁵S]ATPαS (1250 Ci/mmol, 12.5 mCi/mL, 250 Ci) were purchased from Perkin Elmer. Adenosine triphosphate, guanosine triphosphate, adenosine-¹³C₁₀,¹⁵N₅, 5′-triphosphate, guanosine-¹³C₁₀,¹⁵N₅-triphosphate, 4-nitrophenyl phosphate, and bis(4-nitrophenyl) phosphate were purchased from Sigma-Aldrich and are >98% atomically pure. 2′3′-cGAMP was purchased from Invivogen. Caco-2 assay was purchased from Cyprotex. Kinome screens were conducted by Eurofins. PAMPA and MDCK permeability assays were conducted by Quintara Discovery. Total protein content was quantified using the BCA assay (ThermoFisher). Cell viability was quantified using the CellTiterGlo assay (Promega). Full length human ENPP1 was cloned into pcDNA3 vector. A set of 4 ON-TARGETplus ENPP1 siRNA (LQ-003809-00-0002) were purchased from Dharmacon. QS1 was synthesized as previously described²⁵. The following monoclonal antibodies were used for western blotting: rabbit anti-cGAS (D1D3G Cell Signaling, 1:1,000) rabbit anti-mouse cGAS (D2080 Cell Signaling, 1:1,000), mouse anti-tubulin (DM1A Cell Signaling, 1:2,000), and rabbit anti-STING (D2P2F Cell Signaling, 1:1,000), IRDye 800CW goat anti-rabbit (LI-COR, 1:15,000), and IRDye 680RD goat anti-mouse (LI-COR, 1:15,000).

293T cells were purchased from ATCC and viral transfected to stably express mouse cGAS. 293T cGAS ENPP1^{low} cells were created by viral transfection of CRISPR sgRNA targeting human ENPP1 (5′-CACCGCTGGTTCTATGCACGTCTCC-3′), and 293T mcGAS ENPP1^{-/-} cells were selected after single cell cloning from this pool. 4T1 and E0771 cGAS^{-/-} cells were created by viral transfection of CRISPR sgRNA (using lentiCRISPRv2-blast, Addgene plasmid #83480) targeting mouseMb21d1 (5′-CACCGGAAGGGGCGCGCGCTCCACC-3′). Cells were selected after single cell cloning. 4T1-Luc ENPP1^{-/-} cells were created by viral transfection of CRISPR sgRNAs (using lentiCRISPRv2-blast) (Sanjana, N. E., Shalem, O. & Zhang. F. Improved vectors and genome-wide libraries for CRISPR screening. Nat. Methods 11, 783-784 (2014)) targeting mouse Enpp1 (5′-GCTCGCGCCCATGGACCT-3′ and 5′-ATATGACTGTACCCTACGGG-3′) or a scrambled sequence. 4T1-Luc shcGAS cells were created by viral transfection of shRNA (5′-CAGGATTGAGCTACAAGAATAT-3′) using the plasmid pGH188. Cells harboring the shRNA were selected with blasticidin and sorted for GFP expression, and were used as a pool for experiments. MDA-MB-231 were purchased from ATCC, E0771 were purchased from CH3 BioSystems, 4T1-luciferase and HEK293S GnT1⁻ cells expressing secreted mENPP1 were obtained.

### Cell Culture

Cell lines were maintained in DMEM (Corning Cellgro) (293T, MC38) or RPMI (Corning Cellgro) (4T1-Luc, E0771, MDA-MD-231) supplemented with 10% FBS (Atlanta Biologics) (v/v) and 100 U/mL penicillin-streptomycin (ThermoFisher). Primary human peripheral blood mononuclear cells (PBMCs) were isolated by subjecting enriched buffy coat from whole blood to a Percoll density gradient. CD14⁺ PBMCs were isolated using CD14⁺ MicroBeads (Miltenyi). CD14⁺ PBMCs were cultured in RMPT supplemented with 2% human serum and 100 U/mL penicillin-streptomycin.

### Expression and Purification of Recombinant Proteins

sscGAS: The DNA sequence encoding porcine cGAS (residues 135-497) was amplified from a porcine cDNA library using the primer pair fwd: (5′-CTGGAAGTTCTGTTCCAGGGGCCCCATATGGGCGCCTGGAAGCTCCAGAC-3′) and rev: (5′-GATCTCAGTGGTGGTGGTGGTGGTGCTCGAGCCAAAAAACTGGAAATCCATTGT-3′). The PCR product was inserted into pDB-His-MBP via Gibson assembly and expressed in Rosetta cells. Cells were grown in 2×YT medium with kanamycin (100 μg/ml), induced with 0.5 mM IPTG when the OD₆₀₀ reached 1, and were allowed to grow overnight at 16° C. All following procedures involving proteins and cell lysates were conducted at 4° C. Cells were pelleted and lysed in 20 mM HEPES pH 7.5, 400 mM NaCl, 10% glycerol, 10 mM imidazole, 1 mM DTT, and protease inhibitor cocktail (cOmplete EDTA free tablets. Roche). The cell extract was cleared by ultracentrifugation at 50,000×g for 1 h. The cleared supernatant was incubated with HisPur Cobalt resin (ThermoFisher Scientific: 1 mL resin per liter of bacteria culture). Cobalt resin was washed with 20 mM HEPES pH 7.5, 1 M NaCl, 10% glycerol, 10 mM imidazole, 1 mM DTT. Protein was eluted from resin with 300 mM imidazole in 20 mM HEPES pH 7.5, 1 M NaCl, 10% glycerol, and 1 mM DTT. Fractions containing His-MBP-sscGAS were pooled, concentrated and dialyzed against 20 mM HEPES pH 7.5, 400 mM NaCl, 1 mM DTT. The protein was snap frozen in aliquots for future use.

STING: Mouse STING (residues 139-378) was inserted into the pTB146 His-SUMO vector and expressed in Rosetta cells. Cells were grown in 2×YT medium with 100 stg/mL ampicillin and induced when the OD₆₀₀ reached 1 with 0.75 mM IPTG at 16° C. overnight. All subsequent procedures using proteins and cell lysates were performed at 4° C. Cells were pelleted and lysed in 50 mM Tris pH 7.5, 400 mM NaCl, 10 mM imidazole. 2 mM DTT, and protease inhibitors (cOmplete, EDTA-free protease inhibitor cocktail Roche). Cells were lysed by sonication and the lysate was cleared by ultracentrifugation at 50,000 rcf for 1 hour. The cleared supernatant was incubated with HisPur cobalt resin (ThermoFisher Scientific; 1 mL resin per 1 L bacterial culture) for 30 minutes. The resin-bound protein was washed with 50 column volumes of 50 mM Tris pH 7.5, 150 mM NaCl, 2% triton X-114, 50 CV of 50 mM Tris pH 7.5, 1 M NaCl (each wash was set to a drip rate of 1 drop/2-3 seconds and took 2-3 hours), and 20 CV of 50 mM Tris pH 7.5, 150 mM NaCl. Protein was eluted from resin with 600 mM imidazole in 50 mM Tris pH 7.5, 150 mM NaCl. Fractions containing His-SUMO-STING were pooled, concentrated, and dialyzed against 50 mM Tris pH 7.5, 150 mM NaCl while incubating with the SUMOlase enzyme His-ULP1 to remove the His-SUMO tag overnight. The solution was incubated with the HisPur cobalt resin again to remove the His-SUMO tag, and STING was collected from the flowthrough. Protein was dialyzed against 50 mM Tris pH 7.5, loaded onto a HitrapQ anion exchange column (GE Healthcare) using an Äkta FPLC (GE Healthcare), and eluted with a NaCl gradient. Fractions containing STING were pooled and buffer exchanged into PBS and stored at 4° C. until use.

ENPP1: mENPP1 was produced as described by Kato. K. et al. (Expression, purification, crystallization and preliminary X-ray crystallographic analysis of Enpp1. Acta Crystallogr. Sect. F Struct. Biol. Cryst. Commun. 68, 778-782 (2012); and Crystal structure of Enpp1, an extracellular glycoprotein involved in bone mineralization and insulin signaling. Proc. Natl. Acad. Sci. U.S.A. 109, 16876-81 (2012)).

### Liquid Chromatography-Tandem Mass Spectrometry

Cyclic GMP-¹³C₁₀,¹⁵N₅-AMP was used as an internal standard and cyclic ¹³C₁₀,¹⁵₅-GMP-¹³C₁₀,¹⁵N₅-AMP was used as an extraction standard. Isotope-labeled cGAMP standards were synthesized by incubating 1 mM ATP (isotope labeled), 1 mM GTP (isotope labeled), 20 mM MgCl₂, 0.1 mg/mL herring testes DNA (Sigma), and 2 μM sscGAS in 100 mM Tris, pH 7.5 overnight. The reaction was heated at 95° C. and filtered through a 3 kDa centrifuge filter. Water was removed on a rotary evaporator. cGAMP was purified from the crude reaction mixture using a PLRP-S polymeric reversed phase preparatory column (100 Å, 8 μm, 300×25 mm; Agilent Technologies) on a preparatory HPLC (1260 Infinity LC system; Agilent Technologies) connected to UV-vis detector (ProStar; Agilent Technologies) and fraction collector (440-LC; Agilent Technologies). The flow rate was set to 25 mL/min. The mobile phase consisted of 10 mM triethylammonium acetate in water and acetonitrile. The mobile phase started as 2% acetonitrile for the first 5 min. Acetonitrile was then ramped up to 30% from 5-20 min, ramped up to 90% from 20-22 min, maintained at 90% from 22-25 min, and then ramped down to 2% from 25-28 min. Fractions containing cGAMP were lyophilized and resuspended in water. The concentration was determined by measuring absorbance at 280 nm. Samples were analyzed for cGAMP, ATP, and GTP content on a Shimadzu HPLC (San Francisco, CA) with an autosampler set at 4° C. and connected to an AB Sciex 4000 QTRAP (Foster City. CA). A volume of 10 μL was injected onto a Biobasic AX LC column, 5 μm, 50×3 mm (Thermo Scientific). The mobile phase consisted of 100 mM ammonium carbonate (A) and 0.1% formic acid in acetonitrile (B). Initial condition was 90% B, maintained for 0.5 min. The mobile phase was ramped to 30% A from 0.5 min to 2.0 min, maintained at 30% A from 2.0 min to 3.5 min, ramped to 90% B from 3.5 min to 3.6 min, and maintained at 90% B from 3.6 min to 5 min. The flow rate was set to 0.6 mL/min. The mass spectrometer was operated in electrode spray positive ion mode with the source temperature set at 500° C. Declustering and collision-induced dissociation were achieved using nitrogen gas. Declustering potential and collision energy were optimized by direct infusion of standards. For each molecule, the MRM transition(s) (m/z), DP (V), and CE (V) are as follows: ATP (508>136, 341, 55), GTP (524>152, 236, 43), cGAMP (675>136, 121, 97; 675>312, 121, 59; 675>152, 121, 73), internal standard cyclic GMP-¹³C₁₀,¹⁵N₅-AMP (690>146, 111, 101; 690>152, 111, 45; 690>327, 111, 47), extraction standard cyclic ¹³C₁₀,¹⁵N₅-GMP-¹³C₁₀,¹⁵N₅-AMP (705>156, 66, 93; 705>162, 66, 73).

Export Assay in 293T cGAS ENPP1^{-/-} Cells

293T cGAS ENPP1^{-/-} cells were plated in tissue culture treated plates coated with PurCol (Advanced BioMatrix). 24 hours later, the media was gently removed and replaced with serum-free DMEM supplemented with 1% insulin-transferrin-selenium-sodium pyruvate (ThermoFisher) and 100 U/mL penicillin-streptomycin. At indicated times, the media was removed and the cells were washed off the plate with cold PBS. Both the media and cells were centrifuged at 1000 rcf for 10 minutes at 4° C. The cells were lysed in 30 to 100 μL of 50:50 acetonitrile:water supplemented with 500 nM internal standard, and centrifuged at 15,000 ref for 20 minutes at 4° C. to remove the insoluble fraction. If no concentration was necessary, an aliquot of media was removed, supplemented with internal standard at 500 nM and 20% formic acid. If concentration was necessary, the media was acidified with 0.5% acetic acid and supplemented with extraction standard (the appropriate amount for a final concentration of 2 μM in 100 μL). Media was applied to HyperSep Aminopropyl SPE columns (ThermoFisher Scientific) to enrich for cGAMP as described by Gao, D. et al. (Activation of cyclic GMP-AMP synthase by self-DNA causes autoimmune diseases. Proc. Natl. Acad Sci. U.S.A. 112, E5699-705 (2015)). Eluents were evaporated to dryness and reconstituted in 50:50 acetonitrile:water supplemented with 500 nM internal standard. The media and cell extract were submitted for mass spectrometry quantification of cGAMP, ATP, and GTP.

Transfection Stimulation of 293T cGAS ENPP1^{-/-} Cells

293T cGAS ENPP1^{-/-} cells were transfected with Fugene 6 (Promega) according to manufacturer's instructions plus indicated concentrations of pcDNA3 plasmid DNA (empty or containing human ENPP1). 24 hours following transfection, the export assay was conducted as described above.

### Conditioned Media Transfer

293T cGAS ENPP1^{low} cells were plated and transfected with plasmid DNA as described above. 24 hours following transfection, media was changed to RPMI+2% human serum+1% penicillin-streptomycin, +/−2 μM cGAMP, +/−20 nM recombinant mENPP1, or +/−50 uM compound 1. 24 hours following media change, the conditioned media was removed from the 293T cGAS ENPP1^{low} cells and incubated with freshly isolated CD14⁺ PBMCs. Gene expression of CD14⁺ PBMCs was analyzed 14-16 h later.

### RT-PCR Analysis

Total RNA was extracted using Trizol (Thermo Fisher Scientific) and reverse transcribed with Maxima H Minus Reverse Transcriptase (Thermo Fisher Scientific). Real-time RT-PCR was performed in duplicate with AccuPower 2× Greenstar qPCR Master Mix (Bioneer) on a 7900HT Fast Real-Time PCR System (Applied Biosystems). Data were normalized to CD14, ACTB, or GAPDH expression for each sample. Fold induction was calculated using ΔΔCt. Primers for human IFNB1; fwd (5′-AAACTCATGAGCAGTCTGCA-3′), rev (5′-AGGAGATCTTCAGTTTCGGAGG-3′); human CD14: fwd (5′-GCCTTCCGTGTCCCCACTGC-3′), rev (5′-TGAGGGGGCCCTCGACG-3′); human ACTB: fwd (5′-GGCATCCTCACCCTGAAGTA-3′), rev (5′-AGAGGCGTACAGGGATAGCA-3′); human GAPDH: fwd (5′-CCAAGGTCATCCATGACAAC-3′); rev (5′-CAGTGAGCTTCCCGTTCAG-3).

### ³²P-cGAMP Degradation TLC Assay

Radiolabeled ³²P cGAMP was synthesized by incubating unlabeled ATP (1 mM) and GTP (1 mM) doped with ³²P-ATP with 2 μM purified recombinant porcine cGAS in 20 mM Tris pH 7.5, 2 mM MgCl₂, 100 μg/mL herring testes DNA) overnight at room temperature, and the remaining nucleotide starting materials were degraded with alkaline phosphatase for 4 h at 37° C. Cell lysates were generated by scraping and lysing 1×10⁶ cells (293T) or 10×10⁶ cells (4T1-Luc, E0771, and MDA-MB-231) in 100 μL of 10 mM Tris, 150 mM NaCl, 1.5 mM MgCl₂, 1% NP40, pH 9.0. For 4T1-Luc, E0771, and MDA-MB-231, total protein concentration of lysate was measured using the BCA assay (Pierce. Thermo Fisher), and samples were normalized so the same amount of protein was used for each lysate reaction. The probe ³²P-cGAMP (5 μM) was incubated with mENPP1 (20 nM) or whole cell lysates in 100 mM Tris, 150 mM NaCl, 2 mM CaCl₂), 200 μM ZnCl₂, pH 7.5 or pH 9.0 for the indicated amount of time. To generate inhibition curves, 5-fold dilutions of ENPP1 inhibitor was included in the reaction. Degradation was evaluated by TLC (see e.g., Li, L. et al. Hydrolysis of 2′3′-cGAMP by ENPP1 and design of nonhydrolyzable analogs. Nat. Chem. Biol. 10, 1043-8 (2014)). Plates were exposed on a phosphor screen (Molecular Dynamics) and imaged on a Typhoon 9400 and the ³²P signal was quantified using ImageJ. Inhibition curves were fit to obtain IC₅₀ values with Graphpad Prism 7.03. IC₅₀ values were converted to K, values using the Cheng-Prusoff equation K_{i,app}=IC₅₀/(1+[S]/Kₘ).

### ALPL and ENPP2 Inhibition Assays

Inhibition assays for other ectonucleotidases were performed by incubating reaction components in 96-well plate format at room temperature and monitoring production of 4-nitrophenolate by measuring absorbance at 400 nM in a platereader (Tecan). ALPL: 0.1 nM ALPL, 2 μM 4-nitrophenyl phosphate, and various concentrations of inhibitor in buffer pH 9.0 containing 50 mM Tris, 20 μM ZnCl₂, 1 mM MgCl₂ at room temperature. ENPP2: 2 nM ENPP2, 500 μM bis(4-nitrophenyl) phosphate, and various concentrations of inhibitor in buffer pH 9.0 containing 100 mM Tris, 150 mM NaCl, 200 μM ZnCl₂, 2 mM CaCl₂).

### Export Assay in Cancer Cell Lines

4T1-Luc, E0771, and MC38 cells were changed to new media supplemented with 50 μM compound 1. At indicated times, media was collected; cells were scraped off the plate with PBS, pelleted at 1000 rcf, lysed with 4 mL 50:50 acetonitrile:water, and centrifuged at 15.000 rcf, cGAMP was enriched from the media and cell supernatant as described above using the HyperSep Aminopropyl SPE columns and submitted for mass spectrometry quantification.

### 4T1-Luc Tumor Mouse Model

Seven- to nine-week-old female BALB/c mice (Jackson Laboratories) were inoculated with 5×10⁻⁶ or 5×10⁵ 4T1-Luc-luciferase cells suspended in 50 μL of PBS into the mammary fat pad. When tumor volume (determine length²×width/2) reached 80 mm³ to 120 mm³, tumors were irradiated with 20 Gy using a 225 kVp cabinet X-ray irradiator filtered with 0.5 mm Cu (IC-250, Kimtron Inc., CT). Anaesthetized animals were shielded with a 3.2 mm lead shield with a 15×20 mm aperture where the tumor was placed. On day 2, 4, and 7 after IR, 100 μL of 100 μM compound 1 and/or 10 μg cGAMP in PBS or PBS alone were injected intratumorally. Alternatively, 1 mM compound 1 in PBS or PBS alone were injected intratumorally and 200 μg of anti-CTLA-4 antibody or Syrian hamster IgG antibody (both BioXCell) were injected intraperitoneally on day 2, 5, and 7 after IR. Mice from different treatment groups were co-housed in each cage to eliminate cage effects. The experimenter was blinded throughout the entire study. Tumor volumes were recorded every other day. Tumor volumes were analyzed in a generalized estimation equation in order to account for the within mouse correlation. Pair-wise comparisons of the treatment groups at each time point were done using post hoc tests with a Tukey adjustment for multiple comparisons. Animal death was plotted in a Kaplan Meier curve using Graphpad Prism 7.03 and statistical significance was assessed using the Log-rank Mantel-Cox test. All mice were maintained at Stanford University in compliance with the Stanford University Institutional Animal Care and Use Committee regulations, and procedures were approved by the Stanford University administrate panel on laboratory animal care.

### FACS Analysis of Tumors

Seven- to nine-week-old female BALB/c WT (4T1-Luc tumors) or C57BL/6 (E0771 tumors) WT, cGAS^{-/-}, or STING^{gt/gt} (referred to as STING^{-/-}) mice (Jackson Laboratories) were inoculated with 1×10⁶ tumor cells suspended in 50 μL of PBS into the mammary fat pad. Two days after injection, tumors were irradiated as described and intratumorally injected with 100 μL of 1 mM compound 1 in PBS or with PBS alone. For experiments using STING and mENPP1, 100 μL of 100 μM neutralizing STING or non-binding STING (R237A) or 700 nM mENPP1 or PBS were injected intratumorally. On the next day, the tumor was extracted and incubated in RPMI+10% FBS with 20 μg/mL DNase I type IV (Sigma-Aldrich) and 1 mg/mL Collagenase from Clostridium histolyticum (Sigma-Aldrich) at 37° C. for 30 min. Tumors were passed through a 100 μm cell strainer (Sigma-Aldrich) and red blood cells were lysed using red blood cell lysis buffer (155 mM NH₄Cl, 12 mM NaHCO₃, 0.1 mM EDTA) for 5 min at room temperature. Cells were stained with Live/Dead fixable near-IR dead cell staining kit (Thermo Fisher Scientific), Fc-blocked for 10 min using TruStain fcX and subsequently antibody-stained with CD11c, CD45, and I-A/I-E (all Biolegend). Cells were analyzed using an SH800S cell sorter (Sony) or an LSR II (BD Biosciences). Data was analyzed using FlowJo V10 software (Treestar) and Prism 7.04 software (Graphpad) for statistical analysis and statistical significance was assessed using the unpaired t test with Welch's correction.

### In Vivo Imaging

Mice were injected ip with 3 mg XenoLight D-Luciferin (Perkin-Elmer) in 200 μl water and imaged using a Lago X in vivo imaging system (Spectral Instruments Imaging). Object height was set to 1.5 cm, binning to 4, FStop to 1.2, and the exposure time was 120s. Images were analyzed using aura 2.0.1 software (Spectral Instruments Imaging).

### Results

cGAMP is Exported from 293T cGAS ENPP1^{-/-} Cells as a Soluble Factor

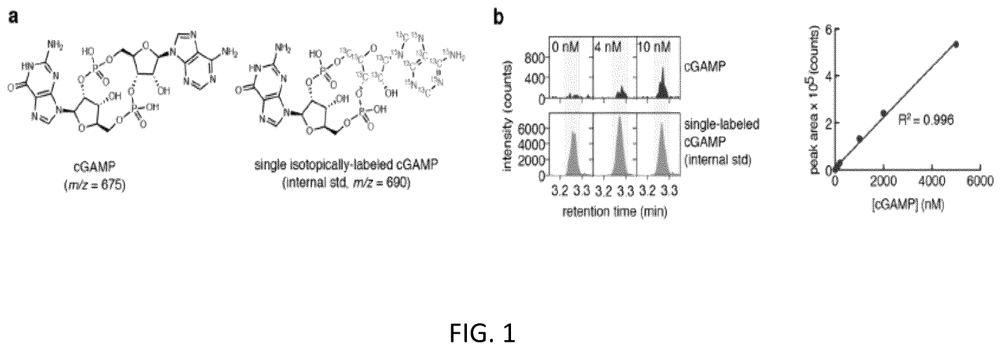

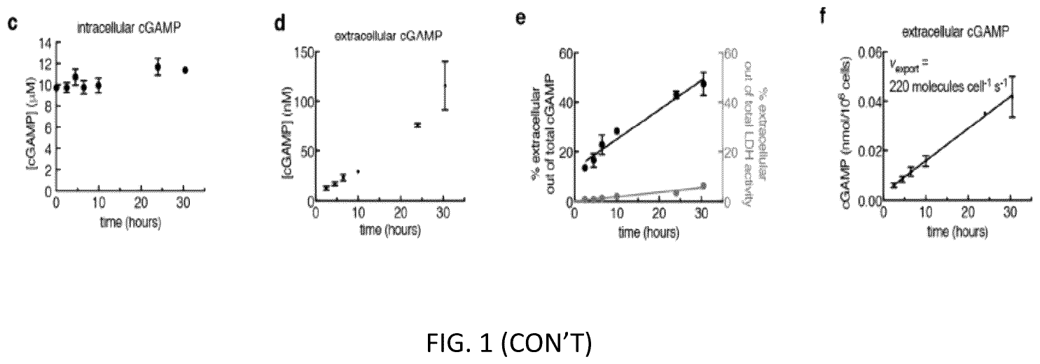

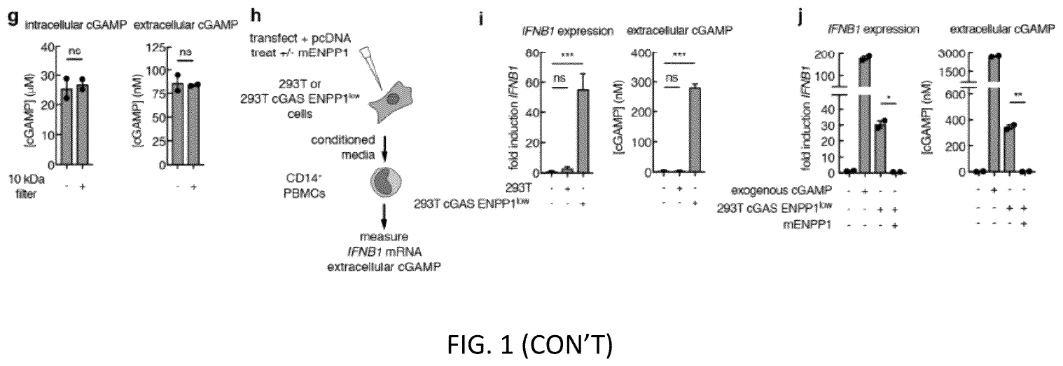

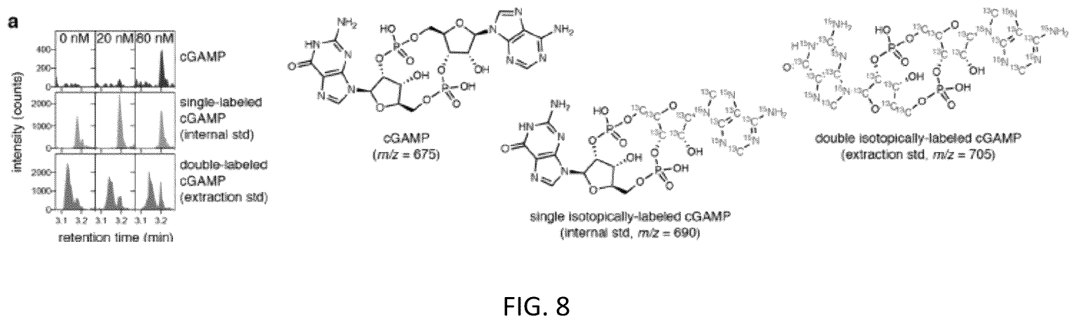

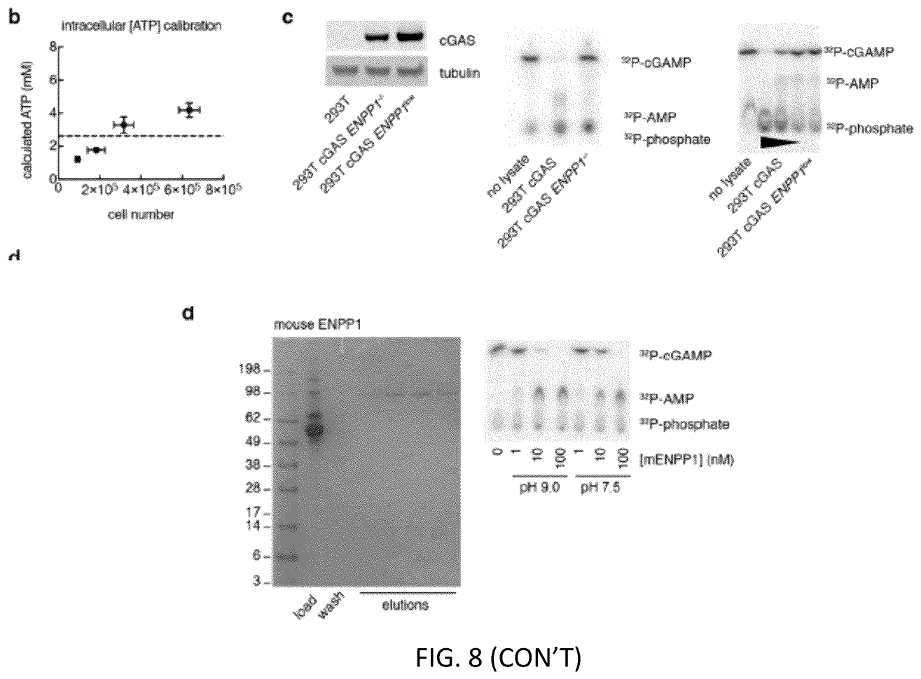

To test the hypothesis that cGAMP is present extracellularly, we first developed a liquid chromatography-tandem mass spectrometry (LC-MS/MS) method to detect cGAMP from complex mixtures. Using two isotopically labeled cGAMP standards (FIG. 1, panel A), we can quantify cGAMP concentrations down to 0.5 nM in both basal cell culture media and serum containing media, and we can quantify intracellular cGAMP concentrations from cell extracts in the same experiment (FIG. 1, panel B and FIG. 8, panels A and B). We chose to use 293T cells, which do not express cGAS or STING. By stably expressing mouse cGAS and knocking out ENPP1 using CRISPR, we created a 293T cGAS ENPP1^{low} cell line (FIG. 8, panel C). We then isolated a single clone to create a 293T cGAS ENPP1^{-/-} cell line. (FIG. 8, panel C). We also used serum-free media because serum contains a proteolytically cleaved soluble form of ENPP1. Using this ENPP1-free cell culture system, we detected constant low micromolar basal intracellular cGAMP concentrations in the 293T cGAS ENPP1^{-/-} cells without any stimulation (FIG. 1, panel C). This is not surprising since there is abundant cytosolic dsDNA in cancer cells as a result of erroneous DNA segregation (see e.g., Mackenzie, K. J, et al. cGAS surveillance of micronuclei links genome instability to innate immunity. Nature 548, 461-465 (2017); Harding, S. M. Mitotic progression following DNA damage enables pattern recognition within micronuclei. Nature 548, 466-470 (2017), and Bakhoum, S. F. et al. Chromosomal instability drives metastasis through a cytosolic DNA response. Nature 553, 467-472 (2018)). After replenishing the cells with fresh media, we measured a linear increase of extracellular cGAMP concentrations to 100 nM after 30 h (FIG. 1, panel D). At 30 h, the number of cGAMP molecules outside the cells was equal to the number inside (FIG. 1, panel E). We detected negligible amount of cell death based on extracellular lactose dehydrogenate (LDH) activity, suggesting that cGAMP in the media is exported by live cells (FIG. 1, panel E). We calculated the export rate (vₑₓₚₒᵣₜ) to be 220 molecules cell⁻¹ s⁻¹ (FIG. 1, panel F). Finally, cGAMP in the media can pass through a 10 kDa filter without any retention, which should retain extracellular vesicles and proteins, suggesting that cGAMP is exported as a freely soluble molecule (FIG. 1, panel H).

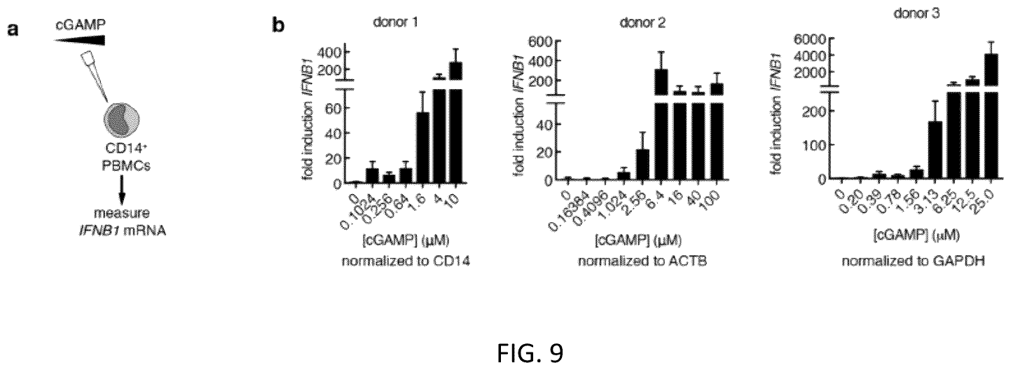

To further confirm that extracellular cGAMP secreted by 293T cells is predominantly in a soluble form, but not in extracellular vesicles, we used CD14⁺ human peripheral blood mononuclear cells (PBMCs) as a reporter. These cells have previously been shown to take up soluble cGAMP, which leads to IFN-β production¹⁷. We observed that CD14⁺ PBMCs respond to submicromolar concentrations of soluble cGAMP by upregulating IFNB1 (FIG. 9). Conditioned media from DNA-transfected cGAS-expressing 293T cGAS ENPP1^{low} cells, but not DNA-transfected cGAS-null 293T cells, induced IFNB1 expression in CD14⁺ cells, suggesting that the activity is a result of extracellular cGAMP produced by 293T cells (FIG. 1, panels H and I). Addition of purified soluble recombinant mouse ENPP1 (mENPP1) (FIG. 8, panel D) depleted detectable cGAMP in the conditioned media and also ablated this activity (FIG. 1, panels H and J). Because soluble ENPP1 (MW=˜100 kDa) cannot permeate membranes and, thus, can only access soluble extracellular cGAMP, we conclude that 293T cells secrete soluble cGAMP. Together, our data demonstrate that this artificial cancer cell line keeps its intracellular cGAMP at a steady state by exporting it into the extracellular media as a soluble factor.

FIG. 1, panels A to J: cGAMP is exported from 293T cGAS ENPP1^{-/-} cells as a soluble factor. a, Chemical structures of cGAMP and single isotopically-labeled cGAMP. b, cGAMP is detected by LC-MS/MS, lower limit of quantification=4 nM. (left) Liquid chromatography traces of cGAMP at 0, 4, and 10 nM and single isotopically-labeled cGAMP (15 Da heavier) at 500 nM as an internal standard: (right) external standard curve of cGAMP, R²=0.996. Data are representative of >10 independent experiments. c-d, Intracellular and extracellular concentrations of cGAMP from 293T cGAS ENPP1^{-/-} cells without exogenous stimulation measured using LC-MS/MS. At time 0, cells were replenished with serum-free media. Mean±SEM (n=2) with some error bars too small to visualize. Data are representative of three independent experiments. e, The fraction of extracellular/total cGAMP molecules (left y-axis) calculated from data in (c) and (d) compared to the fraction of extracellular/total lactose dehydrogenate (LDH) activity (right y-axis). f, The amount of cGAMP exported per cell over time calculated from data in (d). The export rate was found using linear regression. g, Intracellular and extracellular cGAMP concentrations produced by 293T cGAS ENPP1^{-/-} cells measured before and after passing the media through 10 kDa filters. Mean±SEM (n=2). Data are representative of two independent experiments. h, Schematic of the conditioned media transfer experiment for (i) and (j), cGAS-null 293T or 293T cGAS ENPP1^{low} cells were transfected with empty pcDNA vector and treated+/−20 nM recombinant mouse ENPP1 (mENPP1). Conditioned media from these cells was transferred to primary CD14⁺ human PBMCs.i, IFNB1 mRNA levels were normalized to CD14 and the fold induction was calculated relative to untreated CD14⁺ cells. Mean±SEM (n=4). ***P=0.0003 (one-way ANOVA), cGAMP concentrations were measured in the conditioned media. Mean±SEM (n=2). ***P=0.0002 (one-way ANOVA). Data are representative of two independent experiments. j, IFNB1 mRNA levels were normalized to CD14 and the fold induction was calculated relative to untreated CD14⁺ cells. Mean±SEM (n=2). *P=0.04 (one-way ANOVA). cGAMP concentrations were measured in the conditioned media. Mean±SEM (n=2). **P=0.002 (one-way ANOVA). Data are representative of two independent experiments.

FIG. 8, panels A to D: Developing an LC-MS/MS method and building 293T cGAS ENPP1^{low} and 293T cGAS ENPP1^{-/-} cell lines. a, Liquid chromatography traces of cGAMP at 0, 20, and 80 nM: single isotopically-labeled internal standard cGAMP (15 Da heavier) at 500 nM; and double isotopically-labeled extraction standard cGAMP (30 Da heavier) at 2 M. Chemical structures of all analytes. b, Calibration of cell number to ATP concentration measured by LC-MS/MS. Mean f SEM (n=2). c, cGAS expression of 293T, 293T cGAS ENPP1^{-/-}, and 293T cGAS ENPP1^{low} cell lines analyzed by western blot (left). ENPP1 hydrolysis activity of ³²P-cGAMP in whole cell lysates from 1 million each of 293T cGAS, 293T cGAS ENPP1^{-/-}, and 293T cGAS ENPP1^{low} cells, measured by TLC and autoradiography (right). Lysate data are representative of two independent experiments. d, Coomassie gel of recombinant mouse ENPP1 purified from media: elution fractions were pooled before use (left). ³²P-cGAMP degradation by mouse ENPP1 analyzed by TLC (right).

FIG. 9, panel A: CD14⁺ PBMCs respond to extracellular cGAMP. a, Schematic of stimulation of CD14+ PBMCs with extracellular cGAMP. b, IFNB1 induction measured by RT-qPCR for human CD14⁺ PMBCs stimulated with increasing concentrations of extracellular cGAMP for 16 h. Mean±SEM (n=2 technical qPCR replicates).

ENPP1 Regulates Only Extracellular cGAMP

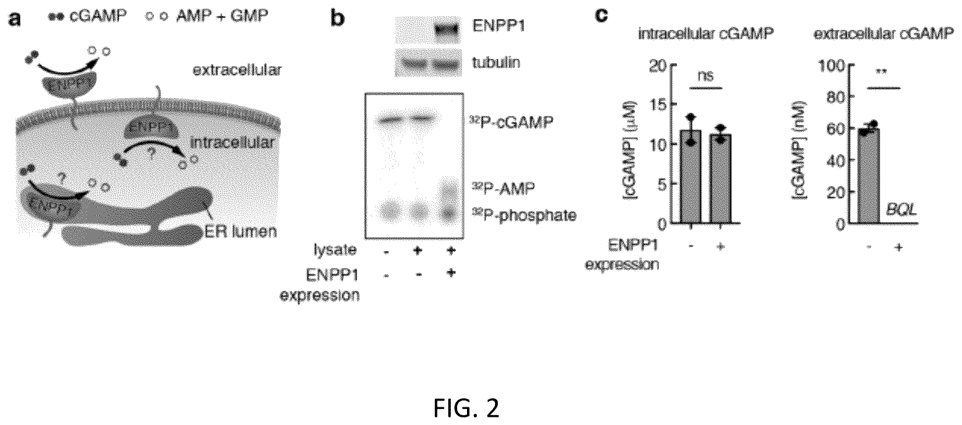

Since we were able to observe extracellular cGAMP for the first time when we knocked out ENPP1 from 293T cells and cultured them in ENPP1-free media, we then investigated whether only extracellular cGAMP is regulated by ENPP1. Despite its extracellular annotation, it is possible that ENPP1 could flip orientation on the membrane, as for enzyme CD38 (see e.g., Zhao, Y. J., Lam, C. M. C. & Lee, H. C. The membrane-bound enzyme CD38 exists in two opposing orientations. Sci. Signal. 5, ra67 (2012)), or it could be active when being synthesized in the ER lumen and cGAMP may cross the ER membrane (FIG. 2, panel A). To investigate the localization of ENPP1 activity, we transfected 293T cGAS ENPP1^{-/-} cells with human ENPP1 expression plasmid and confirmed its activity in whole cell lysates (FIG. 2, panel B). In intact cells, ENPP1 expression depletes extracellular cGAMP, but does not affect the intracellular cGAMP concentration (FIG. 2, panel C). Therefore, extracellular, but not intracellular, cGAMP is regulated by ENPP1 in these cells.

FIG. 2, panels A to C: ENPP1 only regulates extracellular cGAMP. a, Three possible cellular locations of ENPP1 activity. b, 293T cGAS ENPP1^{-/-} cells were transfected with empty vector or vector containing human ENPP1 and analyzed after 24 h for ENPP1 protein expression using western blot (top) and for ENPP1 ³²P-cGAMP hydrolysis activity using thin layer chromatography (TLC) (bottom). Data are representative of two independent experiments. c, Intracellular and extracellular cGAMP concentrations measured using LC-MS/MS. BQL=below quantification limit. Mean±SEM (n=2). **P=0.002 (Student's t test). Data are representative of three independent experiments.

### Development of a Cell Impermeable ENPP1 Inhibitor

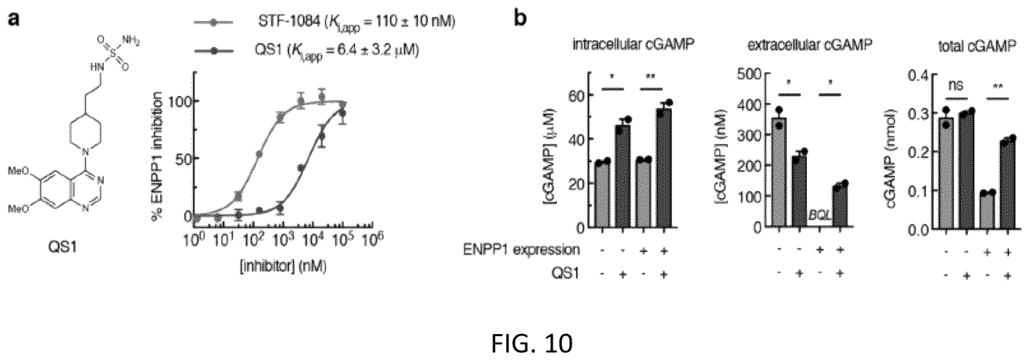

To study the physiological relevance of extracellular cGAMP and why it needs to be regulated by a specific hydrolase, we sought to manipulate its concentration by pharmacologically inhibiting ENPP1. We first tested a nonspecific ENPP1 inhibitor QS1 (FIG. 10, panel A) (Patel, S. D. et al. Quinazolin-4-piperidin-4-methyl sulfamide PC-1 inhibitors: Alleviating hERG interactions through structure based design. Bioorganic Med. Chem. Lett. 19.3339-3343 (2009); and Shayhidin, E. E. et al. Quinazoline-4-piperidine sulfamides are specific inhibitors of human NPP1 and prevent pathological mineralization of valve interstitial cells. Br. J. Pharmacol. 172, 4189-4199 (2015)). Although QS1 can inhibit extracellular cGAMP degradation in cells overexpressing ENPP1, it also partially blocked cGAMP export in ENPP1 knockout cells (FIG. 10, panel B). QS1 treated cells have elevated intracellular cGAMP, demonstrating again that export is a significant mechanism to maintain cGAMP homeostasis in cancer cells. The export blockade activity excludes QS1 as a tool to study extracellular cGAMP in our export studies. Phosphonate analog, compound 1, was designed to chelate Zn²⁺ at the ENPP1 catalytic site and to minimize cell permeability and avoid intracellular off-targets (FIG. 3, panel A). Compound 1 has a K_{i,app} of 110±10 nM (FIG. 3, panel B), which is ˜60 fold more potent than QS1 (FIG. 10, panel A).

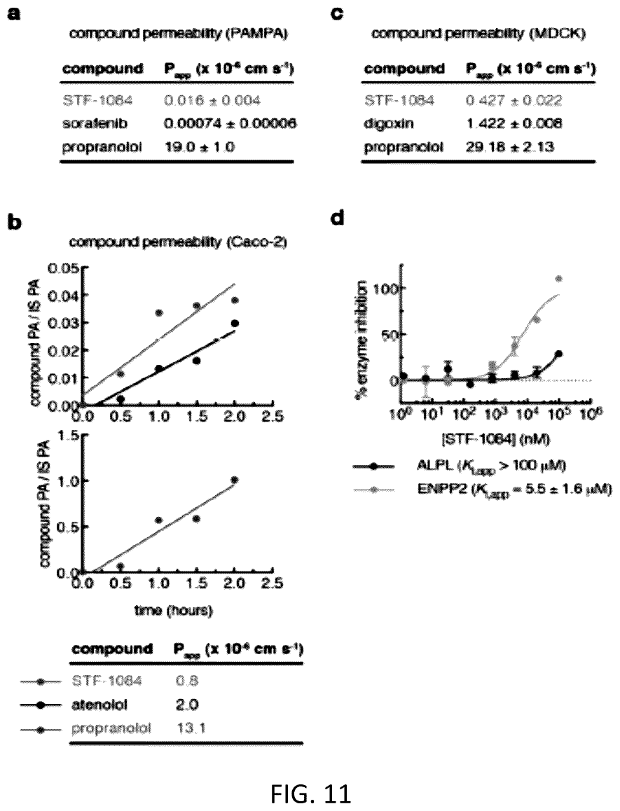

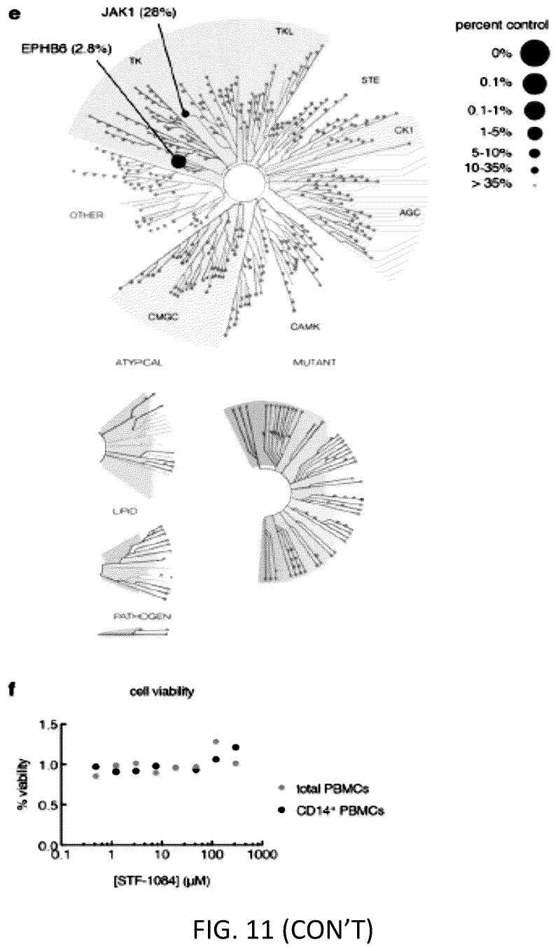

We confirmed that compound 1 is cell impermeable by performing three independent permeability assays; the parallel artificial membrane permeability assay (PAMPA) (FIG. 11, panel A); the intestinal cells Caco-2 permeability assay (FIG. 11, panel B), and the epithelial cells MDCK permeability assay (FIG. 11, panel C). Compared to control compounds with high cell permeability and low cell permeability, compound 1 falls into the category of impermeable compounds in all three assays. In addition, it has low activity towards the closely related ectonucleotidases alkaline phosphatase (K_{i,app}>100 μM) and ENPP2 (K_{i,app}=5.5 M) (FIG. 11, panel D). Although we do not expect compound 1 to have intracellular off-targets due to its low cell permeability, we tested its binding against a panel of 468 kinases to further determine its specificity. Despite its structural similarity to AMP, compound 1 binds to only two kinases at 1 μM (FIG. 11, panel E). Compound 1 also shows high stability (t_{1/2}>159 min) in both human and mouse liver microsomes. Together, we demonstrated that compound 1 is a potent, cell impermeable, specific, and stable ENPP1 inhibitor.

Next, we measured the efficacy of compound 1 in maintaining extracellular cGAMP concentrations of ENPP1 overexpressing 293T cGAS cells and obtained an IC₅₀ value of 340±160 nM (FIG. 3, panel C) with 10 μM being sufficient to completely block extracellular cGAMP degradation (FIG. 3, panel D). Unlike QS1, compound 1 had no effect on intracellular cGAMP, demonstrating that it does not affect cGAMP export (FIG. 3, panel D). Compound 1 is, therefore, an excellent ENPP1 inhibitor tool compound to specifically increase extracellular cGAMP concentrations.

Finally, we tested the efficacy of compound 1 in boosting extracellular cGAMP signal detectable to CD14⁺ PBMCs. We first confirmed that compound 1 is not toxic to PBMCs at the concentrations used (FIG. 11, panel F). Conditioned media from ENPP1 overexpressing 293T cGAS cells failed to induce IFNB1 expression in CD14⁺ cells (FIG. 3, panels E and F). However, compound 1 rescued extracellular cGAMP levels in the media and induction of IFNB1 expression in CD14⁺ cells (FIG. 3, panel F). These results demonstrate that the enzymatic activity of ENPP1, not potential scaffolding effects as a transmembrane protein, dampens response to extracellular cGAMP by CD14⁺ PBMCs. Together, our data suggest that extracellular cGAMP levels can be decreased by ENPP1 expression and increased by ENPP1 inhibition, which affects the activation of CD14⁺ PBMCs in vitro.

FIG. 3, panels A to F: Activity of a cell impermeable ENPP1 inhibitor. a, Chemical structure of compound 1.b, Inhibitory activity of compound 1 against purified mouse ENPP1 with ³²P-cGAMP as the substrate at pH 7.5 (K_{i,app}=110±10 nM). Mean±SEM (n=3 independent experiments) with some error bars too small to visualize. c, Inhibitory activity of compound 1 against human ENPP1 transiently expressed in 293T cGAS ENPP1^{-/-} cells (IC₅₀=340±160 nM). Mean t SEM (n=2). d, Intracellular and extracellular cGAMP concentrations for 293T cGAS ENPP1^{-/-} cells transfected with empty pcDNA vector or vector containing human ENPP1 in the presence or absence of 10 μM compound 1. BQL=below quantification limit. Mean±SEM (n=3). ****P<0.0001 (one-way ANOVA). Data are representative of two independent experiments. e, Schematic of the conditioned media experiment. 293T cGAS ENPP1^{low} cells were transfected with vector containing human ENPP1 and incubated in the presence or absence of compound 1. Conditioned media from these cells was transferred to primary CD14⁺ human PBMCs. f, IFNB1 mRNA levels were normalized to CD14 and the fold induction was calculated relative to untreated CD14⁺ cells. Mean±SEM (n=2). **P=0.007 (one-way ANOVA). cGAMP concentrations were measured in the conditioned media. Mean±SEM (n=2). **P=0.006 (one-way ANOVA). Data are representative of two independent experiments.

FIG. 10, panels A to B: Improvement of compound 1 over QS1.

a, Structure of QS1 and its inhibitory activity (compared to compound 1) against purified mouse ENPP1 with ³²P-cGAMP as the substrate at pH 7.5 (QS1 K_{i,app}=6.4±3.2 M). Mean±SEM (n=2 independent experiments). b, Intracellular, extracellular, and total cGAMP for 293T cGAS ENPP1^{-/-} cells transfected with empty vector or vector containing human ENPP1 in the presence or absence of QS1. Mean±SEM (n=2). *P<0.05. **P<0.01 (one-way ANOVA).

FIG. 11, panels A to F: compound 1 is cell impermeable, specific to ENPP1, and nontoxic. a, Permeability of compound 1 in artificial membrane permeability assay (PAMPA). b, Permeability of compound 1 in intestinal cells Caco-2 assay. PA=peak area, IS=internal standard. Compounds, including compound 1, atenolol (low passive permeability negative control) and propranolol (high passive permeability positive control), were incubated on the apical side of a Caco-2 monolayer for 2 hours. Compound concentration on the basolateral side was monitored by LC-MS/MS. Apparent permeability rates (Pₐₚₚ) were calculated from the slope. Data are representative of two independent experiments. c, Permeability of compound 1 in epithelial cells MDCK permeability assay. d, Inhibitory activity of compound 1 against alkaline phosphatase (ALPL) and ENPP2. Mean±SEM (n=2). e, Kinome interaction map (468 kinases tested) for compound 1 depicting kinase inhibition as a percent of control. Image generated using TREEspot™ Software Tool and reprinted with permission from KINOMEscan®, a division of DiscoveRx Corporation. ©DiscoveRX Corporation 2010. f, Cell viability measured by CellTiterGlo. Total PBMCs and CD14⁺ PBMCs were incubated with compound 1 for 16 hours and then assayed for ATP levels using CellTiterGlo. Data was normalized to no compound 1 to calculate % cell viability.

Cancer Cells Express cGAS and Continuously Export cGAMP in Culture

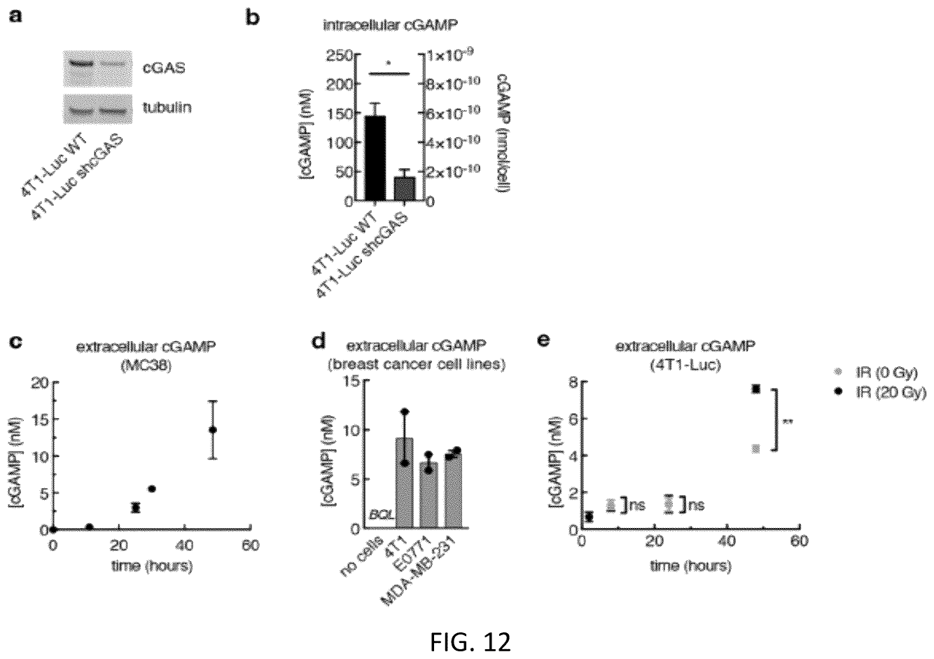

To determine if extracellular cGAMP can function as a danger signal secreted by cancer cells in vivo, we first sought to identify tumor models that export cGAMP. We tested one human (MDA-MB-231) and three mouse cancer cell lines (E0771. MC38, and 4T1-Luc, which is a 4T1 cell line expressing luciferase for in vivo imaging) in culture, all of which express cGAS (FIG. 4, panel A). Intracellular cGAMP concentrations in these cells are difficult to detect. However, with extra concentration and purification steps, we were able to detect 5.8×10⁻¹⁰ nmol/cell (˜150 nM) intracellular cGAMP in 4T1-Luc cells (FIG. 4, panel B). Knocking down cGAS using shRNA leads to decreased cGAS protein levels and decreased intracellular cGAMP levels, demonstrating that cGAS expression controls the amount of cGAMP present in the 4T1-Luc cells (FIG. 12, panels A and B). Using compound 1 to inhibit cell surface and soluble ENPP1 in the cell culture media, we detected continuous cGAMP export in all of these cell lines and extracellular cGAMP levels reached ˜6×10⁻⁹ nmol/cell (˜10 nM when diluted into the media) in 48 h (FIG. 4, panels C and D and FIG. 12, panels C and D). Remarkably, this is about 10-fold the amount of cGAMP present inside cells, suggesting that cancer cells efficiently clear out their cGAMP by export. Ionizing radiation (IR) can increase cytosolic DNA and activate cGAS-dependent IFN-β production in tumor cells (see e.g., Bakhoum, S. F. et al. Numerical chromosomal instability mediates susceptibility to radiation treatment. Nat. Commun. 6, 1-10 (2015); and Vanpouille-Box, C. et al. DNA exonuclease Trex1 regulates radiotherapy-induced tumour immunogenicity. Nat. Commun. 8, 15618 (2017)). Indeed. IR treatment also increased extracellular cGAMP production in 4T1-Luc cells after 2 days (FIG. 4, panel E and FIG. 12, panel E). Together, our data demonstrate that these cancer cell lines constantly produce and efficiently export cGAMP and can be stimulated with IR to produce more extracellular cGAMP.

FIG. 4, panels A to E: Cancer cells express cGAS and continuously export cGAMP in culture. a, cGAS expression of 4T1-Luc, E0771, MDA-MB-231, and MC38 analyzed by western blot. b, Estimate of the concentration of intracellular cGAMP in 4T1-Luc cells without exogenous stimulation. Mean±SEM (n=2). c, Extracellular cGAMP produced by MC38 cells over 48 hours. At time 0, cells were refreshed with media supplemented with 50 μM compound 1. Mean±SEM (n=2). Data are representative of two independent experiments. d, Extracellular cGAMP produced by 4T1-Luc, E0771, and MDA-MB-231 cells measured after 48 h in the presence of 50 μM compound 1. BQL=below quantification limit. Mean±SEM (n=2). e, Extracellular cGAMP produced by 4T1-Luc cells over 48 hours. At time 0, cells were left untreated or treated with 20 Gy IR and refreshed with media supplemented with 50 μM compound 1. Mean±SEM (n=2). *P=0.04 (Student's t test).

FIG. 12, panels A to E: Cancer cells continuously export cGAMP in culture. a, cGAS expression of 4T1-Luc WT and 4T1-Luc shcGAS cell lines analyzed by western blot. b, Intracellular cGAMP of 4T1-Luc WT and 4T1-Luc shcGAS cell lines without exogenous stimulation. Mean f SEM (n=2). 4T1-Luc WT data is repeated from FIG. 4, panel b for comparison. c, Extracellular cGAMP (depicted in media concentration units) of experiment shown in FIG. 4, panel c. d, Extracellular cGAMP (depicted in media concentration units) of experiment shown in FIG. 4, panel d. BQL=below quantification limit. Mean±SEM (n=2). e, Extracellular cGAMP (depicted in media concentration units) of experiment shown in FIG. 4, panel c. Mean±SEM (n=2). **P=0.004 (Student's t test).

Sequestration of Extracellular cGAMP Decreases Tumor-Associated Dendritic Cells Dependent on Tumor cGAS and Host STING

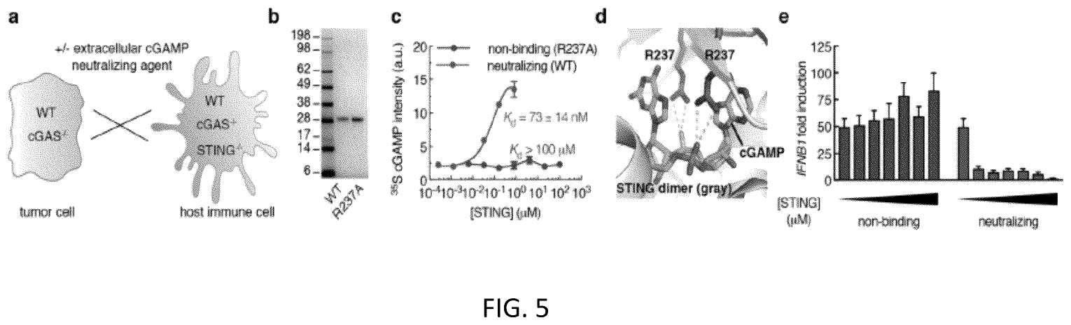

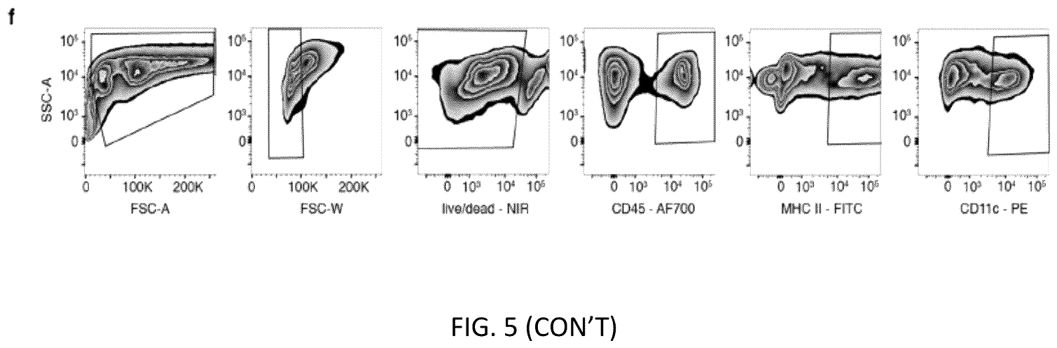

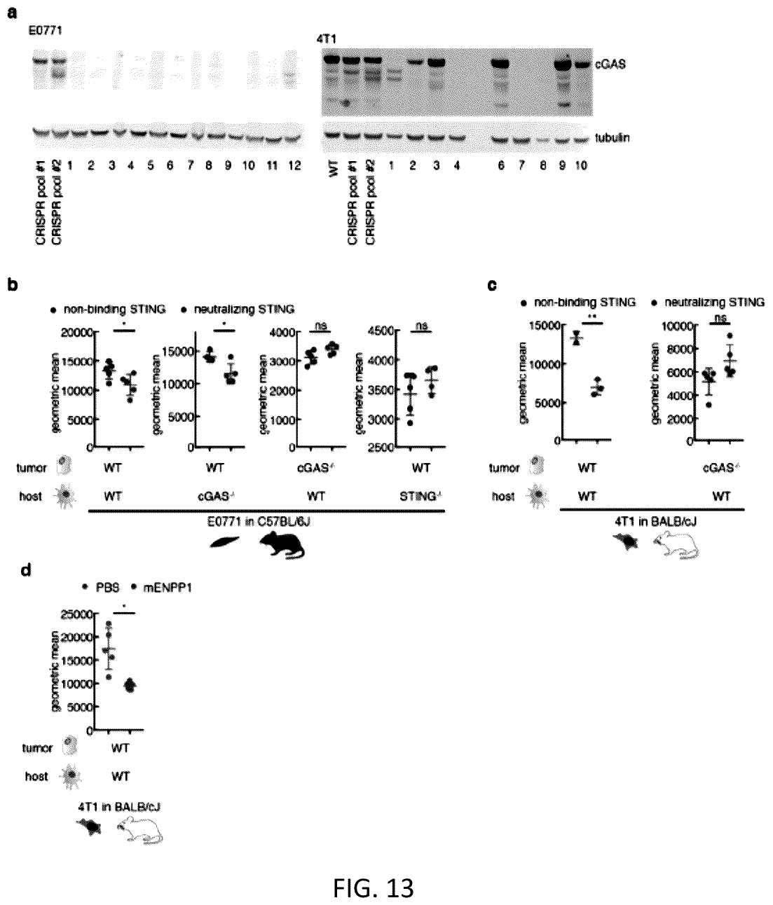

In tumors, the extracellular space is estimated to be 0.3-0.8-fold the volume of the intracellular spaces²⁸. In cell culture, however, the volume of the extracellular space is approximately 250-1000-fold the volume of the intracellular space. We use 1 mL of culture media per 1×10⁶ cells and estimate a cell volume of ˜1-4 pL to perform this calculation. Our cell culture system is, therefore, diluting the extracellular space by 300-3000-fold compared to in the tumor microenvironment. Given this dilution factor and our measurement of nanomolar extracellular cGAMP exported by cancer cells in vitro, we predict that extracellular cGAMP in the tumor microenvironment can reach the micromolar range, which may lead to innate immune recognition of tumor cells. Recognizing the limits of our in vitro cell experiments, we turned to in vivo experiments to investigate the role of extracellular cGAMP (FIG. 5, panel A). First, we sought to determine the importance of tumor versus host cGAMP by knocking out cGAS in tumor cells (FIG. 13, panel A) and utilized cGAS^{-/-} and STING^{-/-} mice in the C57BL/6 background. We also developed a neutralizing agent as a tool to specifically sequester extracellular cGAMP (FIG. 5, panel A). We took advantage of the soluble cytosolic domain of STING (FIG. 5, panel B), which binds cGAMP with a K_{d} of 73±14 nM (FIG. 5, panel C). We also generated an R237A mutant STING (see e.g., Gao, P. et al., Cell 154, 748-762 (2013)) as a non-binding STING control (FIG. 5, panels B-D). To test the neutralizing efficacy of these proteins in cell culture, we used CD14⁺ PBMCs. Wild type (WT) STING (neutralizing STING) was able to neutralize extracellular cGAMP with the predicted 2:1 stoichiometry, while the non-binding STING had no effect even when at a 200-fold higher concentration (FIG. 5, panel E).

We established E0771 orthotopic tumors in mice, followed by intratumoral injection of neutralizing STING to deplete extracellular cGAMP, and excision of the tumors to stain for tumor-associated leukocytes. In WT E0771 tumors, neutralizing STING significantly decreased the CD11c⁺ dendritic cell population in the total CD45⁺/MHC-II⁺ tumor-associated antigen presenting cell (APCs) population, suggesting that extracellular cGAMP can be detected by the immune system (FIG. 5, panels F and G and FIG. 13, panel B). Extracellular cGAMP depletion also diminished the CD11c⁺ population when tumors are grown in cGAS^{-/-} mice, suggesting that host cells do not contribute significantly to extracellular cGAMP production (FIG. 5, panel G and FIG. 13, panel B). In contrast, extracellular cGAMP depletion did not affect the CD11c⁺ population when cGAS^{-/-} E0771 cells (multiple clones were pooled to achieve clean knockout but minimize clonal effects) or STING^{-/-} mice were used. This demonstrates that tumor cells, but not host cells, are the dominant producers of extracellular cGAMP, which is then sensed by host STING (FIG. 5, panel G and FIG. 13, panel B). We also tested the orthotopic 4T1-Luc tumor model in the BALB/c background. Although cGAS and STING knockout strains have not been established in this background, we knocked out cGAS in the 4T1-Luc tumors. Intratumoral injection of neutralizing STING into the WT 4T1-Luc tumors significantly decreased the tumor-associated CD11c⁺ population in the CD45⁺/MHC II⁺ population (FIG. 5, panel H and FIG. 13, panel C). In contrast, extracellular cGAMP depletion had no effect in cGAS^{-/-} 4T1-Luc tumors (FIG. 5, panel H and FIG. 13, panel C). We also depleted extracellular cGAMP by intratumoral injection of mENPP1 protein (FIG. 8, panel D) and again observed diminished CD11c⁺ cells in the CD45⁺/MHC II⁺ population (FIG. 5, panel I and FIG. 13, panel D). Results from our E0771 and 4T1-Luc models together demonstrate that extracellular cGAMP produced by tumor cells activates innate immune responses in a manner dependent on host STING but independent of host cGAS. Together, our data demonstrate that extracellular cGAMP produced by cancer cells is a danger signal that elicits innate immune responses.

FIG. 5, panels A to I: Sequestration of extracellular cGAMP decreases tumor-associated dendritic cells in a tumor cGAS and host STING dependent manner. a, Experimental setup to assess the role of extracellular cGAMP in vivo. b, Coomassie gel of recombinant mouse WT STING and R237A STING.c, Binding curves for the cytosolic domain of mouse WT STING (neutralizing) and R237A STING (non-binding) determined by a membrane binding assay using radiolabeled ³⁵S-cGAMP as the probe. Mean±SEM (n=2 from two independent experiments). d, Crystal structure of mouse WT STING in complex with cGAMP with R²³⁷ highlighted in pink (PDB ID 4LOJ). e, IFNB1 mRNA fold induction in CD14⁺ PBMCs treated with 2 FM cGAMP in the presence of neutralizing or non-binding STING (2 μM to 100 μM, 2.5-fold dilutions). Mean±SEM (n=2 technical qPCR replicates). f, WT or cGAS^{-/-} E0771 cells (1×10⁶) were orthotopically injected into WT, cGAS^{-/-}, or STING-C57BL/6J mice on day 0. Neutralizing (WT mice n=5: cGAS^{-/-} mice n=5; STING^{-/-} mice n=4) or non-binding STING (WT mice n=5: cGAS^{-/-} mice n=4: STING^{-/-} mice n=5) was intratumorally injected on day 2. Tumors were harvested and analyzed by FACS on day 3. Samples were gated on cells in FSC-A/SSC-A, singlets (FSC—W), living cells, CD45, MHC II⁺, CD11c⁺ populations. g, Percent CD11c⁺ cells of total APCs. Mean±SD. *P=0.015. **P=0.008 (Welch's t test). h, The same procedure was performed as in (f) and (g) with WT (neutralizing STING n=3: non-binding STING n=2) or cGAS^{-/-} 4T1-Luc cells (n=5) in WT BALB/cJ mice. Mean±SD. *P=0.011. (Welch's t test). i, 4T1-Luc cells (1×10⁶) were orthotopically injected into WT BALB/cJ mice on day 0. PBS (n=5) or recombinant mouse ENPP1 (mENPP1) (n=6) was intratumorally injected on day 2. Tumors were harvested and analyzed by FACS on day 3. Mean±SD. *P=0.033. (Welch's t test).

FIG. 13, panels A to D: Sequestration of extracellular cGAMP decreases tumor-associated dendritic cells in a tumor cGAS and host STING dependent manner. a, E0771 (left) and 4T1-Luc (right) cGAS^{-/-} cells subcloned from CRISPR knockout pools. E0771 cGAS^{-/-} subclones 1, 2, 4, 6, 8, and 9 were pooled before injection into mice. 4T1-Luc cGAS^{-/-} subclones 4, 7, and 8 were pooled before injection into mice. b, Geometric means of experiments shown in FIG. 5g. Mean±SD. *P=0.049 (WT tumor/WT host). *P=0.015 (WT tumor/cGAS^{-/-} host). (Welch's t test). c, Geometric means of experiments shown in FIG. 5h. Mean±SD. **P=0.009 (Welch's t test). d, Geometric means of experiments shown in FIG. 5i. Mean±SD. *P<0.015 (Welch's t test).

Increasing Extracellular cGAMP by Reducing ENPP1 Activity Increases Dendritic Cell Infiltration and Renders Breast Tumors More Treatable.

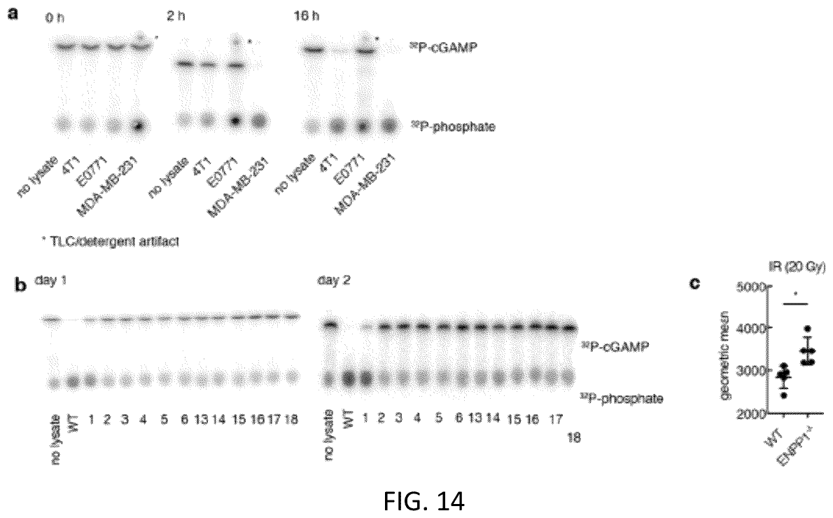

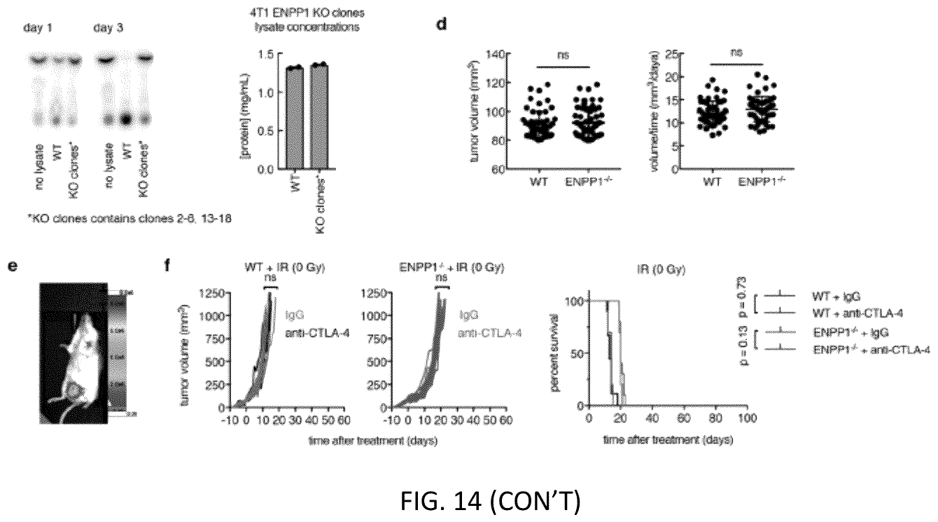

ENPP1 is highly expressed in some breast cancers and its level has been correlated with poor prognosis (see e.g., Lau, W. M. et al. Enpp1: A Potential Facilitator of Breast Cancer Bone Metastasis. PLoS One 8, 1-5 (2013); Takahashi, R. U. et al. Loss of microRNA-27b contributes to breast cancer stem cell generation by activating ENPP1. Nat. Commun. 6, 1-15 (2015); and Umar, A. et al. Identification of a Putative Protein Profile Associated with Tamoxifen Therapy Resistance in Breast Cancer. Mol. Cell. Proteomics 8, 1278-1294 (2009)). High ENPP1 expression may be a mechanism breast cancers utilize to deplete extracellular cGAMP and dampens immune detection. We measured ENPP1 activities in three triple negative breast cancer cells 4T1-Luc, E0771, and MDA-MB-231, with MDA-MB-231 and 4T1-Luc exhibiting high ENPP1 activities (FIG. 14, panel A). We, therefore, chose the triple negative, metastatic, and orthotopic 4T1-Luc mouse model to probe the effect of ENPP1 on tumor immune detection, growth, and responses to treatment.

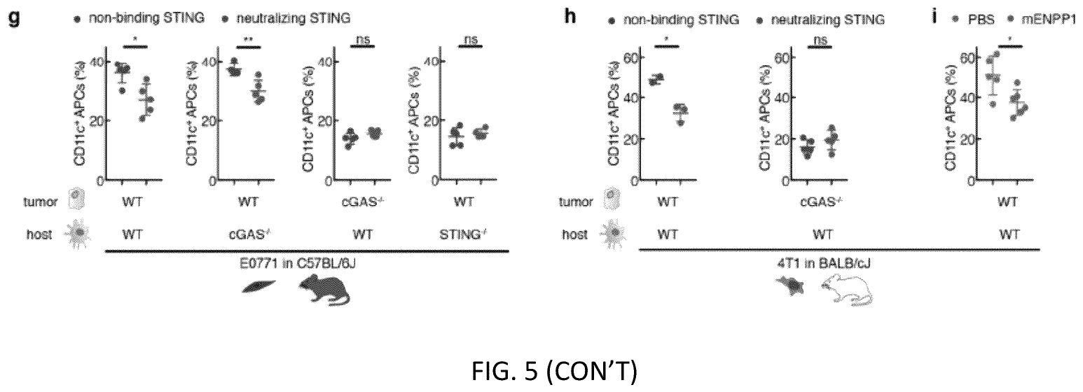

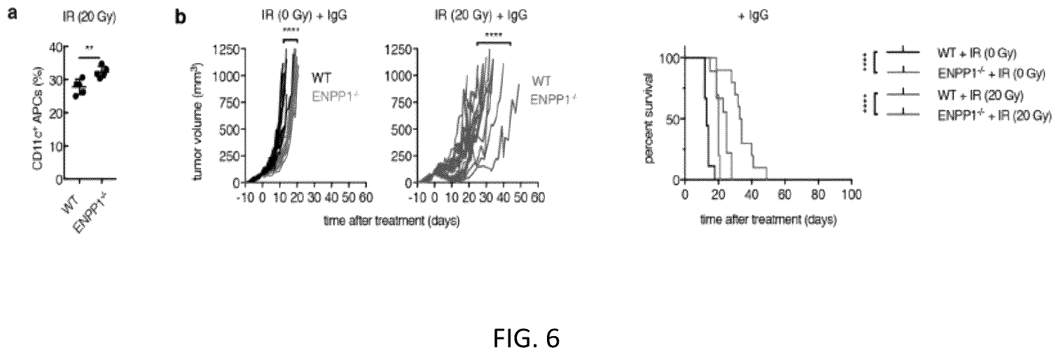

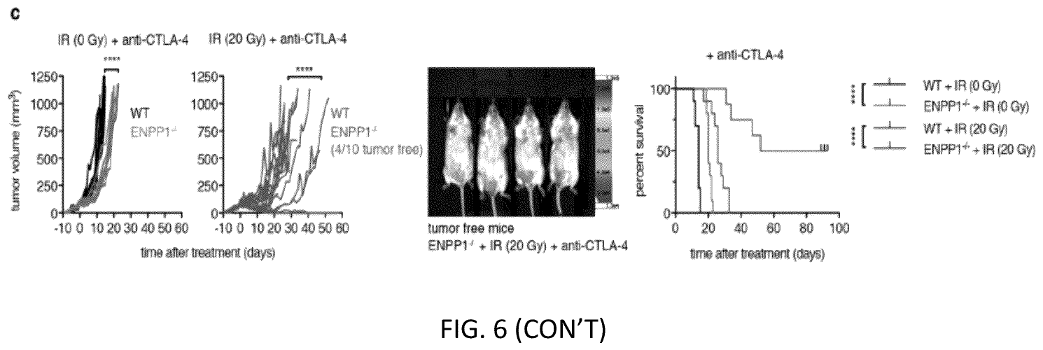

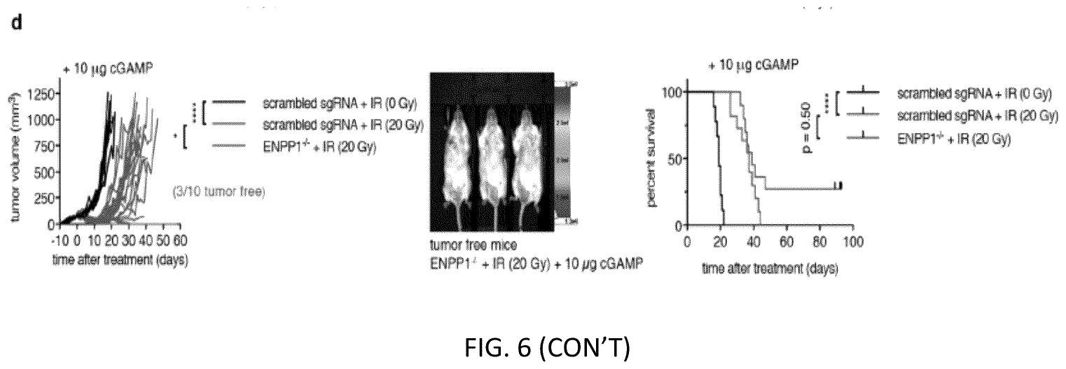

We first tested the effect of ENPP1 on tumor infiltrating dendritic cells. We knocked out ENPP1 in 4T1-Luc cells, validated the clones by their lack of enzymatic activity (commercially available ENPP1 antibodies are not sensitive enough to validate knockout), and pooled multiple clones to minimize clonal effects (FIG. 14, panel B). After 4T1-Luc implantation, we treated tumors with a dose of 20 Gy IR to induce cGAMP production and excised the tumors after 24 hours to analyze their tumor-associated leukocyte compositions. ENPP1^{-/-} tumors have a larger tumor-associated CD11c⁺ population than WT tumors (FIG. 6, panel A and FIG. 14, panel C). We then tested the effect of ENPP1 on immune rejection of 4T1-Luc tumors. This is an aggressive tumor model that typically metastasizes into the lungs within two weeks of tumor implantation (Pulaski. B. A. & Ostrand-Rosenberg, S. Reduction of Established Spontaneous Mammary Carcinoma Metastases following Immunotherapy with Major Histocompatibility Complex Class II and B7.1 Cell-based Tumor Vaccines. Cancer Res. 58, 1486-1493 (1998)). The initial tumor growth rate for ENPP1^{-/-} tumors before they reached 100 mm³ was the same as WT tumors suggesting that we did not select for slow growing clones (FIG. 14, panel D). However, the established ENPP1^{-/-} tumors are less aggressive (FIG. 6, panel B) and are more responsive to IR (FIG. 6, panel B). Without IR, the adaptive immune checkpoint blocker anti-CTLA-4 does not synergize with ENPP1 in shrinking tumors (FIG. 14, panels E and F). Strikingly, when we used IR to induce cGAMP production, anti-CTLA-4 cured 40% of ENPP1^{-/-} tumors, but none of the WT tumors (FIG. 6, panel C). Direct intratumoral injection of extracellular cGAMP is more effective in ENPP1 tumors than in WT tumors, and synergized with IR to cure 30% of mice (FIG. 6, panel D) without the presence of anti-CTLA-4. Together, ENPP1 dampens extracellular cGAMP, innate immune detection of 4T1-Luc tumors, and negatively affects their responses to IR and adaptive immune checkpoint blockade.

FIG. 6, panels A to D: ENPP1^{-/-} tumors recruit innate immune infiltration, are less aggressive, and more susceptible to IR and anti-CTLA-4 therapy. a, WT or ENPP1^{-/-} 4T 1-Luc cells (1×10⁶) were orthotopically injected into WT BALB/cJ mice on day 0 (n=5 for each group) Tumors were treated with IR at 20 Gy on day 2. Tumors were harvested and analyzed by FACS on day 3. Multiple ENPP1^{-/-} 4T1-Luc cell clones were pooled before injection to minimize clonal effects. **P=0.008 (Welch's t test). b, Established WT or ENPP1^{-/-} 4T1-Luc tumors (100±20 mm³) were treated once with 0 Gy or 20 Gy IR followed by three intraperitoneal injections of IgG on day 2, 5, and 7 after IR. (n=9 for WT 4T1-Luc, n=10 for ENPP1^{-/-} 4T1-Luc). Tumor volumes and Kaplan Meier curves are shown. P values determined by pairwise comparisons using post hoc tests with a Tukey adjustment at day 20 (tumor volumes) and the log-rank Mantel-Cox test (Kaplan Meier). ****P<0.0001. c, Established WT or ENPP1^{-/-} 4T1-Luc tumors (100+20 mm³) were treated once with 0 Gy or 20 Gy IR followed by three intraperitoneal injections of anti-CTLA-4 on day 2, 5, and 7 after IR (n=10 for all groups). Tumor volumes and Kaplan Meier curves are shown. P values determined by pairwise comparisons using post hoc tests with a Tukey adjustment at day 20 (tumor volumes) and the log-rank Mantel-Cox test (Kaplan Meier). ****P<0.0001. In the ENPP1^{-/-} 4T1-Luc+IR (20)+anti-CTLA-4 treatment group, 4/10 (40%) mice are tumor-free survivors verified by bioluminescent imaging. d, Established 4T1-Luc tumors infected with a scrambled sgRNA sequence or ENPP1® 4T1-Luc tumors (100±20 mm³) were treated once with 20 Gy IR followed by three intratumoral injections of 10 sg cGAMP on day 2, 4, and 7 after IR (n=10 for both groups). Tumor volumes and Kaplan Meier curves are shown. P values determined by pairwise comparisons using post hoc tests with a Tukey adjustment at day 20 (tumor volumes) and the log-rank Mantel-Cox test (Kaplan Meier). *P<0.05, ****P<0.0001. In the ENPP1^{-/-} 4T1-Luc+IR (20) cGAMP treatment group, 3/10 (30%) mice are tumor-free survivors verified by bioluminescent imaging. Mice from different treatment groups in b-d were co-housed and the experimenter was blinded.

FIG. 14, panels A to F: Established ENPP1^{-/-} tumors lead to increased tumor-associated dendritic cells, are less aggressive, and more susceptible to IR and anti-CTLA-4 therapy. a, ENPP1 activity in 4T1-Luc, E0771, and MDA-MB231 cells using the ³²P-cGAMP degradation assay. Data are representative of three independent experiments. b, Validating ENPP1^{-/-} 4T1-Luc clones using the ³²P-cGAMP degradation assay. Lysates from different clones were normalized by protein concentrations. ENPP1^{-/-} 4T1-Luc clones 2-6 and 13-18 were pooled before injection into mice. c, Geometric means of experiments shown in FIG. 6a. Mean±SD. *P=0.012 (Welch's t test), d, (left) Tumor volume of WT (n=55) vs ENPP1^{-/-} (n=55) 4T1-Luc cells on the day of treatment; (right) initial tumor growth rate expressed as tumor volume/days needed to reach a size of 100 mm³±20 mm³. Mean±SD (Welch's t test). e, Bioluminescent image of a tumor-bearing mouse. f, Replotting of data shown in FIG. 6a, b to highlight comparisons between IgG and anti-CTLA-4 treated groups. Established WT or ENPP1^{-/-} 4T1-Luc tumors (100±20 mm³) were treated with three intraperitoneal injections of IgG or anti-CTLA-4 on day 2, 5, and 7 after tumors reached the requisite size. (n=9 for WT 4T1-Luc+IgG, n=10 for all other groups). Tumor volumes and Kaplan Meier curves are shown. P values determined by pairwise comparisons using post hoc tests with a Tukey adjustment at day 20 (tumor volumes) and the log-rank Mantel-Cox test (Kaplan Meier).

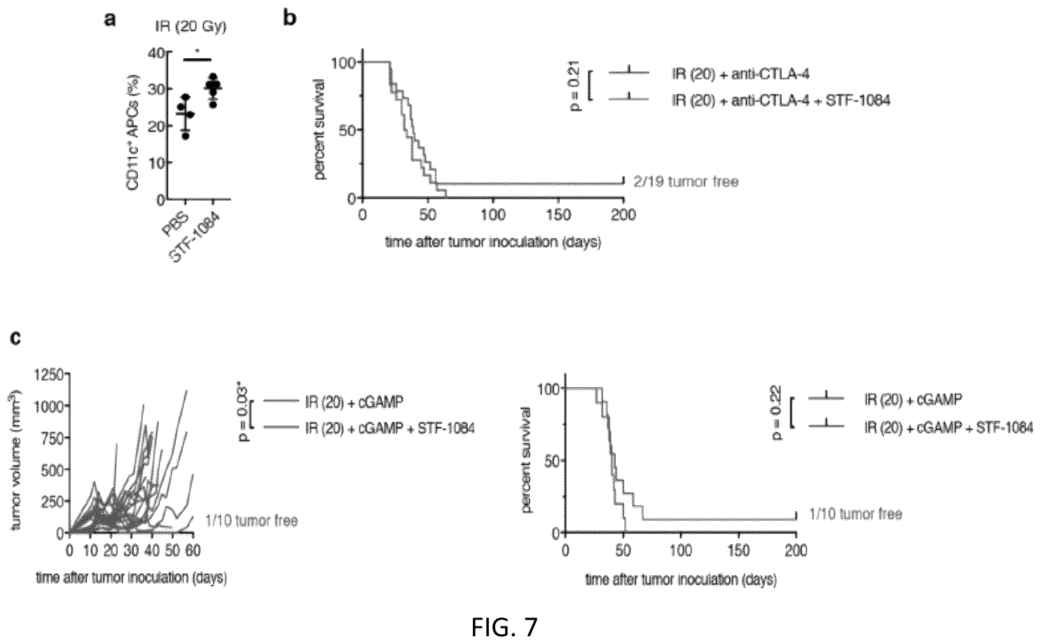

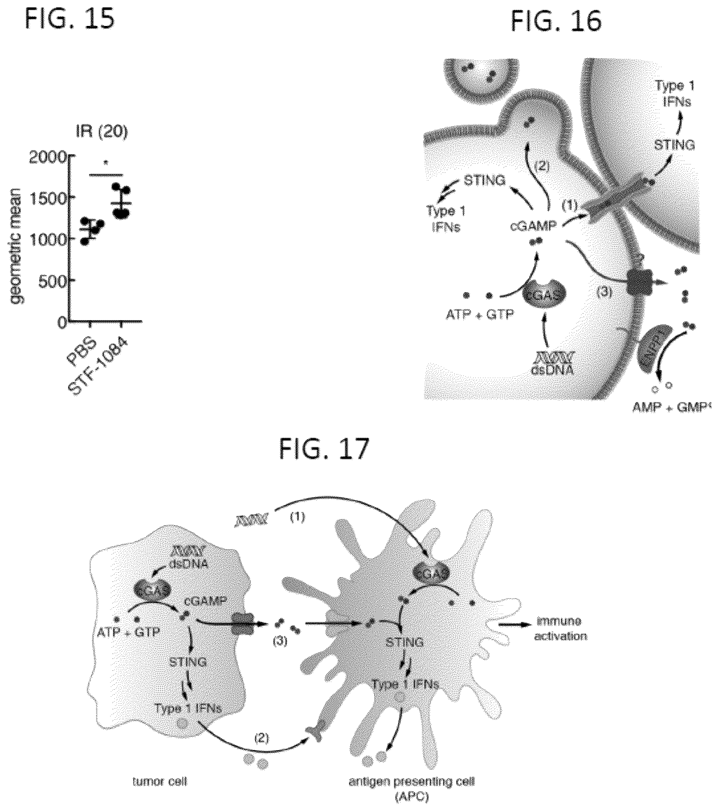

Our genetic results suggest that ENPP1 is a potential target for pharmacological inhibition. The ENPP1 inhibitor we developed, compound 1, exhibits fast clearance when intratumorally injected. Without extensive studies of route of administration and the corresponding formulation optimization that pharmaceutical companies typically perform at a later stage of drug development, we asked whether compound 1 has an effect in vivo. We injected tumors with compound 1 immediately after IR treatment and observed an increase in the tumor-associated CD11c⁺ population after 24 hours (FIG. 7, panel A and FIG. 15). Remarkably, compound 1 synergized with IR and anti-CTLA4 to achieve a 10% cure rate (FIG. 7, panel B). Finally, it was observed that compound 1 synergized with IR and cGAMP to shrink tumors, prolong survival, and achieve a 10% cure rate (FIG. 7, panel C). Together, these results demonstrate that ENPP1 can be targeted pharmacologically to enhance innate immune recognition of cancer.

FIG. 7, panels A to C: ENPP1 inhibition synergizes with IR treatment and anti-CTLA-4 to exert anti-tumor effects. a, 4T1-Luc cells (1×10⁶) were orthotopically injected into WT BALB/cJ mice on day 0. Tumors were treated with 20 Gy IR and intratumorally injected with PBS (n=4) or compound 1 (n=5) on day 2. Tumors were harvested and analyzed by FACS on day 3. *P=0.047 (Welch's t test). b, Established 4T1-Luc tumors (100±20 mm³) were treated once with 20 Gy IR followed by three intratumoral injections of PBS or compound 1 on day 2, 4, and 7 and intraperitoneal injections of anti-CTLA-4 or on days 2, 5, and 7 (n=17-19 for all treatment groups). Tumor volumes and Kaplan Meier curves are shown. P values determined by pairwise comparisons using post hoc tests with a Tukey adjustment at day 40 (tumor volumes) and the log-rank Mantel-Cox test (Kaplan Meier). c, Established 4T1-Luc tumors (100±20 mm³) were treated once with 20 Gy IR followed by three intratumoral injections of cGAMP alone or cGAMP+compound 1 on day 2, 4, and 7 after IR (n=9 per treatment group). Tumor volumes and Kaplan Meier curves are shown. P values determined by pairwise comparisons using post hoc tests with a Tukey adjustment at day 40 (tumor volumes) and the log-rank Mantel-Cox test (Kaplan Meier).

FIG. 15 shows ENPP1 inhibition synergizes with IR treatment to increase tumor-associated dendritic cells. Geometric mean of experiment shown in FIG. 7, panel a. Mean±SD. *P<0.05 (Welch's t test).

FIG. 16: Different modes of cGAMP transmission from the synthesizing cell to target cells. (1) Spread via gap junctions; (2) packaged into budding viral particles and transmitted during the next round of infection; and (3) exported into the extracellular space.

FIG. 17: cGAMP is a cancer danger signal. APCs can sense tumor cells through different cGAS-dependent mechanisms: (1) activation of APC cGAS by tumor-derived dsDNA. (2) APC sensing of type I IFNs secreted by tumor cells, and (3) APC sensing of cGAMP constitutively produced and exported by tumor cells.

### Discussion

The results of this disclosure provide evidence of cGAMP export. Cell-cell cGAMP transfer csn occur through gap junctions and viral particles. This disclosure provides in vitro and in vivo evidence that cGAMP can travel through the extracellular space (see e.g., FIG. 16). cGAMP export is a hallmark of cancer cells since all the cell lines we tested synthesize and export cGAMP without external stimulation. Since chromosomal instability and aberrant cytosolic dsDNA are considered tumor intrinsic properties and tumor cells rarely inactivate cGAS (see e.g., Bakhoum, S. F. et al. Chromosomal instability drives metastasis through a cytosolic DNA response. Nature 553, 467-472 (2018)), we reason that constant cGAMP production and export can also be properties intrinsic to tumor cells. Since no cytosolic cGAMP hydrolase has been identified and ENPP1 cannot degrade intracellular cGAMP, export is currently the only mechanism by which cGAMP is removed from the cytosol, and represents another way to turn off intracellular STING signaling in addition to ubiquitin mediated STING degradation (see e.g. Konno, H., Konno, K. & Barber. G. N. Cyclic dinucleotides trigger ULK1 (ATG1) phosphorylation of STING to prevent sustained innate immune signaling. Cell 155, 688-698 (2013)). This clearance mechanism, however, exposes cancer cells to immune detection.

Indeed, our results demonstrate that cGAMP exported by cancer cells is a danger signal detected by the immune system. Neoantigens from cancer cells are presented by APCs to cross prime cytotoxic CD8⁺ T cells that eventually perform cancer-specific killing. However, it is less understood how APCs initially detect cancer cells. Immunogenic tumors release dsDNA as a danger signal to CD11c⁺ dendritic cells, an important type of APCs (see e.g., Xu. M. M. et al. Dendritic Cells but Not Macrophages Sense Tumor Mitochondrial DNA for Cross-priming through Signal Regulatory Protein a Signaling. Immunity 47, 363-373 (2017)). In addition, cancer cells respond to their own cytosolic dsDNA induced by radiation and produce IFNs as a danger signal (Vanpouille-Box, C. et al. DNA exonuclease Trex1 regulates radiotherapy-induced tumour immunogenicity. Nat. Commun. 8, 15618 (2017)). The catalytic activity of tumor cGAS correlates with tumor immunity in the B16 melanoma model in a host STING dependent manner (see e.g., Marcus. A. et al. Tumor-Derived cGAMP Triggers a STING-Mediated Interferon Response in Non-tumor Cells to Activate the NK Cell Response. Immunity 49, 754-763.e4 (2018)), suggesting that cGAMP could be transferred from tumor cells to host cells, with unknown mechanism. Here, we provide direct evidence that cancer cells produce soluble extracellular cGAMP as a danger signal, which leads to increased numbers of dendritic cells in the tumor microenvironment (FIG. 17), cGAMP export is an important mode of cGAMP communication among cells that are not physically connected, but are in close proximity. Unlike cytokines, cGAMP cannot travel long distance in the extracellular space without being degraded and/or diluted to below its effective concentrations. This property is shared with neurotransmitters and qualifies cGAMP as the first identified immunotransmitter.

Releasing cGAMP into the extracellular space is, therefore, the Achilles heel of cancers if they cannot clear it quickly. We demonstrate that ENPP1 negatively regulates extracellular cGAMP signaling in vitro and its downstream anti-cancer immune activation in mice. Because tumor-derived soluble cGAMP is freely diffusible, overexpression of ENPP1 on one cell surface could certainly clear cGAMP in the nearby microenvironment and provide fitness to its neighbors. In humans, ENPP1 expression levels in breast cancers have been correlated with drug resistance (see e.g., Umar. A. et al. Mol. Cell. Proteomics 8, 1278-1294 (2009)), bone metastases (see e.g., Lau, W. M. et al. PLoS One 8, 1-5 (2013), and poor prognosis (Takahashi, R. U. et al. Nat. Commun. 6, 1-15 (2015)). ENPP1 can be targeted for inhibition as an innate immune checkpoint for applications in cancer immunotherapy.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the an will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the following.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A compound of formula (I): 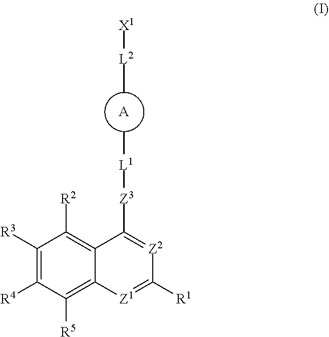
wherein,
X¹ is hydrophilic head group (e.g., a phosphorus-containing group capable of binding zinc ion);
A is a ring system selected from aryl, substituted aryl, heteroaryl, substituted heteroaryl, cycloalkyl, substituted cycloalkyl, heterocycle and substituted heterocycle;
L¹ and L² are independently covalent bond or linker;
Z³ is absent or selected from NR²², O and S;
Z² is CR¹² or N;
Z¹ is CR¹¹ or N;
R¹ is selected from H, alkyl, substituted alkyl, alkenyl, substituted alkenyl, alkylaryl, substituted alkylaryl, alkylheteroaryl, substituted alkylheteroaryl, alkenylaryl (e.g., ethenylaryl), substituted alkenylaryl, alkenylheteroaryl (e.g., ethenylheteroaryl), substituted alkenylheteroaryl, aryl, substituted aryl, heteroaryl, substituted heteroaryl, heterocycle and substituted heterocycle;
R¹¹ and R¹² are independently selected from H, cyano, trifluoromethyl, halogen, alkyl and substituted alkyl;
R²² is selected from H, alkyl and substituted alkyl; and
R² to R⁵ are independently selected from H, OH, alkyl, substituted alkyl, alkenyl, substituted alkenyl, alkoxy, substituted alkoxy, —OCF₃, halogen, cyano, amine, substituted amine, amide, heterocycle and substituted heterocycle; or wherein R² and R³, R³ and R⁴, or R⁴ and R⁵ together with the carbon atoms to which they are attached provide a fused ring (e.g., 5- or 6-membered monocyclic ring) selected from heterocycle, substituted heterocycle, cycloalkyl, substituted cycloalkyl, aryl and substituted aryl;
or a pro-drug, pharmaceutically acceptable salt or solvate thereof.

2. The compound of claim 1, wherein the compound is of formula (II): 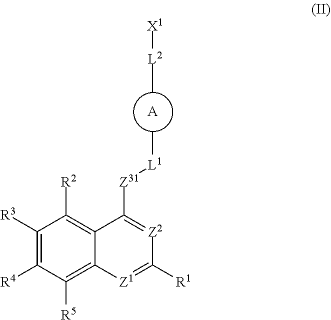
wherein Z³¹ is selected from NR²², O and S.

3. The compound of claim 2, wherein:
Z³¹ is NR²²; and
R²² is selected from H, C₍₁₋₆₎alkyl and substituted C₍₁₋₆₎alkyl.

4. The compound of claim 2, wherein the compound is of formula (III): 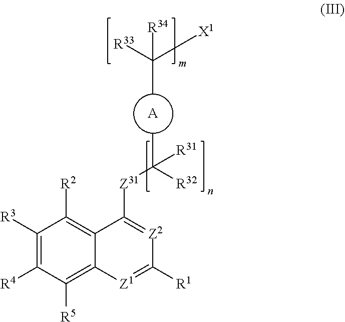
wherein each R³¹ to R³⁴ is independently selected from H, halogen, alkyl and substituted alkyl, or R³¹ and R³² or R³³ and R³⁴ are cyclically linked and together with the carbon atom to which they are attached provide a cycloalkyl, substituted cycloalkyl, heterocyclyl or substituted heterocyclyl ring; and
n and m are each independently an integer from 0 to 6 (e.g., 0-3).

5. The compound of claim 4, wherein n+m is 0 to 3 (e.g., 0, 1, 2 or 3).

6. The compound of claim 1, wherein the ring system A is selected from phenyl, substituted phenyl, pyridyl, substituted pyridyl, pyrimidine, substituted pyrimidine, piperidine, substituted piperidine, piperazine, substituted piperazine, pyridazine, substituted pyridazine, cyclohexyl and substituted cyclohexyl.

7. The compound of claim 6, wherein the ring system A is selected from: 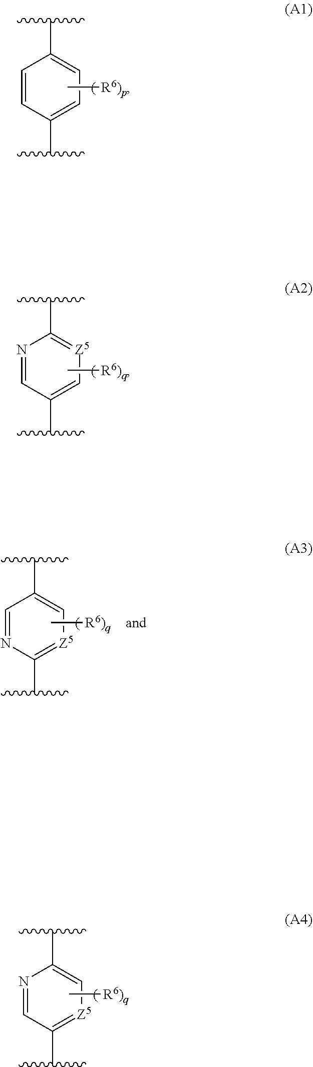
wherein:
Z⁵ is selected from N and CR⁶;
each R⁶ is selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide;
p is an integer from 0 to 4; and
q is an integer from 0 to 2.

8. The compound of claim 4, wherein the compound is of formula (IV): 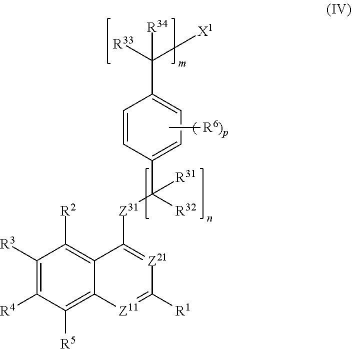
wherein:
Z¹¹ and Z²¹ are independently selected from N and C(CN);
each R⁶ is independently selected from H, alkyl, substituted alkyl, hydroxy, alkoxy, substituted alkoxy, trifluoromethyl and halogen; and
p is an integer from 0 to 4.

9. The compound of claim 8, wherein the compound is of formula (V): 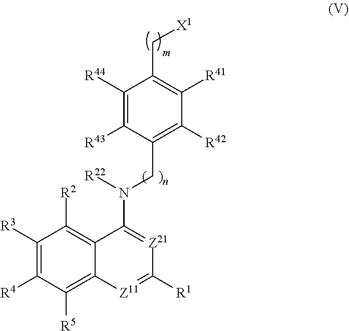
wherein:
R⁴¹ to R⁴⁴ are independently selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide.

10. The compound of claim 9, wherein the compound is of one of formulae (VIa)-(VIb):

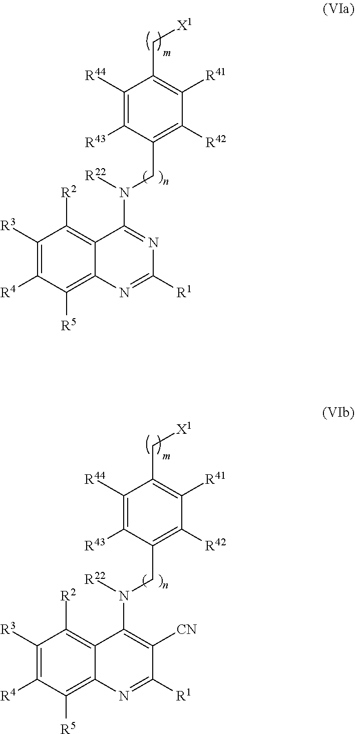

11-23. (canceled)

24. The compound of claim 1, wherein Z³ is absent and Z² is CR¹² wherein R¹² is cyano, wherein the compound is of Formula (X): 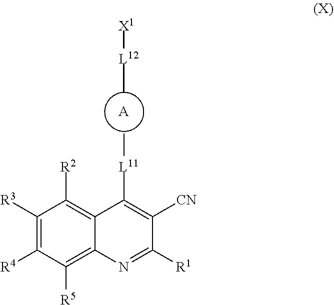
wherein L¹¹ and L¹² are independently covalent bond or linker.

25. The compound of claim 24, wherein the ring system A is selected from phenyl, substituted phenyl, pyridyl, substituted pyridyl, pyrimidine, substituted pyrimidine, piperidine, substituted piperidine, piperazine, substituted piperazine, pyridazine, substituted pyridazine, cyclohexyl and substituted cyclohexyl.

26. The compound of claim 24, wherein the compound is of Formula (XI): 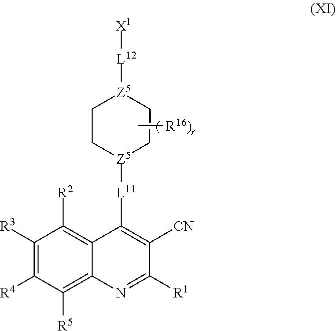
wherein:
each Z⁵ is independently selected from N and CR¹⁶;
each R¹⁶ is independently selected from hydrogen, alkyl, substituted alkyl, hydroxy, alkoxy, substituted alkoxy, trifluoromethyl, halogen, acyl, substituted acyl, carboxy, carboxyamide, substituted carboxyamide, sulfonyl, substituted sulfonyl, sulfonamide and substituted sulfonamide; and
r is an integer from 0 to 8.

27. The compound of claim 26, wherein the compound is of Formula (XII):

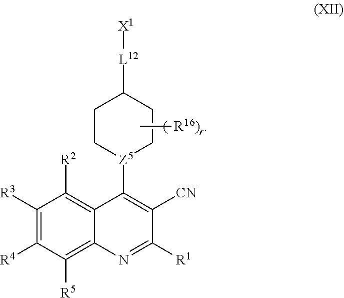

28. The compound of claim 27, wherein Z⁵ is N.

29. The compound of claim 28, wherein the compound is of Formula (XIII): 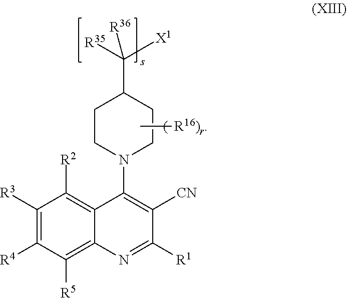
wherein s is an integer from 0 to 6 (e.g., 0 to 3).

30. The compound of claim 29, wherein the compound is of Formula (XIV):

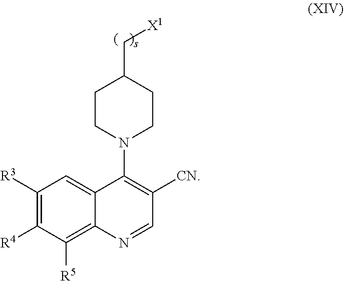

31-49. (canceled)

50. A method of inhibiting ENPP1, the method comprising:
contacting a sample comprising ENPP1 with an ENPP1 inhibitor according to claim 1 to inhibit cGAMP hydrolysis activity of the ENPP1.

51-55. (canceled)

56. A method of treating cancer, the method comprising:
administrating to a subject with cancer a therapeutically effective amount of an ENPP1 inhibitor according to claim 1 to treat the subject for cancer.

57-90. (canceled)
